(19) United States
(12) Patent Application Publication
(10) Pub. No.: US 2024/0383842 A1
(43) Pub. Date: Nov. 21, 2024

(54) METHODS OF PREPARING CHIRAL AMINO ACIDS

(71) Applicants: The University of Hong Kong, Hong Kong (CN); Laboratory for Synthetic Chemistry and Chemical Biology Limited, Hong Kong (CN)

(72) Inventors: Zhongxing Huang, Hong Kong (CN); Weifeng Zheng, Hong Kong (CN)

(21) Appl. No.: 18/658,762

(22) Filed: May 8, 2024

Related U.S. Application Data
(60) Provisional application No. 63/503,061, filed on May 18, 2023.

(51) Int. Cl. C07C 227/18; B01J 31/02; C07C 229/42; C07C 231/12; C07C 235/52; C07C 247/12; C07C 253/30; C07C 255/30; C07C 255/58; C07C 315/04; C07C 317/48; C07D 207/16; C07D 209/24; C07D 209/48; C07D 307/54; C07D 317/30; C07D 333/24; C07D 405/14; C07D 411/14; C07D 493/04; C07J 41/00

(52) U.S. Cl. CPC C07C 227/18; B01J 31/0258; C07C 229/42; C07C 231/12; C07C 235/52; C07C 247/12; C07C 253/30; C07C 255/30; C07C 255/58; C07C 315/04; C07C 317/48; C07D 207/16; C07D 209/24; C07D 209/48; C07D 307/54; C07D 317/30; C07D 333/24; C07D 405/14; C07D 411/14; C07D 493/04; C07J 41/0088; C07C 2601/02; C07C 2601/08; C07C 2601/14

(57) ABSTRACT

Described herein are methods for preparing chiral α-amino acids using chiral phosphoric acids as catalysts. The disclosed methods can use amino-malonic acids as substrates to generate chiral amino acids with a variety of side chains in high optional purity (such as an ee value of at least 70%) and with a high yield (i.e., a yield of at least 80%, such as in a range from about 80% to about 99%), via an asymmetric decarboxylation reaction. The decarboxylation reaction of the methods is catalyzed by chiral phosphoric acids that can achieve a selective protonation during decarboxylation, which is considered one of the most difficult processes in asymmetric catalysis.

### CROSS-REFERENCE TO RELATED APPLICATION

This application claims the benefit of and priority to U.S. Provisional Application No. 63/503,061 filed May 18, 2023, the entire content of which is incorporated herein by reference for all purpose in its entirety.

### FIELD OF THE INVENTION

The disclosed invention is generally in the field of chiral amino acids.

### BACKGROUND OF THE INVENTION

Organic synthesis often uses active methylene compounds to build complex structures. When located between two electron-withdrawing groups, methylene bridges can be easily deprotonated and doubly substituted. These substitution reactions, when coupled with further derivatization of the tetrasubstituted carbon, can rapidly provide polyfunctionalized structures. Malonic ester synthesis is an example of such a paradigm that has been implemented for more than a century to forge α-substituted carboxylic acids via a sequence of substitution, hydrolysis, and decarboxylation (J. Wislicenus, Liebigs Ann. Chem. 186, 161-228 (1877); A. C. Cope, et al., Org. React. 107-331 (1957); and H. O. House, Modern Synthetic 2nd ed W. A. Benjamin, inc., 1972, pp. 492-623). When combined with phthalimide-based Gabriel amine synthesis (S. Gabriel, Ber. 20, 2224-2236 (1887); M. S. Gibson and R. W. Bradshaw, Angew. Chem., Int. Ed. Engl. 7, 919-930 (1968)), this synthetic protocol is further empowered to produce α-amino acids (S. K. Mitra, J. Ind. Chem. Soc. 7, 799-802 (1930)). While the malonate-based approach to this family of biomolecules has the advantages of facile substrate preparation and high modularity, its capability of accessing these acids stereoselectively is limited.

Asymmetric malonic ester synthesis needs a fast and stereoselective decarboxylation pathway that can outcompete self-decarboxylation reaction to racemic acids, to deliver a small proton precisely to one face of the enol intermediate after CO₂ extrusion. These challenges may be addressed by enzymatic catalysis with arylmalonate decarboxylase (AMDase, EC 4.1.1.76, K. Miyamoto and Kourist, R., Appl. Microbiol. Biotechnol. 100, 8621-8631 (2016); M. Wilding, et al., Comprehensive Chirality, H. Yamamoto, & E. M. Carreira, Eds. (Elsevier, 2012) pp. 402-409). In a basic medium, diacid substrates are deprotonated to their conjugate bases that have an inhibited self-decarboxylation and can fit into the active site of AMDase containing two polar pockets of contrasting sizes and a dioxyanion hole (W. H. Richardson and H. E. O'Neal, Comprehensive Chemical Kinetics: Decomposition and Isomerization of Organic Compounds, C. H. Bamford & C. F. H. Tipper Eds. (Elsevier, 1972) pp. 381-565). The anion hole is composed of four residues with six hydrogen bond donors that lower the barrier of decarboxylation by stabilizing negative charges. Subsequently, a nearby cysteine protonates the fixed dianion in a stereoinversive fashion. The enzymatic approach is particularly selective in decarboxylating malonic acids with an aryl and a small alkyl group. However, amino acids (i.e., compounds 10a and 10b in K. Okrasa, et al., Angew. Chem. Int. Ed. 48, 7691-7694 (2009)) obtained using this method are only moderately enantioenriched (i.e., an enantiomeric excess of up to 61%).

The asymmetric decarboxylation was also attempted by chemical catalysis (J. Blanchet, et al., Eur. J. Org. Chem. 5493-5506 (2008)). Early study of malonic acids revealed a higher rate of decarboxylation in basic solvents, such as tertiary amines and pyridines, indicative of possible base catalysis (W. H. Richardson and H. E. O'Neal, Comprehensive Chemical Kinetics: Decomposition and Isomerization of Organic Compounds, C. H. Bamford & C. F. H. Tipper Eds. (Elsevier, 1972) pp. 381-565). As a result, organic bases, particularly cinchona alkaloids, have been heavily studied. However, these only result in limited enantioinduction and thus low ee. For example, the best result of organic base-catalyzed decarboxylation of aminomalonic acids was found in H. Brunner and M. A. Baur, Eur. J. Org. Chem. 2854-2862 (2003), where malonic acid 4 gave the amino acid in 8.1 and 11.9% ee.

There remains a need to develop improved methods of preparing chiral amino acids, such as high enantiomeric excess (ee).

Therefore, it is an object of the present invention to provide methods of preparing chiral amino acids with high enantiomeric excess (ee).

It is a further object of the present invention to provide methods of preparing chiral amino acids that have a variety of side chains.

It is a further object of the present invention to provide chiral amino acids having a variety of side chains.

### SUMMARY OF THE INVENTION

Methods for preparing chiral α-amino acids using chiral phosphoric acids as catalysts have been developed. The disclosed methods can use amino-malonic acids as substrates to generate chiral amino acids with a variety of side chains in high optical purity (such as an ee value of at least 70%) and with a high yield (i.e., a yield of at least 80%, such as in a range from about 80% to about 99%), via an asymmetric decarboxylation reaction. The decarboxylation reaction of the methods is catalyzed by chiral phosphoric acids that can achieve a selective protonation during decarboxylation, which is considered one of the most difficult processes in asymmetric catalysis. In some forms, the catalyst can be a chiral phosphoric acid loaded on a suitable support.

Generally, the method for producing chiral amino acids includes (i) maintaining a first reaction mixture at a first temperature for a first period of time sufficient to form a product containing a chiral amino acid. The first reaction mixture contains a substrate, a catalyst, and a solvent. The solvent is preferably an ether, such as cyclopentylmethylether (CPME). The catalyst is typically a chiral phosphoric acid. In some forms, the catalyst is a chiral phosphoric acid loaded on a support. Generally, the catalyst is present in the first reaction mixture in an amount ranging from about 1 mol % to about 20 mol %, from about 1 mol % to about 10 mol %, from about 2 mol % to about 20 mol %, from about 2 mol % to about 10 mol %, or from about 2.5 mol % to about 10 mol %, such as about 10 mol % or about 2.5 mol %.

Generally, the first reaction mixture can be maintained at a temperature ranging from about 50° C. to about 100° C., from about 60° C. to about 100° C., or from about 70° C. to about 100° C., such as about 80° C., for a period of time ranging from about 1 hour to about 12 hours, from about 1 hour to about 10 hours, from about 1 hour to about 8 hours, from about 1 hour to about 5 hours, or from about 1 hour to about 3 hours, such as about 2 hours, to produce the product containing chiral amino acids.

Typically, the substrate is a malonic acid. In some forms, the malonic acid can have the structure of Formula I or Formula I′:

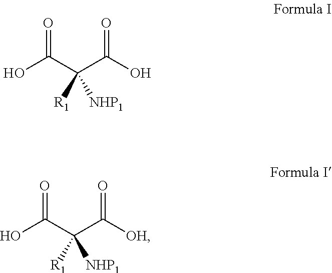

and the chiral amino acid in the product can have the structure of Formula II or Formula II′:

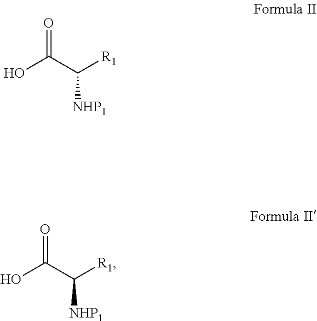

where P₁ can be an amino protecting group and R₁ can be any suitable functional group or biomolecule moiety.

In some forms, the disclosed method also includes: (ii) adding an alkylation reactant (such as a methylation reactant, e.g., trimethylsilyldiazomethane, or ethylation reactant) or an acid (such as HCl) to the product to form a second reaction mixture, and (iii) maintaining the second reaction mixture at a second temperature for a second period of time sufficient to form an alkylation product (such as a methylation or ethylation product) or hydrolysis product. The alkylation product or hydrolysis product contains an alkylated chiral amino acid or hydrolyzed chiral amino acid. In some forms, the alkylated chiral amino acid can have the structure of Formula VIII or Formula VIII′, and the hydrolyzed chiral amino acid can have the structure of Formula V or Formula V′:

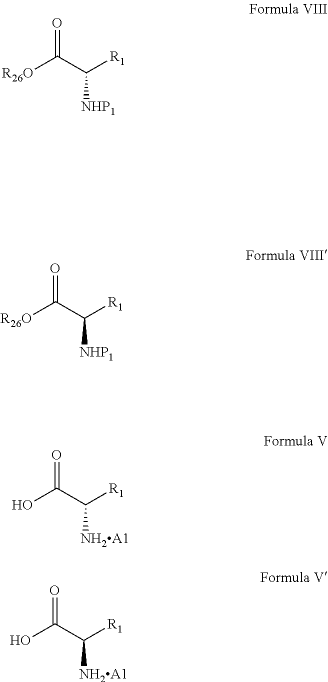

where P₁ and R₁ can be as defined above, R₂₆ can be a substituted or unsubstituted alkyl, such as a C₁-C₁₂ unsubstituted alkyl, a C₁-C₁₀ unsubstituted alkyl, a C₁-C₈ unsubstituted alkyl, a C₁-C₆ unsubstituted alkyl, or a C₁-C₄ unsubstituted alkyl, for example, methyl or ethyl; and A1 can be an acid molecule (such as HCl).

In some forms, the second reaction mixture undergoes a methylation reaction. In these forms, the second reaction mixture can be maintained at a temperature ranging from about 50° C. to about 100° C., from about 60° C. to about 100° C., or from about 70° C. to about 100° C., for a period of time ranging from about 30 minutes to about 2 hours or from about 30 minutes to about 1 hour, to produce methylated chiral amino acid; or can be maintained at a temperature ranging from about 90° C. to about 120° C. or from about 90° C. to about 110° C., for a period of time ranging from about 12 hours to about 36 hours or from about 12 hours to about 24 hours, to produce hydrolyzed chiral amino acid. In some forms, after step (i), the product can be cooled to room temperature or about 0° C. before adding the methylation reactant or the acid.

In some forms, P₁ can be

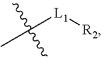

where L₁ can be

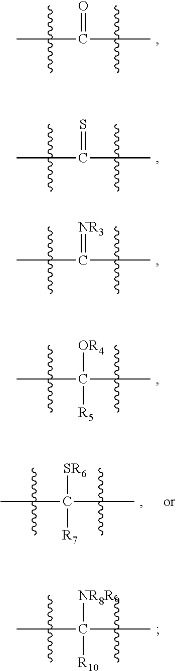

R₂ can be hydrogen, hydroxyl, halide, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, carbonyl, cyano, isocyano, nitro, silyl, sulfinyl, sulfonyl, phosphonium, phosphanyl, phosphoryl, or phosphonyl; R₃-R₁₀ can be independently hydrogen or a substituted or unsubstituted alkyl (such as unsubstituted alkyl, for example, C₁-C₁₀ unsubstituted alkyl, C₁-C₈ unsubstituted alkyl, C₁-C₆ unsubstituted alkyl, or C₁-C₄ unsubstituted alkyl); and the substituents, when present, can be independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl.

In some forms, L₁ can be

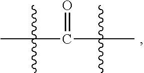 [SMILES: *C(*)=O]

In some forms, R₂ can be substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, or carbonyl.

In some forms, R₂ can be substituted or unsubstituted cycloalkyl, substituted or unsubstituted cycloalkenyl, substituted or unsubstituted cycloalkynyl, substituted or unsubstituted aryl, substituted or unsubstituted polyaryl, or substituted or unsubstituted aralkyl. In some forms, R₂ can be

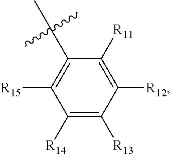

where R₁₁-R₁₅ can be independently hydrogen, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl. In some forms, R₁₁-R₁₅ can be independently hydrogen, halide, unsubstituted alkyl, unsubstituted alkenyl, unsubstituted alkynyl, unsubstituted phenyl, unsubstituted haloalkyl, unsubstituted aralkyl, cyano, isocyano, nitro,

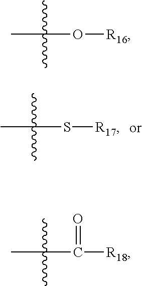

where R₁₆-R₁₈ can be independently hydrogen, hydroxyl, —OR′₁₈, unsubstituted alkyl, unsubstituted phenyl, unsubstituted haloalkyl, or unsubstituted aralkyl, and R′₁₈ can an unsubstituted alkyl.

In some forms, R₂ can be

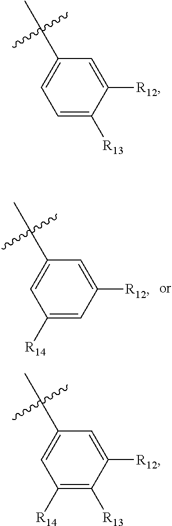

where R₁₂-R₁₄ can be independently halide (such as F, Cl, or I), unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), cyano, isocyano, nitro,

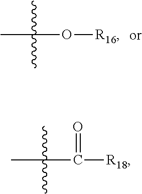

and R₁₆ and R₁₈ can be independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), and R′₁₈ can an unsubstituted alkyl.

In some forms, R₂ can be

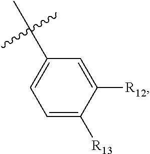

where R₁₂ can be

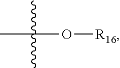

R₁₃ can be halide (such as F, Cl, or I), unsubstituted alkyl, unsubstituted haloalkyl (such as —CF₃), cyano, isocyano, nitro,

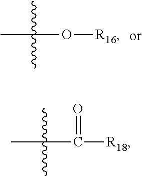

and R₁₆ and R₁₈ can be independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), R′₁₈ can an unsubstituted alkyl. In some form, R₁₃ can be nitro.

In some forms, R₁ can be a substituted or unsubstituted alkyl, a substituted or unsubstituted alkenyl, or a substituted or unsubstituted alkynyl, where the substituents, when present, can be independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl. In some forms, the substituents, when present, can be independently substituted or unsubstituted cycloalkyl, substituted or unsubstituted cycloalkenyl, substituted or unsubstituted cycloalkynyl, substituted or unsubstituted aryl, substituted or unsubstituted polyaryl, substituted or unsubstituted heteroaryl, substituted or unsubstituted heteropolyaryl, substituted or unsubstituted heterocyclyl, azido, carbonyl, alkoxy, halide, hydroxyl, thiol, cyano, isocyano, or nitro. In some forms, the substituents, when present, can be independently a biomolecule moiety.

In some forms, one or more carbons, one or more nitrogen, and/or one or more hydrogens of the substrate can be in the form of ¹³C, ¹⁴C, ¹⁵N, and/or D.

In some forms, the chiral phosphoric acid can be a Binol phosphoric acid or derivative thereof, an H8 Binol phosphoric acid or derivative thereof, a Spinol phosphoric acid or derivative thereof, a Biphenol phosphoric acid or derivative thereof, a dithiophosphoric acid or derivative thereof, a Taddol phosphoric acid or derivative thereof, a paracyclophane or derivative thereof, a TiPSY phosphoric acid or derivative thereof, or a TRIP phosphoric acid or derivative thereof. In some forms, the chiral phosphoric acid can have the structure of Formula III or Formula III′:

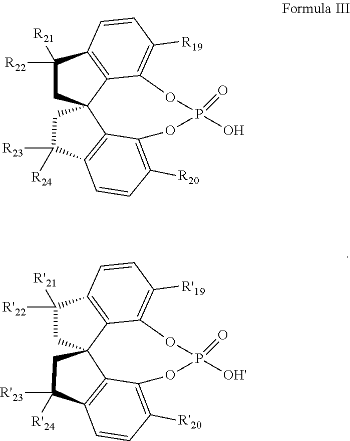

where R₁₉, R₂₀, R′₁₉, and R′₂₀ can be independently substituted or unsubstituted aryl or substituted or unsubstituted polyaryl; R₂₁-R₂₄ and R′₂₁-R′₂₄ can be independently hydrogen or unsubstituted alkyl; and the substituents, when present, can be independently substituted or unsubstituted alkyl, substituted or unsubstituted aryl (such as phenyl), substituted or unsubstituted polyaryl (such as naphthal), substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or

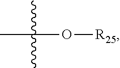

and R₂₅ can be substituted or unsubstituted alkyl, substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), substituted or unsubstituted aryl (such as phenyl), or substituted or unsubstituted aralkyl (such as benzyl).

In some forms, at least one of R₁₉ and R₂₀ and at least one of R′₁₉ and R′₂₀ can be independently a substituted aryl or substituted polyaryl, and optionally wherein the substituted aryl or polyaryl has three or more substituents (such as 2,4,6-substituted phenyl). In some forms, the chiral phosphoric acid can have the structure of:

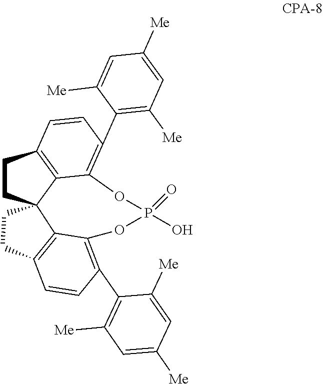

In some forms, the disclosed method can further includes one or more of the following steps: purifying the product after step (i) and/or the alkylation or hydrolysis product after step (iii); preparing the substrate prior to step (i); recycling an amino protecting agent and the catalyst after step (i) and/or step (iii); and derivatizing the chiral amino acid or alkylated or hydrolyzed chiral amino acid to a derivatized compound after step (i) and/or step (iii), such as a cyclic amino acid, a drug-amino acid conjugate, or a DNA gyrase inhibitor.

In some forms, the derivatization step can be performed via an oxidation reaction, a coupling reaction (such as click reaction), a nucleophilic/electrophilic substitution, or a metathesis reaction, or a combination thereof.

In some forms, the substrate can be prepared using a commercially available malonic ester as a starting compound, such as one having the structure of Formula VI; the starting material be converted to an amino protected compound of Formula VII:

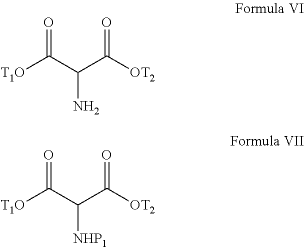

wherein T₁ and T₂ can be independently unsubstituted alkyl, and P₁ can be any amino protecting group such as any one of those described above for the substrate. Subsequent to the protection step, the amino protected compound of Formula VII can be converted to the substrate of Formula I or I′ via substitution and hydrolysis, which can occur sequentially or simultaneously.

### BRIEF DESCRIPTION OF THE DRAWINGS

FIG. 1 is a schematic showing exemplary asymmetric decarboxylation reaction using chiral phosphoric acid.

FIG. 2A is a graph showing enantiomeric excess (ee) of 14 versus yield of 14 using different solvents. FIG. 2B is a schematic showing asymmetric decarboxylation reaction using different amine protecting groups. ^{a}The result was obtained using the same conditions shown in Scheme 19 except using a concentration of 0.05 M aminomalonic acid in the solvent and reaction time of 5 h instead of 0.2 M and 2 h.

FIGS. 3A-3D are schematics showing the synthesis of amino acids with diverse alkyl chains. The yield and enantiomeric excess were obtained after recrystallization.

FIGS. 4A-4C are schematics showing the preparation of diverse functionalized phenylalanines. The yield and enantiomeric excess were obtained after recrystallization.

FIG. 5 is a schematic showing preparation of a DNA gyrase B inhibitor (79) containing an unnatural alanine residue. The yield and enantiomeric excess were obtained after recrystallization.

FIG. 6 is a schematic showing the crystal structure of (+)-17.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C=C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O— aralkyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

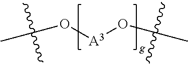

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

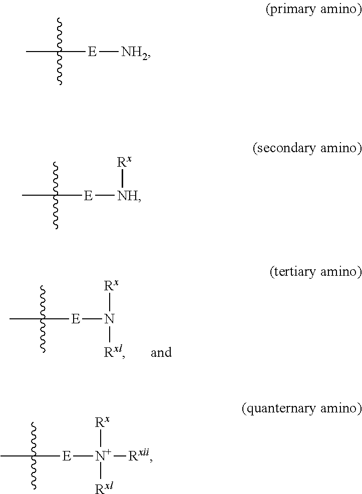

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

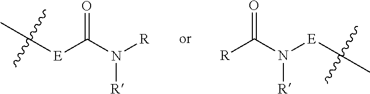

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

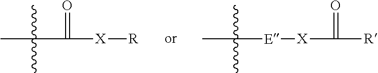

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

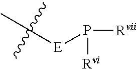

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

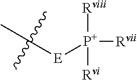

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

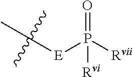

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

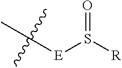

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

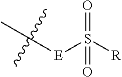

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

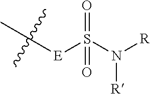

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

The full names of certain abbreviations as used herein is provided in the following table:

| | |
|---|---|
| DMF | N,N-dimethylformamide |
| AA | amino acid |
| Ac | acetyl |
| Ad | adamantyl |
| BpF | biphenylphenylalanine |
| CnF | cyanophenylalanine |
| DCE | dichloroethane |
| EnF | ethynylphenylalanine |
| THF | tetrahydrofuran |
| TBME | tert-butyl methyl ether |
| Bn | benzyl |
| Bz | benzoyl |
| TMS | trimethylsilyl |
| Pr | propyl |
| Cy | cyclohexyl |
| UAA | unnatural amino acid |
| Orn | ornithine |
| DDQ | 2,3-dichloro-5,6-dicyano-1,4-benzoquinone |

### II. Methods for Preparing Chiral Amino Acids

Methods for preparing chiral α-amino acids using chiral phosphoric acids as catalysts have been developed. The chiral α-amino acids produced using the disclosed methods can be natural or unnatural, and have diverse side chains and functionality.

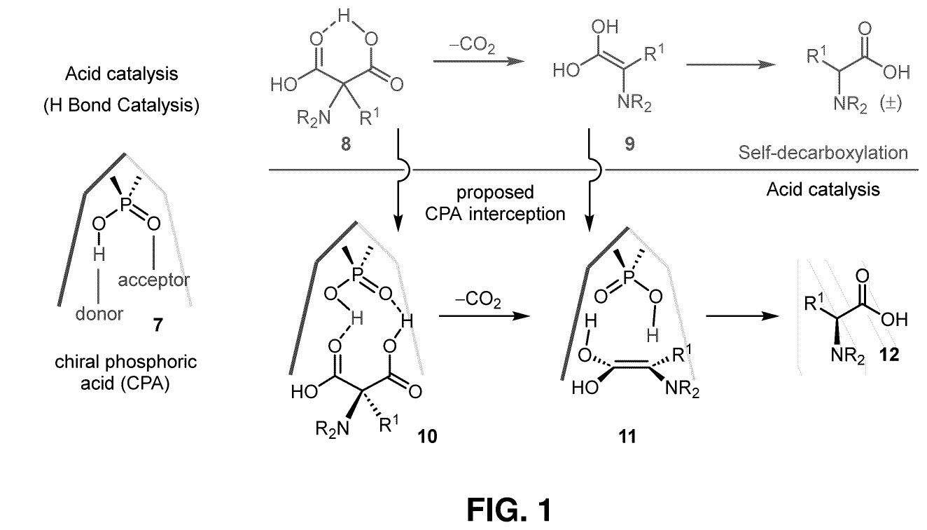

The disclosed methods can use amino-malonic acids as substrates to generate chiral amino acids with a variety of side chains in high optional purity (such as an ee value of at least 70%) via an asymmetric decarboxylation reaction. The decarboxylation reaction of the methods is catalyzed by chiral phosphoric acids that can achieve a selective protonation during decarboxylation (see, e.g., FIG. 1), which is typically one of the most difficult processes in asymmetric catalysis. In some forms, the catalyst can be chiral phosphoric acid loaded on a suitable support.

Without being bound to any theories, it is believed that the selective protonation achieved using chiral phosphoric acids in the methods not only lies in the high acidity of the chiral phosphoric acids that can rapidly protonate, but also in the chiral phosphoric acids' capability to impose stereocontrol by forming a rich network of hydrogen bonds within their pockets, particularly with heteroatom-rich substrates or intermediates (see, e.g., 8 and 9 in FIG. 1). As such, the chiral phosphoric acids may interrupt the internal hydrogen bonding of the malonic acid (see, e.g., 8 in FIG. 1) and initiate the decarboxylation via an intermolecular proton transfer (see, e.g., 10 in FIG. 1). Direct interception of the intermediate (see, e.g., the enol intermediate 9 in FIG. 1) from self-decarboxylation is also possible. In either scenario, a stereoselective protonation inside the chiral pocket (see, e.g., 11 in FIG. 1) would produce the chiral amino acid (see, e.g., 12 in FIG. 1).

In some forms, the amino-malonic acid substrates (also referred to herein as “substrates”) used in the disclosed methods are prepared using commercially available malonic acids/esters as starting material. The starting material can be converted by attaching a suitable functional group or biomolecule moiety to the methylene carbon of the malonic acid/ester via, for example, a substitution reaction between the starting material and an alkyl halide (see, e.g., Scheme 2), which is then hydrolyzed to provide the substrates (see, e.g., Scheme 3). The amino-malonic acid substrate can then be directly converted to a corresponding chiral amino acid via asymmetric decarboxylation reaction using chiral phosphoric acid as catalyst (see, e.g., Scheme 10). As such, the functional group or biomolecule moiety introduced during the preparation of the substrate becomes the side chain or part of the side chain of the chiral amino acid produced therefrom. Using the disclosed methods, a large panel of side chains of different shapes and with distinct functional groups can be attached to and eventually incorporated in the final chiral amino acid products via the decarboxylation reaction. Thus, the disclosed methods provide a general, selective, and efficient approach to provide chiral α-amino acids with diverse side chains and functionality, natural or unnatural, that is currently lacking using existing methods.

Generally, the disclosed method for producing chiral amino acids includes: (i) maintaining a first reaction mixture at a first temperature for a first period of time sufficient to form a product, where the product contains a chiral amino acid. The first reaction mixture contains a substrate, a catalyst, and a solvent. The catalyst is preferably a chiral phosphoric acid. Typically, in step (i), the chiral amino acid is produced via an asymmetric decarboxylation of the substrate catalyzed by the chiral phosphoric acid.

In some forms, the chiral amino acid in the product formed in step (i) further reacts with a reactant to produce an alkylated chiral amino acid or hydrolyzed amino acid. In these forms, the disclosed method also includes: (ii) adding an alkylation reactant (such as a methylation or ethylation reactant) or an acid to the product to form a second reaction mixture, and (iii) maintaining the second reaction mixture at a second temperature for a second period of time sufficient to form an alkylation product (such as a methylation or ethylation product) or hydrolysis product. The alkylation product or hydrolysis product contains an alkylated chiral amino acid (such as a methylated or ethylated chiral amino acid) or hydrolyzed chiral amino acid.

In some forms, the disclosed method further includes: purifying the product after step (i) and/or the alkylation or hydrolysis product after step (iii); preparing the substrate prior to step (i); recycling an amino protecting agent and the catalyst after step (i) and/or step (iii); and/or derivatizing the chiral amino acid or alkylated or hydrolyzed chiral amino acid to a derivatized compound after step (i) and/or step (iii).

### A. Maintaining a Reaction Mixture at a Temperature for a Period of Time Sufficient to Form a Product

Generally, a first reaction mixture containing a substrate and a catalyst, preferably a chiral phosphoric acid, in a suitable solvent is maintained at a suitable temperature for a period of time sufficient to form a product containing a chiral amino acid formed by asymmetric decarboxylation of the substrate. Typically, the reaction conditions for performing the reaction are simple. For example, the temperature for performing the asymmetric decarboxylation reaction can range from about 50° C. to about 100° C., from about 60° C. to about 100° C., or from about 70° C. to about 100° C., such as about 80° C., at 1 atm; and the time period for performing the reaction at any of the temperature ranges described above can range from about 1 hour to about 12 hours, from about 1 hour to about 10 hours, from about 1 hour to about 8 hours, from about 1 hour to about 5 hours, or from about 1 hour to about 3 hours, such as about 2 hours.

The first reaction mixture can be formed by mixing the substrate and the chiral phosphoric acid in the solvent prior to reaction. Examples of solvents suitable for use in step (i) of the disclosed method include, but are not limited to, ethyl acetate, ^{t}BuOMe, THF, 2-MeTHF, 1,4-dioxane, cyclopentylmethylether (CPME), and (MeOCH₂CH₂)₂O, and a combination thereof. In preferred forms, the solvent forming the first reaction mixture in step (i) is an ether, such as CPME.

### 1. Substrates

In step (i) of the disclosed method, the catalyst catalyzes an asymmetric decarboxylation reaction of the substrate to produce the chiral amino acid. Typically, the substrate is a malonic acid substrate. In some forms, the malonic acid substrate has the structure of Formula I or Formula I′:

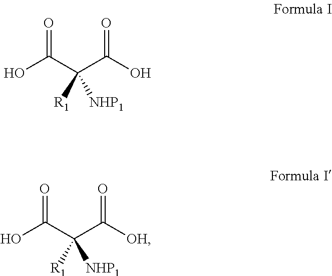

where P₁ can be an amino protecting group and R₁ can be any suitable functional group or biomolecule moiety, as long as it can be attached to the methylene carbon of the substrate. The term “biomolecule moiety” refers to a portion of a biomolecule that is incorporated in the chiral amino acid structure. The biomolecule can be any natural or synthetic chemical compound having a biological activity. Typically, the biomolecule moiety in the chiral amino acid is the portion of a biomolecule responsible for the activity of the biomolecule.

In some forms, the amino protecting group P₁ can be

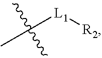

where L₁ can be

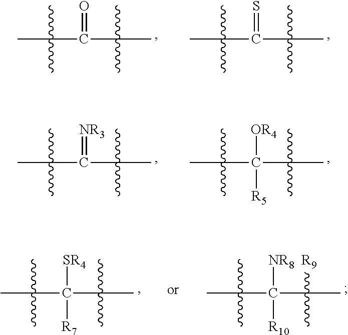

R₂ can be hydrogen, hydroxyl, halide, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, carbonyl, cyano, isocyano, nitro, silyl, sulfinyl, sulfonyl, phosphonium, phosphanyl, phosphoryl, or phosphonyl; R₃-R₁₀ can be independently hydrogen or a substituted or unsubstituted alkyl (such as unsubstituted alkyl, for example, C₁-C₁₀ unsubstituted alkyl, C₁-C₈ unsubstituted alkyl, C₁-C₆ unsubstituted alkyl, or C₁-C₄ unsubstituted alkyl); and the substituents, when present, can be independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl.

In preferred forms, L₁ is

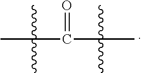 [SMILES: *C(*)=O]

In some forms, R₂ can be substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, or carbonyl. In some forms, R₂ is substituted or unsubstituted cycloalkyl, substituted or unsubstituted cycloalkenyl, substituted or unsubstituted cycloalkynyl, substituted or unsubstituted aryl, substituted or unsubstituted polyaryl, or substituted or unsubstituted aralkyl.

In some forms, R₂ can be

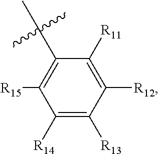

where R₁₁-R₁₅ can be independently hydrogen, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl (such as aldehyde, ketone, carboxylic acid, ester, etc.), alkoxy, halide, hydroxyl, thiol, cyano, isocyano, nitro, amino, amido, oxo, silyl, sulfinyl, sulfonyl, phosphonium, phosphanyl, phosphoryl, or phosphonyl.

In some forms, R₁₁-R₁₅ can be independently hydrogen, halide, unsubstituted alkyl, unsubstituted alkenyl, unsubstituted alkynyl, unsubstituted phenyl, unsubstituted haloalkyl unsubstituted aralkyl, cyano, isocyano, nitro,

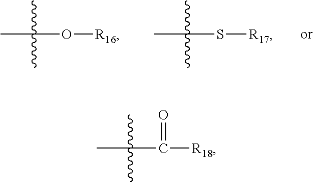

where R₁₆-R₁₈ can be independently hydrogen, hydroxyl, —OR′₁₈, unsubstituted alkyl, unsubstituted phenyl, unsubstituted haloalkyl, or unsubstituted aralkyl, where R′₁₈ is an unsubstituted alkyl.

In some forms, R₂ can be

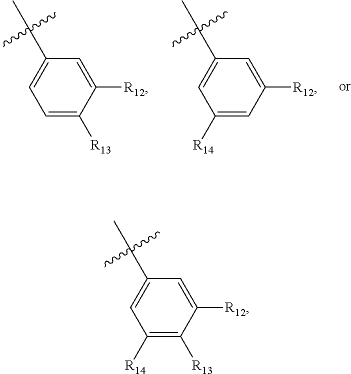

where R₁₂-R₁₄ can be independently halide (such as F, Cl, or I), unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), cyano, isocyano, nitro,

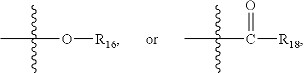

and R₁₆ and R₁₈ can be independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), where R′₁₈ can be an unsubstituted alkyl.

In some forms, R₂ can be

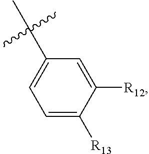

where R₁₂ can be

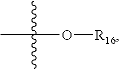

R₁₃ can be halide (such as F, Cl, or I), unsubstituted alkyl, unsubstituted haloalkyl (such as —CF₃), cyano, isocyano, nitro,

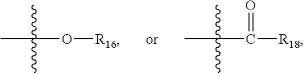

and R₁₆ and R₁₈ can be independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), where R′₁₈ can be unsubstituted alkyl. In some forms, R₁₃ can be nitro.

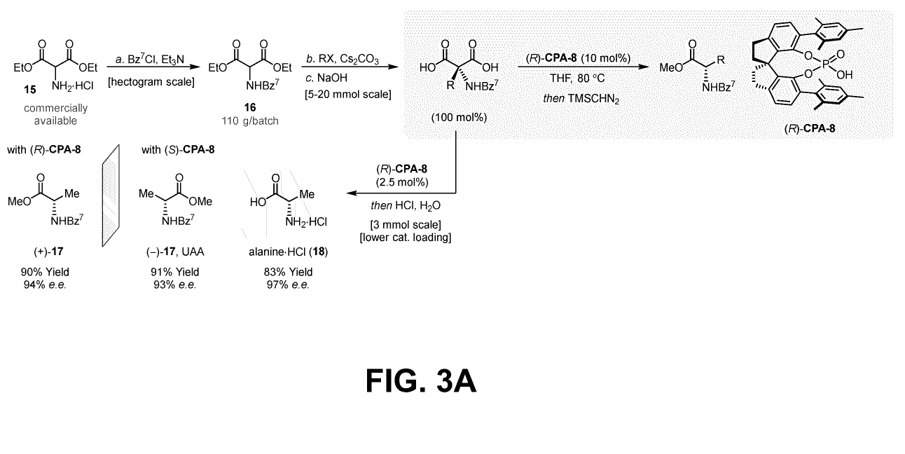

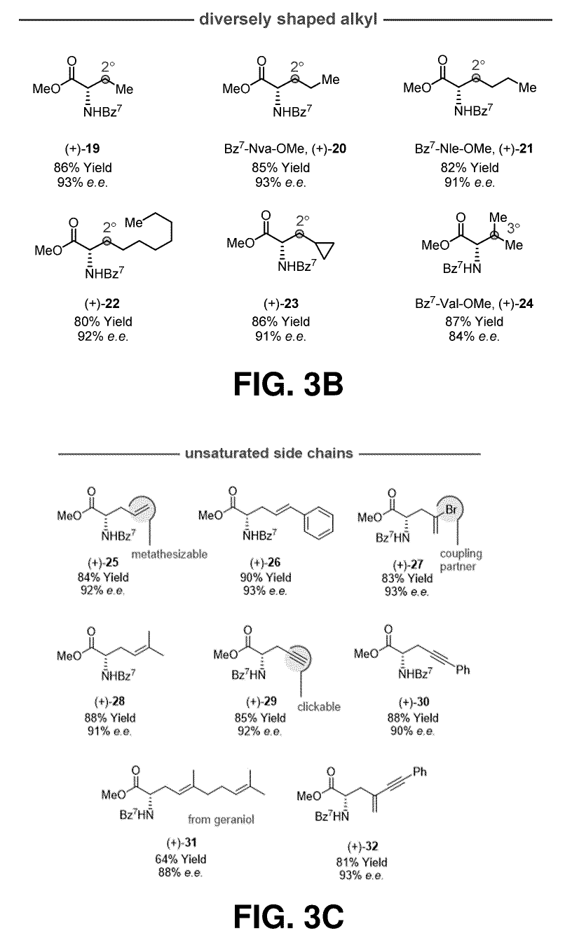

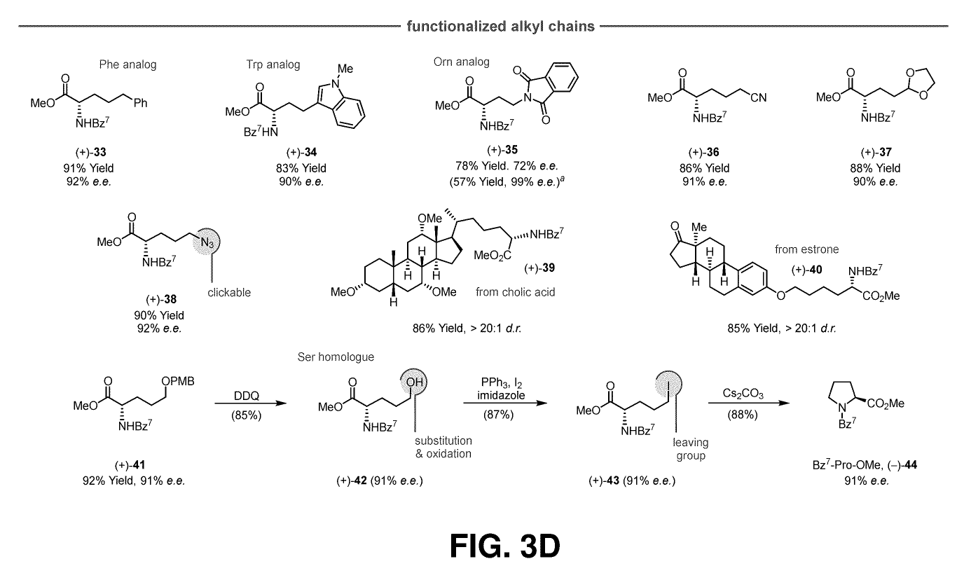

In some forms, the functional group or biomolecule moiety R₁ attached to the methylene carbon of the malonic ester substrate can be a substituted or unsubstituted alkyl, a substituted or unsubstituted alkenyl, or a substituted or unsubstituted alkynyl, where the substituents, when present, can be independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl. In some forms, the substituents, when present, can be independently substituted or unsubstituted cycloalkyl, substituted or unsubstituted cycloalkenyl, substituted or unsubstituted cycloalkynyl, substituted or unsubstituted aryl, substituted or unsubstituted polyaryl, substituted or unsubstituted heteroaryl, substituted or unsubstituted heteropolyaryl, substituted or unsubstituted heterocyclyl, azido, carbonyl, alkoxy, halide, hydroxyl, thiol, cyano, isocyano, or nitro. In some forms, the substituents, when present, are independently a biomolecule moiety, such as a cholic acid moiety (see, e.g., (+)-39 in FIG. 3D) or estrone moiety (see, e.g., (+)-40 in FIG. 3D).

In some forms, the substrate, such as the malonic acid substrate of Formula I or I′, contains one or more isotopes. For example, one or more carbons, one or more nitrogen, and/or one or more hydrogens of the substrate, such as the malonic acid substrate of Formula I or I′, can be in the form of ¹³C, ¹⁴C, ¹⁵N, and/or D.

For any forms of the substrate described above, the alkyl, when present, can be a linear alkyl, a branched alkyl, or a cyclic alkyl (either monocyclic or polycyclic). The terms “cyclic alkyl” and “cycloalkyl” are used interchangeably herein. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkyl group.

For any forms of the substrate described above, the alkenyl, when present, can be a linear alkenyl, a branched alkenyl, or a cyclic alkenyl (either monocyclic or polycyclic). The terms “cyclic alkenyl” and “cycloalkenyl” are used interchangeably herein. Exemplary alkenyl include a linear C₂-C₃₀ alkenyl, a branched C₄-C₃₀ alkenyl, a cyclic C₃-C₃₀ alkenyl, a linear C₂-C₂₀ alkenyl, a branched C₄-C₂₀ alkenyl, a cyclic C₃-C₂₀ alkenyl, a linear C₂-C₁₀ alkenyl, a branched C₄-C₁₀ alkenyl, a cyclic C₃-C₁₀ alkenyl, a linear C₂-C₆ alkenyl, a branched C₄-C₆ alkenyl, a cyclic C₃-C₆ alkenyl, a linear C₂-C₄ alkenyl, cyclic C₃-C₄ alkenyl, such as a linear C₂-C₁₀, C₂-C₉, C₂-C₈, C₂-C₇, C₂-C₆, C₂-C₅, C₂-C₄, C₂-C₃, C₂ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group. The cyclic alkenyl can be a monocyclic or polycyclic alkenyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄- C₈, C₄-C₇, C₄-C₆, or C₄-C₅ monocyclic or polycyclic alkenyl group.

For any forms of the substrate described above, the alkynyl can be a linear alkynyl, a branched alkynyl, or a cyclic alkynyl (either monocyclic or polycyclic). The terms “cyclic alkynyl” and “cycloalkynyl” are used interchangeably herein. Exemplary alkynyl include a linear C₂-C₃₀ alkynyl, a branched C₄-C₃₀ alkynyl, a cyclic C₃-C₃₀ alkynyl, a linear C₂-C₂₀ alkynyl, a branched C₄-C₂₀ alkynyl, a cyclic C₃-C₂₀ alkynyl, a linear C₂-C₁₀ alkynyl, a branched C₄-C₁₀ alkynyl, a cyclic C₃-C₁₀ alkynyl, a linear C₂-C₆ alkynyl, a branched C₄-C₆ alkynyl, a cyclic C₃-C₆ alkynyl, a linear C₂-C₄ alkynyl, cyclic C₃-C₄ alkynyl, such as a linear C₂-C₁₀, C₂-C₉, C₂-C₈, C₂-C₇, C₂-C₆, C₂-C₅, C₂-C₄, C₂-C₃, C₂ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group. The cyclic alkynyl can be a monocyclic or polycyclic alkynyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄- C₇, C₄-C₆, or C₄-C₅ monocyclic or polycyclic alkynyl group.

It is understood that any of the exemplary alkyl, alkenyl, and alkynyl groups can be heteroalkyl, heteroalkenyl, and heteroalkynyl, respectively.

For any forms of the substrate described above, the aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl.

For any forms of the substrate described above, the polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

### 2. Catalyst

The catalyst in the first reaction mixture of step (i) for catalyzing asymmetric decarboxylation of the substrate to produce chiral amino acids is preferably a chiral phosphoric acid. Without being bound to any theories, it is believed that the selective protonation achieved using chiral phosphoric acids in the methods not only lies in the high acidity of the chiral phosphoric acids that can rapidly protonate, but also in the chiral phosphoric acids' capability to impose stereocontrol by forming a rich network of hydrogen bonds within their pockets, particularly with heteroatom-rich substrates or intermediates (see, e.g., 8 and 9 in FIG. 1). As such, the chiral phosphoric acids may interrupt the internal hydrogen bonding of the malonic acid (see, e.g., 8 in FIG. 1) and initiate the decarboxylation via an intermolecular proton transfer (see, e.g., 10 in FIG. 1). Direct interception of the intermediate (see, e.g., the enol intermediate 9 in FIG. 1) from self-decarboxylation is also possible. In either scenario, a stereoselective protonation inside the chiral pocket (see, e.g., 11 in FIG. 1) would produce the chiral amino acid (see, e.g., 12 in FIG. 1).

Any suitable chiral phosphoric acids ((R)- and (S)-chiral phosphoric acids), such as those that are commercially available, can be used in the disclosed method. Examples of chiral phosphoric acid suitable for use in step (i) of the disclosed method include, but are not limited to, Binol phosphoric acids and derivatives thereof, H8 Binol phosphoric acids and derivatives thereof, Spinol phosphoric acids and derivatives thereof, Biphenol phosphoric acids and derivatives thereof, dithiophosphoric acids and derivatives thereof, Taddol phosphoric acids and derivatives thereof, paracyclophane and derivatives thereof, TiPSY phosphoric acids and derivatives thereof, and TRIP phosphoric acids and derivatives thereof. Exemplary structures of chiral phosphoric acids are disclosed on https://www.sigmaaldrich.com/US/en/technical-documents/technical-article/chemistry-and-synthesis/reaction-design-and-optimization/chiral-phosphoric and website daicelchiraltech.cn/en/reagents/list02.aspx?pid=72621643502977024&cid=72620543991349248&sid=72626041549488128.

In some forms, the catalyst can be chiral phosphoric acid loaded on a support to facilitate recycling following the asymmetric decarboxylation and/or a subsequent alkylation reaction or hydrolysis. The support can be any material suitable for loading the chiral phosphoric acid that do not interfere with the asymmetric decarboxylation reaction, such as polystyrene, metal-organic frameworks, covalent organic framework, etc.

In some forms, the chiral phosphoric acid used in the first reaction mixture for catalyzing asymmetric decarboxylation of the substrate to produce chiral amino acids can have the structure of Formula III or Formula III′:

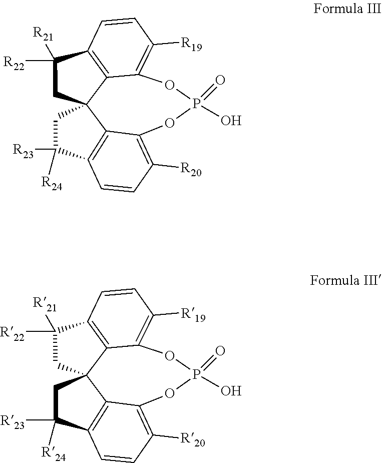

where R₁₉, R_{2M}, R′₁₉, and R′₂₀ can be independently substituted or unsubstituted aryl or substituted or unsubstituted polyaryl; R₂₁-R₂₄ and R′₂₁-R′₂₄ can be independently hydrogen or unsubstituted alkyl; the substituents, when present, can be independently substituted or unsubstituted alkyl, substituted or unsubstituted aryl (such as phenyl), substituted or unsubstituted polyaryl (such as naphthal), substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or

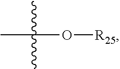

and R₂₅ can be substituted or unsubstituted alkyl, substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), substituted or unsubstituted aryl (such as phenyl), or substituted or unsubstituted aralkyl (such as benzyl).

In some forms, at least one of R₁₉ and R₂₀ and at least one of R′₁₉ and R′₂₀ can be independently a substituted aryl or substituted polyaryl, and optionally wherein the substituted aryl or polyaryl has three or more substituents (such as 2,4,6-substituted phenyl). In some forms, both R₁₉ and R₂₀ and both R′₁₉ and R′₂₀ can be independently a substituted aryl or substituted polyaryl, and optionally the substituted aryl or polyaryl has three or more substituents (such as 2,4,6-substituted phenyl).

In some forms, the substituents on the aryl or polyaryl of R₁₉, R₂₀, R′₁₉, and R′₂₀ can be unsubstituted alkyl (such as methyl, ethyl, isopropyl, n-propyl, etc.), unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or an alkoxy (such as methoxy, ethoxy, aryloxy, benzoether, etc.). In some forms, the substituents on the aryl or polyaryl of R₁₉, R₂₀, R′₁₉, and R′₂₀ can be unsubstituted alkyl (such as methyl, ethyl, isopropyl, n-propyl, etc.).

In some forms, the chiral phosphoric acid can have the structure of:

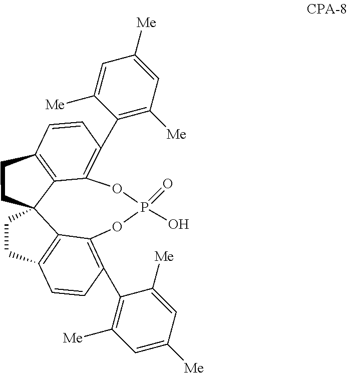

Generally, the catalyst is present in the first reaction mixture in an amount ranging from about 1 mol % to about 20 mol %, from about 1 mol % to about 10 mol %, from about 2 mol % to about 20 mol %, from about 2 mol % to about 10 mol %, or from about 2.5 mol % to about 10 mol %, such as about 10 mol % or about 2.5 mol %.

### 3. Chiral Amino Acids

The product formed in step (i) contains a chiral amino acid, which is produced by asymmetric decarboxylation of the substrate. As such, the functional group or biomolecule moiety (besides the protected amino group) that attaches to the methylene carbon of a malonic acid substrate becomes the side chain or part of the side chain of the chiral amino acid produced therefrom.

In some forms, when the substrate is a malonic acid substrate of Formula I or Formula I′, the chiral amino acid produced therefrom has the structure of Formula II or Formula II′:

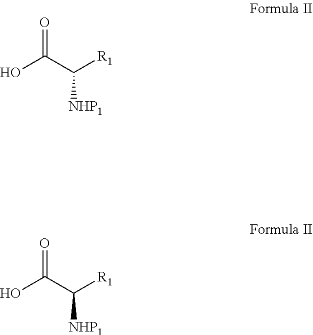

where P₁ can be any of the amino protecting group as defined above for the substrate, and R₁ can be any of the functional group or biomolecule moiety as defined above for the substrate.

When the chiral amino acid produced in step (i), such as the chiral amino acid of Formula II or Formula II′, is further reacted with an alkylation reactant (such as a methylation or ethylation reactant), the carboxyl group of the chiral amino acid is alkylated to produce an alkylated chiral amino acid (such as a methylated chiral amino acid or an ethylated amino acid). In some forms, the alkylated chiral amino acid can have the structure of Formula VIII or Formula VIII′:

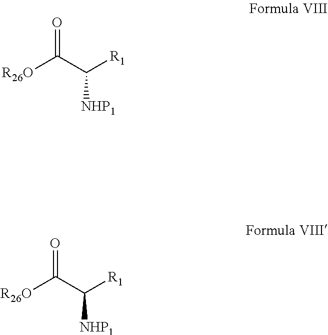

where R₂₆ can be a substituted or unsubstituted alkyl, such as a C₁-C₁₂ unsubstituted alkyl, a C₁-C₁₀ unsubstituted alkyl, a C₁-C₈ unsubstituted alkyl, a C₁-C₆ unsubstituted alkyl, or a C₁-C₄ unsubstituted alkyl, for example, methyl or ethyl; P₁ can be any of the amino protecting group as defined above for the substrate; and R₁ can be any of the functional group or biomolecule moiety as defined above for the substrate.

In some forms, the alkylated chiral amino acid can be a methylated chiral amino acid having the structure of Formula IV or Formula IV′:

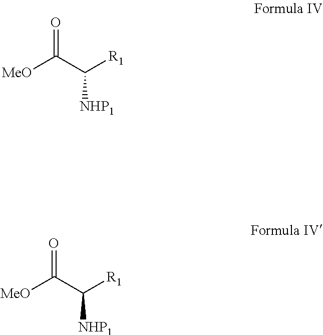

where P₁ can be any of the amino protecting group as defined above for the substrate, and R₁ can be any of the functional group or biomolecule moiety as defined above for the substrate.

In some forms, P₁ of Formula VIII, VIII′, IV, and IV′ can be

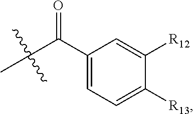

where R₁₂ can be

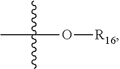

R₁₃ can be cyano, isocyano, nitro, or

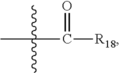

and R₁₆ and R₁₈ can be independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), where R′₁₈ can be unsubstituted alkyl. In some forms, R₁₃ can be nitro and R₁₂ can be

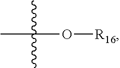

where R₁₆ can be an unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃). In some forms, R₁₃ can be nitro and R₁₂ can be

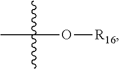

where R₁₆ can be —CH₂—CH₂—CF₃, —CH₂—CF₂—CF₃, or —CH₂—CF₂—CF₂—CF₃.

In some forms, the functional group or biomolecule moiety R₁ of Formula VIII, VIII′, IV, and IV′ can be a substituted or unsubstituted alkyl, a substituted or unsubstituted alkenyl, or a substituted or unsubstituted alkynyl, where the substituents, when present, can be independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl. In some forms, the substituents, when present, can be independently substituted or unsubstituted cycloalkyl, substituted or unsubstituted cycloalkenyl, substituted or unsubstituted cycloalkynyl, substituted or unsubstituted aryl, substituted or unsubstituted polyaryl, substituted or unsubstituted heteroaryl, substituted or unsubstituted heteropolyaryl, substituted or unsubstituted heterocyclyl, azido, carbonyl, alkoxy, halide, hydroxyl, thiol, cyano, isocyano, or nitro. In some forms, the substituents, when present, are independently a biomolecule moiety, such as a cholic acid moiety (see, e.g., (+)-39 in FIG. 3D) or estrone moiety (see, e.g., (+)-40 in FIG. 3D).

In some forms, the alkylated chiral amino acid produced using the disclosed method can have the structure of any one of the following:

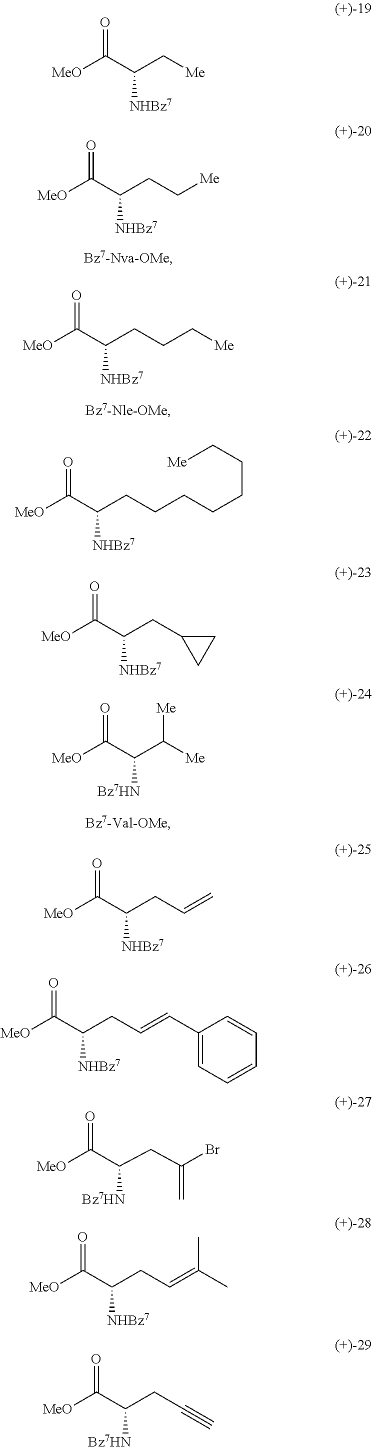

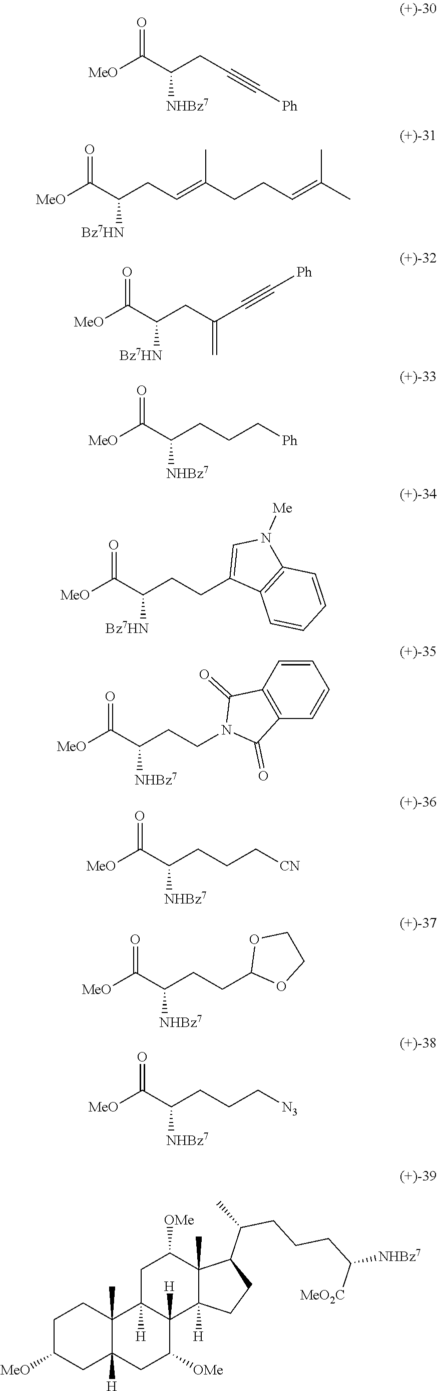

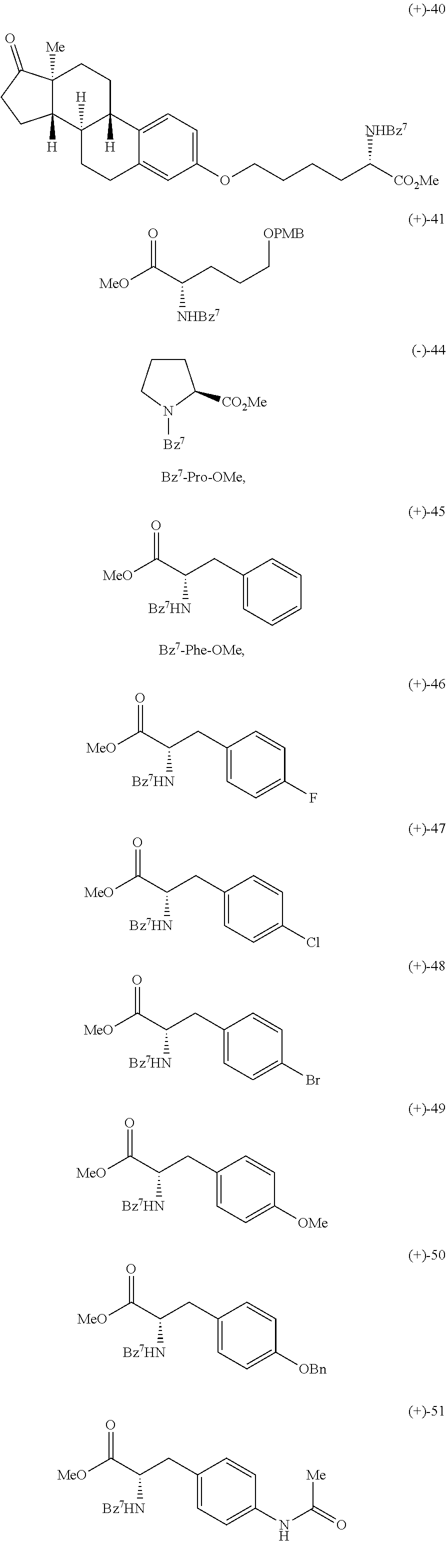

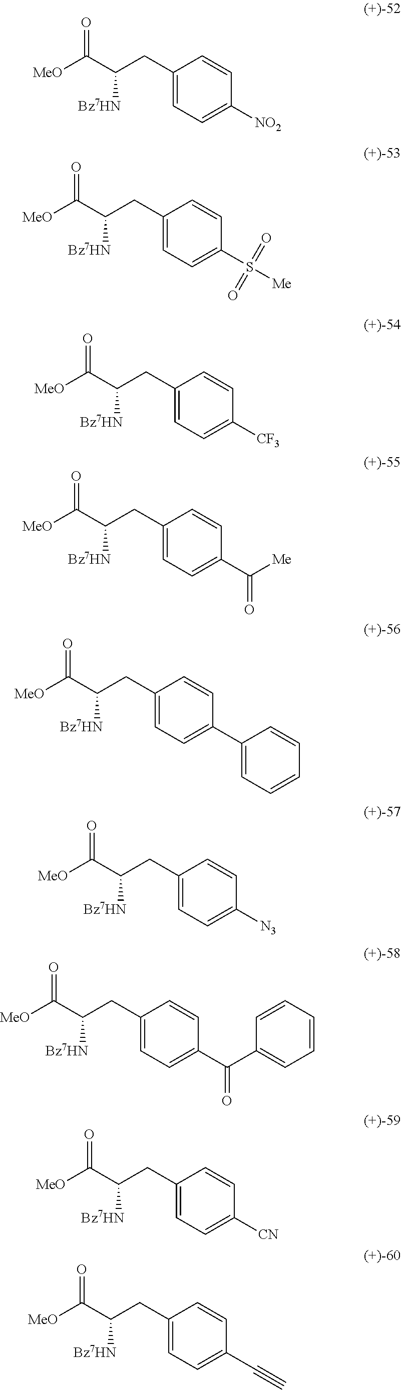

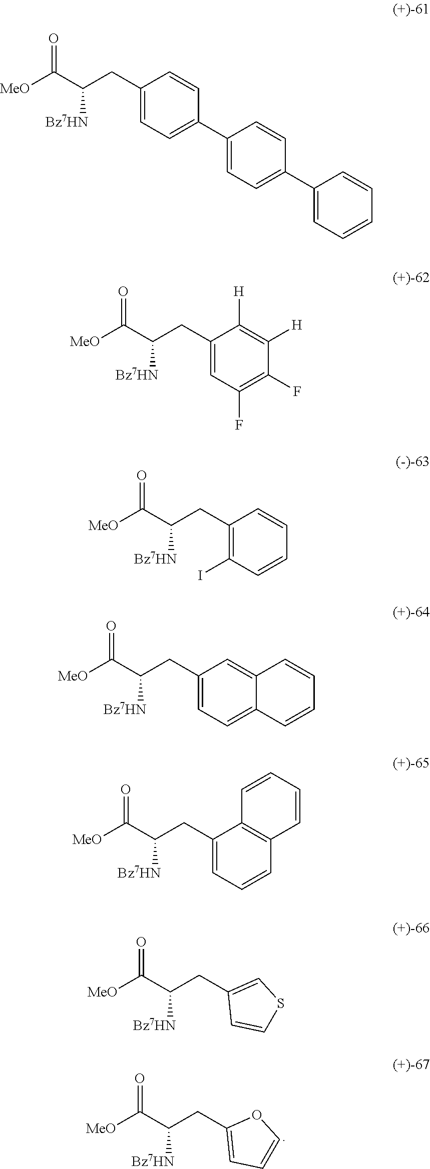

When the chiral amino acid produced in step (i), such as the chiral amino acid of Formula II or Formula II′, is further reacted with an acid, the protected amino group of the chiral amino acid is hydrolyzed to produce a hydrolyzed chiral amino acid. In some forms, the hydrolyzed chiral amino acid can have the structure of Formula V or Formula V′:

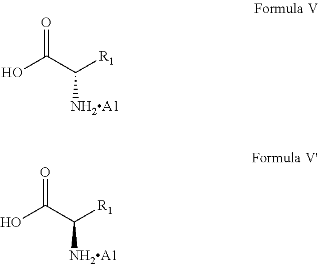

where R₁ can be any of the functional group or biomolecule moiety as defined above for the substrate, and A1 can be any suitable acid molecule. Examples of acid suitable for hydrolyzing the chiral amino acid include, but are not limited to, hydrochloric acid (HCl), sulfuric acid, nitric acid, carbonic acid, formic acid, acetic acid, citric acid, and acetylsalicylic acid. In some forms, A1 is HCl.
   a. Enantiomeric Excess

Generally, the chiral amino acid in the product formed from the asymmetric decarboxylation reaction using the disclosed methods can have an enantiomeric excess (“ee”) of at least 70%, at least 75%, at least 80%, at least 85%, at least 90%, in a range from about 70% to 100%, from about 75% to 100%, from about 80% to 100%, from about 85% to 100%, from about 90% to 100%, from about 75% to 99%, from about 80% to 99%, from about 85% to 99%, or from about 90% to 99%, as determined by chiral HPLC. The ee of chiral amino acid in the product can be calculated using the formula: ee=[(moles of enantiomer−moles of another enantiomer)/total moles of both enantiomers]×100%. The ee of the chiral amino acid can be determined using known methods, such as using chiral HPLC or a polarimeter. In some forms, the ee of the chiral amino acid is determined using chiral HPLC.

In some forms, the chiral amino acid of Formula II, the alkylated chiral amino acid of Formula VIII, the methylated chiral amino acid of Formula IV, or the hydrolyzed chiral amino acid of Formula V in the product formed from the asymmetric decarboxylation reactions catalyzed by a chiral phosphoric acid (such as a chiral phosphoric acid of Formula III or CPA-8) using the disclosed methods can have an ee of at least 70%, at least 75%, at least 80%, at least 85%, at least 90%, in a range from about 70% to 100%, from about 75% to 100%, from about 80% to 100%, from about 85% to 100%, from about 90% to 100%, from about 75% to 99%, from about 80% to 99%, from about 85% to 99%, or from about 90% to 99%, as determined by chiral HPLC.

In some forms, the chiral amino acid of Formula II′, the alkylated chiral amino acid of Formula VIII′, the methylated chiral amino acid of Formula IV′, or the hydrolyzed chiral amino acid of Formula V′ in the product formed from the asymmetric decarboxylation reactions catalyzed by a chiral phosphoric acid (such as a chiral phosphoric acid of Formula III′) using the disclosed methods can have an ee of at least 70%, at least 75%, at least 80%, at least 85%, at least 90%, in a range from about 70% to 100%, from about 75% to 100%, from about 80% to 100%, from about 85% to 100%, from about 90% to 100%, from about 75% to 99%, from about 80% to 99%, from about 85% to 99%, or from about 90% to 99%, as determined by chiral HPLC.

b. Yield

Generally, the chiral amino acid in the product formed from the asymmetric decarboxylation reaction using the disclosed methods can have a yield of at least 80%, such as in a range from about 80% to about 99%. The yield of the chiral amino acid in the product can be calculated using the formula: chiral amino acid yield=(experimentally obtained total no. of mole of chiral amino acid)/(theoretical total no. of mole of chiral amino acid)×100%. The experimentally obtained total no. of mole of the chiral amino acid can be determined using known methods, such as using NMR (e.g. ¹H NMR, ¹⁹F NMR, and/or ³¹P NMR) spectroscopy with an internal standard of known quantity, such as using a known quantity of PhTMS as the internal standard.

In some forms, the chiral amino acid of Formula II, the alkylated chiral amino acid of Formula VIII, the methylated chiral amino acid of Formula IV, or the hydrolyzed chiral amino acid of Formula V in the product formed from the asymmetric decarboxylation reactions catalyzed by a chiral phosphoric acid (such as a chiral phosphoric acid of Formula III or CPA-8) using the disclosed methods can have a yield of at least 80%, such as in a range from about 80% to about 99%.

In some forms, the chiral amino acid of Formula II′, the alkylated chiral amino acid of Formula VIII′, the methylated chiral amino acid of Formula IV′, or the hydrolyzed chiral amino acid of Formula V′ in the product formed from the asymmetric decarboxylation reactions catalyzed by a chiral phosphoric acid (such as a chiral phosphoric acid of Formula III′) using the disclosed methods can have a yield of at least 80%, such as in a range from about 80% to about 99%.

### B. Optional Steps

The disclosed methods can include one or more optional steps, such as (ii) adding an alkylation reactant (such as a methylation or ethylation reactant) or an acid to the product to form a second reaction mixture, and (iii) maintaining the second reaction mixture at a second temperature for a second period of time sufficient to form an alkylation product (such as a methylation or ethylation product) or hydrolysis product; purifying the product after step (i) and/or the alkylation or hydrolysis product after step (iii); preparing the substrate prior to step (i); recycling an amino protecting agent and the catalyst after step (i) and/or step (iii); and/or derivatizing the chiral amino acid or alkylated or hydrolyzed chiral amino acid to a derivatized compound after step (i) and/or step (iii).

In some forms, the disclosed method includes a step of purifying the product to remove impurities, such as the catalyst, the undesired isomer(s), and/or unreacted substrates, in the product, and thereby obtain purified chiral amino acid, subsequent to the asymmetric decarboxylation reaction (i.e., step (i)). The product can be purified by known methods, such as using column chromatography on silica gel or recrystallization in a suitable solvent.

In some forms, the catalyst and/or an amino protecting agent can be recycled from the product, subsequent to the asymmetric decarboxylation reaction (i.e., step (i)), and/or subsequent to the alkylation/hydrolysis reaction (i.e., step (ii)). For example, the catalyst and an amino protecting agent can be recycled using extraction subsequent to hydrolysis of the chiral amino acid produced in an asymmetric decarboxylation reaction.

### 1. Forming Alkylation or Hydrolysis Products

In some forms, subsequent to the asymmetric decarboxylation reaction (i.e., step (i)), the chiral amino acid produced in the asymmetric decarboxylation reaction is further treated with an alkylation reactant (such as a methylation or ethylation reactant) or an acid to form an alkylated chiral amino acid (such as a methylated or ethylated chiral amino acid) or hydrolyzed chiral amino acid. Alkylation reactants and reaction conditions for converting carboxylic acid group to esters are known, see, for example, those described in Matsumoto et al., “Recent Advances in the Synthesis of Carboxylic Acid Esters” DOI: 10.5772/intechopen.74543.

For example, the chiral amino acid of Formula II or Formula II′ (as described above) is reacted with a methylation reactant to form a methylated chiral amino acid of Formula IV or Formula IV′ (as described above) via a methylation reaction. The methylation reactant can be any suitable chemical that can react with the chiral amino acid and convert its carboxyl group to an ester. Examples of methylation reactant suitable for use in the methylation reaction include, but are not limited to, trimethylsilyldiazomethane, iodomethane, dimethyl sulfate, dimethyl carbonate, or tetramethylammonium chloride, methyl triflate, diazomethane, and methyl fluorosulfonate. In preferred forms, the methylation reactant used in the methylation reaction to form the methylated chiral amino acid is trimethylsilyldiazomethane. Generally, the methylation reaction is performed at a temperature ranging from about 50° C. to about 100° C., from about 60° C. to about 100° C., or from about 70° C. to about 100° C., for a period of time ranging from about 30 minutes to about 2 hours or from about 30 minutes to about 1 hour.

For example, the chiral amino acid of Formula II or Formula II′ (as described above) is reacted with an acid to form a hydrolyzed chiral amino acid of Formula V or Formula V′ (as described above) via hydrolysis. The acid can be any suitable acid having an acidity that can deprotect the protected amino group of the chiral amino acid, such as any one of those described above for A1. In preferred forms, the acid used in the hydrolysis reaction to deprotect the protected amino group and form the hydrolyzed chiral amino acid is HCl. Generally, the hydrolysis reaction is performed at a temperature ranging from about 90° C. to about 120° C. or from about 90° C. to about 110° C., for a period of time ranging from about 12 hours to about 36 hours or from about 12 hours to about 24 hours.

For example, the chiral amino acid of Formula II or Formula II′ (as described above) is reacted with an ethylation reactant to form an ethylated chiral amino acid via an ethylation reaction (such as reaction between the chiral amino acid of Formula II or II′ and thionyl chloride in ethanol).

In some forms, prior to addition of the alkylation reactant (such as a methylation or ethylation reactant) or acid (i.e., step (ii)), the product containing chiral amino acid is cooled to room temperature or about 0° C., following which the alkylation reactant (such as a methylation or ethylation reactant) or acid is added into the cooled product at room temperature (such as for methylation reaction) or 0° C. (for hydrolysis) before heating the reaction mixture to a suitable temperature (such as those described above) for reaction.

### 2. Preparing Substrates

In some forms, prior to the asymmetric decarboxylation reaction (i.e., step (i)), the substrate is prepared by converting a starting material to the substrate using known reactions, such as substitution reaction and hydrolysis. Typically, the starting material is readily available, such as commercially available malonic acids or esters.

In some forms, the starting material is a commercially available malonic ester having the structure of Formula VI:

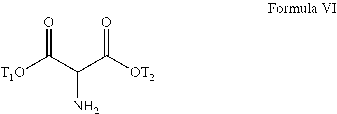

where T₁ and T₂ can independently be unsubstituted alkyl, such as methyl, ethyl, n-propyl, isopropyl, n-butyl, isobutyl, tert-butyl, etc. For example, T₁ and T₂ are both methyl or ethyl.

In these forms, the starting material can react with an amino protecting agent P₁X₁ via a substitution reaction to form an amino-protected malonic ester having the structure of Formula VII:

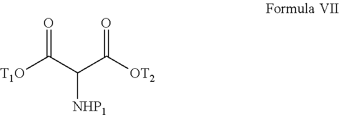

where X₁ is a halide (such as fluoride, chloride, iodide, etc.) and P₁ can be any suitable amino protecting group, such as any one of those described above for the substrate.

The amino protected malonic ester of Formula VII can then undergo a substitution reaction and hydrolysis to form the substrate of Formula I or Formula I′ described above. For example, the amino protected malonic ester of Formula VII reacts with a substitution reactant R₁X₂, where X₂ is a halide (such as fluoride, chloride, iodide, etc.) and R₁ is a functional or biomolecule moiety (such as any one of those described above for the substrate), to attach R₁ to the methylene carbon of the malonic ester, which is then hydrolyzed to form the malonic acid substrate of Formula I or Formula I′. In some forms, the substitution reaction and hydrolysis can occur simultaneously, i.e., the substitution reaction to attach R₁ and hydrolysis of the ester groups to form carboxyl groups are achieved in a single step.

### 3. Further Derivatization of Chiral Amino Acids

Because of the various functionalities introduced through a sidechain in the chiral amino acids, the chiral amino acids can be used for further derivatization to generate a multitude of useful compounds, such as cyclic amino acids, drug-amino acid conjugates, DNA gyrase inhibitors, etc.

Further derivatization of the chiral amino acids can be performed using any known reactions, such as oxidation reactions, coupling reactions (such as click reaction), nucleophilic/electrophilic substitutions, and metathesis reactions, and combinations thereof.

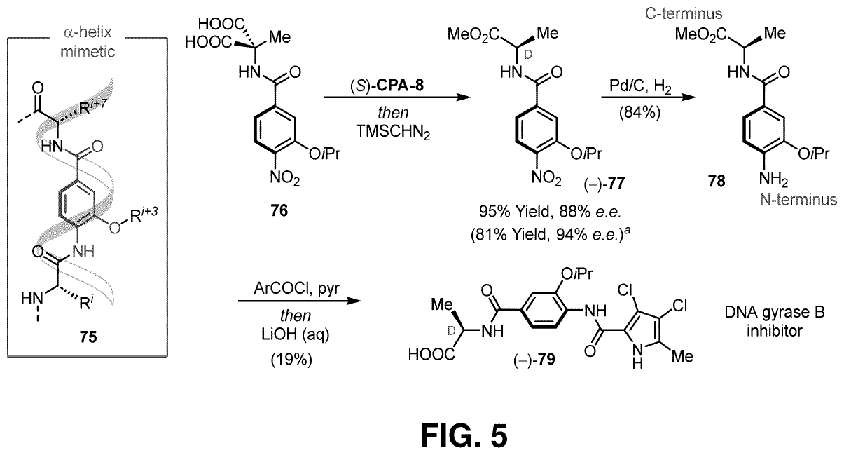

For example, the chiral amino acids produced using the disclosed method containing a p-methoxybenzyl group in the sidechain can be derived to form a cyclic amino acid via a series of substitution and oxidation reactions (see, e.g., FIG. 3D, 41-44). For example, the chiral amino acids produced using the disclosed method contain alkyne or azido group that can react with a drug containing an azido or alkyne group to form drug-amino acid conjugates via click chemistry. For example, when the amino protecting group of the chiral amino acid produced using the disclosed method is 3-alkoxy-4-nitrobenzamides, it can be further derived to form DNA gyrase B inhibitor via hydrogenation, acylation, and ester hydrolysis (see, e.g., FIG. 5).

Specific exemplary substrates, chiral phosphoric acids, chiral amino acids, alkylated chiral amino acids (such as methylated chiral amino acids), hydrolyzed chiral amino acids, chiral amino acids with further derivatization, and their corresponding yields and enantiomeric ratios, are described in the Examples below.

The disclosed methods and compositions can be further understood through the following numbered paragraphs.

1. A method for producing chiral amino acids, comprising:
(i) maintaining a first reaction mixture at a first temperature for a first period of time sufficient to form a product,
wherein the first reaction mixture comprises a substrate, a catalyst, and a solvent,
wherein the catalyst is a chiral phosphoric acid, and
wherein the product comprises a chiral amino acid.

2. The method of paragraph 1, wherein the substrate is a malonic acid.

3. The method of paragraph 2, wherein the malonic acid has the structure of Formula I or Formula I′:

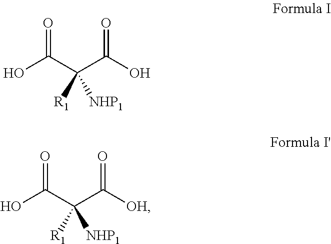

wherein the chiral amino acid in the product has the structure of Formula II or Formula II′:

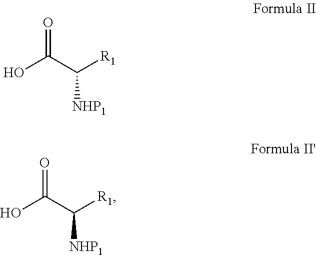

wherein P₁ is an amino protecting group and R₁ is a functional group or biomolecule moiety.

4. The method of any one of paragraphs 1-3, further comprising:
(ii) adding an alkylation reactant or an acid to the product to form a second reaction mixture, and
(iii) maintaining the second reaction mixture at a second temperature for a second period of time sufficient to form an alkylated product or hydrolysis product,
wherein the alkylation product or hydrolysis product comprises an alkylated chiral amino acid or hydrolyzed chiral amino acid.

5. The method of paragraph 4, wherein the alkylated chiral amino acid has the structure of Formula VIII or Formula VIII′, and the hydrolyzed chiral amino acid has the structure of Formula V or Formula V′:

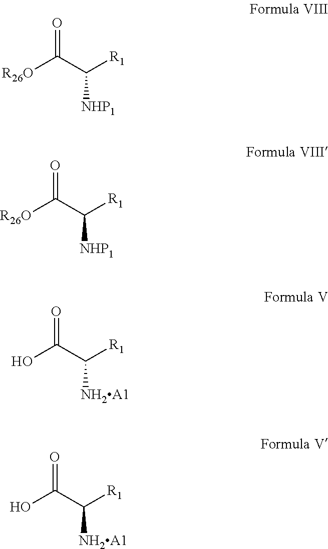

wherein P₁ and R₁ are as defined above in paragraph 3, R₂₆ is a substituted or unsubstituted alkyl, such as a C₁-C₁₂ unsubstituted alkyl, a C₁-C₁₀ unsubstituted alkyl, a C₁-C₈ unsubstituted alkyl, a C₁-C₆ unsubstituted alkyl, or a C₁-C₄ unsubstituted alkyl, for example, methyl or ethyl, and A1 is an acid molecule (such as HCl),
optionally wherein the alkylated chiral amino acid is a methylated chiral amino acid having the structure of Formula IV or Formula IV′:

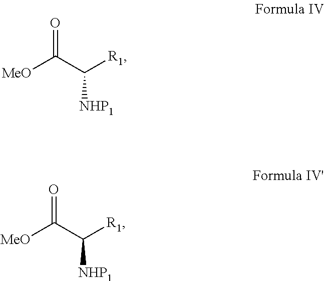

wherein P₁ and R₁ are as defined above in paragraph 3.

6. The method of paragraph 4 or 5, wherein the alkylation reactant in the second reaction mixture is a methylation reactant (such as trimethylsilyldiazomethane) and the acid in the second reaction mixture is HCl.

7. The method of any one of paragraphs 3-6, wherein P₁ is

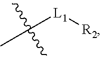

and wherein:
L₁ is

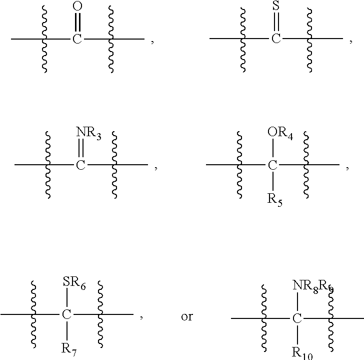

R₂ is hydrogen, hydroxyl, halide, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, carbonyl, cyano, isocyano, nitro, silyl, sulfinyl, sulfonyl, phosphonium, phosphanyl, phosphoryl, or phosphonyl;
R₃-R₁₀ are independently hydrogen or a substituted or unsubstituted alkyl (such as unsubstituted alkyl, for example, C₁-C₁₀ unsubstituted alkyl, C₁-C₈ unsubstituted alkyl, C₁-C₆ unsubstituted alkyl, or C₁-C₄ unsubstituted alkyl); and
the substituents, when present, are independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl.

8. The method of paragraph 7, wherein L₁ is

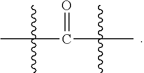 [SMILES: *C(*)=O]

9. The method of paragraph 7 or 8, wherein R₂ is substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, or carbonyl.

10. The method of any one of paragraphs 7-9, wherein R₂ is substituted or unsubstituted cycloalkyl, substituted or unsubstituted cycloalkenyl, substituted or unsubstituted cycloalkynyl, substituted or unsubstituted aryl, substituted or unsubstituted polyaryl, or substituted or unsubstituted aralkyl.

11. The method of any one of paragraphs 7-10, wherein R₂ is

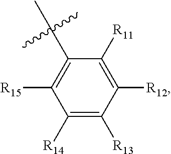

and wherein R₁₁-R₁₅ are independently hydrogen, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl.

12. The method of paragraph 11, wherein R₁₁-R₁₅ are independently hydrogen, halide, unsubstituted alkyl, unsubstituted alkenyl, unsubstituted alkynyl, unsubstituted phenyl, unsubstituted haloalkyl, unsubstituted aralkyl, cyano, isocyano, nitro,

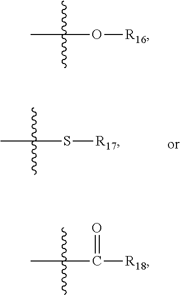

and wherein R₁₆-R₁₈ are independently hydrogen, hydroxyl, —OR′₁₈, unsubstituted alkyl, unsubstituted phenyl, unsubstituted haloalkyl, or unsubstituted aralkyl, and R′₁₈ is unsubstituted alkyl.

13. The method of any one of paragraphs 7-12, wherein R₂ is:

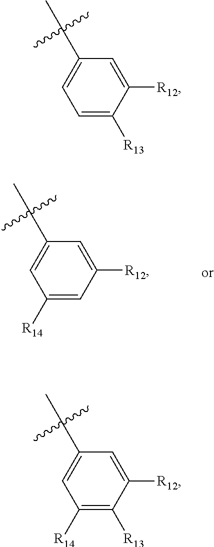

and
wherein R₁₂-R₁₄ are independently halide (such as F, Cl, or I), unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), cyano, isocyano, nitro,

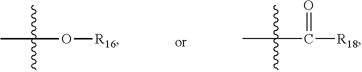

and wherein R₁₆ and R₁₈ are independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), and R′₁₈ is unsubstituted alkyl.

14. The method of any one of paragraphs 7-13, wherein R₂ is

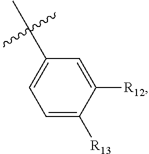

and wherein R₁₂ is

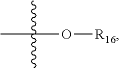

R₁₃ is halide (such as F, Cl, or I), unsubstituted alkyl, unsubstituted haloalkyl (such as —CF₃), cyano, isocyano, nitro,

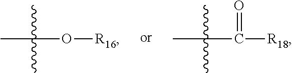

and R₁₆ and R₁₈ are independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), and R′₁₈ is unsubstituted alkyl.

15. The method of paragraph 14, wherein R₁₃ is nitro.

16. The method of any one of paragraphs 3-15, wherein R₁ is a substituted or unsubstituted alkyl, a substituted or unsubstituted alkenyl, or a substituted or unsubstituted alkynyl, and
wherein the substituents, when present, are independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl.

17. The method of paragraph 16, wherein the substituents, when present, are independently substituted or unsubstituted cycloalkyl, substituted or unsubstituted cycloalkenyl, substituted or unsubstituted cycloalkynyl, substituted or unsubstituted aryl, substituted or unsubstituted polyaryl, substituted or unsubstituted heteroaryl, substituted or unsubstituted heteropolyaryl, substituted or unsubstituted heterocyclyl, azido, carbonyl, alkoxy, halide, hydroxyl, thiol, cyano, isocyano, or nitro.

18. The method of paragraph 16 or 17, wherein the substituents, when present, are independently a biomolecule moiety.

19. The method of any one of paragraphs 1-18, wherein one or more carbons, one or more nitrogen, and/or one or more hydrogens of the substrate are in the form of ¹³C, ¹⁴C, ¹⁵N, and/or D.

20. The method of any one of paragraphs 1-19, wherein the chiral phosphoric acid is a Binol phosphoric acid or derivative thereof, an H8 Binol phosphoric acid or derivative thereof, a Spinol phosphoric acid or derivative thereof, a Biphenol phosphoric acid or derivative thereof, a dithiophosphoric acid or derivative thereof, a Taddol phosphoric acid or derivative thereof, a paracyclophane or derivative thereof, a TiPSY phosphoric acid or derivative thereof, or a TRIP phosphoric acid or derivative thereof.

21. The method of any one of paragraphs 1-20, wherein the chiral phosphoric acid has the structure of Formula III or Formula III′:

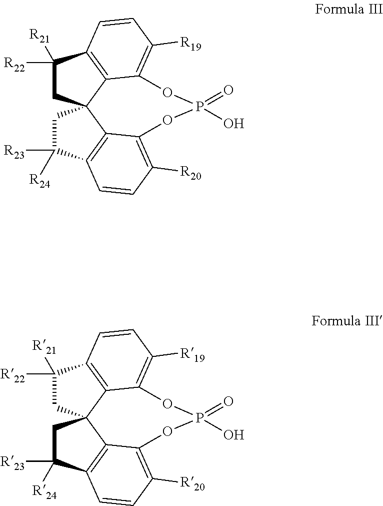

wherein:
R₁₉, R₂₀, R′₁₉, and R′₂₀ are independently substituted or unsubstituted aryl or substituted or unsubstituted polyaryl;
R₂₁-R₂₄ and R′₂₁-R′₂₄ are independently hydrogen or unsubstituted alkyl; and
the substituents, when present, are independently substituted or unsubstituted alkyl, substituted or unsubstituted aryl (such as phenyl), substituted or unsubstituted polyaryl (such as naphthal), substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or

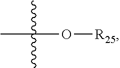

and R₂₅ is substituted or unsubstituted alkyl, substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), substituted or unsubstituted aryl (such as phenyl), or substituted or unsubstituted aralkyl (such as benzyl).

22. The method of paragraph 21, wherein at least one of R₁₉ and R₂₀ and at least one of R′₁₉ and R′₂₀ is independently a substituted aryl or substituted polyaryl, and optionally wherein the substituted aryl or polyaryl has three or more substituents (such as 2,4,6-substituted phenyl).

23. The method of any one of paragraphs 1-22, wherein the chiral phosphoric acid has the structure of

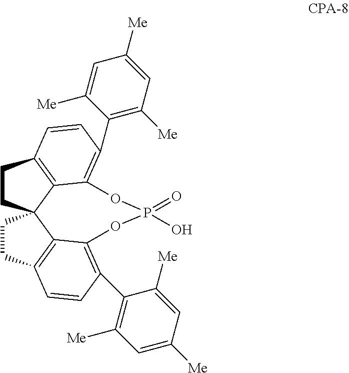

24. The method of any one of paragraphs 1-23, wherein the catalyst is present in the first reaction mixture in an amount ranging from about 1 mol % to about 20 mol %, from about 1 mol % to about 10 mol %, from about 2 mol % to about 20 mol %, from about 2 mol % to about 10 mol %, or from about 2.5 mol % to about 10 mol %, such as about 10 mol % or about 2.5 mol %.

25. The method of any one of paragraphs 1-24, wherein the solvent is an ether.

26. The method of paragraph 25, wherein the ether is ethyl acetate, ^{t}BuOMe, THF, 2-MeTHF, 1,4-dioxane, cyclopentylmethylether (CPME), or (MeOCH₂CH₂)₂O, or a combination thereof.

27. The method of any one of paragraphs 1-26, wherein the first reaction mixture is maintained at a temperature ranging from about 50° C. to about 100° C., from about 60° C. to about 100° C., or from about 70° C. to about 100° C., such as about 80° C., for a period of time ranging from about 1 hour to about 12 hours, from about 1 hour to about 10 hours, from about 1 hour to about 8 hours, from about 1 hour to about 5 hours, or from about 1 hour to about 3 hours, such as about 2 hours.

28. The method of any one of paragraphs 4-27, wherein the second reaction mixture (such as for methylation reaction) is maintained at a temperature ranging from about 50° C. to about 100° C., from about 60° C. to about 100° C., or from about 70° C. to about 100° C., for a period of time ranging from about 30 minutes to about 2 hours or from about 30 minutes to about 1 hour, or
wherein the second reaction mixture (for hydrolysis) is maintained at a temperature ranging from about 90° C. to about 120° C. or from about 90° C. to about 110° C., for a period of time ranging from about 12 hours to about 36 hours or from about 12 hours to about 24 hours.

29. The method of any one of paragraphs 4-28, wherein after step (i), the product is cooled to room temperature or about 0° C. before adding the alkylation reactant (such as the methylation reactant) or the acid.

30. The method of any one of paragraphs 1-29, wherein the chiral amino acid has a yield of at least 80% or in a range from about 80% to about 99%.

31. The method of any one of paragraphs 1-30, wherein the chiral amino acid has an enantiometric excess (ee) of at least 70%, at least 75%, at least 80%, at least 85%, at least 90%, in a range from about 70% to 100%, from about 75% to 100%, from about 80% to 100%, from about 85% to 100%, from about 90% to 100%, from about 75% to 99%, from about 80% to 99%, from about 85% to 99%, or from about 90% to 99%, as determined by chiral HPLC.

32. The method of any one of paragraphs 1-31, further comprising purifying the product after step (i) and/or the alkylation or hydrolysis product after step (iii); preparing the substrate prior to step (i); recycling an amino protecting agent and the catalyst after step (i) and/or step (iii); and/or derivatizing the chiral amino acid or alkylated or hydrolyzed chiral amino acid to a derivatized compound after step (i) and/or step (iii).

33. The method of paragraph 32, wherein the derivatization step is performed via an oxidation reaction, a coupling reaction (such as click reaction), a nucleophilic/electrophilic substitution, or a metathesis reaction, or a combination thereof.

34. The method of paragraph 32 or 33, wherein the derivatized compound is a cyclic amino acid, a drug-amino acid conjugate, or a DNA gyrase inhibitor.

35. The method of any one of paragraphs 32-34, wherein the substrate is prepared by:
(a) converting a starting compound having the structure of Formula VI to an amino protected compound of Formula VII:

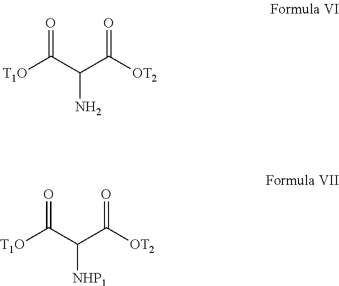

wherein T₁ and T₂ are independently unsubstituted alkyl, and P₁ is an amino protecting group; and
(b) converting the amino protected compound of Formula VII to the substrate of Formula I or I′.

36. A chiral amino acid having the structure of Formula VIII or Formula VIII′:

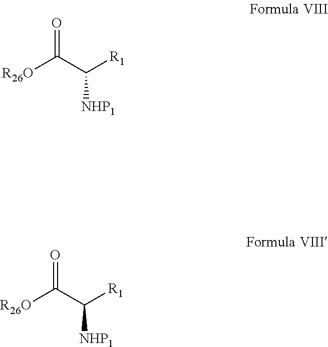

wherein R₂₆ is a substituted or unsubstituted alkyl, such as a C₁-C₁₂ unsubstituted alkyl, a C₁-C₁₀ unsubstituted alkyl, a C₁-C₈ unsubstituted alkyl, a C₁-C₆ unsubstituted alkyl, or a C₁-C₄ unsubstituted alkyl, e.g., methyl or ethyl;
P₁ is

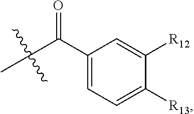

wherein R₁₂ is

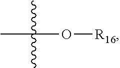

R₁₃ is cyano, isocyano, nitro, or

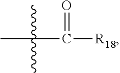

and R₁₆ and R₁₈ are independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), wherein R′₁₈ is unsubstituted alkyl;
R₁ is a substituted or unsubstituted alkyl, a substituted or unsubstituted alkenyl, or a substituted or unsubstituted alkynyl, wherein the substituents, when present, are independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl.

37. The chiral amino acid of paragraph 36, wherein R₂₆ is methyl or ethyl; R₁₃ is nitro; and R₁₂ is

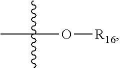

and R₁₆ is an unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), optionally wherein R₁₆ is —CH₂—CH₂—CF₃, —CH₂—CF₂—CF₃, or —CH₂—CF₂—CF₂—CF₃.

38. The chiral amino acid of paragraph 36 or 37, wherein the substituents, when present, are independently substituted or unsubstituted cycloalkyl, substituted or unsubstituted cycloalkenyl, substituted or unsubstituted cycloalkynyl, substituted or unsubstituted aryl, substituted or unsubstituted polyaryl, substituted or unsubstituted heteroaryl, substituted or unsubstituted heteropolyaryl, substituted or unsubstituted heterocyclyl, azido, carbonyl, alkoxy, halide, hydroxyl, thiol, cyano, isocyano, or nitro.

39. The chiral amino acid of any one of paragraphs 36-38, wherein the substituents, when present, are independently a biomolecule moiety (such as a cholic acid moiety, estrone moiety, etc.).

40. The chiral amino acid of any one of paragraphs 36-39, wherein R₂₆ is methyl; R₁₃ is nitro; and R₁₂ is

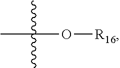

and R₁₆ is —CH₂—CF₂—CF₃.

41. The chiral amino acid of any one of paragraphs 36-40, having any one of the structure of:

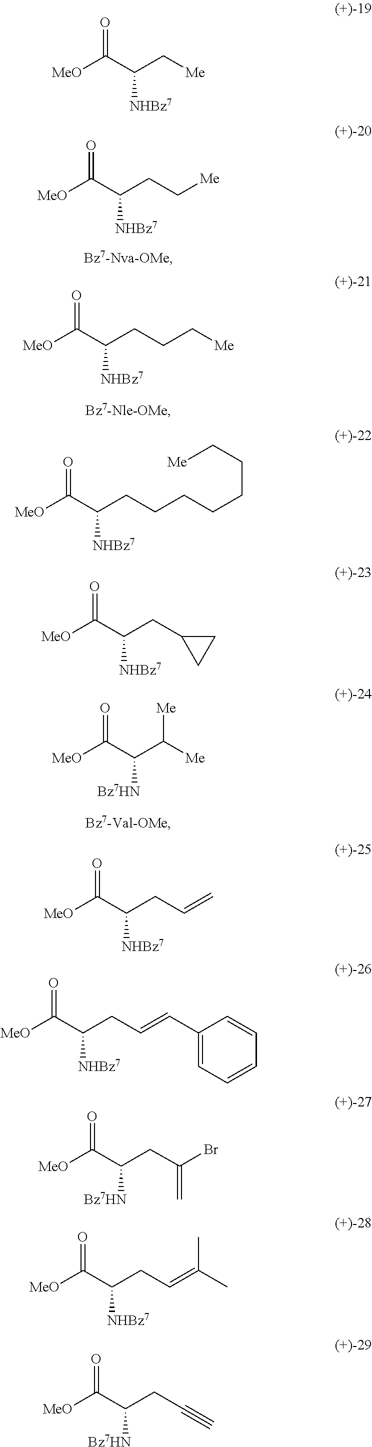

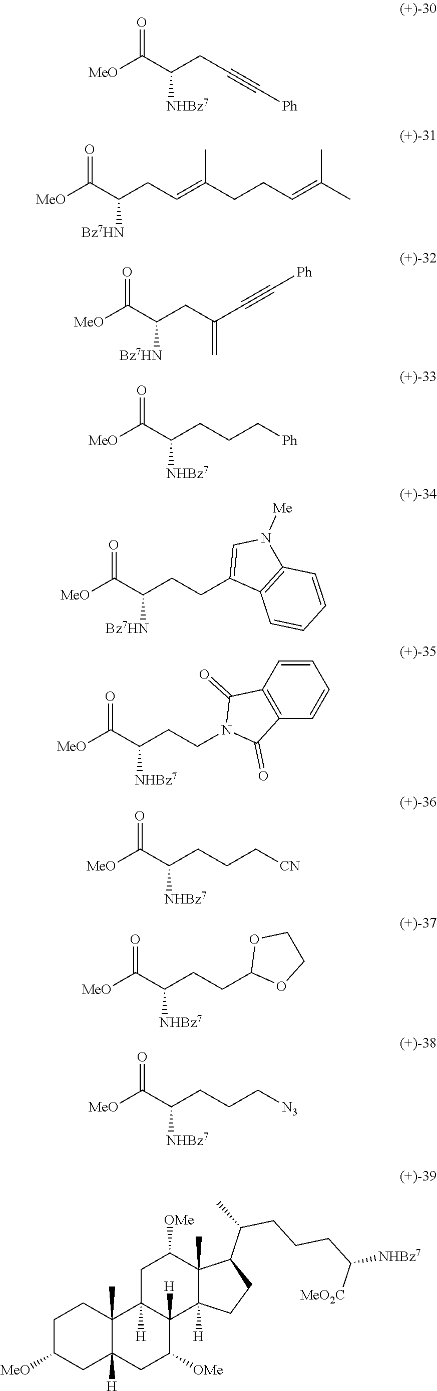

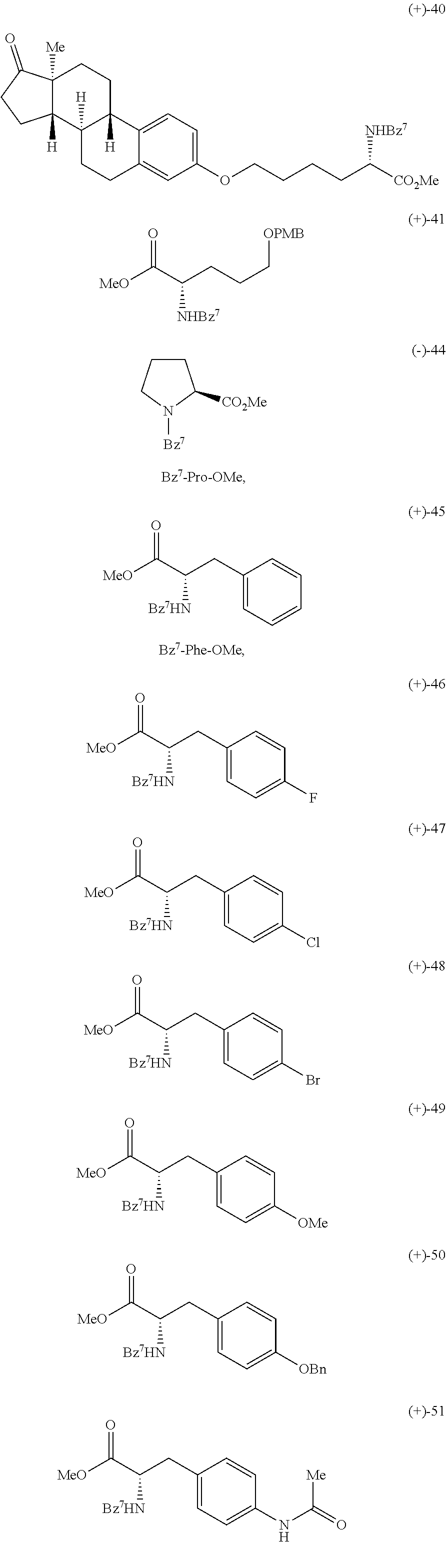

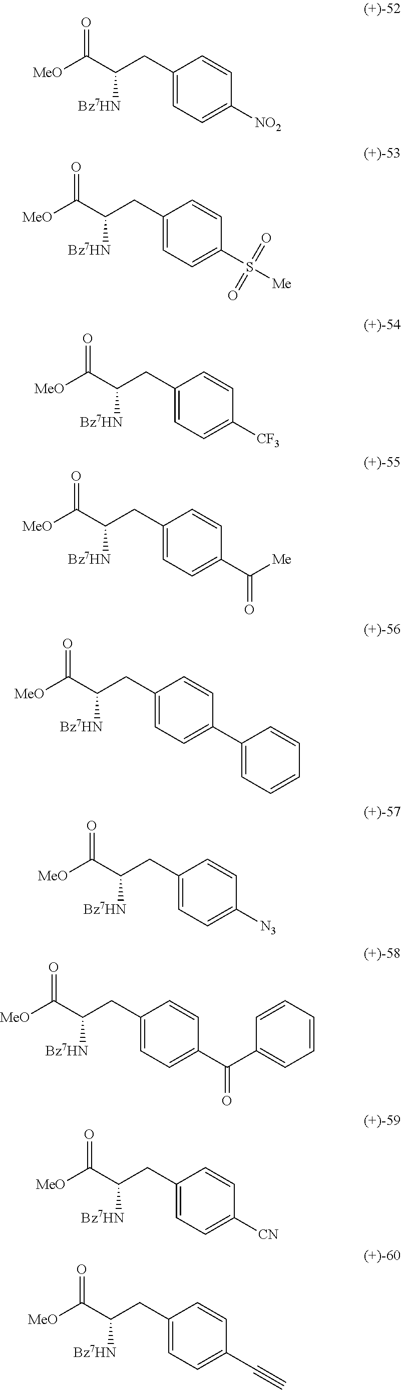

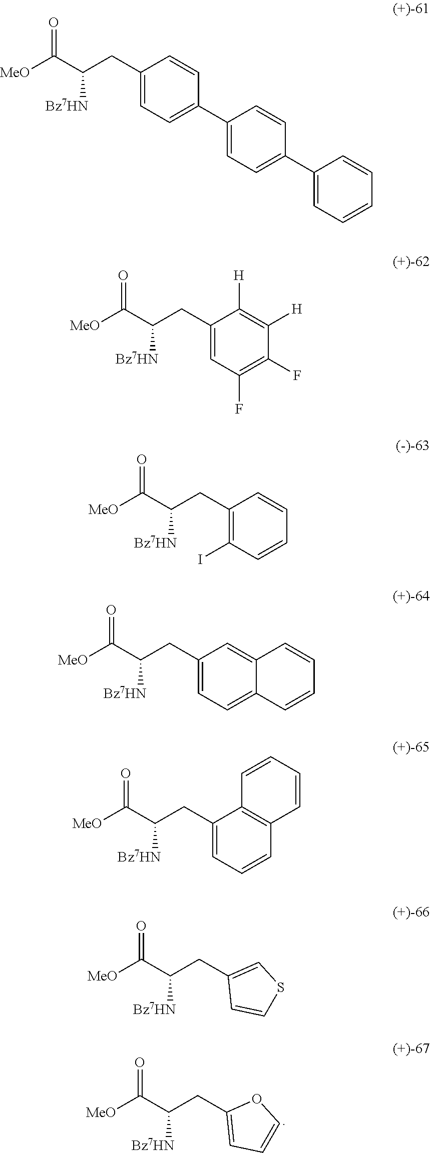

### EXAMPLES

### Example 1: Synthesis of α-Amino Acids using Asymmetric Malonic Ester

### Materials and Methods

### Reagents and Instruments

Unless otherwise noted, all reactions were run under nitrogen atmosphere. (R)- or (S)-chiral phosphoric acids (CPAs) (>98% purity) were purchased from Daicel Chiral Technologies (China) Co., Ltd., and used as received. Cyclopentyl methyl ether (CPME) was purchased from Shanghai Macklin Biochemical Co., Ltd. and used as received. Thin layer chromatography (TLC) was run on silica gel plates purchased from Yantai Huanghai Silica gel Development Co., Ltd.

¹H NMR, ¹³C NMR, and ¹⁹F NMR spectra were obtained on a Bruker 400, 500, or 600 spectrometer (400/500/600 MHz for ¹H, 100/125/150 MHz for ¹³C, 376/471 MHz for ¹⁹F). ¹⁵N NMR spectra were obtained on a Bruker 400 spectrometer (40 MHz for ¹⁵N). All ¹H NMR experiments were measured with tetramethylsilane (0 ppm) in CDCl₃, using the signal of residual DMSO (2.50 ppm) in DMSO-d₆, the signal of residual CH₃OH (3.31 ppm) in CD₃OD, or the signal of residual CH₃CN (1.94 ppm) in CD₃CN as internal reference. All ¹³C NMR experiments were measured in relative to the signal of CDCl₃ (77.0 ppm), the signal of DMSO-d₆ (39.52 ppm), the signal of CD₃OD (49.00 ppm), or the signal of CD₃CN (1.32, 118.3 ppm). Data for ¹H NMR, ¹³C NMR, and ¹⁹F NMR were presented as follows: chemical shifts (δ, ppm), multiplicity (br=broad, s=singlet, d=doublet, t=triplet, q=quartet, hept=heptet, dd=doublet of doublets, tt=triplet of triplets, td=triplet of doublets, m=multiplet), coupling constant (Hz), and integration. Chiral HPLC spectra were measured on an Agilent 1260 Infinity II. The [α]_{D} was recorded using INESA SGW-531 Automatic Polarimeter. Infrared absorption spectra were recorded on a PerkinElmer UATR two FT-IR and the data were presented as per centimeter (cm⁻¹). High-resolution EI mass spectra were recorded on a Thermo Scientific DFS high resolution magnetic sector MS. High-resolution ESI-MS measurements were performed on a Bruker impact II high-resolution LC-QTOF mass spectrometer. Accurate masses from high-resolution mass spectra were reported for the molecular ion [M+H]⁺.

### General Procedures for the Synthesis of Substrates

### Preparation of Common Intermediate 16

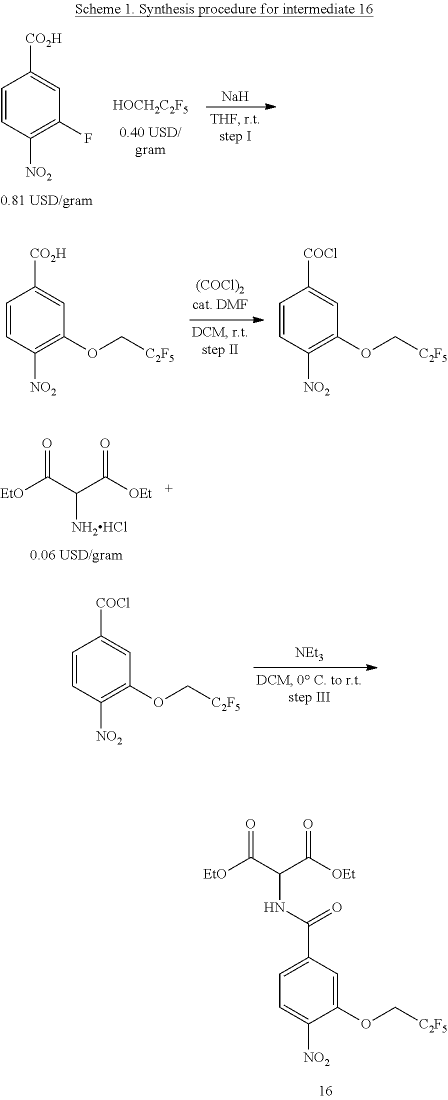

Step I: To a suspension of NaH (32.4 g, 810 mmol, 311 mol %, 60% dispersion in mineral oil) in THF (600 mL), 2,2,3,3,3-pentafluoropropan-1-ol (58.5 g, 390 mmol, 150 mol %) was added dropwise at room temperature. The resulting mixture was stirred at room temperature for 30 min and then 3-fluoro-4-nitrobenzoic acid (60.0 g, 324 mmol, 125 mol %) was added in small batches. The mixture was stirred at room temperature for 4 h, monitored by TLC. When the acid starting material was fully consumed, 1 M HCl (500 mL) aqueous solution was added slowly to the reaction mixture. The organic phase (THF) was separated, concentrated under vacuum, and redissolved in EtOAc. The aqueous phase was extracted with EtOAc (100 mL×3). The organics were combined, washed with water and brine, dried with Na₂SO₄, filtered, and concentrated to give 4-nitro-3-(2,2,3,3,3-pentafluoropropoxy) benzoic acid that was directly used for the next step without further purification.

Step II: To a solution of crude 4-nitro-3-(2,2,3,3,3-pentafluoropropoxy) benzoic acid synthesized above in DCM (600 mL), oxalyl chloride (33.4 mL, 390 mmol, 150 mol %) was added at room temperature. 2 mL DMF was added dropwise and slowly to the reaction mixture. The reaction mixture was stirred at room temperature until all the solid was dissolved and the mixture became clear. The reaction mixture was concentrated under vacuum to give acid chloride that was directly used for the next step without purification.

Step III: To a solution of diethyl aminomalonate hydrochloride (55.1 g, 260 mmol, 100 mol %) in DCM (600 mL), triethylamine (150 mL) was added at 0° C. under vigorous stirring. The above-synthesized 4-nitro-3-(2,2,3,3,3-pentafluoropropoxy) benzoyl chloride in 200 mL DCM was then added dropwise to the reaction mixture. The reaction mixture was stirred under the same temperature for 1 h, monitored by TLC. After completion, the mixture was poured into 500 mL water and vigorously stirred for 5 mins. The organic phase was separated and washed with water (500 mL×3). The organic solution was concentrated under vacuum to around 200 mL. The precipitation was collected by filtration and washed with hexane (200 mL×3). The solid was dried under vacuum to give 16 (109.69 g, 89% Yield) as a light-yellow solid that can be used for the next step without further purification.

### Representative Procedure for Substitution

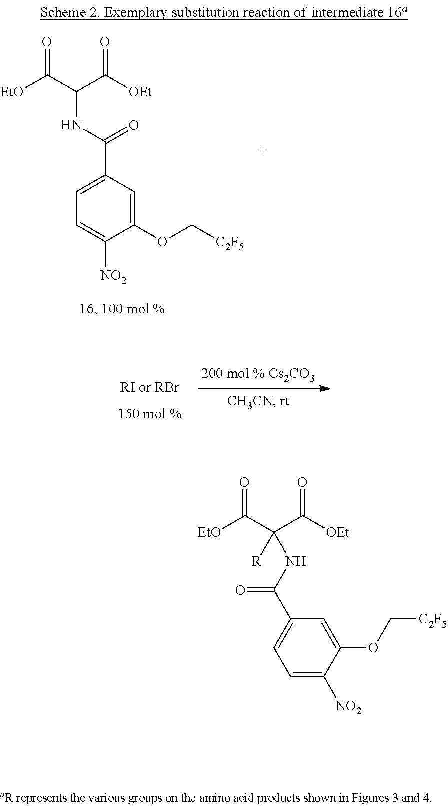

To a solution of 16 (100 mol %) in CH₃CN (0.1 M), Cs₂CO₃ (200 mol %) and RI or RBr (150 mol %) were added at room temperature. The resulting mixture was stirred at room temperature to full conversion, as monitored by TLC. CH₃CN was then removed under vacuum, and aqueous 1 M HCl solution (0.1 M as to 16) was added to the residue. The mixture was stirred for 5 mins and extracted with EtOAc. The combined organic solvents were washed with water and brine, dried with Na₂SO₄, filtered, and concentrated. The residue was subjected to flash column chromatography (hexane/ethyl acetate) to give the disubstituted malonic ester.

### Representative Procedure for Hydrolysis

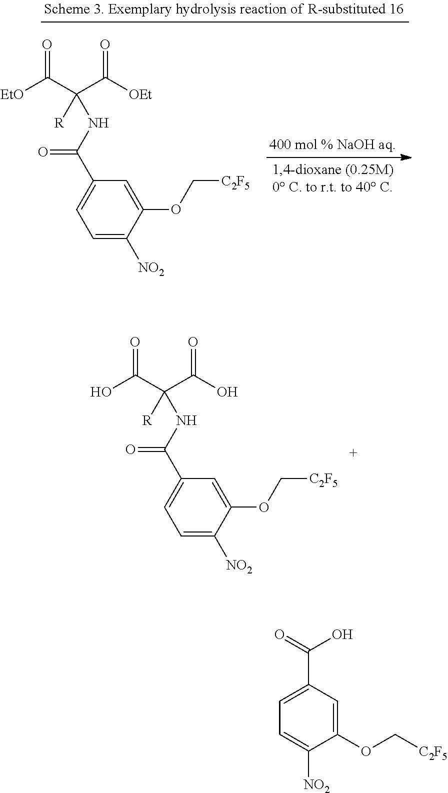

To a solution of disubstituted diethyl 2-(4-nitro-3-(2,2,3,3,3-pentafluoropropoxy)benzamide) malonate (100 mol %) in 1,4-dioxane (0.5 M), 2 M NaOH aqueous solution (400 mol %) was added dropwise at 0° C. After the resulting mixture was warmed to room temperature, it was stirred at 40° C. until hydrolysis finished, as monitored by TLC. 1,4-dioxane was removed under vacuum, and the aqueous layer was acidified by 1 M HCl aqueous solution to pH 6-7 and washed with diethyl ether to remove the unreacted malonic acid half esters and 4-nitro-3-(2,2,3,3,3-pentafluoropropoxy) benzoic acid until only malonic acid remained in aqueous, as monitored by TLC. Then the aqueous phase was acidified by 1 M HCl aqueous to pH 2-3 and extracted with diethyl ether for three times. The organic phase was washed with brine, dried over MgSO₄, and filtered and evaporated under vacuum without further purification to give aminomalonic acid.

### Synthesis of Exemplary Substrates

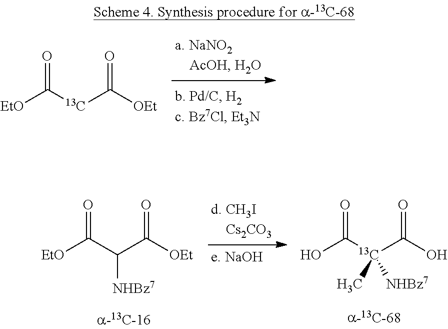

Step a: Diethyl malonate-2-¹³C (1.0 g, 6.3 mmol) was dissolved in a mixture of water (2.0 mL) and acetic acid (1.5 mL), and cooled to 0° C. Sodium nitrite (2.20 g, 31.5 mmol, 500 mol %) was added portion wise to the reaction mixture. The mixture was stirred at room temperature overnight. After reaction completion as indicated by TLC, the reaction mixture was poured into 30 mL water and extracted with diethyl ether (30 mL×3). The combined organic phase was treated with saturated sodium bicarbonate solution carefully and stirred until the aqueous layer became lightly yellow. The organic layer was separated (retention factor (“R_{f}”)=0.4 (hexane/EtOAc 5:1)), washed with brine, dried with Na₂SO₄, filtered, and concentrated to give corresponding oxime (1.2267 g, quantitative yield). The oxime was used directly in the next step without further purification.

Step b: The above-synthesized oxime and Pd/C (10%, 100 mg) was mixed in a 50 mL round bottom flask. The flask was sealed with a rubber septum and purged with nitrogen for 10 mins. 15 mL methanol was added into the reaction mixture under a nitrogen balloon. The reaction mixture was purged with hydrogen for 15 mins and stirred at room temperature overnight under hydrogen. The reaction mixture was filtered through a plug of Celite, and eluted with EtOAc. The filtrate was concentrated to give diethyl aminomalonate-2-¹³C (0.9067 g, 82% Yield). The amine was used directly in the next step without further purification. R_{f}=0.2 (hexane/EtOAc 1:1).

Step c: The above-synthesized aminomalonic ester (0.9067 g, 5.2 mmol) and triethylamine (2.0 mL, 14 mmol, 270 mol %) were dissolved in 50 mL DCM. Bz⁷Cl (prepared from 7.3 mmol Bz⁷OH as described in the general procedure) in 20 mL DCM was added to the reaction mixture dropwise at 0° C. The reaction mixture was stirred at 0° C., and monitored by TLC. Once completed, the mixture was poured into 50 mL water and extracted with DCM (25 mL×3). The combined organics were washed with water and brine, dried with Na₂SO₄, filtered, and concentrated. The residue was purified by flash column chromatography to give α-¹³C-16 (1.7428 g, 71% Yield) as colorless oil. R_{f}=0.2 (hexane/EtOAc 4:1).

Step d: To a solution of α-¹³C-16 (1.7428 g, 3.7 mmol) in 50 mL CH₃CN, Cs₂CO₃ (2.41 g, 7.4 mmol, 200 mol %) and iodomethane (781 mg, 5.5 mmol, 150 mol %) were added at room temperature. The resulting mixture was stirred at room temperature to full conversion, as monitored by TLC. The CH₃CN was then removed under vacuum, and aqueous 1 M HCl solution (50 mL) was added to the residue. The mixture was stirred for 5 mins and extracted with EtOAc (25 mL×3). The combined organic solvents were washed with water and brine, dried with Na₂SO₄, filtered, and concentrated. The residue was submitted to flash column chromatography to give diester of α-¹³C-68 (1.3762 g, 77% Yield) as colorless oil. R_{f}=0.5 (hexane/EtOAc 3:1).

α-¹³C-68 was obtained from the above diester using the general hydrolysis procedure (2.5 mmol scale of hydrolysis, 0.8426 g, 78% Yield) white solid. R_{f}=0.1 (DCM/MeOH 5:1).

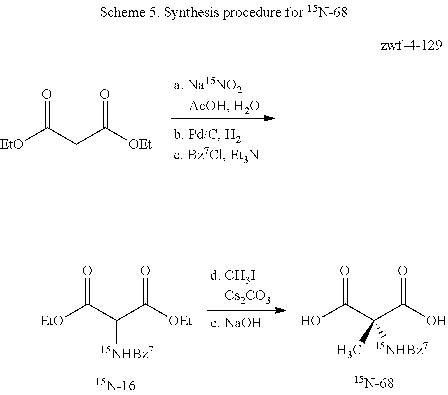

Step a: Diethyl malonate (1.0 g, 6.3 mmol) was dissolved in a mixture of water (2.0 mL) and acetic acid (1.5 mL), and cooled to 0° C. Sodium nitrite-¹⁵N (2.0 g, 28.6 mmol, 450 mol %) was added portion wise to the reaction mixture. The mixture was stirred at room temperature overnight. After reaction completion as indicated by TLC, the reaction mixture was poured into 30 mL water and extracted with diethyl ether (30 mL×3). The combined organic phase was treated with saturated sodium bicarbonate solution carefully and stirred until the aqueous layer became lightly yellow. The organic layer was separated, washed with brine, dried with Na₂SO₄, filtered, and concentrated to give corresponding oxime (1.1771 g, 99% Yield). The oxime was used directly in the next step without further purification. R_{f}=0.4 (hexane/EtOAc 5:1).

Step b: The above-synthesized oxime and Pd/C (10%, 100 mg) were mixed in a 50 mL round bottom flask. The flask was sealed with a rubber septum and purged with nitrogen for 10 mins. 15 mL methanol was added under a nitrogen balloon. The reaction mixture was purged with hydrogen for 15 mins and stirred at room temperature overnight under hydrogen. The reaction mixture was filtered through a plug of Celite, and eluted with EtOAc. The filtrate was concentrated to give diethyl aminomalonate-¹⁵N (0.8805 g, 80% Yield). The amine was used directly in the next step without further purification. R_{f}=0.2 (hexane/EtOAc 1:1).

Step c: The above-synthesized aminomalonic ester (0.8805 g, 5.0 mmol) and triethylamine (2.0 mL, 14 mmol, 270 mol %) were dissolved in 50 mL DCM. Bz⁷Cl (prepared from 7.3 mmol Bz⁷OH as described in the general procedure) in 20 mL DCM was added to the reaction mixture dropwise at 0° C. The reaction mixture was stirred at 0° C., and monitored by TLC. Once completed, the mixture was poured into 50 mL water and extracted with DCM (25 mL×3). The combined organics were washed with water and brine, dried with Na₂SO₄, filtered, and concentrated. The residue was purified by flash column chromatography to give ¹⁵N-16 (1.4331 g, 61% Yield) as colorless oil. R_{f}=0.2 (hexane/EtOAc 4:1).

Step d: To a solution of α-¹³C-16 (1.4331 g, 3.0 mmol) in 40 mL CH₃CN, Cs₂CO₃ (1.96 g, 6.0 mmol, 200 mol %) and iodomethane (639 mg, 4.5 mmol, 150 mol %) were added at room temperature. The resulting mixture was stirred at room temperature to full conversion, as monitored by TLC. The CH₃CN was then removed under vacuum, and aqueous 1 M HCl solution (50 mL) was added to the residue. The mixture was stirred for 5 mins and extracted with EtOAc (25 mL×3). The combined organic solvents were washed with water and brine, dried with Na₂SO₄, filtered, and concentrated. The residue was submitted to flash column chromatography to give diester of ¹⁵N-68 (897.3 mg, 61% Yield) as colorless oil. R_{f}=0.5 (hexane/EtOAc 3:1).

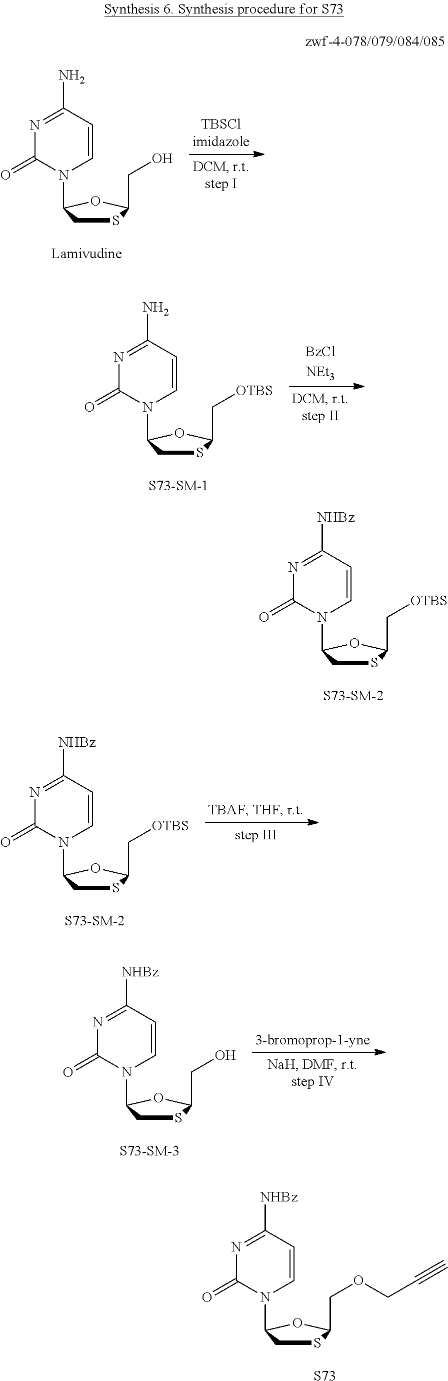

Step I: To a solution of Lamivudine (2.2972 g, 10 mmol, 100 mol %) and TBSCl (tert-butyldimethylsilyl chloride) (1.8097 g, 12 mmol, 120 mol %) in DCM (80 mL), imidazole (1.0332 g, 15 mmol, 150 mol %) was added. The reaction mixture was stirred at room temperature for 12 h and monitored by TLC. After reaction completion, the mixture was poured into 100 mL water, and the aqueous phase was extracted with DCM (50 mL×3). The combined organic phase was washed with brine, dried over MgSO₄, and filtered, and evaporated under vacuum without further purification to give S73-SM-1 as a light-yellow oil that can be used for the next step without further purification. R_{f}=0.7 (DCM/MeOH 5:1).

Step II: To a solution of all S73-SM-1 from step I in DCM (50 mL), triethylamine (2.8 mL, 20 mmol, 200 mol %) was added at 0° C. under vigorous stirring. BzCl (1.4 mL, 12 mmol, 120 mol %) was added dropwise to the reaction mixture. The reaction was warmed to the room temperature and stirred for 2 h, and monitored by TLC. After reaction completion, the mixture was poured into 50 mL water, and the aqueous phase was extracted with DCM (50 mL×3). The combined organic phase was washed with brine, dried over MgSO₄, and filtered, and evaporated under vacuum without further purification to afford S73-SM-2 as a light-yellow oil that can be used for the next step without further purification. R_{f}=0.2 (Hexane/EtOAc 1:1).

Step III: To a solution of all S73-SM-2 from step II in THF (20 mL), TBAF (tetrabutylammonium fluoride) (20 mL, 1.0 M in THF) was added dropwise within 10 min at 0° C. under argon. The resulting mixture was stirred at rt for 18 h, monitored by TLC. and concentrated under reduced pressure to give S73-SM-3 as a light-yellow oil that can be used for the next step without further purification.

Step IV: To a solution of all S73-SM-3 from step III in DMF (50 mL), NaH (0.6005 g, 15 mmol, 150 mol % (60%, disperse in mineral oil)) was added at 0° C. under vigorous stirring. The reaction mixture was warmed to the room temperature and stirred for 0.5 h. 3-bromopropyne (1.3 mL, 15 mmol, 150 mol %) was added dropwise to the reaction mixture at 0° C. The reaction mixture was warmed to the room temperature and stirred for 2 h, and monitored by TLC. After reaction completion, the mixture was quenched by 10 mL saturated NH₄Cl aqueous and extracted with EtOAc (50 mL×3). The combined organic phase was washed with brine, dried over MgSO₄, filtered, and evaporated under vacuum without further purification to give S73 as a light-yellow oil.

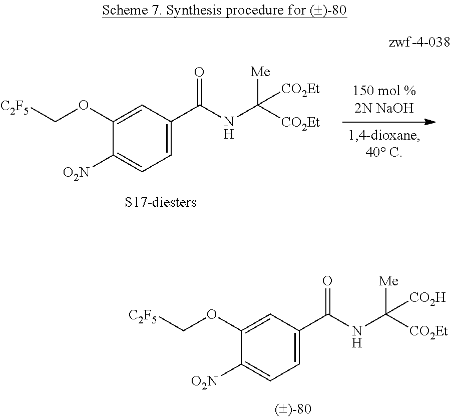

To a solution of S17-diesters (1.4592 g, 3 mmol, 100 mol %) in 1,4-dioxane (0.5 M), 2 M NaOH aqueous solution (2.25 mL, 4.5 mmol, 150 mol %) was added dropwise at 0° C. After the resulting mixture was warmed to room temperature, it was stirred at 40° C. until hydrolysis finished, as monitored by TLC. 1,4-dioxane was removed under vacuum. The aqueous layer was washed three times with diethyl ether to remove the unreacted starting material, acidified by 1 M HCl aqueous to pH=6-7, and washed with diethyl ether to remove 4-nitro-3-(2,2,3,3,3-pentafluoropropoxy) benzoic acid until only malonic acid remained in aqueous phase as monitored by TLC. Then the aqueous phase was acidified by 1 M HCl aqueous to pH=3-4 and extracted with diethyl ether for three times. The organic phase was washed with brine, dried over MgSO₄, filtered, and evaporated under vacuum without further purification to afford (±)-80.

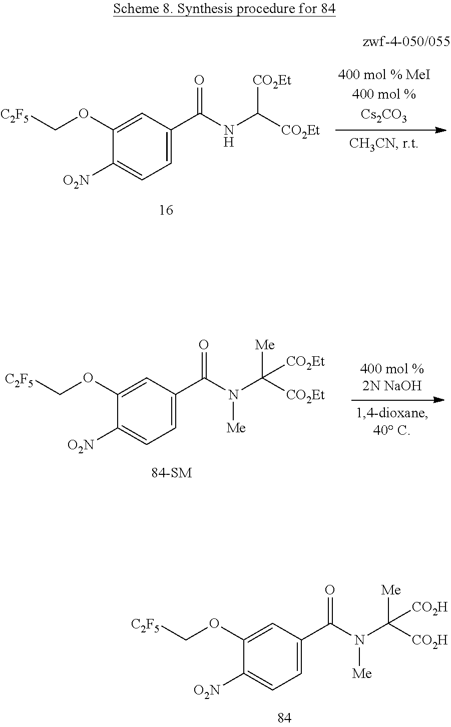

To a solution of 16 (0.9966 g, 2.1 mmol, 100 mol %) in CH₃CN (0.1 M), Cs₂CO₃ (2.6125 g, 8.0 mmol, 400 mol %) and Mel (0.5 mL, 8.0 mmol, 400 mol %) were added at room temperature. The resulting mixture was stirred at room temperature to full conversion, as monitored by TLC. CH₃CN was then removed under vacuum, and aqueous 1 M HCl solution (0.1 M as to 16) was added to the residue. The mixture was stirred for 5 mins and extracted with EtOAc. The combined organic solvents were washed with water and brine, dried with Na₂SO₄, filtered, and concentrated. The residue was submitted to flash column chromatography (hexane/ethyl acetate) to afford the disubstituted malonic ester 84-SM (1.0012 g, 95% Yield). R_{f}=0.5 (Hexane/EtOAc 2:1).

To a solution of 84-SM (1.0012 g, 2 mmol, 100 mol %) in 1,4-dioxane (0.5 M), 2 M NaOH aqueous solution (4 mL, 8 mmol, 400 mol %) was added dropwise at 0° C. After the resulting mixture was warmed to room temperature, it was stirred at 40° C. until hydrolysis finished, as monitored by TLC. 1,4-dioxane was removed under vacuum, and the aqueous layer was acidified by 1 M HCl aqueous to pH=6-7 and washed with diethyl ether to remove the unreacted malonic acid half esters and 4-nitro-3-(2,2,3,3,3-pentafluoropropoxy) benzoic acid until only malonic acid remained in aqueous phase as monitored by TLC. Then the aqueous phase was acidified by 1 M HCl aqueous to pH=2-3 and extracted with diethyl ether for three times. The organic phase was washed with brine, dried over MgSO₄, filtered, and evaporated under vacuum without further purification to afford 84.

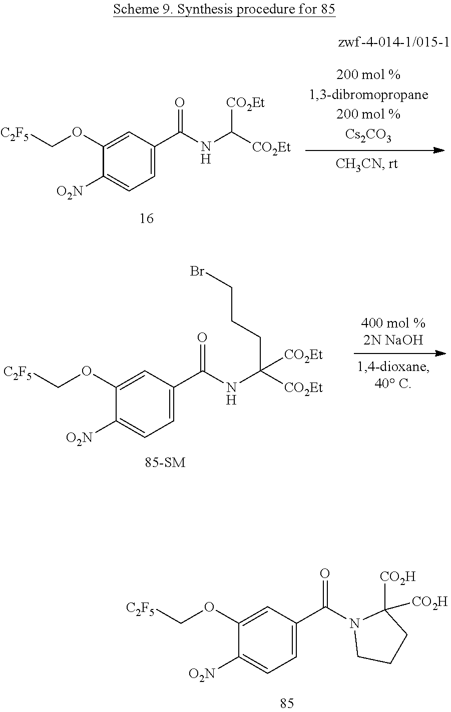

To a solution of 16 (7.0845 g, 15 mmol, 100 mol %) in CH₃CN (0.1 M), Cs₂CO₃ (9.7850 g, 30 mmol, 200 mol %) and 1,3-dibromopropane (3 mL, 30 mmol, 200 mol %) were added at room temperature. The resulting mixture was stirred at room temperature to full conversion, as monitored by TLC. The CH₃CN was then removed under vacuum, and aqueous 1 M HCl solution (0.1 M as to 16) was added to the residue. The mixture was stirred for 5 mins and extracted with EtOAc. The combined organic solvents were washed with water and brine, dried with Na₂SO₄, filtered, and concentrated. The residue was subjected to flash column chromatography (hexane/ethyl acetate) to give the disubstituted malonic ester 85-SM (6.5103 g, 73% Yield). R_{f}=0.7 (Hexane/EtOAc 2:1).

To a solution of 85-SM (2.1522 g, 3.6 mmol, 100 mol %) in 1,4-dioxane (0.5 M), 2 M NaOH aqueous solution (7.2 mL, 14.4 mmol, 400 mol %) was added dropwise at 0° C. After the resulting mixture was warmed to room temperature, it was stirred at 40° C. until hydrolysis was finished, as monitored by TLC. 1,4-dioxane was removed under vacuum, and the aqueous layer was acidified by 1 M HCl aqueous to pH=6-7 and washed with diethyl ether to remove the unreacted malonic acid half esters and 4-nitro-3-(2,2,3,3,3-pentafluoropropoxy) benzoic acid until only malonic acid remained in aqueous phase as monitored by TLC. Then the aqueous phase was acidified by 1 M HCl aqueous to pH=2-3 and extracted with diethyl ether for three times. The organic phase was washed with brine, dried over MgSO₄, filtered, and evaporated under vacuum without further purification to afford 85.

### General Procedure for Asymmetric Catalytic Reactions

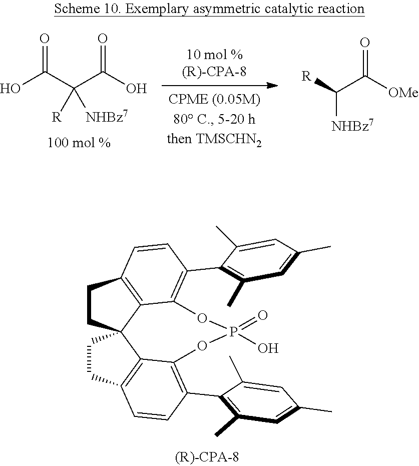

Malonic acid (0.2 mmol, 100 mol %) and (R)-CPA-8 (11.0 mg, 0.02 mmol, 10 mol %) were added into an oven-dried 25 mL Schlenk tube. The tube was sealed with a rubber septum and evacuated/refilled with nitrogen for three times. 4 mL CPME was added to the tube via syringe under nitrogen atmosphere, and the reaction mixture was stirred at 80° C. for 5-20 h. After the reaction mixture was cooled to room temperature, 0.8 mL MeOH and 0.8 mL 2 M TMSCHN₂ (800 mol %) were added to the reaction mixture, and the reaction mixture was stirred for an additional hour. The solvent was removed under vacuum and the filtrate was purified by flash column chromatography (hexanes/ethyl acetate) to obtain the decarboxylation product.

### Large-Scale Synthesis of Alanine Hydrochloride Salt

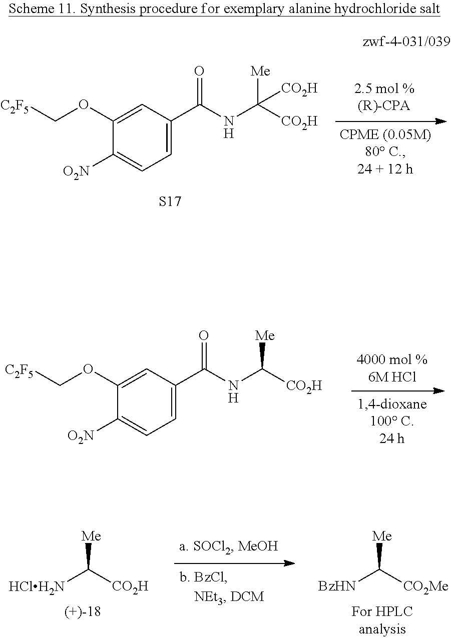

To an oven-dried 100 mL round bottom flask, (R)-CPA-8 (41.8 mg, 0.075 mmol, 2.5 mol %) was added. The flask was sealed with a rubber septum and evacuated/refilled with nitrogen for three times. 12 mL of CPME was added to the flask via syringe under nitrogen atmosphere, which was then stirred at 80° C. in an oil bath.

S17 (3.0 mmol, 1.2912 g, 100 mol %) was dissolved in 48 mL CPME and added to the reaction mixture dropwise using a syringe pump for 24 h, and the reaction mixture was stirred at the same temperature for another 12 h. The reaction mixture was cooled to room temperature and the solvent was removed under vacuum. The residue was directly used in the next step.

The residue was dissolved in 1,4-dioxane (6 mL). 6 M HCl (20 mL, 4000 mol %) was added to the reaction mixture at 0° C. The mixture was warmed to room temperature and then stirred at 100° C. for 24 h. The reaction mixture was cooled to room temperature and washed with diethyl ether (10 mL×3). The aqueous phase was concentrated under vacuum to give amino acid hydrochloride (+)-18⁵⁸ as a solid (0.3136 g, 83% Yield). R_{f}=0.2 (DCM/MeOH 1:1).

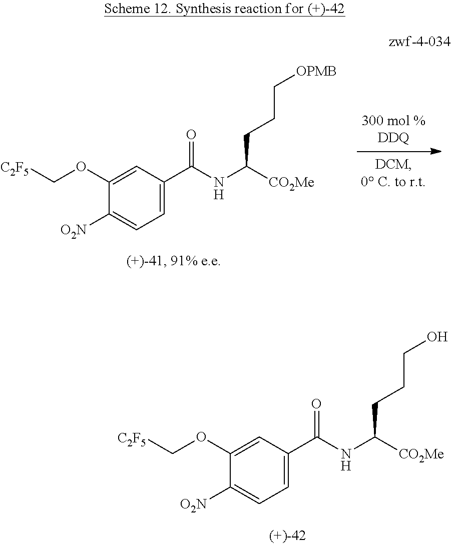

To an oven-dried 25 mL Schlenk tube, (+)-41 (226.0 mg, 0.4 mmol, 91% e.e., 100 mol %) was added and sealed with a rubber septum and evacuated/refilled with nitrogen for three times. 2 mL DCM was added to the tube via syringe under nitrogen atmosphere. The reaction mixture was cooled to 0° C., then DDQ (273.2 mg, 1.2 mmol, 300 mol %) was added and the reaction mixture was stirred at room temperature for 10 h. Then 1 mL saturated NaHCO₃ aqueous was added to quench the reaction, which was then extracted with DCM (5 mL×6), washed with brine, dried over MgSO₄, and filtered. The filtrate was concentrated under vacuum and purified by flash column chromatography to give (+)-42 (151.5 mg, 85% Yield) as colorless oil. R_{f}=0.2 (Hexane/EtOAc 1:1).

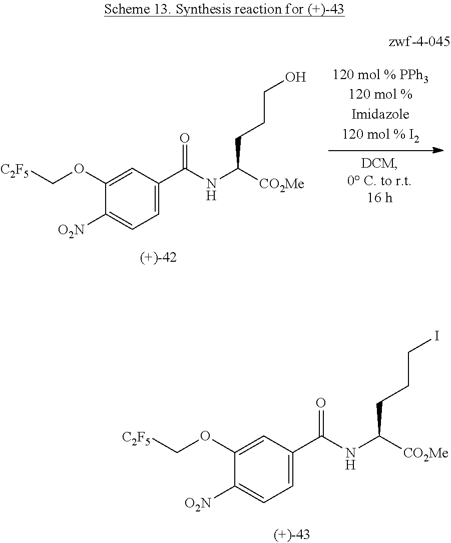

To an oven-dried 25 mL Schlenk tube, (+)-42 (88.6 mg, 0.2 mmol, 91% e.e., 100 mol %), PPh₃ (63.2 mg, 0.24 mmol, 120 mol %), imidazole (16.4 mg, 0.24 mmol, 120 mol %), were added and sealed with a rubber septum and evacuated/refilled with nitrogen for three times. 1 mL DCM was added to the tube via syringe under nitrogen atmosphere. The reaction mixture was cooled to 0° C., then I₂ (60.8 mg, 0.24 mmol, 120 mol %) was added and the reaction mixture was stirred at room temperature for 16 h. Then 1 mL saturated Na₂S₂O₃ aqueous solution was added to quench the reaction, which was extracted with DCM (5 mL×6), washed with brine, dried over MgSO₄, and filtered. The filtrate was concentrated under vacuum and purified by flash column chromatography to afford the (+)-43 (96.6 mg, 87% Yield) as white solid. R_{f}=0.6 (Hexane/EtOAc 1:1).

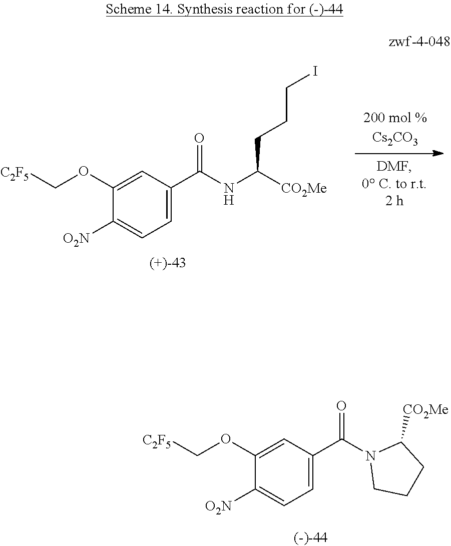

To an oven-dried 25 mL Schlenk tube, (+)-43 (110.5 mg, 0.2 mmol, 91% e.e., 100 mol %), was added and sealed with a rubber septum and evacuated/refilled with nitrogen for three times. 1 mL DMF was added to the tube via syringe under nitrogen atmosphere. The reaction mixture was cooled to 0° C., then Cs₂CO₃ (130.8 mg, 0.4 mmol, 200 mol %) was added and the reaction mixture was stirred at room temperature for 2 h. Then 1 mL H₂O was added to quench the reaction, which was extracted with ethyl acetate (5 mL×3), washed with brine, dried over MgSO₄, and filtered. The filtrate was concentrated under vacuum and purified by flash column chromatography to give the (−)-44 (75.2 mg, 88% Yield) as white solid. R_{f}=0.3 (Hexane/EtOAc 2:1).

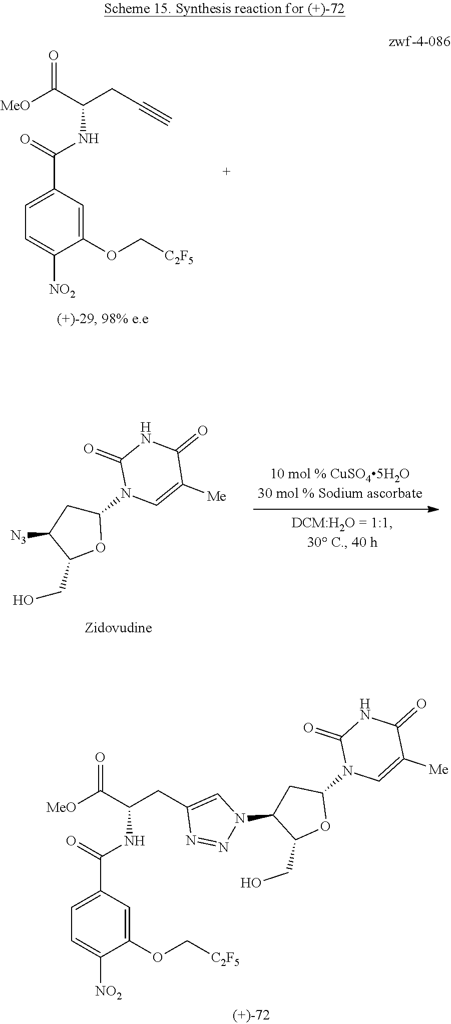

To an oven-dried 25 mL Schlenk tube, (+)-29 (63.7 mg, 0.15 mmol, 98% e.e., 100 mol %) and Zidovudine (54.1 mg, 0.225 mmol, 150 mol %) were added and sealed with a rubber septum and evacuated/refilled with nitrogen for three times. 3 mL DCM was added to the tube via syringe under nitrogen atmosphere. Sodium ascorbate (9 mg dissolved in 1.5 mL H₂O, 0.045 mmol, 30 mol %) and CuSO₄·5H₂O (3.8 mg dissolved in 1.5 mL H₂O, 0.015 mmol, 10 mol %) were added to the reaction sequentially. The reaction mixture was stirred at 30° C. for 40 h, extracted with DCM (5 mL×3), washed with brine, dried over MgSO₄, and filtered. The filtrate was concentrated under vacuum and purified by flash column chromatography to give (+)-72 (95.4 mg, 92% Yield) as colorless oil. The d.r. was determined as >20:1 using ¹H NMR analysis of crude product with CH₂Br₂ (10.5 μL) as the internal standard. R_{f}=0.3 (EtOAc 100%).

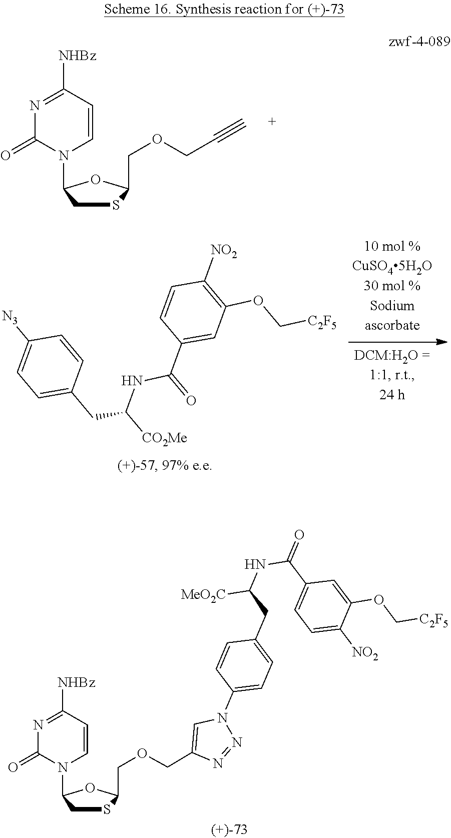

To an oven-dried 25 mL Schlenk tube, (+)-57 (77.6 mg, 0.15 mmol, 97% e.e., 100 mol %) and Lamivudine derived terminal alkyne (67.5 mg, 0.18 mmol, 120 mol %) were added and sealed with a rubber septum and evacuated/refilled with nitrogen for three times. 1.5 mL DCM was added to the tube via syringe under nitrogen atmosphere. Sodium ascorbate (9.1 mg dissolved in 0.75 mL H₂O, 0.045 mmol, 30 mol %) and CuSO₄·H₂O (3.9 mg dissolved in 0.75 mL H₂O, 0.015 mmol, 10 mol %) were added to the reaction sequentially. The reaction mixture was stirred at room temperature for 24 h, extracted with DCM (5 mL×3), washed with brine, dried over MgSO₄, and filtered. The filtrate was concentrated under vacuum and purified by flash column chromatography to afford the (+)-73 (115.3 mg, 86% Yield) as colorless oil. The d.r. was determined as >20:1 using ¹H NMR analysis of crude product with CH₂Br₂ (10.5 μL) as the internal standard. R_{f}=0.4 (Hexane/EtOAc 1:3).

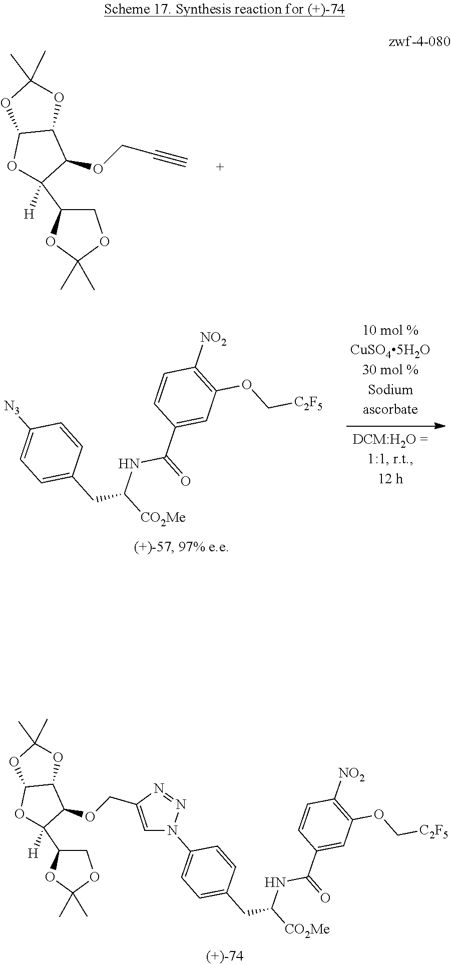

To an oven-dried 25 mL Schlenk tube, (+)-57 (77.5 mg, 0.15 mmol, 97% e.e., 100 mol %) and diacetone-D-glucose derived terminal alkyne (53.5 mg, 0.18 mmol, 120 mol %) were added and sealed with a rubber septum and evacuated/refilled with nitrogen for three times. 1.5 mL of DCM was added to the tube via syringe under nitrogen atmosphere. Sodium ascorbate (9 mg dissolved in 0.75 mL H₂O, 0.045 mmol, 30 mol %) and CuSO₄·H₂O (3.8 mg dissolved in 0.75 mL H₂O, 0.015 mmol, 10 mol %) were added to the reaction sequentially. The reaction mixture was stirred at room temperature for 12 h, extracted with DCM (5 mL×3), washed with brine, dried over MgSO₄, and filtered. The filtrate was concentrated under vacuum and purified by flash column chromatography to give (+)-74 (114.9 mg, 94% Yield) as a colorless oil. The d.r. was determined as >20:1 using ¹H NMR analysis of crude product with CH₂Br₂ (10.5 μL) as the internal standard. R_{f}=0.2 (Hexane/EtOAc 2:1).

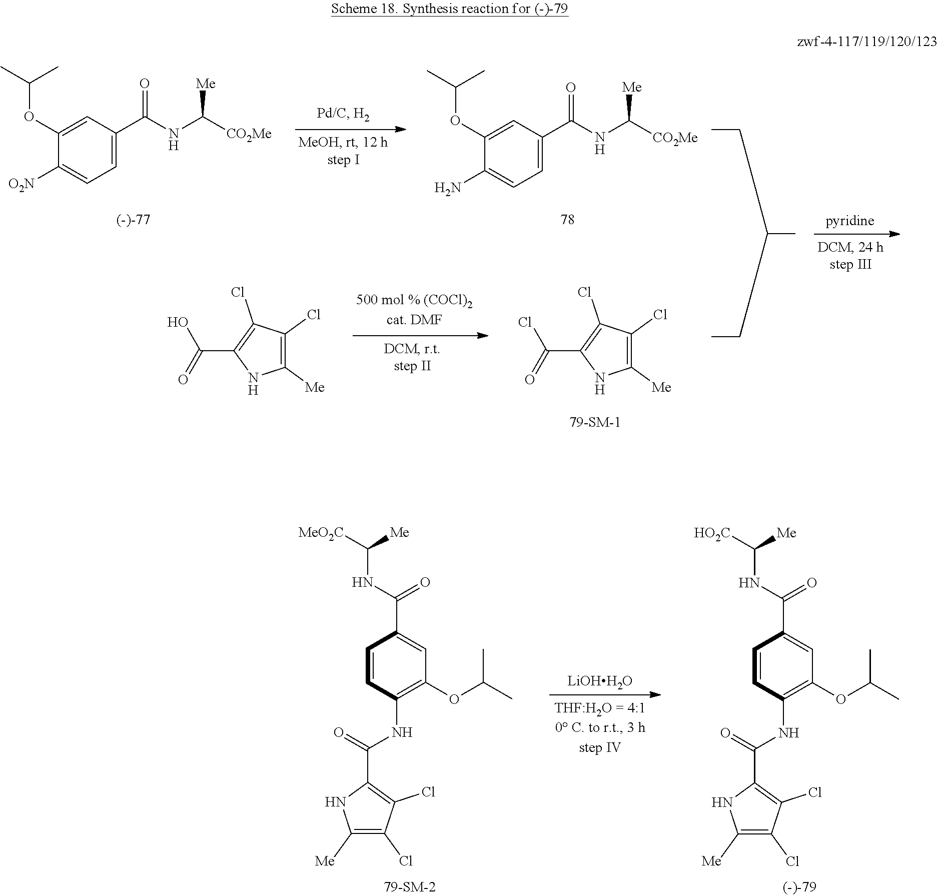

Step I: To an oven-dried 100 mL flask, (−)-77 (232.5 mg, 0.75 mmol, 94% e.e.) and Pd/C dry powder (10%, 50 mg) were added and sealed with a rubber septum and evacuated/refilled with nitrogen for three times. 40 mL of MeOH was added to the flask via syringe under nitrogen atmosphere. The reaction mixture was purged by H₂ for 5 min with a H₂ balloon, then sealed and stirred under H₂ atmosphere with a H₂ balloon. After stirred at room temperature for 12 h, the reaction mixture was filtrated with Celite and the filtrate was concentrated under vacuum and purified by flash column chromatography to afford 78 (176.2 mg, 84% Yield) as a colorless oil, which was directly use in next step. R_{f}=0.3 (Hexane/EtOAc 1:1).

Step II: To an oven-dried 100 mL flask, 3,4-dichloro-5-methyl-1H-pyrrole-2-carboxylic acid (116.7 mg, 0.6 mmol, 100 mol %) and dry DCM (18 mL) were added and sealed with a rubber septum and evacuated/refilled with nitrogen for three times. Oxalyl chloride (254 μL dissolved in 4 mL dry DCM, 3 mmol, 500 mol %) was added to the flask via syringe under nitrogen atmosphere. 5 drops of DMF were added carefully into the reaction mixture, which was stirred at room temperature for 24 h. After that, the solvent was removed under vacuum, and the residue (79-SM-1) was redissolved in 4 mL DCM, which was directly use in next step without further purification.

Step III: To an oven-dried 50 mL flask, 78 (168.2 mg, 0.6 mmol, 100 mol %) and dry DCM (4 mL) were added and sealed with a rubber septum and evacuated/refilled with nitrogen for three times. 79-SM-1 (dissolved in 4 mL dry DCM, from step II) and anhydrous pyridine (4 mL) were added to the flask sequentially under nitrogen atmosphere. After stirred at room temperature for 24 h, the reaction mixture was concentrated under vacuum, then redissolved in DCM (50 mL), washed with 1 M HCl, brine, dried over MgSO₄, and filtered. The filtrate was concentrated under vacuum and purified by flash column chromatography to give 79-SM-2 (72.7 mg, 27% Yield) as a yellow oil, which was directly use in next step. R_{f}=0.5 (Hexane/EtOAc 1:1).

Step IV: To a stirred solution of 79-SM-2 (60.0 mg, 0.13 mmol, 100 mol %) in THF/H₂O (1.2 mL 4:1 mixture), LiOH·H₂O (11.5 mg, 0.26 mmol, 200 mol %) was added. After stirred at room temperature for 3 h, the reaction mixture was concentrated under vacuum to remove THF, then redissolved in H₂O (5 mL), and washed with diethyl ether (5 mL×3). The aqueous layer was acidified by 1 M HCl aqueous to pH=2-3 and extracted with diethyl ether (5 mL×3). The organic layer was washed with brine, dried over MgSO₄, and filtered. The filtrate was concentrated under vacuum and purified by flash column chromatography to afford the (−)-79 (41.1 mg, 71% Yield) as white solid. R_{f}=0.3 (DCM/MeOH 5:1).

### Results and Discussions

### Characterization Data

### Characterization of Substrates

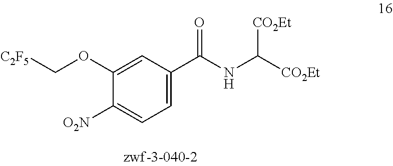

16 (260 mmol scale, 109.69 g, 89% Yield) light-yellow solid. R_{f}=0.4 (Hexane/EtOAc 2:1). ¹H NMR (400 MHz, CDCl₃) δ 7.94 (d, J=8.0 Hz, 1H), 7.66 (s, 1H), 7.57 (d, J=8.0 Hz, 1H), 7.26 (s, 1H), 5.30 (d, J=6.4 Hz, 1H), 4.61 (t, J=11.8 Hz, 2H), 4.46-4.20 (m, 4H), 1.34 (t, J=6.8 Hz, 6H). ¹³C NMR (150 MHz, CDCl₃) δ 166.0, 164.3, 150.2, 142.4, 138.1, 126.0, 120.7, 115.1, 66.2 (t, J=28.5 Hz), 63.0, 56.9, 13.9. ¹⁹F NMR (376 MHz, CDCl₃) δ −83.4 (s), −123.2 (t, J=11.8 Hz). IR (neat, cm⁻¹) 3293, 2992, 1748, 1530, 1190. HRMS (ESI) calcd C₁₇H₁₈F₅N₂O₈⁺ [M+H]⁺: 473.0978. Found: 473.0977.

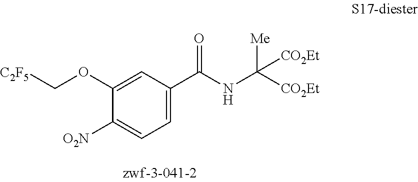

S17-diester (13.0 mmol scale, 5.4544 g, 88% Yield) white solid. R_{f}=0.6 (Hexane/EtOAc 2:1). ¹H NMR (400 MHz, CDCl₃) δ 7.94 (d, J=8.4 Hz, 1H), 7.75-7.60 (m, 2H), 7.54 (d, J=8.0 Hz, 1H), 4.62 (t, J=11.8 Hz, 2H), 4.30 (q, J=7.1 Hz, 4H), 1.87 (s, 3H), 1.29 (t, J=7.0 Hz, 6H). ¹³C NMR (150 MHz, CDCl₃) δ 168.3, 163.2, 150.3, 142.3, 138.7, 126.1, 120.4, 115.0, 66.2 (t, J=28.2 Hz), 63.4, 62.9, 20.5, 13.9. ¹⁹F NMR (376 MHz, CDCl₃) δ −83.4 (s), −123.2 (t, J=12.4 Hz). IR (neat, cm⁻¹) 3405, 2987, 1743, 1534, 1190. HRMS (ESI) calcd C₁₀H₂₀F₅N₂O₈⁺ [M+H]⁺: 487.1134. Found: 487.1135.

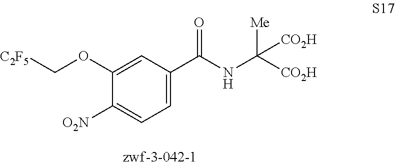

S17 (11.2 mmol scale of hydrolysis, 3.8477 g, 80% Yield) as white solid. R_{f}=0.1 (DCM/MeOH 5:1). ¹H NMR (400 MHz, DMSO-d₆) δ 8.55 (s, 1H), 8.02 (d, J=8.4 Hz, 1H), 7.87 (s, 1H), 7.66 (d, J=8.4 Hz, Hz, 1H), 5.19 (t, J=13.0 Hz, 2H), 1.69 (s, 3H). ¹³C NMR (150 MHz, DMSO-d₆) δ 170.0, 163.7, 149.0, 141.4, 138.7, 125.3, 121.7, 118.4 (qt, J₁=284.6 Hz, J₂=34.8 Hz), 115.0, 112.7 (tq, J₁=252.8 Hz, J₂=37.4 Hz), 65.1 (t, J=27.5 Hz), 63.2, 21.0. ¹⁹F NMR (376 MHz, CDCl₃) δ −82.7, −122.5 (t, J=11.7 Hz). IR (neat, cm⁻¹) 3460 (br), 3414, 1693, 1520, 1196. HRMS (ESI) calcd C₁₄H₁₂F₅N₂O₈⁺ [M+H]⁺: 431.0508. Found: 431.0506.

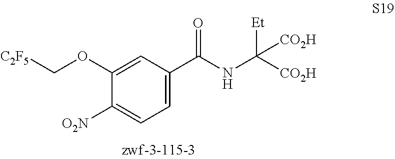

S19 (5.9 mmol scale of hydrolysis, 1.8778 g, 73% Yield) white solid. R_{f}=0.1 (DCM/MeOH 5:1). ¹H NMR (400 MHz, CD₃CN) δ 7.92 (d, J=8.4 Hz, 1H), 7.83 (s, 1H), 7.69 (s, 1H), 7.62 (d, J=8.0 Hz, 1H), 4.84 (t, J=12.6 Hz, 2H), 2.30 (q, J=7.1 Hz, 2H), 0.91 (t, J=7.2 Hz, 3H). ¹³C NMR (100 MHz, CD₃CN) δ 170.6, 165.6, 150.6, 143.2, 139.2, 126.6, 122.4, 115.8, 67.6, 66.7 (t, J=27.8 Hz), 27.4, 8.4. ¹⁹F NMR (376 MHz, CD₃CN) δ −84.2 (s), −124.1 (t, J=13.2 Hz). IR (neat, cm⁻¹) 3384, 3091, 1733, 1516, 1149. HRMS (ESI) calcd C₁₅H₁₄F₅N₂O₈⁺ [M+H]⁺: 445.0665. Found: 445.0668.

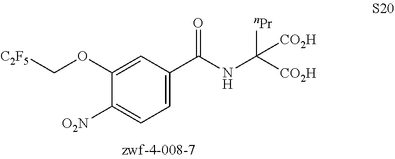

S20 (5.3 mmol scale of hydrolysis, 1.6774 g, 69% Yield) white solid. R_{f}=0.1 (DCM/MeOH 5:1). ¹H NMR (400 MHz, CD₃CN) δ 7.92 (d, J=8.4 Hz, 1H), 7.81 (s, 1H), 7.69 (s, 1H), 7.62 (d, J=8.0 Hz, 1H), 4.84 (t, J=12.6 Hz, 2H), 2.23 (t, J=8.2 Hz, 2H), 1.38-1.25 (m, 2H), 0.93 (t, J=7.2 Hz, 3H). ¹³C NMR (100 MHz, CD₃CN) δ 170.8, 165.6, 150.6, 143.1, 139.2, 126.6, 122.3, 115.8, 67.1, 66.7 (t, J=28.2 Hz), 36.1, 18.0, 14.1. ¹⁹F NMR (376 MHz, CD₃CN) δ −84.2 (s), −124.1 (t, J=12.4 Hz). IR (neat, cm⁻¹) 3395, 2977, 1739, 1530, 1194. HRMS (ESI) calcd C₁₆H₁₆F₅N₂O₈⁺ [M+H]⁺: 459.0821. Found: 459.0819.

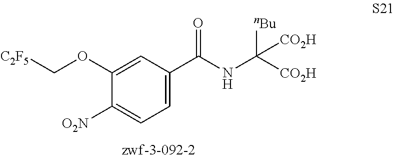

S21 (9.3 mmol scale of hydrolysis, 3.4190 g, 78% Yield) white solid. R_{f}=0.1 (DCM/MeOH 5:1). ¹H NMR (500 MHz, CD₃CN) δ 7.92 (d, J=8.0 Hz, 1H), 7.84 (s, 1H), 7.69 (s, 1H), 7.62 (d, J=8.5 Hz, 1H), 4.84 (t, J=12.5 Hz, 2H), 2.26 (t, J=8.0 Hz, 2H), 1.41-1.20 (m, 4H), 0.88 (t, J=7.0 Hz, 3H). ¹³C NMR (100 MHz, CD₃CN) δ 170.7, 165.5, 150.7, 143.2, 139.2, 126.6, 122.4, 115.8, 67.1, 66.7 (t, J=27.5 Hz), 33.7, 26.6, 23.2, 14.1. ¹⁹F NMR (376 MHz, CD₃CN) δ −84.2 (s), −124.1 (t, J=12.4 Hz). IR (neat, cm⁻¹) 3312, 2973 1756, 1538, 1207. HRMS (ESI) calcd C₁₇H₁₈F₅N₂O₈⁺ [M+H]⁺: 473.0978. Found: 473.0976.

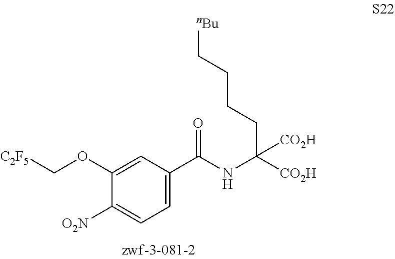

S22 (8.3 mmol scale of hydrolysis, 2.3683 g, 54% Yield) white solid. R_{f}=0.1 (DCM/MeOH 5:1). ¹H NMR (400 MHz, CD₃CN) δ 7.92 (d, J=8.4 Hz, 1H), 7.84 (s, 1H), 7.69 (s, 1H), 7.62 (d, J=8.4 Hz, 1H), 4.84 (t, J=12.6 Hz, 2H), 2.37-2.12 (m, 2H), 1.37-1.16 (m, 12H), 0.85 (t, J=6.6 Hz, 3H). ¹³C NMR (100 MHz, CD₃CN) δ 170.7, 165.5, 150.6, 143.2, 139.2, 126.6, 122.3, 115.8, 67.1, 66.7 (t, J=27.8 Hz), 33.9, 32.5, 30.0, 29.9, 29.8, 24.4, 23.3, 14.4. ¹⁹F NMR (376 MHz, CD₃CN) δ −84.2 (s), −124.1 (t, J=12.6 Hz). IR (neat, cm⁻¹) 3316, 2929, 1760, 1541 1203. HRMS (ESI) calcd C₂₁H₂₆F₅N₂O₈[M+H]⁺: 529.1604. Found: 529.1604.

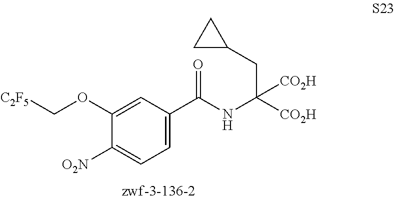

S23 (3.9 mmol scale of hydrolysis, 0.7891 g, 43% Yield) white solid. R_{f}=0.1 (DCM/MeOH 5:1). ¹H NMR (400 MHz, CD₃CN) δ 8.05-7.84 (m, 2H), 7.69 (s, 1H), 7.62 (d, J=8.4 Hz, 1H), 4.84 (t, J=12.6 Hz, 2H), 2.21 (d, J=7.2 Hz, 2H), 0.76-0.62 (m, 1H), 0.44 (q, J=6.0 Hz, 2H), 0.11 (q, J=4.9 Hz, 2H). ¹³C NMR (100 MHz, CD₃CN) δ 171.0, 165.4, 150.6, 143.1, 139.3, 126.6, 122.3, 115.8, 67.0, 66.7 (t, J=28.9 Hz), 38.6, 6.5, 4.4. ¹⁹F NMR (376 MHz, CD₃CN) δ −84.2 (s), −124.1 (t, J=12.4 Hz). IR (neat, cm⁻¹) 3400, 3116, 1731, 1531, 1193. HRMS (ESI) calcd C₁₇H₁₆F₅N₂O₈⁺ [M+H]⁺: 471.0821. Found: 471.0819.

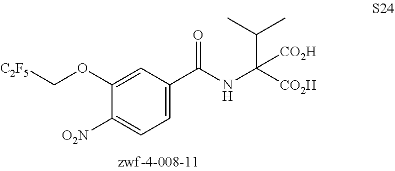

S24 (10.9 mmol scale of hydrolysis, 1.0992 g, 22% Yield) white solid. R_{f}=0.1 (DCM/MeOH 5:1). ¹H NMR (400 MHz, CD₃CN) δ 7.92 (d, J=8.0 Hz, 1H), 7.85 (s, 1H), 7.71-7.61 (m, 2H), 4.83 (t, J=12.4 Hz, 2H), 2.64-2.46 (m, 1H), 1.09 (d, J=6.8 Hz, 6H). ¹³C NMR (100 MHz, CD₃CN) δ 171.8, 166.7, 150.6, 143.1, 138.9, 126.6, 122.5, 115.8, 69.7, 66.7 (t, J=27.5 Hz), 34.6, 18.2. ¹⁹F NMR (376 MHz, CD₃CN) δ −84.2 (s), −124.1 (t, J=12.4 Hz). IR (neat, cm⁻¹) 3274, 2977, 1734, 1534, 1199. HRMS (ESI) calcd C₁₆H₁₆F₅N₂O₈⁺ [M+H]⁺: 459.0821. Found: 459.0818.

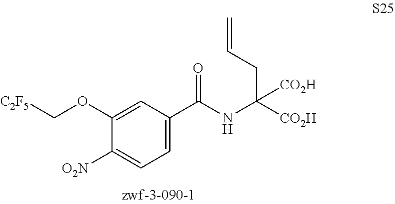

S25 (19.2 mmol scale of hydrolysis, 5.4429 g, 62% Yield) white solid. R_{f}=0.1 (DCM/MeOH 5:1). ¹H NMR (400 MHz, CD₃CN) δ 7.91 (d, J=8.4 Hz, 1H), 7.74 (s, 1H), 7.68 (s, 1H), 7.59 (d, J=8.0 Hz, 1H), 5.79-5.68 (m, 1H), 5.21-5.11 (m, 2H), 4.83 (t, J=12.6 Hz, 2H), 3.05 (d, J=7.6 Hz, 2H). ¹³C NMR (100 MHz, CD₃CN) δ 169.5, 165.4, 150.6, 143.2, 139.3, 132.1, 126.6, 122.3, 120.8, 115.8, 67.0, 66.7 (t, J=27.8 Hz), 38.0. ¹⁹F NMR (376 MHz, CD₃CN) δ −84.2 (s), −124.1 (t, J=12.4 Hz). IR (neat, cm⁻¹) 3383, 2951, 1730, 1524, 1195. HRMS (ESI) calcd C₁₆H₁₄F₅N₂O₈⁺ [M+H]⁺: 457.0665. Found: 457.0664.

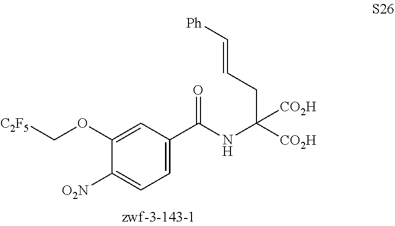

S26 (4.9 mmol scale of hydrolysis, 1.8927 g, 73% Yield) white solid. R_{f}=0.1 (DCM/MeOH 5:1). ¹H NMR (400 MHz, CD₃CN) δ 7.89 (d, J=8.4 Hz, 1H), 7.81 (s, 1H), 7.66 (s, 1H), 7.60 (d, J=8.4 Hz, 1H), 7.35 (d, J=7.2 Hz, 2H), 7.29 (t, J=7.4 Hz, 2H), 7.23 (t, J=7.0 Hz, 1H), 6.52 (d, J=16.0 Hz, 1H), 6.20-6.10 (m, 1H), 4.77 (t, J=12.6 Hz, 2H), 3.21 (d, J=7.2 Hz, 2H). ¹³C NMR (100 MHz, CD₃CN) δ 169.5, 165.6, 150.6, 143.1, 139.4, 137.9, 135.7, 129.6, 128.7, 127.2, 126.6, 123.5, 122.4, 115.8, 67.3, 66.7 (t, J=27.8 Hz), 37.4. ¹⁹F NMR (376 MHz, CD₃CN) δ −84.1 (s), −124.0 (t, J=12.6 Hz). IR (neat, cm⁻¹) 3379, 2979, 1729, 1529, 1193. HRMS (ESI) calcd C₂₂H₁₈F₅N₂O₈⁺ [M+H]⁺: 533.0978. Found: 533.0975.

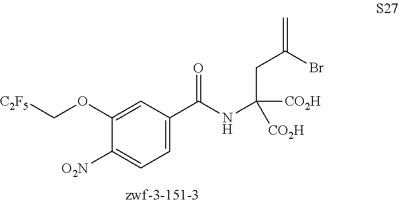

S27 (19.7 mmol scale of hydrolysis, 3.3933 g, 32% Yield) white solid. R_{f}=0.1 (DCM/MeOH 5:1). ¹H NMR (400 MHz, CD₃CN) δ 7.91 (d, J=8.4 Hz, 1H), 7.81 (s, 1H), 7.67 (s, 1H), 7.60 (d, J=8.4 Hz, 1H), 5.69 (s, 1H), 5.60 (s, 1H), 4.84 (t, J=12.6 Hz, 2H), 3.57 (s, 2H). ¹³C NMR (100 MHz, CD₃CN) δ 168.4, 165.3, 150.6, 143.2, 139.4, 126.9, 126.6, 123.7, 122.3, 115.8, 66.8, 66.7 (t, J=27.8 Hz), 43.3. ¹⁹F NMR (376 MHz, CD₃CN) δ −84.2 (s), −124.1 (t, J=12.6 Hz). IR (neat, cm⁻¹) 3390, 2992, 1734, 1530, 1199. HRMS (ESI) calcd C₁₆H₁₃F₅N₂O₈⁷⁹Br⁺ [M+H]⁺: 534.9770. Found: 534.9766.

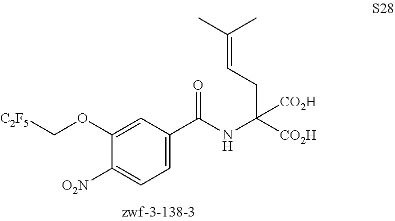

S28 (7.2 mmol scale of hydrolysis, 2.8503 g, 82% Yield) white solid. R_{f}=0.1 (DCM/MeOH 5:1). ¹H NMR (400 MHz, CD₃CN) δ 7.91 (d, J=8.4 Hz, 1H), 7.77 (s, 1H), 7.67 (s, 1H), 7.58 (d, J=8.0 Hz, 1H), 5.05 (t, J=7.4 Hz, 1H), 4.83 (t, J=12.6 Hz, 2H), 3.02 (d, J=7.6 Hz, 2H), 1.69 (s, 3H), 1.55 (s, 3H). ¹³C NMR (100 MHz, CD₃CN) δ 170.0, 165.5, 150.7, 143.2, 139.3, 138.8, 126.6, 122.3, 116.9, 115.8, 66.8, 66.7 (t, J=27.8 Hz), 32.7, 26.0, 18.1. ¹⁹F NMR (376 MHz, CD₃CN) δ −84.2 (s), −124.1 (t, J=12.6 Hz). IR (neat, cm⁻¹) 3392, 2978, 1729, 1529, 1193. HRMS (ESI) calcd C₁₈H₁₈F₅N₂O₈⁺ [M+H]⁺: 485.0978. Found: 485.0977.

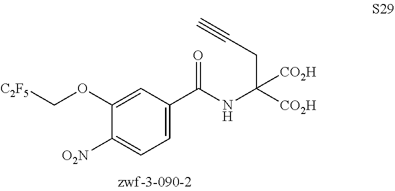

S29 (17.3 mmol scale of hydrolysis, 5.3877 g, 69% Yield) white solid. R_{f}=0.1 (DCM/MeOH 5:1). ¹H NMR (400 MHz, CD₃CN) δ 7.99-7.82 (m, 2H), 7.69 (s, 1H), 7.61 (d, J=8.4 Hz, 1H), 4.84 (t, J=12.6 Hz, 2H), 3.01 (d, J=1.6 Hz, 2H), 2.32 (s, 1H). ¹³C NMR (100 MHz, CD₃CN) δ 168.0, 165.4, 150.7, 143.3, 139.0, 126.7, 122.3, 115.8, 78.7, 73.4, 66.7 (t, J=27.8 Hz), 66.2, 24.3. ¹⁹F NMR (376 MHz, CD₃CN) δ −84.2 (s), −124.1 (t, J=12.6 Hz). IR (neat, cm⁻¹) 3391, 3293, 1953, 1733, 1521, 1198. HRMS (ESI) calcd C₁₆H₁₂F₅N₂O₈⁺ [M+H]⁺: 455.0510. Found: 455.0508.

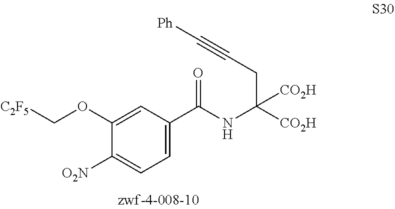

S30 (7.4 mmol scale of hydrolysis, 2.6497 g, 67% Yield) white solid. R_{f}=0.1 (DCM/MeOH 5:1). ¹H NMR (400 MHz, CD₃CN) δ 7.99 (s, 1H), 7.91 (d, J=8.4 Hz, 1H), 7.70 (s, 1H), 7.63 (d, J=8.4 Hz, 1H), 7.39-7.25 (m, 5H), 4.80 (t, J=12.4 Hz, 2H), 3.53 (s, 2H). ¹³C NMR (100 MHz, CD₃CN) δ 168.3, 165.4, 150.7, 143.2, 139.2, 132.5, 129.51, 129.46, 126.7, 123.7, 122.3, 115.8, 84.5, 84.4, 66.7 (t, J=27.9 Hz), 66.6, 25.3. ¹⁹F NMR (376 MHz, CD₃CN) δ −84.2 (s), −124.1 (t, J=12.4 Hz). IR (neat, cm⁻¹) 3405, 2924, 1739, 1530, 1199. HRMS (ESI) calcd C₂₂H₁₆F₅N₂O₈⁺ [M+H]⁺: 531.0821. Found: 531.0823.

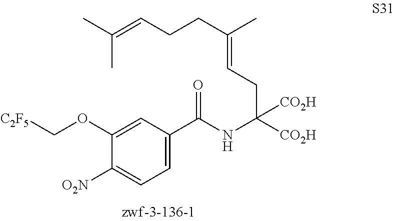

S31 (5.8 mmol scale of hydrolysis, 1.9438 g, 60% Yield) white solid. R_{f}=0.1 (DCM/MeOH 5:1). ¹H NMR (400 MHz, CD₃CN) δ 7.90 (d, J=8.4 Hz, 1H), 7.79 (s, 1H), 7.68 (s, 1H), 7.57 (d, J=8.4 Hz, 1H), 5.12-4.97 (m, 2H), 4.83 (t, J=12.6 Hz, 2H), 3.03 (d, J=7.6 Hz, 2H), 2.06-1.94 (m, 4H), 1.63 (s, 3H), 1.55 (s, 6H). ¹³C NMR (100 MHz, CD₃CN) δ 170.1, 165.6, 150.7, 143.2, 142.4, 139.2, 132.5, 126.6, 124.9, 122.2, 116.9, 115.8, 66.8, 66.7 (t, J=27.8 Hz), 40.5, 32.6, 27.2, 25.8, 17.8, 16.4. ¹⁹F NMR (376 MHz, CD₃CN) δ −84.1 (s), −124.0 (t, J=12.4 Hz). IR (neat, cm⁻¹) 3383, 2951, 1731, 1525, 1195. HRMS (ESI) calcd C₂₃H₂₆F₅N₂O₈⁺ [M+H]⁺: 553.1604. Found: 553.1606.

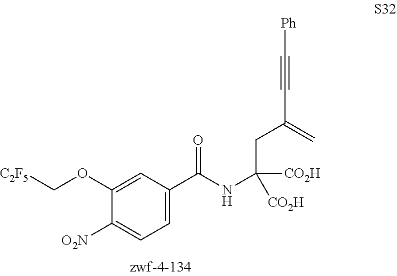

S32 (4.0 mmol scale of hydrolysis, 0.7862 g, 35% Yield) white solid. R_{f}=0.1 (DCM/MeOH 5:1). ¹H NMR (400 MHz, CD₃CN) δ 7.82 (s, 1H), 7.73 (d, J=8.4 Hz, 1H), 7.61 (s, 1H), 7.55 (d, J=8.0 Hz, 1H), 7.32-7.17 (m, 3H), 7.11 (d, J=7.2 Hz, 2H), 5.55 (s, 1H), 5.42 (s, 1H), 4.66 (t, J=12.4 Hz, 2H), 3.34 (s, 2H). ¹³C NMR (100 MHz, CD₃CN) δ 168.7, 164.9, 150.5, 143.0, 139.0, 132.1, 129.5, 129.3, 127.4, 126.5, 126.4, 123.4, 122.3, 115.7, 90.7, 90.2, 67.3, 66.5 (t, J=27.8 Hz), 39.4. ¹⁹F NMR (376 MHz, CD₃CN) δ −84.1 (s), −124.0 (t, J=12.6 Hz). IR (neat, cm⁻¹) 3396, 2928, 2157, 1737, 1530, 1195. HRMS (ESI) calcd C₂₄H₁₈F₅N₂O₈⁺ [M+H]⁺: 557.0978. Found: 557.0979.

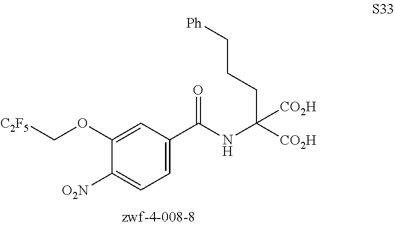

S33 (8.3 mmol scale of hydrolysis, 2.5487 g, 57% Yield) white solid. R_{f}=0.1 (DCM/MeOH 5:1). ¹H NMR (400 MHz, CD₃CN) δ 7.91 (d, J=8.0 Hz, 1H), 7.81 (s, 1H), 7.67 (s, 1H), 7.60 (d, J=8.0 Hz, 1H), 7.32-7.22 (m, 2H), 7.21-7.08 (m, 3H), 4.83 (t, J=12.6 Hz, 2H), 2.64 (t, J=6.8 Hz, 2H), 2.27 (t, J=7.8 Hz, 2H), 1.71-1.48 (m, 2H). ¹³C NMR (100 MHz, CD₃CN) δ 170.4, 165.5, 150.6, 143.1, 142.7, 139.1, 129.4, 126.9, 126.6, 122.4, 115.8, 67.0, 66.7 (t, J=27.8 Hz), 35.8, 33.4, 26.4. ¹⁹F NMR (376 MHz, CD₃CN) δ −84.1 (s), −124.1 (t, J=12.4 Hz). IR (neat, cm⁻¹) 3385, 2936, 1729, 1531, 1193. HRMS (ESI) calcd C₂₂H₂₀F₅N₂O₈⁺ [M+H]⁺: 535.1134. Found: 535.1131.

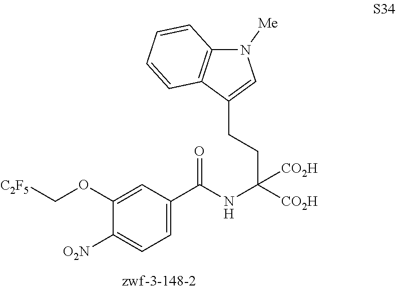

S34 (1.2 mmol scale of hydrolysis, 0.4657 g, 68% Yield) white solid. R_{f}=0.1 (DCM/MeOH 5:1). ¹H NMR (400 MHz, CD₃CN) δ 7.83 (d, J=8.4 Hz, 1H), 7.72 (s, 1H), 7.52 (s, 1H), 7.47 (d, J=8.0 Hz, 1H), 7.39 (d, J=8.4 Hz, 1H), 7.21 (d, J=8.4 Hz, 1H), 7.12 (t, J=7.6 Hz, 1H), 7.01 (t, J=7.4 Hz, 1H), 6.92 (s, 1H), 4.77 (t, J=12.6 Hz, 2H), 3.62 (s, 3H), 2.81-2.74 (m, 2H), 2.74-2.66 (m, 2H). ¹³C NMR (100 MHz, CD₃CN) δ 170.1, 165.2, 150.5, 143.0, 138.9, 138.1, 128.4, 127.6, 126.4, 122.3, 121.9, 119.51, 119.47, 115.5, 113.8, 110.3, 67.3, 66.6 (t, J=27.8 Hz), 33.9, 32.8, 20.4. ¹⁹F NMR (376 MHz, CD₃CN) δ −84.1 (s), −124.0 (t, J=12.4 Hz). IR (neat, cm⁻¹) 3405, 2929, 1736, 1534, 1199. HRMS (ESI) calcd C₂₄H₂₁F₅N₃O₈⁺ [M+H]⁺: 574.1243. Found: 574.1246.

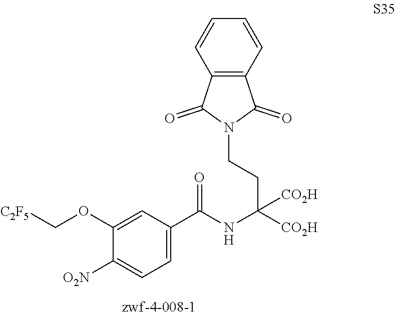

S35 (1.8 mmol scale of hydrolysis, 0.5831 g, 55% Yield) white solid. R_{f}=0.1 (DCM/MeOH 5:1). ¹H NMR (400 MHz, CD₃CN) δ 8.58 (s, 1H), 7.90-7.84 (m, 2H), 7.71-7.66 (m, 1H), 7.59-7.49 (m, 2H), 7.42 (d, J=7.2 Hz, 1H), 7.32-7.23 (m, 1H), 4.79 (t, J=12.2 Hz, 2H), 3.49-3.33 (m, 2H), 2.66-2.52 (m, 2H). ¹³C NMR (100 MHz, CD₃CN) δ 172.0, 170.0, 168.3, 166.2, 150.6, 143.0, 138.9, 138.1, 133.2, 131.3, 131.0, 130.1, 128.7, 126.6, 122.5, 115.8, 66.6 (t, J=27.8 Hz), 66.2, 36.2, 33.4. ¹⁹F NMR (376 MHz, CD₃CN) δ −84.2 (s), −124.0 (t, J=12.4 Hz). IR (neat, cm⁻¹) 3347, 2943, 1714, 1534, 1199. HRMS (ESI) calcd C₂₃H₁₇F₅N₃O₁₀⁺ [M+H]⁺: 590.0829. Found: 590.0833.

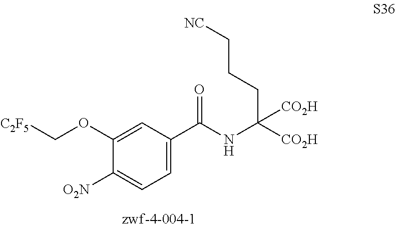

S36 (9.5 mmol scale of hydrolysis, 2.9939 g, 60% Yield) white solid. R_{f}=0.1 (DCM/MeOH 5:1). ¹H NMR (400 MHz, CD₃CN) δ 7.92 (d, J=8.0 Hz, 1H), 7.79 (s, 1H), 7.69 (s, 1H), 7.62 (d, J=8.0 Hz, 1H), 4.84 (t, J=12.6 Hz, 2H), 2.60-2.16 (m, 4H), 1.75-1.45 (m, 2H). ¹³C NMR (100 MHz, CD₃CN) δ 169.5, 165.5, 150.6, 143.2, 139.2, 126.6, 122.4, 120.8, 115.8, 66.9, 66.7 (t, J=27.5 Hz), 32.5, 21.0, 17.3. ¹⁹F NMR (376 MHz, CD₃CN) δ −84.2 (s), −124.1 (t, J=12.4 Hz). IR (neat, cm⁻¹) 3395, 2982, 2190, 1738, 1520, 1194. HRMS (ESI) calcd C₁₇H₁₅F₅N₃O₈⁺ [M+H]⁺: 484.0774. Found: 484.0774.

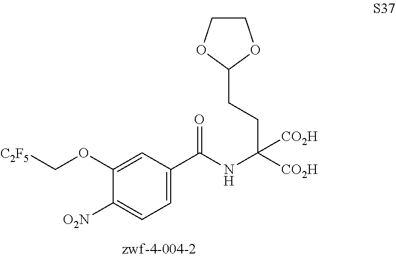

S37 (9.7 mmol scale of hydrolysis, 3.8063 g, 76% Yield) white solid. R_{f}=0.1 (DCM/MeOH 5:1). ¹H NMR (400 MHz, CD₃CN) δ7.97-7.86 (m, 2H), 7.70 (d, J=1.2 Hz, 1H), 7.62 (dd, J₁=8.2, J₁=1.4 Hz, 1H), 4.92-4.75 (m, 3H), 3.95-3.84 (m, 2H), 3.83-3.71 (m, 2H), 2.44-2.30 (m, 2H), 1.69-1.54 (m, 2H). ¹³C NMR (100 MHz, CD₃CN) δ 170.0, 165.6, 150.6, 143.2, 139.1, 126.6, 122.3, 115.8, 104.0, 66.8, 66.7 (t, J=27.5 Hz), 65.8, 28.8, 28.0. ¹⁹F NMR (376 MHz, CD₃CN) δ −84.2 (s), −124.1 (t, J=13.2 Hz). IR (neat, cm⁻¹) 3395, 2290, 1739, 1534, 1194. HRMS (ESI) calcd C₁₈H₁₈F₅N₂O₁₀⁺ [M+H]⁺: 517.0876. Found: 517.0878.

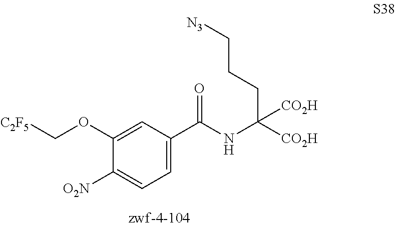

S38 (2.0 mmol scale of hydrolysis, 0.4997 g, 50% Yield) white solid. R_{f}=0.1 (DCM/MeOH 5:1). ¹H NMR (400 MHz, CD₃CN) δ 7.91 (d, J=8.4 Hz, 1H), 7.84 (s, 1H), 7.69 (s, 1H), 7.62 (d, J=8.0 Hz, 1H), 4.84 (t, J=12.4 Hz, 2H), 3.33 (t, J=6.6 Hz, 2H), 2.51-2.21 (m, 2H), 1.63-1.42 (m, 2H). ¹³C NMR (100 MHz, CD₃CN) δ 170.0, 165.5, 150.6, 143.1, 139.2, 126.6, 122.4, 115.8, 67.0, 66.7 (t, J=27.8 Hz), 51.6, 30.9, 24.2. ¹⁹F NMR (376 MHz, CD₃CN) δ −84.2 (s), −124.1 (t, J=12.6 Hz). IR (neat, cm⁻¹) 3390, 2943, 2098, 1739, 1534, 1199. HRMS (ESI) calcd C₁₆H₁₅F₅N₅O₈⁺ [M+H]⁺: 500.0835. Found: 500.0837.

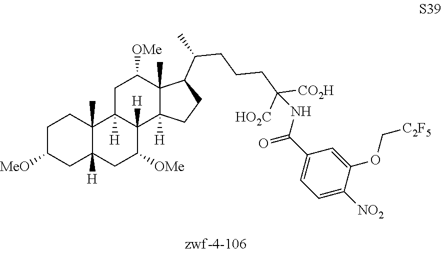

S39 (0.46 mmol scale of hydrolysis, 0.2571 g, 68% Yield) white solid. R_{f}=0.1 (DCM/MeOH 5:1). ¹H NMR (400 MHz, CD₃CN) δ 7.93 (d, J=8.0 Hz, 1H), 7.84 (s, 1H), 7.70 (s, 1H), 7.63 (d, J=8.4 Hz, 1H), 4.84 (t, J=12.6 Hz, 2H), 3.37 (s, 1H), 3.27 (s, 3H), 3.22 (s, 3H), 3.19 (s, 3H), 3.14 (s, 1H), 3.04-3.93 (m, 1H), 2.36-2.08 (m, 2H), 2.02-1.94 (m, 3H), 1.88-0.96 (m, 21H), 0.91-0.82 (m, 6H), 0.64 (s, 3H). ¹³C NMR (100 MHz, CD₃CN) δ 170.7, 170.6, 165.4, 150.7, 143.2, 139.2, 126.6, 122.3, 115.8, 83.0, 81.4, 78.2, 67.1, 66.7 (t, J=27.8 Hz), 56.2, 56.0, 55.5, 47.7, 46.9, 43.5, 42.7, 40.4, 36.4, 36.1, 35.8, 35.6, 35.5, 34.3, 28.6, 28.5, 28.2, 27.7, 23.9, 23.2, 22.7, 21.1, 18.2, 12.8. ¹⁹F NMR (376 MHz, CD₃CN) δ −84.1 (s), −124.1 (t, J=12.4 Hz). IR (neat, cm⁻¹) 3395, 2934, 1739, 1530, 1199. HRMS (ESI) calcd C₄₀H₅₆F₅N₂O₁₁⁺ [M+H]⁺: 835.3799. Found: 835.3795.

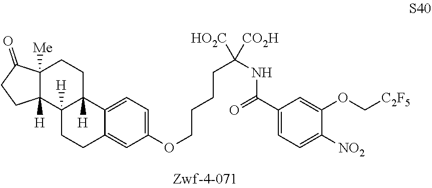

S40 (1.7 mmol scale of hydrolysis, 0.7305 g, 57% Yield) white solid. R_{f}=0.1 (DCM/MeOH 5:1). ¹H NMR (400 MHz, DMSO-d₆) δ 8.40 (s, 1H), 8.01 (d, J=8.0 Hz, 1H), 7.86 (s, 1H), 7.66 (d, J=8.0 Hz, 1H), 7.10 (d, J=8.0 Hz, 1H), 6.61 (d, J=7.2 Hz, 1H), 6.54 (s, 1H), 5.20 (t, J=12.8 Hz, 2H), 3.87-3.84 (m, 2H), 2.75 (s, 2H), 2.46-2.38 (m, 1H), 2.36-2.22 (m, 3H), 2.16-2.02 (m, 2H), 1.98-1.86 (m, 2H), 1.78-1.62 (m, 3H), 1.56-1.26 (m, 8H), 0.81 (s, 3H). ¹³C NMR (100 MHz, DMSO-d₆) δ 219.7, 169.3, 163.4, 156.5, 148.9, 141.4, 138.4, 137.4, 131.7, 126.2, 125.2, 121.7, 115.0, 114.3, 112.1, 67.0, 66.7, 65.1 (t, J=28.3 Hz), 49.6, 47.4, 43.4, 37.9, 35.4, 32.0, 31.4, 29.1, 28.7, 26.1, 25.5, 21.2, 20.3, 13.5. ¹⁹F NMR (376 MHz, DMSO-d₆) δ −82.5 (s), −122.4 (t, J=12.6 Hz). IR (neat, cm⁻¹) 3400, 2939, 1743, 1539, 1199. HRMS (ESI) calcd C₃₅H₃₈F₅N₂O₁₀⁺ [M+H]⁺: 741.2441. Found: 741.2444.

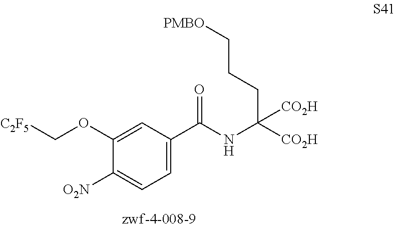

S41 (6.0 mmol scale of hydrolysis, 2.6009 g, 73% Yield) white solid. R_{f}=0.1 (DCM/MeOH 5:1). ¹H NMR (400 MHz, CD₃CN) δ 8.01 (s, 1H), 7.86 (d, J=8.0 Hz, 1H), 7.65 (s, 1H), 7.55 (d, J=8.0 Hz, 1H), 7.20 (d, J=7.2 Hz, 2H), 6.83 (d, J=7.2 Hz, 2H), 4.80 (t, J=12.2 Hz, 2H), 4.37 (s, 2H), 3.74 (s, 3H), 3.53-3.42 (m, 2H), 3.45-3.23 (m, 2H), 1.70-1.50 (m, 2H). ¹³C NMR (100 MHz, CD₃CN) δ 170.5, 165.7, 160.2, 150.6, 143.1, 139.2, 131.5, 130.4, 126.6, 122.3, 115.8, 114.6, 73.1, 70.1, 66.9, 66.7 (t, J=27.9 Hz), 55.8, 31.3, 24.8. ¹⁹F NMR (376 MHz, CD₃CN) δ −84.1 (s), −124.1 (t, J=12.4 Hz). IR (neat, cm⁻¹) 3395, 2934, 1743, 1515, 1194. HRMS (ESI) calcd C₂₄H₂₄F₅N₂O₁₀⁺ [M+H]⁺: 595.1346. Found: 595.1349.

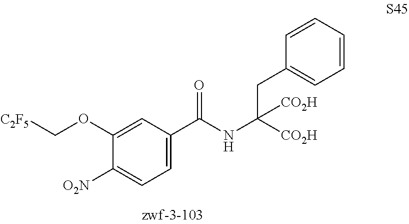

S45 (6.1 mmol scale of hydrolysis, 1.9894 g, 64% Yield) white solid. R_{f}=0.1 (DCM/MeOH 5:1). ¹H NMR (400 MHz, CD₃CN) δ 7.88 (d, J=8.0 Hz, 1H), 7.62 (d, J=1.2 Hz, 1H), 7.52 (s, 1H), 7.45 (dd, J₁=8.2, J₂=1.4 Hz, 1H), 7.30-7.22 (m, 3H), 7.15-7.04 (m, 2H), 4.81 (t, J=12.6 Hz, 2H), 3.65 (s, 2H). ¹³C NMR (100 MHz, CD₃CN) δ 169.2, 165.6, 150.6, 143.1, 139.4, 135.9, 131.0, 129.4, 128.4, 126.7, 122.1, 115.8, 68.1, 66.7 (t, J=27.8 Hz), 38.6. ¹⁹F NMR (376 MHz, CD₃CN) δ −84.1 (s), −124.0 (t, J=12.4 Hz). IR (neat, cm⁻¹) 3382, 2983, 1729, 1525, 1195. HRMS (ESI) calcd C₂₀H₁₆F₅N₂O₈⁺ [M+H]⁺: 507.0821. Found: 507.0819.

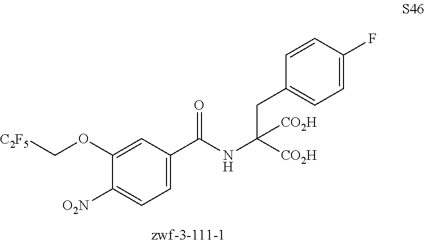

S46 (5.0 mmol scale of hydrolysis, 1.9277 g, 75% Yield) white solid. R_{f}=0.1 (DCM/MeOH 5:1). ¹H NMR (400 MHz, CD₃CN) δ 7.89 (d, J=8.4 Hz, 1H), 7.62 (s, 1H), 7.52-7.45 (m, 2H), 7.14-7.07 (m, 2H), 7.04-6.96 (m, 2H), 4.81 (t, J=12.6 Hz, 2H), 3.64 (s, 2H). ¹³C NMR (100 MHz, CD₃CN) δ 169.0, 165.5, 163.2 (d, J=241.9 Hz), 150.6, 143.2, 139.4, 132.8 (d, J=8.0 Hz), 132.0 (d, J=2.9 Hz), 126.6, 122.2, 116.2, 115.9 (d, J=19.5 Hz), 68.1, 66.7 (t, J=27.5 Hz), 37.7. ¹⁹F NMR (376 MHz, CD₃CN) δ −84.2 (s), −117.0-−117.1 (m), −124.1 (t, J=12.4 Hz). IR (neat, cm⁻¹) 3387, 2953, 1730, 1510, 1193. HRMS (ESI) calcd C₂₀H₁₅F₆N₂O₈⁺ [M+H]⁺: 525.0727. Found: 525.0726.

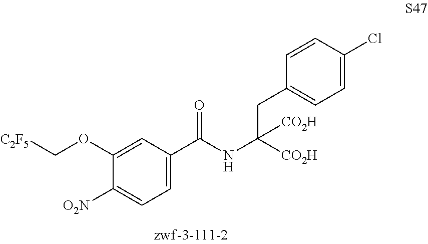

S47 (4.8 mmol scale of hydrolysis, 1.7392 g, 67% Yield) white solid. R_{f}=0.1 (DCM/MeOH 5:1). ¹H NMR (400 MHz, CD₃CN) δ 7.89 (d, J=8.4 Hz, 1H), 7.61 (s, 1H), 7.52-7.43 (m, 2H), 7.27 (d, J=8.4 Hz, 2H), 7.07 (d, J=8.4 Hz, 2H), 4.81 (t, J=12.6 Hz, 2H), 3.64 (s, 2H). ¹³C NMR (100 MHz, CD₃CN) δ 168.8, 165.5, 150.6, 143.2, 139.4, 134.9, 133.8, 132.7, 129.4, 126.6, 122.3, 115.8, 68.0, 66.7 (t, J=27.8 Hz), 37.8. ¹⁹F NMR (376 MHz, CD₃CN) δ −84.2 (s), −124.1 (t, J=12.4 Hz). IR (neat, cm⁻¹) 3389, 2926, 1729, 1526, 1193. HRMS (ESI) calcd C₂₀H₁₅F₅N₂O₈³⁵Cl⁺ [M+H]⁺: 541.0432. Found: 541.0437.

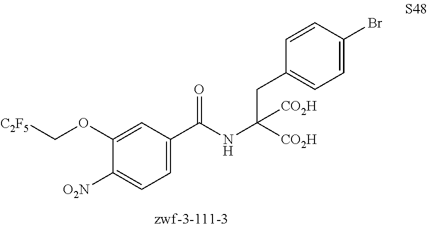

S48 (5.6 mmol scale of hydrolysis, 2.0157 g, 62% Yield) white solid. R_{f}=0.1 (DCM/MeOH 5:1). ¹H NMR (400 MHz, CD₃CN) δ 7.88 (d, J=8.4 Hz, 1H), 7.61 (s, 1H), 7.52-7.45 (m, 2H), 7.41 (d, J=8.0 Hz, 2H), 7.01 (d, J=8.0 Hz, 2H), 4.81 (t, J=12.6 Hz, 2H), 3.62 (s, 2H). ¹³C NMR (100 MHz, CD₃CN) δ 168.8, 165.5, 150.6, 143.2, 139.3, 135.4, 133.0, 132.4, 126.6, 122.3, 121.9, 115.8, 67.9, 66.7 (t, J=27.8 Hz), 37.9. ¹⁹F NMR (376 MHz, CD₃CN) δ −84.1 (s), −124.1 (t, J=13.2 Hz). IR (neat, cm⁻¹) 3386, 2927, 1730, 1526, 1194. HRMS (ESI) calcd C₂₀H₁₅F₅N₂O₈⁷⁹Br⁺ [M+H]⁺: 584.9926. Found: 584.9923.

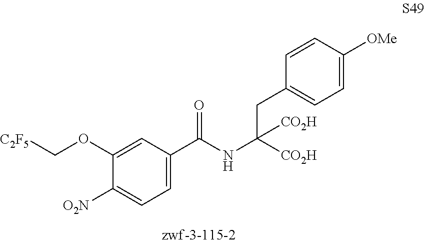

S49 (4.2 mmol scale of hydrolysis, 1.4281 g, 64% Yield) white solid. R_{f}=0.1 (DCM/MeOH 5:1). ¹H NMR (400 MHz, CD₃CN) δ 7.89 (d, J=8.4 Hz, 1H), 7.62 (s, 1H), 7.52-7.40 (m, 2H), 7.01 (d, J=8.0 Hz, 2H), 6.81 (d, J=8.0 Hz, 2H), 4.81 (t, J=12.6 Hz, 2H), 3.74 (s, 3H), 3.57 (s, 2H). ¹³C NMR (100 MHz, CD₃CN) δ 169.3, 165.4, 160.1, 150.6, 143.1, 139.5, 132.1, 127.6, 126.7, 122.2, 115.8, 114.7, 68.1, 66.7 (t, J=27.8 Hz), 55.8, 37.9. ¹⁹F NMR (376 MHz, CD₃CN) δ −84.2 (s), −124.1 (t, J=11.8 Hz). IR (neat, cm⁻¹) 3366, 2964, 1733, 1511, 1202. HRMS (ESI) calcd C₂₁H₁₈F₅N₂O₉⁺ [M+H]⁺: 537.0927. Found: 537.0928.

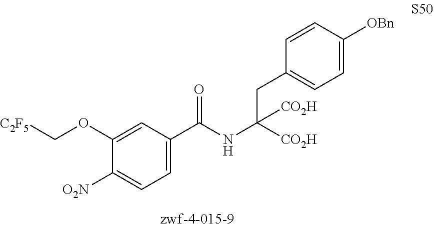

S50 (5.2 mmol scale of hydrolysis, 2.1458 g, 67% Yield) white solid. R_{f}=0.1 (DCM/MeOH 5:1). ¹H NMR (400 MHz, CD₃CN) δ 7.88 (d, J=8.4 Hz, 1H), 7.64 (s, 1H), 7.56-7.26 (m, 7H), 7.02 (d, J=8.0 Hz, 2H), 6.88 (d, J=8.0 Hz, 2H), 5.03 (s, 2H), 4.82 (t, J=12.4 Hz, 2H), 3.59 (s, 2H). ¹³C NMR (100 MHz, CD₃CN) δ 169.3, 165.4, 159.2, 150.6, 143.1, 139.5, 138.3, 132.1, 129.5, 128.9, 128.7, 128.0, 126.7, 122.1, 115.8, 115.7, 70.6, 68.1, 66.7 (t, J=27.8 Hz), 37.8. ¹⁹F NMR (376 MHz, CD₃CN) δ −84.1 (s), -124.0 (t, J=12.4 Hz). IR (neat, cm⁻¹) 3390, 2939, 1739, 1515, 1199. HRMS (ESI) calcd C₂₇H₂₂F₅N₂O₉⁺ [M+H]⁺: 613.1240. Found: 613.1241.

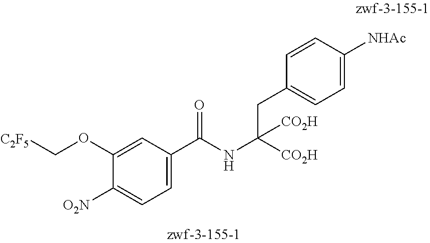

S51 (6.5 mmol scale of hydrolysis, 2.0763 g, 56% Yield) white solid. R_{f}=0.1 (DCM/MeOH 5:1). ¹H NMR (400 MHz, CD₃OD) δ 8.05 (s, 1H), 7.90 (d, J=8.4 Hz, 1H), 7.63 (s, 1H), 7.49 (d, J=7.6 Hz, 1H), 7.40 (d, J=7.6 Hz, 2H), 7.03 (d, J=7.2 Hz, 2H), 4.86 (t, J=12.0 Hz, 2H), 3.69 (s, 2H), 2.08 (s, 3H). ¹³C NMR (100 MHz, CD₃OD) δ 171.6, 170.4, 166.6, 151.0, 143.5, 140.1, 139.0, 132.5, 131.4, 126.7, 122.3, 121.1, 115.8, 69.1, 66.8 (t, J=27.5 Hz), 38.1, 23.7. ¹⁹F NMR (376 MHz, CD₃OD) δ −84.8 (s), −124.7 (t, J=12.4 Hz). IR (neat, cm⁻¹) 3313, 2929, 1763, 1515, 1209. HRMS (ESI) calcd C₂₂H₁₉F₅N₃O₉⁺ [M+H]⁺: 564.1036. Found: 564.1034.

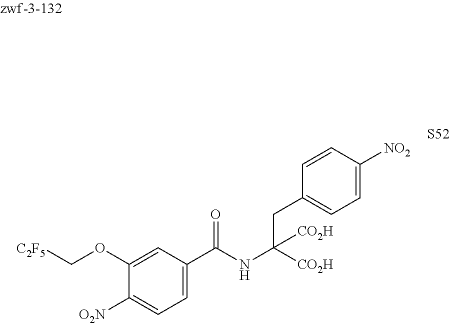

S52 (4.3 mmol scale of hydrolysis, 1.3536 g, 57% Yield) white solid. R_{f}=0.1 (DCM/MeOH 5:1). ¹H NMR (400 MHz, CD₃CN) δ 8.09 (d, J=8.4 Hz, 2H), 7.88 (d, J=8.4 Hz, 1H), 7.60 (s, 1H), 7.52-7.43 (m, 2H), 7.31 (d, J=8.0 Hz, 2H), 4.81 (t, J=12.4 Hz, 2H), 3.79 (s, 2H). ¹³C NMR (100 MHz, CD₃CN) δ 168.5, 165.6, 150.6, 148.5, 144.1, 143.2, 139.3, 132.2, 126.6, 124.4, 122.3, 115.8, 67.9, 66.7 (t, J=27.8 Hz), 38.2. ¹⁹F NMR (376 MHz, CD₃CN) δ −84.2 (s), −124.1 (t, J=13.2 Hz). IR (neat, cm⁻¹) 3356, 2977, 1749, 1521, 1349, 1199. HRMS (ESI) calcd C₂₀H₁₅F₅N₃O₁₀⁺ [M+H]⁺: 552.0672. Found: 552.0675.

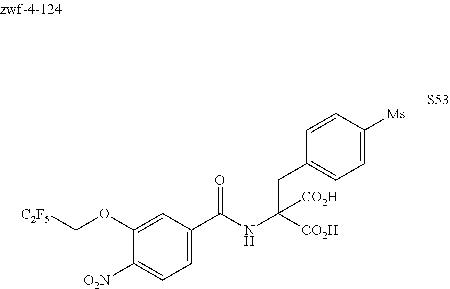

S53 (4.0 mmol scale of hydrolysis, 0.7493 g, 32% Yield) white solid. R_{f}=0.1 (DCM/MeOH 5:1). ¹H NMR (400 MHz, CD₃CN) δ 7.88 (d, J=8.4 Hz, 1H), 7.80 (d, J=7.6 Hz, 2H), 7.62 (s, 1H), 7.53-7.44 (m, 2H), 7.33 (d, J=7.6 Hz, 2H), 4.82 (t, J=12.4 Hz, 2H), 3.77 (s, 2H), 3.03 (s, 3H). ¹³C NMR (100 MHz, CD₃CN) δ 168.7, 165.5, 150.6, 143.2, 142.6, 141.0, 139.3, 132.0, 128.2, 126.6, 122.3, 115.8, 68.0, 66.7 (t, J=27.8 Hz), 44.5, 38.3. ¹⁹F NMR (376 MHz, CD₃CN) δ −84.1 (s), −124.0 (t, J=13.2 Hz). IR (neat, cm⁻¹) 3390, 2934, 1734, 1539, 1204. HRMS (ESI) calcd C₂₁H₁₈F₅N₂O₁₀S⁺ [M+H]⁺: 585.0597. Found: 585.0596.

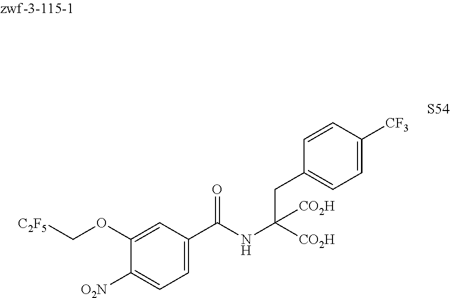

S54 (4.7 mmol scale of hydrolysis, 1.5121 g, 56% Yield) white solid. R_{f}=0.1 (DCM/MeOH 5:1). ¹H NMR (400 MHz, CD₃CN) δ 7.89 (d, J=8.4 Hz, 1H), 7.63-7.55 (m, 3H), 7.51-7.46 (m, 2H), 7.28 (d, J=8.0 Hz, 2H), 4.81 (t, J=12.6 Hz, 2H), 3.74 (s, 2H). ¹³C NMR (100 MHz, CD₃CN) δ 168.7, 165.6, 150.6, 143.2, 140.8, 139.3, 131.8, 129.9 (q, J=32.0 Hz), 126.6, 126.2 (q, J=3.8 Hz), 125.5 (q, J=269.6 Hz), 122.3, 115.8, 68.0, 66.7 (t, J=27.8 Hz), 38.2. ¹⁹F NMR (376 MHz, CD₃CN) δ −63.0 (s), −84.2 (s), −124.1 (t, J=12.4 Hz). IR (neat, cm⁻¹) 3389, 2957, 1730, 1530, 1195. HRMS (ESI) calcd C₂₁H₁₅F₈N₂O₈⁺ [M+H]⁺: 575.0695. Found: 575.0697.

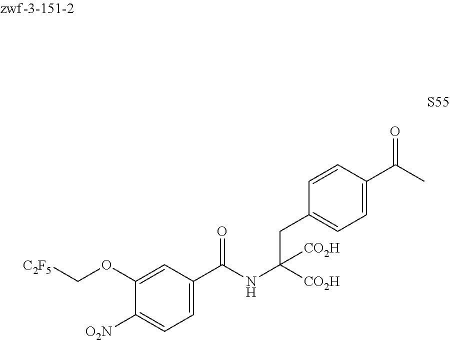

S55 (6.6 mmol scale of hydrolysis, 1.7377 g, 48% Yield) white solid. R_{f}=0.1 (DCM/MeOH 5:1). ¹H NMR (400 MHz, CD₃CN) δ 7.92-7.80 (m, 3H), 7.62 (s, 1H), 7.53-7.42 (m, 2H), 7.21 (d, J=8.0 Hz, 2H), 4.81 (t, J=12.6 Hz, 2H), 3.73 (s, 2H), 2.52 (s, 3H). ¹³C NMR (100 MHz, CD₃CN) δ 198.9, 168.8, 165.6, 150.6, 143.2, 141.6, 139.4, 137.3, 131.3, 129.3, 126.6, 122.2, 115.8, 67.9, 66.7 (t, J=27.8 Hz), 38.4, 27.0. ¹⁹F NMR (376 MHz, CD₃CN) δ −84.1 (s), −124.0 (t, J=12.6 Hz). IR (neat, cm⁻¹) 3395, 2934, 1739, 1534, 1194. HRMS (ESI) calcd C₂₂H₁₈F₅N₂O₉⁺ [M+H]⁺: 549.0927. Found: 549.0928.

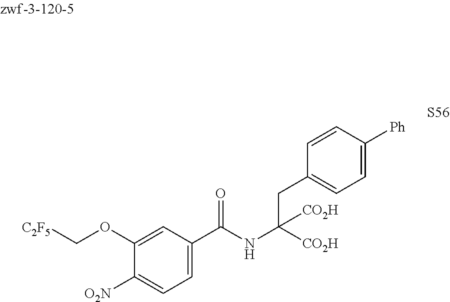

S56 (4.0 mmol scale of hydrolysis, 1.4263 g, 61% Yield) white solid. R_{f}=0.1 (DCM/MeOH 5:1). ¹H NMR (400 MHz, CD₃OD) δ 7.90 (d, J=8.0 Hz, 1H), 7.68 (s, 1H), 7.60-7.43 (m, 5H), 7.43-7.33 (m, 2H), 7.33-7.24 (m, 1H), 7.18 (d, J=7.2 Hz, 2H), 4.85 (t, J=12.2 Hz, 2H), 3.78 (s, 2H). ¹³C NMR (100 MHz, CD₃OD) δ 170.4, 166.6, 151.0, 143.6, 141.8, 141.4, 140.1, 136.0, 131.6, 129.8, 128.3, 127.9, 127.8, 126.8, 122.3, 115.8, 69.1, 66.8 (t, J=28.6 Hz), 38.3. ¹⁹F NMR (376 MHz, CD₃OD) δ −84.8 (s), −124.6 (t, J=11.8 Hz). IR (neat, cm⁻¹) 3385, 3074, 1760, 1510, 1193. HRMS (ESI) calcd C₂₆H₂₀F₅N₂O₈⁺ [M+H]⁺: 583.1134. Found: 583.1138.

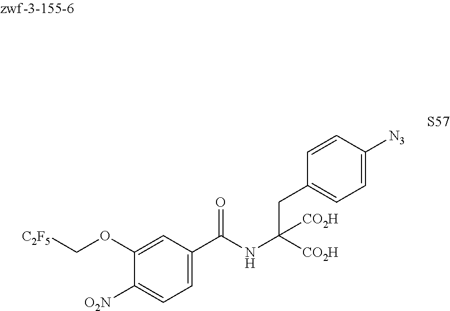

S57 (6.0 mmol scale of hydrolysis, 2.0061 g, 61% Yield) white solid. R_{f}=0.1 (DCM/MeOH 5:1). ¹H NMR (400 MHz, CD₃CN) δ 7.89 (d, J=8.0 Hz, 1H), 7.61 (s, 1H), 7.52-7.42 (m, 2H), 7.10 (d, J=7.6 Hz, 2H), 6.96 (d, J=7.6 Hz, 2H), 4.81 (t, J=12.2 Hz, 2H), 3.63 (s, 2H). ¹³C NMR (100 MHz, CD₃CN) δ 169.0, 165.5, 150.6, 143.2, 140.3, 139.4, 132.7, 132.5, 126.6, 122.2, 120.0, 115.8, 68.0, 66.7 (t, J=27.8 Hz), 37.9. ¹⁹F NMR (376 MHz, CD₃CN) δ −84.2 (s), −124.1 (t, J=12.4 Hz). IR (neat, cm⁻¹) 3381, 2929, 2117, 1734, 1510, 1209. HRMS (ESI) calcd C₂₀H₁₅F₅N₅O₈⁺ [M+H]⁺: 548.0835. Found: 548.0832.

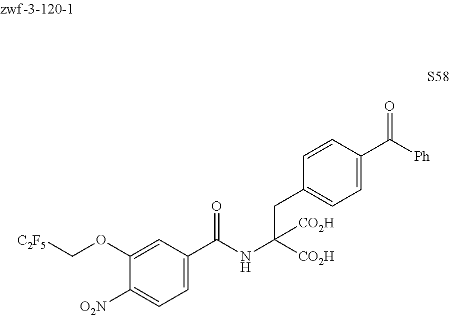

S58 (4.0 mmol scale of hydrolysis, 1.3662 g, 56% Yield) white solid. R_{f}=0.1 (DCM/MeOH 5:1). ¹H NMR (400 MHz, CD₃CN) δ 7.87 (d, J=8.0 Hz, 1H), 7.77-7.59 (m, 6H), 7.57-7.43 (m, 4H), 7.24 (d, J=6.8 Hz, 2H), 4.80 (t, J=12.0 Hz, 2H), 3.77 (s, 2H). ¹³C NMR (100 MHz, CD₃CN) δ 197.1, 168.8, 165.6, 150.6, 143.2, 141.1, 139.4, 138.5, 137.6, 133.5, 131.1, 130.9, 130.7, 129.4, 126.6, 122.3, 115.8, 68.0, 66.7 (t, J=27.8 Hz), 38.4. ¹⁹F NMR (376 MHz, CD₃CN) δ −84.1 (s), −124.0 (t, J=12.6 Hz). IR (neat, cm⁻¹) 3400, 3056, 1735, 1528, 1195. HRMS (ESI) calcd C₂₇H₂₀F₅N₂O₉⁺ [M+H]⁺: 611.1083. Found: 611.1084.

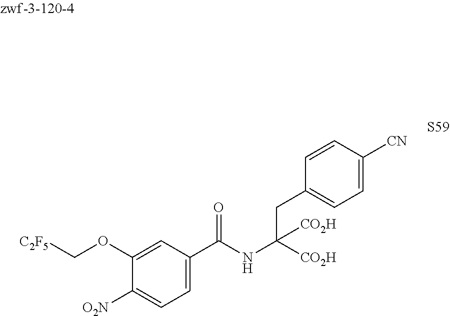

S59 (4.7 mmol scale of hydrolysis, 1.0209 g, 41% Yield) white solid. R_{f}=0.1 (DCM/MeOH 5:1). ¹H NMR (400 MHz, CD₃CN) δ 7.88 (d, J=8.4 Hz, 1H), 7.68-7.56 (m, 3H), 7.51-7.42 (m, 2H), 7.25 (d, J=8.0 Hz, 2H), 4.81 (t, J=12.6 Hz, 2H), 3.73 (s, 2H). ¹³C NMR (100 MHz, CD₃CN) δ 168.6, 165.5, 150.6, 143.2, 141.9, 139.3, 133.2, 132.0, 126.6, 122.3, 119.6, 115.8, 112.0, 67.9, 66.7 (t, J=27.5 Hz), 38.5. ¹⁹F NMR (376 MHz, CD₃CN) δ −84.2 (s), −124.1 (t, J=12.4 Hz). IR (neat, cm⁻¹) 3380, 2959, 1730, 1531, 1199. HRMS (ESI) calcd C₂₁H₁₅F₅N₃O₈⁺ [M+H]⁺: 532.0774. Found: 532.0773.

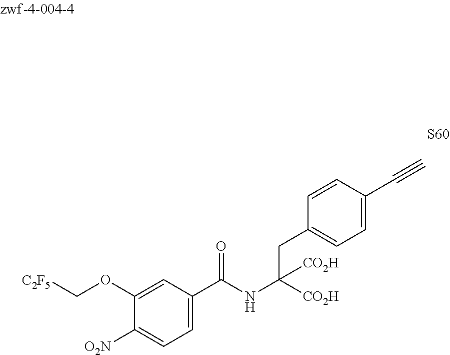

S60 (8.3 mmol scale of hydrolysis, 2.8275 g, 65% Yield) white solid. R_{f}=0.1 (DCM/MeOH 5:1). ¹H NMR (400 MHz, CD₃CN) δ 7.88 (d, J=8.4 Hz, 1H), 7.61 (s, 1H), 7.56-7.43 (m, 2H), 7.37 (d, J=7.6 Hz, 2H), 7.08 (d, J=8.0 Hz, 2H), 4.81 (t, J=12.6 Hz, 2H), 3.66 (s, 2H), 3.37 (s, 1H). ¹³C NMR (100 MHz, CD₃CN) δ 168.9, 165.5, 150.6, 143.2, 139.4, 137.2, 132.9, 131.3, 126.6, 122.2, 122.0, 115.7, 83.8, 79.2, 68.0, 66.7 (t, J=27.8 Hz), 38.3. ¹⁹F NMR (376 MHz, CD₃CN) δ −84.1 (s), −124.1 (t, J=12.6 Hz). IR (neat, cm⁻¹) 3298, 2939, 1743, 1534, 1199. HRMS (ESI) calcd C₂₂H₁₆F₅N₂O₈⁺ [M+H]⁺: 531.0821. Found: 531.0824.

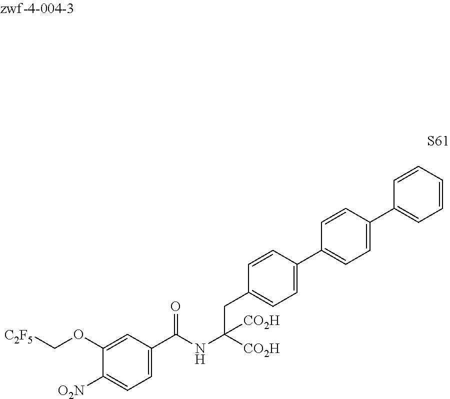

S61 (5.1 mmol scale of hydrolysis, 1.5484 g, 46% Yield) white solid. R_{f}=0.1 (DCM/MeOH 5:1). ¹H NMR (400 MHz, CD₃CN) δ 7.88 (d, J=7.6 Hz, 1H), 7.78-7.62 (m, 7H), 7.62-7.54 (m, 3H), 7.52-7.42 (m, 3H), 7.41-7.33 (m, 1H), 7.19 (d, J=6.4 Hz, 2H), 4.80 (t, J=12.2 Hz, 2H), 3.72 (s, 2H). ¹³C NMR (100 MHz, CD₃CN) δ 169.1, 165.5, 150.6, 143.2, 141.2, 140.9, 140.23, 140.20, 139.5, 135.3, 131.7, 129.9, 128.5, 128.4, 128.2, 127.8, 127.7, 126.7, 122.2, 115.8, 68.1, 66.7 (t, J=27.8 Hz), 38.3. ¹⁹F NMR (376 MHz, CD₃CN) δ −84.1 (s), −124.0 (t, J=12.6 Hz). IR (neat, cm⁻¹) 3385, 2924, 1739, 1530, 1204. HRMS (ESI) calcd C₃₂H₂₄F₅N₂O₈⁺ [M+H]⁺: 659.1447. Found: 659.1443.

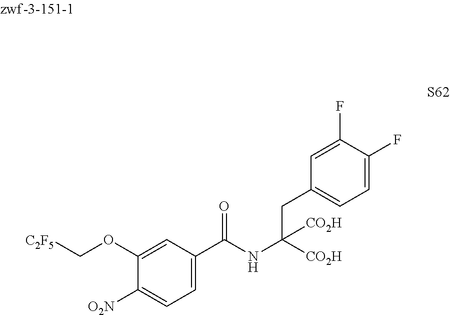

S62 (5.0 mmol scale of hydrolysis, 1.6554 g, 61% Yield) white solid. R_{f}=0.1 (DCM/MeOH 5:1). ¹H NMR (400 MHz, CD₃CN) δ 7.89 (d, J=8.0 Hz, 1H), 7.62 (s, 1H), 7.55-7.43 (m, 2H), 7.19-7.11 (m, 1H), 6.99 (t, J=8.6 Hz, 1H), 6.89 (s, 1H), 4.81 (t, J=12.2 Hz, 2H), 3.63 (s, 2H). ¹³C NMR (100 MHz, CD₃CN) δ 168.7, 165.5, 150.7 (ddd, J₁=244.1, J₂=19.9, J₃=12.6 Hz), 150.6, 143.2, 139.3, 133.6 (dd, J₁=5.5, J₂=4.0 Hz), 127.7 (dd, J₁=6.2, J₂=3.3 Hz), 126.6, 122.3, 119.0 (dd, J₁=156.0, J₂=16.6 Hz), 115.8, 68.0, 66.7 (t, J=27.8 Hz), 37.7. ¹⁹F NMR (376 MHz, CD₃CN) δ −84.2 (s), −124.1 (t, J=12.6 Hz), −140.1-−140.3 (m), −142.0-−142.2 (m). IR (neat, cm⁻¹) 3381, 2987, 1743, 15225 1204. HRMS (ESI) calcd C₂₀H₁₄F₇N₂O₈⁺ [M+H]⁺: 543.0633. Found: 543.0634.

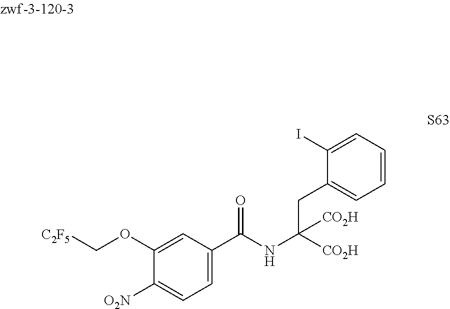

S63 (4.8 mmol scale of hydrolysis, 0.9836 g, 33% Yield) white solid. R_{f}=0.1 (DCM/MeOH 5:1). ¹H NMR (400 MHz, CD₃CN) δ 7.92-7.80 (m, 2H), 7.66 (s, 1H), 7.56 (s, 1H), 7.52 (d, J=8.4 Hz, 1H), 7.29 (t, J=7.4 Hz, 1H), 7.23 (d, J=7.2 Hz, 1H), 6.96 (t, J=7.4 Hz, 1H), 4.81 (t, J=12.6 Hz, 2H), 3.83 (s, 2H). ¹³C NMR (100 MHz, CD₃CN) δ 169.0, 165.5, 150.6, 143.1, 141.2, 139.5, 139.4, 132.0, 130.2, 129.4, 126.5, 122.4, 116.0, 102.5, 67.6, 66.7 (t, J=27.8 Hz), 42.5. ¹⁹F NMR (376 MHz, CD₃CN) δ −84.2 (s), −124.1 (t, J=12.4 Hz). IR (neat, cm⁻¹) 3394, 2926, 1729, 1525, 1193. HRMS (ESI) calcd C₂₀H₁₅F₅N₂O₈⁺ [M+H]⁺: 632.9788. Found: 632.9789.

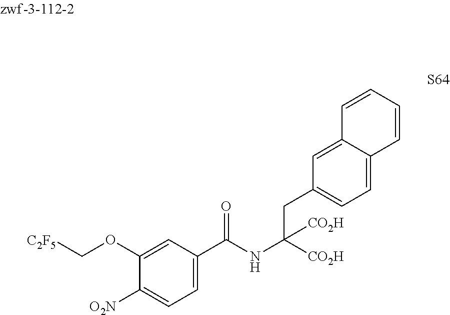

S64 (4.7 mmol scale of hydrolysis, 1.5007 g, 57% Yield) white solid. R_{f}=0.1 (DCM/MeOH 5:1). ¹H NMR (400 MHz, CD₃CN) δ 7.89-7.82 (m, 2H), 7.80-7.72 (m, 2H), 7.59 (d, J=13.2 Hz, 2H), 7.53-7.41 (m, 4H), 7.23 (d, J=8.0 Hz, 1H), 4.72 (t, J=12.2 Hz, 2H), 3.83 (s, 2H). ¹³C NMR (100 MHz, CD₃CN) δ 169.0, 165.6, 150.6, 143.1, 139.5, 134.3, 133.7, 133.6, 129.9, 129.1, 128.9, 128.53, 128.48, 127.3, 127.0, 126.6, 122.3, 115.7, 68.2, 66.6 (t, J=27.8 Hz), 38.7. ¹⁹F NMR (376 MHz, CD₃CN) δ −84.1 (s), −124.0 (t, J=12.6 Hz). IR (neat, cm⁻¹) 3411, 3061, 1729, 1512, 1193. HRMS (ESI) calcd C₂₄H₁₈F₅N₂O₈⁺ [M+H]⁺: 557.0978. Found: 557.0980.

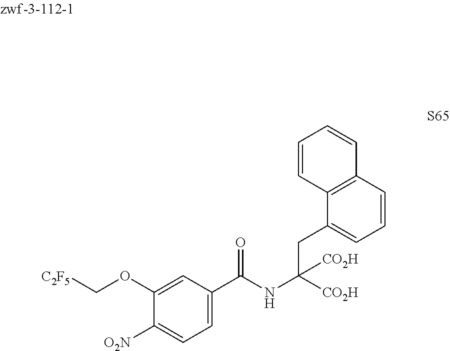

S65 (4.3 mmol scale of hydrolysis, 0.8075 g, 34% Yield) white solid. R_{f}=0.1 (DCM/MeOH 5:1). ¹H NMR (400 MHz, CD₃CN) δ 8.82 (d, J=8.4 Hz, 1H), 7.88-7.75 (m, 3H), 7.43-7.30 (m, 6H), 7.25 (d, J=8.0 Hz, 1H), 4.68 (t, J=12.6 Hz, 2H), 4.17 (s, 2H). ¹³C NMR (100 MHz, CD₃CN) δ 169.2, 166.0, 150.4, 143.0, 139.4, 134.8, 134.0, 132.5, 129.6, 129.5, 129.1, 127.1, 126.6, 126.5, 126.3, 124.8, 122.2, 115.6, 68.3, 66.6 (t, J=27.5 Hz), 34.7. ¹⁹F NMR (376 MHz, CD₃CN) δ −84.1 (s), −124.1 (t, J=12.4 Hz). IR (neat, cm⁻¹) 3385, 2945, 1738, 1526, 1197. HRMS (ESI) calcd C₂₄H₁₈F₅N₂O₈⁺ [M+H]⁺: 557.0978. Found: 557.0979.

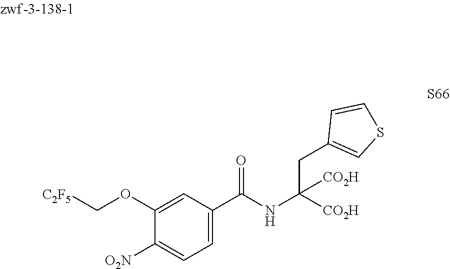

S66 (5.1 mmol scale of hydrolysis, 1.8568 g, 71% Yield) white solid. R_{f}=0.1 (DCM/MeOH 5:1). ¹H NMR (400 MHz, CD₃CN) δ 7.89 (d, J=8.0 Hz, 1H), 7.62 (s, 1H), 7.56 (s, 1H), 7.48 (d, J=8.4 Hz, 1H), 7.31 (s, 1H), 7.08 (s, 1H), 7.85 (d, J=4.8 Hz, 1H), 4.82 (t, J=12.4 Hz, 2H), 3.70 (s, 2H). ¹³C NMR (100 MHz, CD₃CN) δ 169.1, 165.5, 150.6, 143.2, 139.4, 135.9, 130.0, 126.9, 126.7, 125.2, 122.2, 115.8, 67.8, 66.7 (t, J=27.8 Hz), 33.3. ¹⁹F NMR (376 MHz, CD₃CN) δ −84.1 (s), −124.1 (t, J=12.6 Hz). IR (neat, cm⁻¹) 3385, 3062, 1730, 1527, 1193. HRMS (ESI) calcd C₁₈H₁₄F₅N₂O₈S⁺ [M+H]⁺: 513.0386. Found: 513.0387.

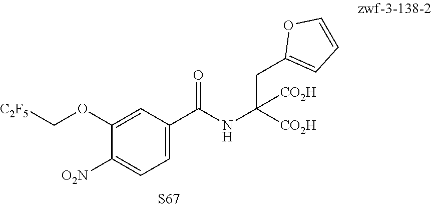

S67 (6.8 mmol scale of hydrolysis, 2.2831 g, 68% Yield) white solid. R_{f}=0.1 (DCM/MeOH 5:1). ¹H NMR (400 MHz, CD₃CN) δ 7.89 (d, J=8.0 Hz, 1H), 7.68-7.57 (m, 2H), 7.47 (d, J=8.4 Hz, 1H), 7.37 (s, 1H), 6.31 (s, 1H), 6.13 (d, J=2.0 Hz, 1H), 4.82 (t, J=12.6 Hz, 2H), 3.73 (s, 2H). ¹³C NMR (100 MHz, CD₃CN) δ 168.8, 165.5, 150.65, 150.60, 143.7, 143.1, 139.5, 126.7, 122.2, 115.7, 111.5, 109.9, 66.9, 66.7 (t, J=27.5 Hz), 31.9. ¹⁹F NMR (376 MHz, CD₃CN) δ −84.2 (s), −124.1 (t, J=12.4 Hz). IR (neat, cm⁻¹) 3392, 3075, 1731, 1530, 1194. HRMS (ESI) calcd C₁₈H₁₄F₅N₂O₉⁺ [M+H]⁺: 497.0614. Found: 497.0617.

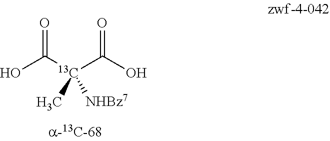

¹H NMR (400 MHz, DMSO-d₆) δ 8.55 (s, 1H), 8.03 (d, J=8.0 Hz, 1H), 7.87 (s, 1H), 7.66 (d, J=8.0 Hz, 1H), 5.19 (t, J=12.6 Hz, 2H), 1.69 (s, 3H). ¹³C NMR (100 MHz, DMSO-d₆) δ 170.0 (d, J=57.8 Hz), 163.7, 149.0, 141.4, 138.6, 125.3, 121.7, 115.0, 65.1 (t, J=27.8 Hz), 63.2, 21.0 (d, J=36.9 Hz). ¹⁹F NMR (376 MHz, DMSO-d₆) δ −82.6 (s), −122.5 (t, J=13.2 Hz). IR (neat, cm⁻¹) 3385, 2977, 1743, 1534, 1199. HRMS (ESI) calcd C₁₃¹³CH₁₂F₅N₂O₈⁺ [M+H]⁺: 432.0542. Found: 432.0546.

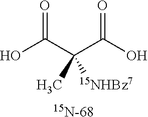

¹⁵N-68 (1.6 mmol scale of hydrolysis, 0.6461 g, 91% Yield) white solid. R_{f}=0.1 (DCM/MeOH 5:1). ¹H NMR (400 MHz, CD₃CN) δ 7.92 (d, J=8.4 Hz, 1H), 7.91 (d, J=93.2 Hz, 1H), 7.70 (s, 1H), 7.61 (d, J=8.4 Hz, 1H), 4.83 (t, J=12.4 Hz, 2H), 1.79 (s, 3H). ¹³C NMR (100 MHz, CD₃CN) δ 171.0, 165.8 (d, J=15.9 Hz), 150.6, 143.1, 139.1 (d, J=8.6 Hz), 126.6, 122.3, 115.8, 66.7 (t, J=27.8 Hz), 63.5 (d, J=12.3 Hz), 21.7. ¹⁹F NMR (376 MHz, CD₃CN) δ −84.2 (s), −124.1 (t, J=12.4 Hz). ¹⁵N NMR (40 MHz, CD₃CN) δ 121.9. IR (neat, cm⁻¹) 3390, 2943, 1739, 1539, 1194. HRMS (ESI) calcd C₁₄H₁₂F₅N¹⁵NO₈⁺ [M+H]⁺: 432.0479. Found: 432.0477.

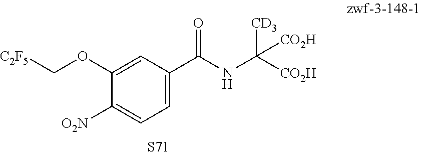

S71 (4.0 mmol scale of hydrolysis, 1.5972 g, 92% Yield) white solid. R_{f}=0.1 (DCM/MeOH 5:1). ¹H NMR (400 MHz, CD₃CN) δ 8.01-7.84 (m, 2H), 7.70 (s, 1H), 7.61 (d, J=8.4 Hz, 1H), 4.83 (t, J=12.6 Hz, 2H). ¹³C NMR (150 MHz, CD₃CN) δ 171.0, 165.8, 150.6, 143.1, 139.1, 126.6, 122.3, 119.6 (qt, J₁=283.9 Hz, J₂=34.6 Hz), 115.8, 113.6 (tq, J₁=253.1 Hz, J₂=37.8 Hz), 66.7 (t, J=27.5 Hz), 63.4, 20.9 (hept, J=19.7 Hz). ¹⁹F NMR (376 MHz, CD₃CN) δ −84.2 (s), −124.1 (t, J=12.4 Hz). IR (neat, cm⁻¹) 3395, 2987, 1734, 1530, 1194. HRMS (ESI) calcd C₁₄H₉D₃F₅N₂O₈⁺ [M+H]⁺: 434.0697. Found: 434.0693.

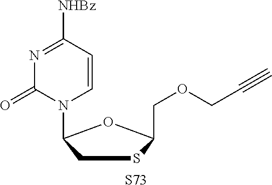

S73 (10.0 mmol scale of hydrolysis, 2.5373 g, 68% Yield, 4 steps) white solid. R_{f}=0.7 (DCM/MeOH 5:1). ¹H NMR (400 MHz, CDCl₃) δ 8.16 (d, J=7.2 Hz, 2H), 7.78 (d, J=8.0 Hz, 1H), 7.58-7.48 (m, 1H), 7.47-7.34 (m, 2H), 6.41 (d, J=8.0 Hz, 1H), 6.37-6.26 (m, 1H), 5.26 (s, 1H), 4.92 (s, 2H), 4.09-3.94 (m, 1H), 3.92-3.77 (m, 1H), 3.57-3.38 (m, 2H), 3.13 (dd, J₁=12.0, J₂=3.6 Hz, 1H), 2.28 (s, 1H). ¹³C NMR (100 MHz, CDCl₃) δ 177.4, 153.9, 149.3, 136.4, 135.4, 132.7, 129.8, 128.2, 98.0, 87.1, 86.6, 78.0, 71.0, 63.1, 37.6, 32.2. IR (neat, cm⁻¹) 3417, 3289, 2927, 2248, 1646, 1555, 1250, 1263. HRMS (ESI) calcd C₁₈H₁₈N₃O₄S⁺ [M+H]⁺: 372.1013. Found: 372.1009.

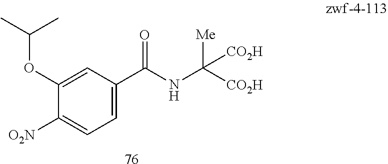

76 (9.3 mmol scale of hydrolysis, 2.9922 g, 94% Yield) was obtained as white solid from the general procedure by using isopropyl alcohol instead of 2,2,3,3,3-pentafluoropropan-1-ol. R_{f}=0.1 (DCM/MeOH 5:1). ¹H NMR (400 MHz, CD₃CN) δ 9.38 (br, 2H), 7.98 (s, 1H), 7.73 (d, J=8.4 Hz, 1H), 7.58 (s, 1H), 7.40 (d, J=8.4 Hz, 1H), 4.90-4.71 (m, 1H), 1.77 (s, 3H), 1.28 (d, J=6.0 Hz, 6H). ¹³C NMR (100 MHz, CD₃CN) δ 171.2, 167.0, 151.5, 144.1, 138.4, 126.0, 120.0, 116.4, 74.0, 63.6, 22.0, 21.6. IR (neat, cm⁻¹) 3395, 2977, 1734, 1525, 1253. HRMS (ESI) calcd C₁₄H₁₇N₂O₈⁺ [M+H]⁺: 341.0979. Found: 341.0981.

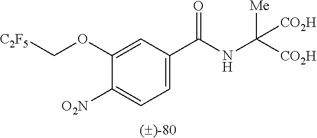

(±)-80 (3.0 mmol scale of hydrolysis, 0.3556 g, 26% Yield) white solid. R_{f}=0.3 (DCM/MeOH 5:1). ¹H NMR (400 MHz, DMSO-d₆) δ 8.71 (s, 1H), 8.03 (d, J=8.4 Hz, 1H), 7.86 (s, 1H), 7.66 (d, J=8.4 Hz, 1H), 5.19 (t, J=12.8 Hz, 2H), 4.15 (q, J=7.1 Hz, 2H), 1.70 (s, 3H), 1.16 (t, J=7.0 Hz, 3H). ¹³C NMR (100 MHz, DMSO-d₆) δ 169.7, 168.2, 163.9, 148.9, 141.4, 138.4, 125.2, 121.7, 115.0, 65.1 (t, J=27.8 Hz), 63.3, 61.5, 21.1, 13.9. ¹⁹F NMR (376 MHz, DMSO-d₆) δ −82.6 (s), −122.5 (t, J=12.4 Hz). IR (neat, cm⁻¹) 3400, 2924, 1739, 1539, 1194. HRMS (ESI) calcd C₁₆H₁₆F₅N₂O₈⁺ [M+H]⁺: 459.0821. Found: 459.0824.

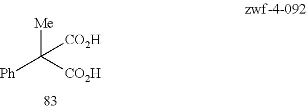 [SMILES: CC(C(=O)O)(C(=O)O)c1ccccc1]

83 (8.5 mmol scale of hydrolysis, 1.4693 g, 89% Yield) was prepared according to a reported procedure as white solid. R_{f}=0.1 (DCM/MeOH 5:1). ¹H NMR (400 MHz, CD₃OD) δ 7.43 (d, J=8.0 Hz, 2H), 7.37-7.23 (m, 3H), 5.08 (br, 2H), 1.83 (s, 3H). ¹³C NMR (100 MHz, CD₃OD) δ 175.3, 140.4, 129.0, 128.6, 128.4, 59.9, 23.0.

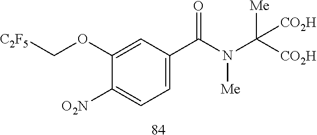

84 (2.0 mmol scale of hydrolysis, 0.2127 g, 24% Yield) white solid. R_{f}=0.1 (DCM/MeOH 5:1). ¹H NMR (500 MHz, DMSO-d₆) δ 8.02 (d, J=8.0 Hz, 1H), 7.49 (s, 1H), 7.23 (d, J=8.0 Hz, 1H), 5.17 (t, J=13.0 Hz, 2H), 2.88 (s, 3H), 1.71 (s, 3H). ¹³C NMR (125 MHz, DMSO-d₆) δ 170.0, 169.1, 149.3, 141.7, 140.2, 125.6, 120.9, 114.3, 68.0, 65.0 (t, J=27.6 Hz), 35.1, 20.4. ¹⁹F NMR (376 MHz, DMSO-d₆) δ −82.6 (s), -122.5 (t, J=13.2 Hz). IR (neat, cm⁻¹) 2929, 1748, 1593, 1530, 1199. HRMS (ESI) calcd C₁₅H₁₄F₅N₂O₈⁺ [M+H]⁺: 445.0665. Found: 445.0664.

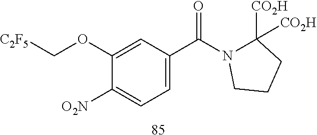

85 (3.6 mmol scale of hydrolysis, 1.2227 g, 74% Yield) white solid. R_{f}=0.1 (DCM/MeOH 5:1). ¹H NMR (400 MHz, CD₃CN) δ 7.93 (d, J=8.0 Hz, 1H), 7.41 (s, 1H), 7.33 (d, J=8.4 Hz, 1H), 4.80 (t, J=12.6 Hz, 2H), 3.67 (t, J=6.6 Hz, 2H), 2.50 (t, J=6.8 Hz, 2H), 2.09-1.99 (m, 2H). ¹³C NMR (100 MHz, CD₃CN) δ 171.7, 169.7, 150.7, 142.2, 141.2, 126.9, 121.9, 115.2, 72.5, 66.7 (t, J=27.5 Hz), 52.2, 36.9, 25.6. ¹⁹F NMR (376 MHz, CD₃CN) δ −84.2 (s), −124.1 (t, J=12.6 Hz). IR (neat, cm⁻¹) 3463, 2977, 1753, 1530, 1442, 1194. HRMS (ESI) calcd C₁₆H₁₄F₅N₂O₈⁺ [M+H]⁺: 457.0665. Found: 457.0664.

### Characterization of Products

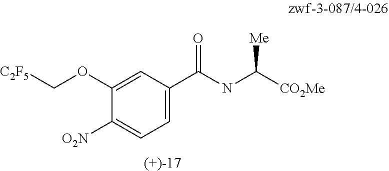

(+)-17 was obtained as white solid from the asymmetric decarboxylation reaction of malonic acid S17 (86.1 mg, 0.2 mmol) for 10 h using the general procedure (71.9 mg, 90% Yield). R_{f}=0.3 (Hexane/EtOAc 2:1). HPLC analysis (Chiralpak IA-3, hexane/PrOH=80/20, 0.8 mL/min, 254 nm; tᵣ (minor)=8.59 min, tᵣ (major)=11.56 min) gave the isomeric composition of the product: 94% e.e., [□]17 D=+21.7 (c=1.05, CHCl₃). ¹H NMR (400 MHz, CDCl₃) δ 7.93 (d, J=8.4 Hz, 1H), 7.64 (d, J=1.6 Hz, 1H), 7.49 (dd, J₁=8.4, J₂=1.6 Hz, 1H), 6.84 (d, J=7.2 Hz, 1H), 4.86-4.71 (m, 1H), 4.61 (t, J=11.8, 2H), 3.83 (s, 3H), 1.56 (d, J=7.2 Hz, 3H). ¹³C NMR (150 MHz, CDCl₃) δ 173.8, 164.2, 149.9, 141.9, 138.6, 125.7, 120.5, 118.3 (qt, J₁=284.3 Hz, J₂=34.3 Hz), 114.4, 111.8 (tq, J₁=254.6 Hz, J₂=37.8 Hz), 65.9 (t, J=28.7 Hz), 52.7, 48.8, 17.7. ¹⁹F NMR (376 MHz, CDCl₃) δ −83.3 (s), −123.1 (t, J=11.8 Hz). IR (neat, cm⁻¹) 3373, 2964, 1711, 1529, 1193. HRMS (ESI) calcd C₁₄H₁₄F₅N₂O₆⁺ [M+H]⁺: 401.0767. Found: 401.0768.

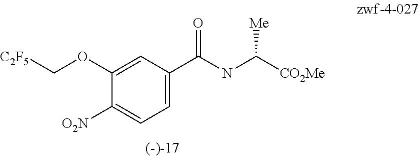

(−)-17 was obtained as white solid from the asymmetric decarboxylation reaction of malonic acid S17 (86.2 mg, 0.2 mmol) for 10 h using the general procedure with (S)-CPA-8 as catalyst (73.1 mg, 91% Yield). R_{f}=0.3 (Hexane/EtOAc 2:1). HPLC analysis (Chiralpak IA-3, hexane/PrOH=80/20, 0.8 mL/min, 254 nm; tᵣ (major)=8.54 min, tᵣ(minor)=11.80 min) gave the isomeric composition of the product: 93% e.e., [α]_{D}¹⁹=−18.7 (c=1.13, CHCl₃). ¹H NMR (400 MHz, CDCl₃) δ 7.81 (d, J=8.4 Hz, 1H), 7.57 (s, 1H), 7.47 (d, J=8.4 Hz, 1H), 7.32 (d, J=6.8 Hz, 1H), 4.85-4.72 (m, 1H), 4.59 (t, J=12.0 Hz, 2H), 3.83 (s, 3H), 1.54 (d, J=7.6 Hz, 3H). ¹³C NMR (100 MHz, CDCl₃) δ 174.0, 164.1, 150.0, 142.0, 138.6, 125.7, 120.5, 114.5, 66.0 (t, J=28.2 Hz), 52.8, 48.8, 17.9. ¹⁹F NMR (376 MHz, CDCl₃) δ −83.4 (s), −123.3 (t, J=11.8 Hz). IR (neat, cm⁻¹) 3332, 2968, 1739, 1530, 1199. HRMS (ESI) calcd C₁₄H₁₄F₅N₂O₆⁺ [M+H]⁺: 401.0767. Found: 401.0766.

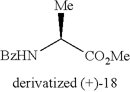

HPLC analysis of derivatized (+)-18 shown above (Chiralpak IA-3, hexane/PrOH=80/20, 0.8 mL/min, 254 nm; tᵣ (minor)=7.17 min, tᵣ (major)=8.01 min) gave the isomeric composition of the product: 97% e.e., [α]_{D}¹⁹=+12.9 (amino acid hydrochloride (+)-18, c=1.00, MeOH). ¹H NMR (400 MHz, DMSO-d₆) δ 8.38 (br, 2 H), 3.92 (q, J=7.1 Hz, 1H), 1.40 (d, J=7.2 Hz, 3H). ¹³C NMR (100 MHz, DMSO-d₆) δ 171.6, 47.8, 15.8. IR (neat, cm⁻¹) 3356, 2929, 1734, 1623, 1121. HRMS (ESI) calcd C₃H₈NO₂⁺ [M+H]⁺: 90.0550. Found: 90.0551.

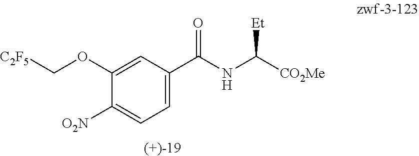

(+)-19 was obtained as white solid from the asymmetric decarboxylation reaction of malonic acid S19 (88.9 mg, 0.2 mmol) for 10 h using the general procedure (71.7 mg, 86% Yield). R_{f}=0.3 (Hexane/EtOAc 2:1). HPLC analysis (Chiralpak IA-3, hexane/PrOH=80/20, 0.8 mL/min, 254 nm; tᵣ (minor)=8.02 min, tᵣ (major)=11.44 min) gave the isomeric composition of the product: 93% e.e., [α]_{D}²³=+22.4 (c=1.00, CHCl₃). ¹H NMR (400 MHz, CDCl₃) δ 7.89 (d, J=8.4 Hz, 1H), 7.62 (s, 1H), 7.49 (d, J=8.4, 1H), 6.96 (d, J=7.2 Hz, 1H), 4.83-4.71 (m, 1H), 4.61 (t, J=11.8 Hz, 2H), 3.82 (s, 3H), 2.12-1.97 (m, 1H), 1.93-1.78 (m, 1H), 0.99 (t, J=7.4 Hz, 3H). ¹³C NMR (100 MHz, CDCl₃) δ 173.2, 164.4, 150.1, 142.0, 138.8, 125.8, 120.4, 114.7, 66.1 (t, J=28.6 Hz), 54.1, 52.7, 25.3, 9.7. ¹⁹F NMR (376 MHz, CDCl₃) δ −83.4 (s), −123.2 (t, J=11.7 Hz). IR (neat, cm⁻¹) 3312, 2974, 1737, 1531, 1199. HRMS (ESI) calcd C₁₅H₁₆F₅N₂O₆+[M+H]⁺: 415.0923. Found: 415.0926.

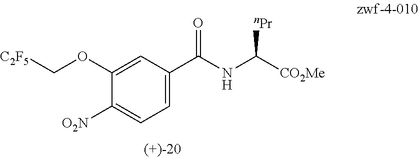

(+)-20 was obtained as white solid from the asymmetric decarboxylation reaction of malonic acid S20 (91.8 mg, 0.2 mmol) for 8 h using the general procedure (72.5 mg, 85% Yield). R_{f}=0.5 (Hexane/EtOAc 2:1). HPLC analysis (Chiralpak IA-3, hexane/PrOH=80/20, 0.8 mL/min, 254 nm; tᵣ (minor)=7.47 min, tᵣ (major)=11.24 min) gave the isomeric composition of the product: 93% e.e., [α]_{D}¹⁹=+20.9 (c=1.04, CHCl₃). ¹H NMR (400 MHz, CDCl₃) δ 7.88 (d, J=8.4 Hz, 1H), 7.61 (s, 1H), 7.48 (d, J=8.4, 1H), 6.94 (d, J=7.6 Hz, 1H), 4.87-4.74 (m, 1H), 4.60 (t, J=11.8 Hz, 2H), 3.82 (s, 3H), 2.03-1.87 (m, 1H), 1.85-1.71 (m, 1H), 1.51-1.33 (m, 2H), 0.97 (t, J=7.4 Hz, 3H). ¹³C NMR (100 MHz, CDCl₃) δ 173.4, 164.3, 150.2, 142.1, 138.8, 125.9, 120.4, 114.8, 66.2 (t, J=28.5 Hz), 52.8, 52.7, 34.3, 18.7, 13.6. ¹⁹F NMR (376 MHz, CDCl₃) δ −83.3 (s), −123.2 (t, J=11.8 Hz). IR (neat, cm⁻¹) 3303, 2968, 1748, 1539, 1194. HRMS (ESI) calcd C₁₆H₁₈F₅N₂O₆⁺ [M+H]⁺: 429.1080. Found: 429.1079.

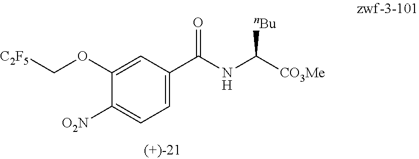

(+)-21 was obtained as colorless oil from the asymmetric decarboxylation reaction of malonic acid S21 (94.6 mg, 0.2 mmol) for 8 h using the general procedure (72.8 mg, 82% Yield). R_{f}=0.5 (Hexane/EtOAc 2:1). HPLC analysis (Chiralpak IA-3, hexane/ⁱPrOH=80/20, 0.8 mL/min, 254 nm; tᵣ (minor)=7.56 min, tᵣ (major)=10.98 min) gave the isomeric composition of the product: 91% e.e., [α]_{D}²²=+18.5 (c=1.01, CHCl₃). ¹H NMR (400 MHz, CDCl₃) δ 7.90 (d, J=8.4 Hz, 1H), 7.64 (s, 1H), 7.49 (d, J=8.4, 1H), 6.90 (d, J=7.2 Hz, 1H), 4.84-4.75 (m, 1H), 4.61 (t, J=11.8 Hz, 2H), 3.81 (s, 3H), 2.04-1.91 (m, 1H), 1.87-1.74 (m, 1H), 1.45-1.27 (m, 4H), 0.91 (t, J=6.6 Hz, 3H). ¹³C NMR (100 MHz, CDCl₃) δ 173.1, 164.3, 150.3, 142.2, 139.1, 126.0, 120.3, 115.1, 66.3 (t, J=28.6 Hz), 53.0, 52.7, 32.1, 27.4, 22.2, 13.8. ¹⁹F NMR (376 MHz, CDCl₃) δ −83.3 (s), −123.2 (t, J=11.8 Hz). IR (neat, cm⁻¹) 3393, 2959, 1742, 1532, 1195. HRMS (ESI) calcd C₁₇H₂₀F₅N₂O₆⁺ [M+H]⁺: 443.1236. Found: 443.1234.

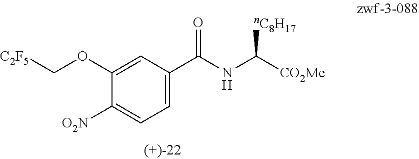

(+)-22 was obtained as colorless oil from the asymmetric decarboxylation reaction of malonic acid S22 (158.5 mg, 0.3 mmol) for 8 h using the general procedure (120.3 mg, 80% Yield). R_{f}=0.6 (Hexane/EtOAc 2:1). HPLC analysis (Chiralpak IA-3, hexane/PrOH=80/20, 0.8 mL/min, 254 nm; tᵣ (minor)=7.10 min, tᵣ (major)=8.92 min) gave the isomeric composition of the product: 92% e.e., [α]_{D}²²=+19.6 (c=0.92, CHCl₃). ¹H NMR (400 MHz, CDCl₃) δ 7.92 (d, J=8.4 Hz, 1H), 7.64 (s, 1H), 7.49 (d, J=8.4 Hz, 1H), 6.83 (d, J=8.0 Hz, 1H), 4.85-4.75 (m, 1H), 4.61 (t, J=11.8 Hz, 2H), 3.81 (s, 3H), 2.03-1.92 (m, 1H), 1.85-1.75 (m, 1H), 1.42-1.21 (m, 12H), 0.87 (t, J=6.8 Hz, 3H). ¹³C NMR (100 MHz, CDCl₃) δ 173.6, 164.3, 150.1, 142.1, 138.7, 125.8, 120.4, 114.7, 66.1 (t, J=28.2 Hz), 53.0, 52.7, 32.1, 31.7, 29.3, 29.11, 29.09, 25.4, 22.6, 14.0. ¹⁹F NMR (376 MHz, CDCl₃) δ −83.4 (s), −123.2 (t, J=12.6 Hz). IR (neat, cm⁻¹) 3260, 2928, 1744, 1533, 1196. HRMS (ESI) calcd C₂₁H₂₈F₅N₂O₆⁺ [M+H]⁺: 499.1862. Found: 499.1860.

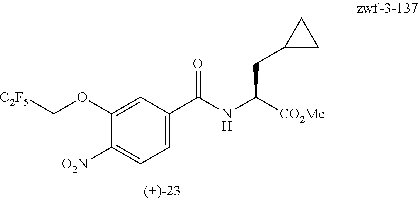

(+)-23 was obtained as colorless oil from the asymmetric decarboxylation reaction of malonic acid S23 (94.0 mg, 0.2 mmol) for 14 h using the general procedure (75.9 mg, 86% Yield). R_{f}=0.6 (Hexane/EtOAc 2:1). HPLC analysis (Chiralpak IA-3, hexane/PrOH=80/20, 0.8 mL/min, 254 nm; tᵣ (minor)=8.94 min, tᵣ (major)=13.33 min) gave the isomeric composition of the product: 91% e.e., [α]_{D}²⁰=+27.4 (c=1.01, CHCl₃). ¹H NMR (400 MHz, CDCl₃) δ 7.93 (d, J=8.4 Hz, 1H), 7.65 (s, 1H), 7.50 (d, J=7.6 Hz, 1H), 6.98 (d, J=7.6 Hz, 1H), 4.95-4.81 (m, 1H), 4.61 (t, J=11.8 Hz, 2H), 3.82 (s, 3H), 1.96-1.74 (m, 2H), 0.80-0.66 (m, 1H), 0.60-0.44 (m, 2H), 0.18-0.06 (m, 2H). ¹³C NMR (100 MHz, CDCl₃) δ 173.0, 164.1, 150.2, 142.1, 139.0, 125.9, 120.3, 114.9, 66.2 (t, J=28.5 Hz), 53.4, 52.6, 36.8, 6.9, 4.2, 4.1. ¹⁹F NMR (376 MHz, CDCl₃) δ −83.3 (s), −123.2 (t, J=11.7 Hz). IR (neat, cm⁻¹) 3233, 3050, 1750, 1538, 1197. HRMS (ESI) calcd C₁₇H₁₈F₅N₂O₆⁺[M+H]⁺: 441.1080. Found: 441.1081.

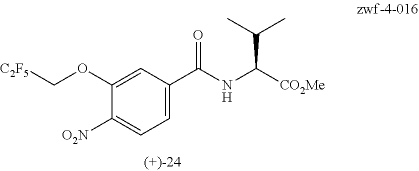

(+)-24 was obtained as white solid from the asymmetric decarboxylation reaction of malonic acid S24 (91.6 mg, 0.2 mmol) for 8 h using the general procedure (74.8 mg, 87% Yield). R_{f}=0.3 (Hexane/EtOAc 4:1). HPLC analysis (Chiralpak IA-3, hexane/PrOH=80/20, 0.8 mL/min, 254 nm; tᵣ (minor)=7.57 min, tᵣ (major)=11.69 min) gave the isomeric composition of the product: 84% e.e., [α]_{D}¹⁹=+21.4 (c=1.00, CHCl₃). ¹H NMR (400 MHz, CDCl₃) δ 7.93 (d, J=8.4 Hz, 1H), 7.65 (s, 1H), 7.50 (d, J=8.4 Hz, 1H), 6.76 (d, J=8.4 Hz, 1H), 4.82-4.71 (m, 1H), 4.62 (t, J=11.8 Hz, 2H), 3.81 (s, 3H), 2.38-2.23 (m, 1H), 1.02 (d, J=7.2 Hz, 3H), 1.00 (d, J=6.8 Hz, 3H). ¹³C NMR (125 MHz, CDCl₃) δ 172.4, 164.6, 150.3, 142.2, 139.3, 126.0, 120.2, 115.2, 66.3 (t, J=28.0 Hz), 57.9, 52.5, 31.5, 18.9, 17.9. ¹⁹F NMR (471 MHz, CDCl₃) δ −83.3 (s), -123.2 (t, J=11.3 Hz). IR (neat, cm⁻¹) 3369, 2968, 1743, 1530, 1199. HRMS (ESI) calcd C₁₆H₁₈F₅N₂O₆⁺ [M+H]⁺: 429.1080. Found: 429.1078.

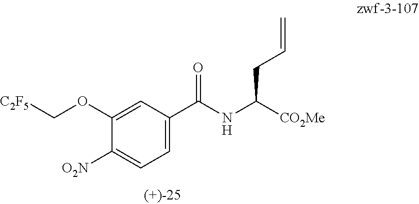

(+)-25 was obtained as colorless oil from the asymmetric decarboxylation reaction of malonic acid S25 (91.5 mg, 0.2 mmol) for 6 h using the general procedure (71.5 mg, 84% Yield). R_{f}=0.4 (Hexane/EtOAc 2:1). HPLC analysis (Chiralpak IA-3, hexane/PrOH=80/20, 0.8 mL/min, 254 nm; tᵣ (minor)=8.41 min, tᵣ (major)=14.32 min) gave the isomeric composition of the product: 92% e.e., [α]_{D}²²=+23.5 (c=1.05, CHCl₃). ¹H NMR (400 MHz, CDCl₃) δ 7.90 (d, J=8.4 Hz, 1H), 7.62 (d, J=0.8 Hz, 1H), 7.46 (dd, J₁=8.2, J₂=1.4 Hz, 1H), 6.87 (d, J=7.6 Hz, 1H), 5.84-5.63 (m, 1H), 5.25-5.10 (m, 2H), 4.94-4.78 (m, 1H), 4.61 (t, J=11.8 Hz, 2H), 3.82 (s, 3H), 2.83-2.55 (m, 2H). ¹³C NMR (100 MHz, CDCl₃) δ 172.0, 164.2, 150.3, 142.2, 139.1, 131.8, 126.0, 120.3, 119.8, 115.1, 66.3 (t, J=28.2 Hz), 52.7, 52.3, 36.3. ¹⁹F NMR (376 MHz, CDCl₃) δ −83.3 (s), −123.2 (t, J=11.8 Hz). IR (neat, cm⁻¹) 3228, 2961, 1748, 1641, 1534, 1195. HRMS (ESI) calcd C₁₆H₁₆F₅N₂O₆⁺ [M+H]⁺: 427.0923. Found: 427.0921.

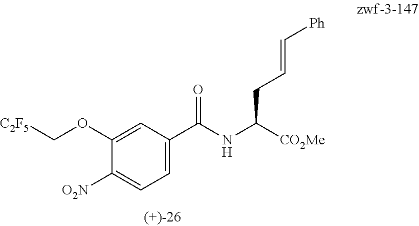

(+)-26 was obtained as yellow oil from the asymmetric decarboxylation reaction of malonic acid S26 (106.7 mg, 0.2 mmol) for 10 h using the general procedure (90.9 mg, 90% Yield). R_{f}=0.3 (Hexane/EtOAc 4:1). HPLC analysis (Chiralpak IA-3, hexane/PrOH=80/20, 0.8 mL/min, 254 nm; tᵣ (minor)=16.08 min, tᵣ (major)=16.67 min) gave the isomeric composition of the product: 93% e.e., [□]21 D=+44.0 (c=1.00, CHCl₃). ¹H NMR (400 MHz, CDCl₃) δ 7.88 (d, J=8.4 Hz, 1H), 7.55 (s, 1H), 7.44 (d, J=8.4 Hz, 1H), 7.36-7.28 (m, 4H), 7.27-7.22 (m, 1H), 6.81 (d, J=7.2 Hz, 1H), 6.48 (d, J=16.0 Hz, 1H), 6.17-6.01 (m, 1H), 5.01-4.87 (m, 1H), 4.51 (t, J=11.8 Hz, 2H), 3.84 (s, 3H), 2.97-2.86 (m, 1H), 2.84-2.74 (m, 1H). ¹³C NMR (100 MHz, CDCl₃) δ 172.1, 164.4, 150.2, 142.1, 139.1, 136.5, 134.6, 128.6, 127.9, 126.2, 126.0, 123.0, 120.3, 114.8, 66.1 (t, J=28.6 Hz), 52.8, 52.6, 35.7. ¹⁹F NMR (376 MHz, CDCl₃) δ −83.3 (s), −123.1 (t, J=11.8 Hz). IR (neat, cm⁻¹) 3225, 2961, 1750, 1642, 1538, 1197. HRMS (ESI) calcd C₂₂H₂₀F₅N₂O₆⁺ [M+H]⁺: 503.1236. Found: 503.1235.

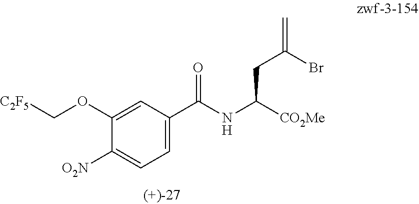

(+)-27 was obtained as white solid from the asymmetric decarboxylation reaction of malonic acid S27 (107.2 mg, 0.2 mmol) for 15 h using the general procedure (83.7 mg, 83% Yield). R_{f}=0.5 (Hexane/EtOAc 2:1). HPLC analysis (Chiralpak IA-3, hexane/PrOH=80/20, 0.8 mL/min, 254 nm; tᵣ (minor)=9.89 min, tᵣ (major)=17.82 min) gave the isomeric composition of the product: 93% e.e., [α]_{D}¹⁹=+37.7 (c=1.02, CHCl₃). ¹H NMR (500 MHz, CDCl₃) δ 7.92 (d, J=8.5 Hz, 1H), 7.64 (d, J=1.5 Hz, 1H), 7.50 (dd, J₁=8.5, J₂=1.5 Hz, 1H), 6.96 (d, J=7.0 Hz, 1H), 5.69 (s, 1H), 5.59 (d, J=1.5 Hz, 1H), 5.06-5.92 (m, 1H), 4.61 (t, J=12.0 Hz, 2H), 3.84 (s, 3H), 3.20 (dd, J₁=14.5, J₂=5.0 Hz, 1H), 3.08 (dd, J₁=14.8, J₂=6.3 Hz, 1H). ¹³C NMR (125 MHz, CDCl₃) δ 171.5, 164.4, 150.2, 142.2, 138.6, 127.2, 125.9, 121.3, 120.4, 114.9, 66.2 (t, J=28.4 Hz), 52.9, 51.8, 42.8. ¹⁹F NMR (471 MHz, CDCl₃) δ −83.4 (s), −123.2 (t, J=12.0 Hz). IR (neat, cm⁻¹) 3240, 3065, 1753, 1540, 1194. HRMS (ESI) calcd C₁₆H₁₅F₅N₂O₆⁷⁹Br⁺ [M+H]⁺: 505.0028. Found: 505.0027.

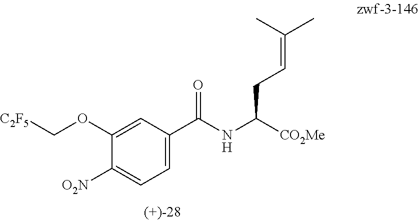

(+)-28 was obtained as yellow oil from the asymmetric decarboxylation reaction of malonic acid S28 (96.9 mg, 0.2 mmol) for 10 h using the general procedure (80.3 mg, 88% Yield). R_{f}=0.3 (Hexane/EtOAc 4:1). HPLC analysis (Chiralpak IA-3, hexane/PrOH=80/20, 0.8 mL/min, 254 nm; tᵣ (minor)=7.88 min, tᵣ (major)=12.60 min) gave the isomeric composition of the product: 91% e.e., [α]_{D}²¹=+37.1 (c=1.02, CHCl₃). ¹H NMR (500 MHz, CDCl₃) δ 7.92 (d, J=8.5 Hz, 1H), 7.63 (s, 1H), 7.45 (d, J=6.5 Hz, 1H), 6.80 (d, J=7.0 Hz, 1H), 5.06 (t, J=7.5 Hz, 1H), 4.87-4.76 (m, 1H), 4.61 (t, J=11.8 Hz, 2H), 3.81 (s, 3H), 2.78-2.67 (m, 1H), 2.64-2.54 (m, 1H), 1.72 (s, 3H), 1.62 (s, 3H). ¹³C NMR (125 MHz, CDCl₃) δ 172.3, 164.2, 150.4, 142.2, 139.3, 137.2, 126.1, 120.2, 116.9, 115.2, 66.3 (t, J=27.9 Hz), 52.8, 52.7, 30.5, 25.8, 17.9. ¹⁹F NMR (471 MHz, CDCl₃) δ −83.3 (s), −123.1 (t, J=12.0 Hz). IR (neat, cm⁻¹) 3251, 3051, 1748, 1647, 1534, 1194. HRMS (ESI) calcd C₁₈H₂₀F₅N₂O₆⁺ [M+H]⁺: 455.1236. Found: 455.1237.

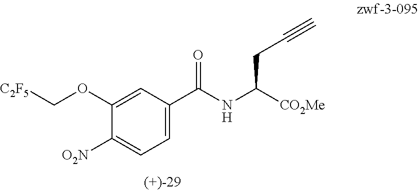

(+)-29 was obtained as white solid from the asymmetric decarboxylation reaction of malonic acid S29 (91.2 mg, 0.2 mmol) for 6 h using the general procedure (72.4 mg, 85% Yield). R_{f}=0.5 (Hexane/EtOAc 2:1). HPLC analysis (Chiralpak IA-3, hexane/ⁱPrOH=80/20, 0.8 mL/min, 254 nm; tᵣ (minor)=12.33 min, tᵣ (major)=24.54 min) gave the isomeric composition of the product: 92% e.e., [α]_{D}²²=+64.7 (c=1.09, CHCl₃). ¹H NMR (400 MHz, CDCl₃) δ 7.95 (d, J=8.4 Hz, 1H), 7.65 (s, 1H), 7.54 (d, J=8.4 Hz, 1H), 7.04 (d, J=6.8 Hz, 1H), 4.98-4.86 (m, 1H), 4.62 (t, J=11.8 Hz, 2H), 3.87 (s, 3H), 3.04-2.81 (m, 2H), 2.09 (t, J=2.4 Hz, 1H). ¹³C NMR (100 MHz, CDCl₃) δ 170.6, 164.4, 150.3, 142.3, 138.8, 126.0, 120.5, 115.2, 77.9, 72.1, 66.2 (t, J=28.5 Hz), 53.1, 51.3, 22.2. ¹⁹F NMR (376 MHz, CDCl₃) δ −83.3 (s), −123.2 (t, J=11.7 Hz). IR (neat, cm⁻¹) 3306, 2973, 1732, 1524, 1198. HRMS (ESI) calcd C₁₆H₁₄F₅N₂O₆⁺ [M+H]⁺: 425.0767. Found: 425.0771.

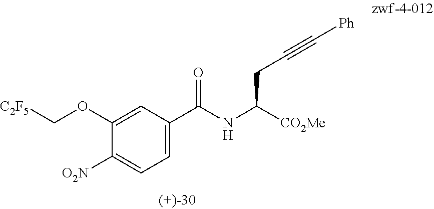

(+)-30 was obtained as colorless oil from the asymmetric decarboxylation reaction of malonic acid S30 (106.3 mg, 0.2 mmol) for 5 h using the general procedure (88.5 mg, 88% Yield). R_{f}=0.3 (Hexane/EtOAc 2:1). HPLC analysis (Chiralpak IA-3, hexane/ⁱPrOH=80/20, 0.8 mL/min, 254 nm; tᵣ (major)=12.71 min, tᵣ (minor)=14.87 min) gave the isomeric composition of the product: 90% e.e., [α]_{D}¹⁹=+108.4 (c=1.02, CHCl₃). ¹H NMR (400 MHz, CDCl₃) δ 7.91 (d, J=8.4 Hz, 1H), 7.63 (s, 1H), 7.53 (d, J=8.4 Hz, 1H), 7.40-7.25 (m, 5H), 7.11 (d, J=7.2 Hz, 1H), 5.05-4.92 (m, 1H), 4.57 (t, J=11.8 Hz, 2H), 3.88 (s, 3H), 3.25-3.03 (m, 2H). ¹³C NMR (100 MHz, CDCl₃) δ 170.7, 164.3, 150.3, 142.3, 139.1, 131.6, 128.5, 128.4, 126.1, 122.5, 120.5, 115.2, 84.2, 83.0, 66.3 (t, J=28.2 Hz), 53.1, 51.6, 23.4. ¹⁹F NMR (376 MHz, CDCl₃) δ −83.3 (s), −123.1 (t, J=11.7 Hz). IR (neat, cm⁻¹) 3313, 2953, 1753, 1534, 1199. HRMS (ESI) calcd C₂₂H₁₈F₅N₂O₆⁺ [M+H]⁺: 501.1080. Found: 501.1076.

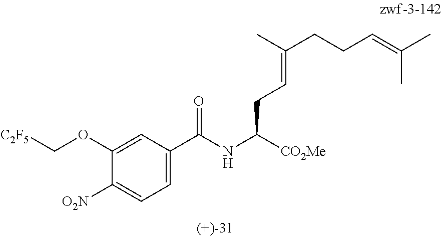

(+)-31 was obtained as yellow oil from the asymmetric decarboxylation reaction of malonic acid S31 (110.4 mg, 0.2 mmol) for 8 h using the general procedure (66.5 mg, 64% Yield). R_{f}=0.6 (Hexane/EtOAc 2:1). HPLC analysis (Chiralpak IA-3, hexane/PrOH=80/20, 0.8 mL/min, 254 nm; tᵣ (minor)=6.62 min, tᵣ (major)=10.41 min) gave the isomeric composition of the product: 88% e.e., [α]_{D}²⁰=+40.0 (c=1.00, CHCl₃). ¹H NMR (400 MHz, CDCl₃) δ 7.92 (d, J=8.4 Hz, 1H), 7.63 (s, 1H), 7.43 (d, J=8.4 Hz, 1H), 6.77 (d, J=7.2 Hz, 1H), 5.15-4.96 (m, 2H), 4.89-4.78 (m, 1H), 4.60 (t, J=11.8 Hz, 2H), 3.81 (s, 3H), 2.81-2.68 (m, 1H), 2.66-2.55 (m, 1H), 2.12-1.95 (m, 4H), 1.65 (s, 3H), 1.62 (s, 3H), 1.58 (s, 3H). ¹³C NMR (100 MHz, CDCl₃) δ 172.4, 164.1, 150.3, 142.1, 140.6, 139.1, 131.8, 126.0, 123.6, 120.2, 117.0, 115.0, 66.2 (t, J=28.2 Hz), 52.8, 52.6, 39.7, 30.3, 26.4, 25.6, 17.6, 16.1. ¹⁹F NMR (376 MHz, CDCl₃) δ −83.3 (s), −123.2 (t, J=11.8 Hz). IR (neat, cm⁻¹) 3224, 2961, 1750, 1643, 1538, 1196. HRMS (ESI) calcd C₂₃H₂₈F₅N₂O₆⁺ [M+H]⁺: 523.1862. Found: 523.1859.

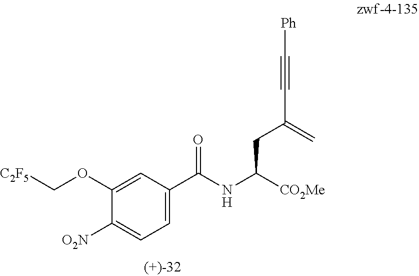

(+)-32 was obtained as white solid from the asymmetric decarboxylation reaction of malonic acid S32 (111.2 mg, 0.2 mmol) for 12 h using the general procedure (85.7 mg, 81% Yield). R_{f}=0.6 (Hexane/EtOAc 2:1). HPLC analysis (Chiralpak IA-3, hexane/PrOH=80/20, 0.8 mL/min, 254 nm; tᵣ (minor)=9.12 min, tᵣ (major)=19.17 min) gave the isomeric composition of the product: 93% e.e., [α]_{D}¹⁶=+44.2 (c=1.00, CHCl₃). ¹H NMR (400 MHz, CDCl₃) δ 7.69 (d, J=8.4 Hz, 1H), 7.57 (s, 1H), 7.42 (d, J=8.4 Hz, 1H), 7.35-7.27 (m, 5H), 7.10 (d, J=7.2 Hz, 1H), 5.59 (s, 1H), 5.40 (s, 1H), 5.09-4.98 (m, 1H), 4.46 (t, J=11.8 Hz, 2H), 3.81 (s, 3H), 3.01 (dd, J₁=14.0, J₂=4.8 Hz, 1H), 2.89 (dd, J₁=14.0, J₂=5.6 Hz, 1H). ¹³C NMR (100 MHz, CDCl₃) δ 171.5, 164.2, 150.1, 142.1, 138.9, 131.4, 128.8, 128.4, 125.8, 125.73, 125.67, 122.2, 120.3, 114.8, 90.7, 88.8, 66.0 (t, J=28.6 Hz), 52.8, 52.6, 38.6. ¹⁹F NMR (376 MHz, CDCl₃) δ −83.3 (s), −123.2 (t, J=11.8 Hz). IR (neat, cm⁻¹) 3230, 3056, 2960, 2150, 1746, 1531, 1199. HRMS (ESI) calcd C₂₄H₂₀F₅N₂O₆⁺ [M+H]⁺: 527.1236. Found: 527.1238.

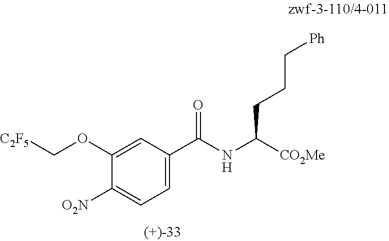

(+)-33 was obtained as white solid from the asymmetric decarboxylation reaction of malonic acid S33 (107.0 mg, 0.2 mmol) for 8 h using the general procedure (91.4 mg, 91% Yield). R_{f}=0.5 (Hexane/EtOAc 2:1). HPLC analysis (Chiralpak IA-3, hexane/PrOH=80/20, 0.8 mL/min, 254 nm; tᵣ (minor)=11.72 min, tᵣ (major)=13.77 min) gave the isomeric composition of the product: 92% e.e., [α]_{D}¹⁸=+25.2 (c=1.00, CHCl₃). ¹H NMR (400 MHz, CDCl₃) δ 7.89 (d, J=8.4 Hz, 1H), 7.61 (s, 1H), 7.45 (d, J=8.4 Hz, 1H), 7.31-7.25 (m, 2H), 7.24-7.06 (m, 3H), 6.80 (d, J=7.6 Hz, 1H), 4.90-4.75 (m, 1H), 4.60 (t, J=11.8 Hz, 2H), 3.79 (s, 3H), 2.76-2.56 (m, 2H), 2.08-1.93 (m, 1H), 1.90-1.64 (m, 3H). ¹³C NMR (100 MHz, CDCl₃) δ 173.2, 164.3, 150.1, 142.1, 141.3, 138.7, 128.4, 128.3, 126.1, 125.8, 120.4, 114.8, 66.1 (t, J=28.6 Hz), 52.8, 52.7, 35.2, 31.7, 27.2. ¹⁹F NMR (376 MHz, CDCl₃) δ −83.3 (s), −123.2 (t, J=11.8 Hz). IR (neat, cm⁻¹) 3338, 2956, 1741, 1531, 1194. HRMS (ESI) calcd C₂₂H₂₂F₅N₂O₆⁺ [M+H]⁺: 505.1393. Found: 505.1394.

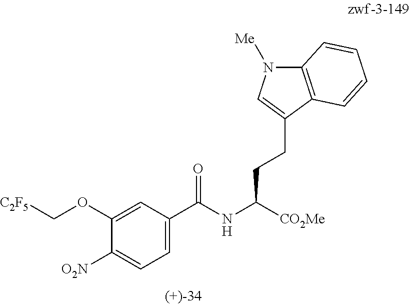

(+)-34 was obtained as yellow oil from the asymmetric decarboxylation reaction of malonic acid S34 (114.2 mg, 0.2 mmol) for 12 h using the general procedure (89.7 mg, 83% Yield). R_{f}=0.4 (Hexane/EtOAc 2:1). HPLC analysis (Chiralpak IA-3, hexane/PrOH=80/20, 0.8 mL/min, 254 nm; tᵣ (minor)=14.09 min, tᵣ (major)=18.80 min) gave the isomeric composition of the product: 90% e.e., [α]_{D}¹⁹=+15.9 (c=1.05, CHCl₃). ¹H NMR (400 MHz, CDCl₃) δ 7.67 (d, J=8.4 Hz, 1H), 7.57 (d, J=8.0 Hz, 1H), 7.38 (s, 1H), 7.27-7.20 (m, 2H), 7.14-7.06 (m, 1H), 6.86 (s, 1H), 6.85-6.71 (m, 2H), 4.95-4.78 (m, 1H), 4.49 (t, J=12.0 Hz, 2H), 3.78 (s, 3H), 3.65 (s, 3H), 2.99-2.80 (m, 2H), 2.49-2.38 (m, 1H), 2.32-2.21 (m, 1H). ¹³C NMR (100 MHz, CDCl₃) δ 172.8, 164.0, 150.0, 141.9, 138.7, 137.2, 127.2, 126.8, 125.6, 121.9, 119.9, 119.1, 118.8, 114.6, 112.9, 109.4, 66.1 (t, J=28.6 Hz), 53.3, 52.6, 32.5, 31.8, 21.5. ¹⁹F NMR (376 MHz, CDCl₃) δ −83.3 (s), −123.2 (t, J=11.8 Hz). IR (neat, cm⁻¹) 3342, 2963, 1748, 1530, 1199. HRMS (ESI) calcd C₂₄H₂₃F₅N₃O₆⁺ [M+H]⁺: 544.1502. Found: 544.1500.

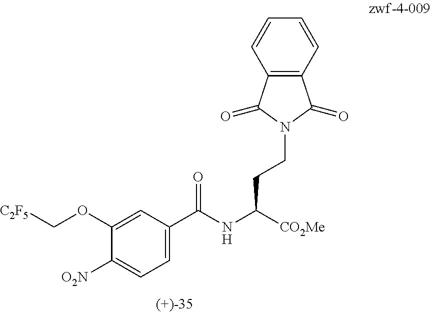

(+)-35 was obtained as white solid from the asymmetric decarboxylation reaction of malonic acid S35 (117.8 mg, 0.2 mmol) for 12 h using the general procedure (87.5 mg, 78% Yield). R_{f}=0.3 (Hexane/EtOAc 2:1). HPLC analysis (Chiralpak ID-3, hexane/ⁱPrOH=70/30, 0.8 mL/min, 230 nm; tᵣ (minor)=49.50 min, tᵣ (major)=136.27 min) gave the isomeric composition of the product: 72% e.e., (57% Yield, 99% e.e., single recrystallized from DCM/hexane, Chiralpak IB-3, hexane/PrOH=70/30, 0.8 mL/min, 254 nm; tᵣ (major)=28.09 min, tᵣ (minor)=32.20 min), [α]_{D}¹⁹=+124.8 (c=0.97, CHCl₃ (99% e.e.)). ¹H NMR (400 MHz, CDCl₃) δ 7.97 (d, J=8.4 Hz, 1H), 7.91-7.81 (m, 3H), 7.81-7.68 (m, 3H), 7.55 (d, J=8.0 Hz, 1H), 4.99-4.89 (m, 1H), 4.75 (t, J=12.2 Hz, 2H), 3.97-3.76 (m, 2H), 3.49 (s, 3H), 2.64-2.48 (m, 1H), 2.40-2.31 (m, 1H). ¹³C NMR (100 MHz, CDCl₃) δ 171.8, 168.6, 164.3, 150.2, 142.0, 138.9, 134.5, 131.7, 126.2, 123.4, 121.2, 114.4, 66.1 (t, J=28.2 Hz), 52.6, 50.1, 33.5, 29.2. ¹⁹F NMR (376 MHz, CDCl₃) δ −83.3 (s), −123.2 (t, J=11.8 Hz). IR (neat, cm⁻¹) 3245, 3065, 1714, 1534, 1204. HRMS (ESI) calcd C₂₃H₁₉F₅N₃O₈⁺ [M+H]⁺: 560.1087. Found: 560.1083.

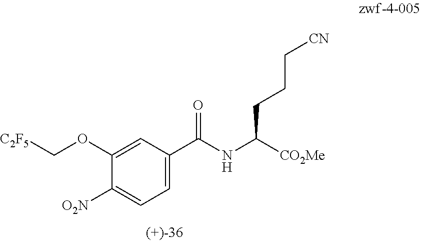

(+)-36 was obtained as colorless oil from the asymmetric decarboxylation reaction of malonic acid S36 (96.7 mg, 0.2 mmol) for 18 h using the general procedure (77.9 mg, 86% Yield). R_{f}=0.3 (Hexane/EtOAc 2:1). HPLC analysis (Chiralpak ID-3, hexane/ⁱPrOH=70/30, 0.8 mL/min, 254 nm; tᵣ (minor)=12.48 min, tᵣ (major)=21.42 min) gave the isomeric composition of the product: 91% e.e., [α]_{D}¹⁹=+23.0 (c=1.01, CHCl₃). ¹H NMR (400 MHz, CDCl₃) δ 7.90 (d, J=8.0 Hz, 1H), 7.62 (s, 1H), 7.51 (d, J=8.0 Hz, 1H), 7.10 (d, J=7.6 Hz, 1H), 4.89-4.76 (m, 1H), 4.62 (t, J=11.8 Hz, 2H), 3.84 (s, 3H), 2.46 (t, J=7.0 Hz, 2H), 2.24-2.10 (m, 1H), 2.02-1.90 (m, 1H), 1.88-1.75 (m, 2H). ¹³C NMR (100 MHz, CDCl₃) δ 172.4, 164.6, 150.1, 142.2, 138.5, 125.9, 120.5, 119.1, 114.8, 66.1 (t, J=28.2 Hz), 53.0, 52.2, 31.2, 21.7, 16.8. ¹⁹F NMR (376 MHz, CDCl₃) δ −83.3 (s), −123.2 (t, J=11.7 Hz). IR (neat, cm⁻¹) 3337, 2958, 2180, 1743, 1530, 1199. HRMS (ESI) calcd C₁₇H₁₇F₅N₃O₆⁺ [M+H]⁺: 454.1032. Found: 454.1036.

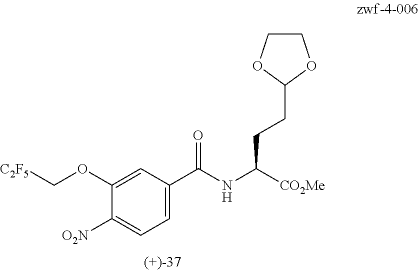

(+)-37 was obtained as colorless oil from the asymmetric decarboxylation reaction of malonic acid S37 (103.5 mg, 0.2 mmol) for 18 h using the general procedure (85.9 mg, 88% Yield). R_{f}=0.3 (Hexane/EtOAc 2:1). HPLC analysis (Chiralpak IA-3, hexane/ⁱPrOH=80/20, 0.8 mL/min, 254 nm; tᵣ (minor)=25.48 min, tᵣ (major)=33.43 min) gave the isomeric composition of the product: 90% e.e., [α]_{D}¹⁸=+8.2 (c=1.03, CHCl₃). ¹H NMR (400 MHz, CDCl₃) δ 7.88 (d, J=8.4 Hz, 1H), 7.65 (s, 1H), 7.61-7.47 (m, 2H), 4.98-4.89 (m, 1H), 4.80-4.69 (m, 1H), 4.61 (t, J=11.8 Hz, 2H), 4.05-3.75 (m, 7H), 2.17-1.80 (m, 4H). ¹³C NMR (100 MHz, CDCl₃) δ 172.6, 164.5, 150.2, 142.1, 139.0, 125.9, 120.4, 115.0, 103.3, 66.2 (t, J=28.6 Hz), 65.0, 52.8, 52.6, 29.0, 25.1. ¹⁹F NMR (376 MHz, CDCl₃) δ −83.3 (s), −123.2 (t, J=11.8 Hz). IR (neat, cm⁻¹) 3314, 2968, 1743, 1534, 1194. HRMS (ESI) calcd C₁₈H₂₀F₅N₂O₈⁺ [M+H]⁺: 487.1134. Found: 487.1132.

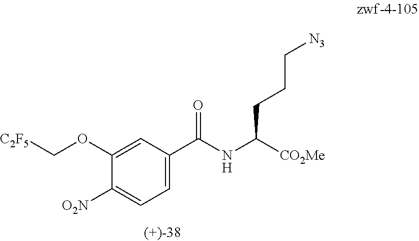

(+)-38 was obtained as colorless oil from the asymmetric decarboxylation reaction of malonic acid S38 (99.9 mg, 0.2 mmol) for 12 h using the general procedure (84.3 mg, 90% Yield). R_{f}=0.4 (Hexane/EtOAc 2:1). HPLC analysis (Chiralpak IA-3, hexane/PrOH=80/20, 0.8 mL/min, 254 nm; tᵣ (minor)=14.33 min, tᵣ (major)=16.43 min) gave the isomeric composition of the product: 92% e.e., [α]_{D}¹⁹=+19.1 (c=1.00, CHCl₃). ¹H NMR (400 MHz, CDCl₃) δ 7.89 (d, J=8.0 Hz, 1H), 7.61 (s, 1H), 7.49 (d, J=8.4 Hz, 1H), 7.08 (d, J=7.6 Hz, 1H), 4.90-4.76 (m, 1H), 4.60 (t, J=11.8 Hz, 2H), 3.84 (s, 3H), 3.45-3.28 (m, 2H), 2.16-2.03 (m, 1H), 1.97-1.84 (m, 1H), 1.77-1.66 (m, 2H). ¹³C NMR (100 MHz, CDCl₃) δ 172.8, 164.5, 150.1, 142.1, 138.6, 125.9, 120.4, 114.8, 66.1 (t, J=28.2 Hz), 52.9, 52.5, 50.6, 29.4, 24.9. ¹⁹F NMR (376 MHz, CDCl₃) δ −83.4 (s), −123.2 (t, J=12.6 Hz). IR (neat, cm⁻¹) 3245, 2968, 2103, 1739, 1534, 1194. HRMS (ESI) calcd C₁₆H₁₇F₅N₆O₆⁺ [M+H]⁺: 470.1094. Found: 470.1093.

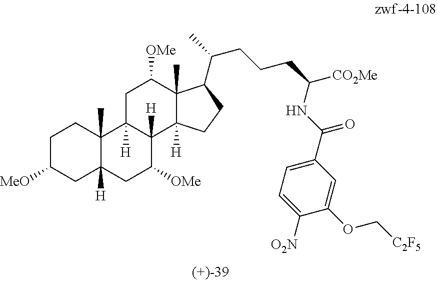

(+)-39 was obtained as colorless oil from the asymmetric decarboxylation reaction of malonic acid S39 (167.0 mg, 0.2 mmol) for 15 h using the general procedure (137.0 mg, 86% Yield). >20:1 d.r., as determined by ¹H NMR analysis of crude product with CH₂Br₂ (14 μL) as the internal standard. R_{f}=0.5 (Hexane/EtOAc 2:1). HPLC analysis (Chiralpak ID-3, hexane/ⁱPrOH=80/20, 0.8 mL/min, 254 nm; tᵣ (minor)=11.35 min, tᵣ (major)=18.56 min) gave the isomeric composition of the product: 91% e.e., [α]_{D}¹⁹=+33.6 (c=1.00, CHCl₃). ¹H NMR (400 MHz, CDCl₃) δ 7.85 (d, J=8.0 Hz, 1H), 7.60 (s, 1H), 7.49 (d, J=8.4 Hz, 1H), 7.13 (d, J=7.2 Hz, 1H), 4.82-4.71 (m, 1H), 4.60 (t, J=11.8 Hz, 2H), 3.82 (s, 3H), 3.39-3.28 (m, 4H), 3.24 (s, 3H), 3.20 (s, 3H), 3.14 (s, 1H), 3.05-2.93 (m, 1H), 2.24-1.64 (m, 13H), 1.63-1.56 (m, 1H), 1.55-1.38 (m, 4H), 1.32-0.96 (m, 8H), 0.92-0.84 (m, 6H), 0.64 (s, 3H). ¹³C NMR (100 MHz, CDCl₃) δ 173.6, 164.3, 150.1, 142.1, 138.8, 125.8, 120.4, 114.8, 82.0, 80.7, 76.9, 66.1 (t, J=28.3 Hz), 55.8, 55.6, 55.3, 53.2, 52.6, 46.4, 46.0, 42.7, 41.9, 39.5, 35.4, 35.3, 35.2, 34.8, 34.4, 32.6, 27.9, 27.7, 27.5, 26.7, 23.1, 22.8, 22.0, 21.9, 17.6, 12.4. ¹⁹F NMR (376 MHz, CDCl₃) δ −83.3 (s), −123.2 (t, J=11.8 Hz). IR (neat, cm⁻¹) 2939, 2866, 1739, 1539, 1194, 1102. HRMS (ESI) calcd C₄₀H₅₈F₅N₂O₉⁺ [M+H]⁺: 805.4057. Found: 805.4055.

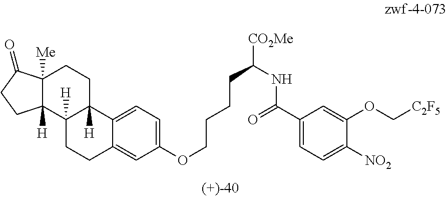

(+)-40 was obtained as colorless oil from the asymmetric decarboxylation reaction of malonic acid S40 (148.0 mg, 0.2 mmol) for 15 h using the general procedure (120.4 mg, 85% Yield). >20:1 d.r., as determined by ¹H NMR analysis of crude product with CH2Br2 (14 μL) as the internal standard. R_{f}=0.3 (Hexane/EtOAc 2:1). HPLC analysis (Chiralpak IA-3, hexane/ⁱPrOH=80/20, 0.8 mL/min, 254 nm; tᵣ (minor)=29.47 min, tᵣ (major)=37.40 min) gave the isomeric composition of the product: 94% e.e., [α]_{D}¹⁸=+72.1 (c=1.08, CHCl₃). ¹H NMR (400 MHz, CDCl₃) δ 7.84 (d, J=8.4 Hz, 1H), 7.61 (s, 1H), 7.49 (d, J=7.2 Hz, 1H), 7.24-7.10 (m, 2H), 6.67 (d, J=8.8 Hz, 1H), 6.60 (s, 1H), 4.89-4.76 (m, 1H), 4.58 (t, J=11.8 Hz, 2H), 3.94 (t, J=5.8 Hz, 2H), 3.81 (s, 3H), 2.94-2.79 (m, 2H), 2.55-2.45 (m, 1H), 2.43-2.33 (m, 1H), 2.28-1.78 (m, 10H), 1.67-1.42 (m, 7H), 0.90 (s, 3H). ¹³C NMR (100 MHz, CDCl₃) δ 221.0, 173.1, 164.4, 156.8, 150.1, 142.0, 138.8, 137.8, 132.1, 126.3, 125.8, 120.5, 114.8, 114.4, 111.9, 67.2, 66.1 (t, J=28.3 Hz), 52.9, 52.7, 50.3, 47.9, 43.9, 38.3, 35.8, 31.7, 31.5, 29.6, 28.6, 26.4, 25.8, 22.2, 21.5, 13.7. ¹⁹F NMR (376 MHz, CDCl₃) δ −83.3 (s), −123.1 (t, J=11.8 Hz). IR (neat, cm⁻¹) 3351, 2943, 1729, 1534, 1194. HRMS (ESI) calcd C₃₅H₄₀F₅N₂O₈⁺ [M+H]⁺: 711.2699. Found: 711.2700.

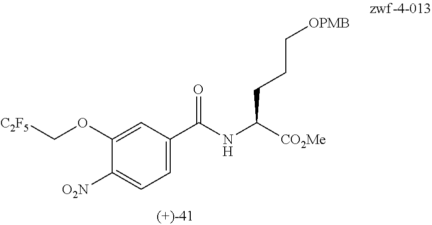

(+)-41 was obtained as colorless oil from the asymmetric decarboxylation reaction of malonic acid S41 (118.9 mg, 0.2 mmol) for 5 h using the general procedure (104.4 mg, 92% Yield). R_{f}=0.3 (Hexane/EtOAc 2:1). HPLC analysis (Chiralpak IA-3, hexane/ⁱPrOH=80/20, 0.8 mL/min, 254 nm; tᵣ (minor)=16.08 min, tᵣ (major)=22.40 min) gave the isomeric composition of the product: 91% e.e., [α]_{D}¹⁹=+14.7 (c=1.05, CHCl₃). ¹H NMR (500 MHz, CDCl₃) δ 7.78-7.63 (m, 2H), 7.55 (s, 1H), 7.29 (d, J=8.5 Hz, 1H), 7.22 (d, J=8.0 Hz, 2H), 6.81 (d, J=8.0 Hz, 2H), 4.77-4.67 (m, 1H), 4.54 (t, J=11.8 Hz, 2H), 4.47 (d, J=11.0 Hz, 1H), 4.40 (d, J=11.5 Hz, 1H), 3.79 (s, 3H), 3.77 (s, 3H), 3.56 (t, J=5.3 Hz, 2H), 2.06 (q, J=6.5 Hz, 2H), 1.80-1.68 (m, 2H). ¹³C NMR (125 MHz, CDCl₃) δ 172.6, 164.6, 159.4, 150.0, 141.8, 139.0, 129.6, 129.5, 125.8, 120.4, 114.9, 113.8, 73.0, 69.7, 66.1 (t, J=28.4 Hz), 55.2, 52.8, 52.5, 28.9, 25.2. ¹⁹F NMR (471 MHz, CDCl₃) δ −83.3 (s), −123.2 (t, J=12.0 Hz). IR (neat, cm⁻¹) 3356, 2929, 1661, 1632, 1277. HRMS (ESI) calcd C₂₄H₂₆F₅N₂O₈⁺ [M+H]⁺: 565.1604. Found: 565.1606.

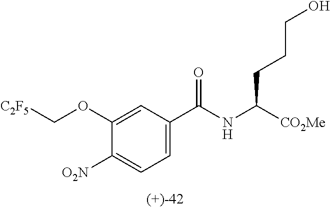

HPLC analysis (Chiralpak IA-3, hexane/ⁱPrOH=80/20, 0.8 mL/min, 254 nm; tᵣ(minor)=12.82 min, tᵣ (major)=22.73 min) gave the isomeric composition of the product: 91% e.e., [α]_{D}²⁴=+9.6 (c=1.00, CHCl₃). ¹H NMR (400 MHz, CDCl₃) δ 8.03 (d, J=6.8 Hz, 1H), 7.83 (d, J=8.4 Hz, 1H), 7.64 (s, 1H), 7.54 (d, J=8.4 Hz, 1H), 4.76-4.67 (m, 1H), 4.62 (t, J=12.0 Hz, 2H), 3.88-3.60 (m, 5H), 2.87 (br, 1H), 2.12-1.94 (m, 2H), 1.77-1.59 (m, 2H). ¹³C NMR (100 MHz, CDCl₃) δ 172.8, 164.8, 150.0, 141.8, 138.9, 125.8, 120.6, 114.7, 66.0 (t, J=28.6 Hz), 61.9, 52.8, 52.6, 28.7, 27.9. ¹⁹F NMR (376 MHz, CDCl₃) δ −83.4 (s), −123.3 (t, J=12.6 Hz). IR (neat, cm⁻¹) 3308, 2943, 1743, 1539, 1194. HRMS (ESI) calcd C₁₆H₁₈F₅N₂O₇⁺ [M+H]⁺: 445.1029. Found: 445.1031.

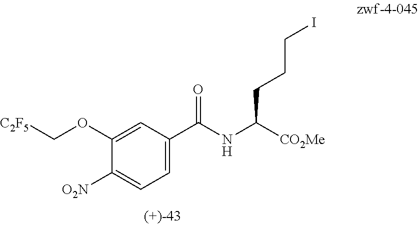

HPLC analysis (Chiralpak IA-3, hexane/ⁱPrOH=80/20, 0.8 mL/min, 254 nm; tᵣ(minor)=13.92 min, tᵣ (major)=15.77 min) gave the isomeric composition of the product: 91% e.e., [α]_{D}²²=+10.1 (c=1.00, CHCl₃). ¹H NMR (400 MHz, CDCl₃) δ 7.89 (d, J=8.4 Hz, 1H), 7.62 (s, 1H), 7.49 (d, J=8.4 Hz, 1H), 7.05 (d, J=7.6 Hz, 1H), 4.87-4.76 (m, 1H), 4.61 (t, J=11.8 Hz, 2H), 3.84 (s, 3H), 3.30-3.15 (m, 2H), 2.20-2.05 (m, 1H), 2.03-1.84 (m, 3H). ¹³C NMR (100 MHz, CDCl₃) δ 172.6, 164.4, 150.2, 142.2, 138.7, 126.0, 120.3, 115.0, 66.2 (t, J=28.2 Hz), 52.9, 52.1, 33.2, 29.2, 5.1. ¹⁹F NMR (376 MHz, CDCl₃) δ −83.3 (s), −123.2 (t, J=11.8 Hz). IR (neat, cm⁻¹) 3393, 2958, 1748, 1530, 1199. HRMS (ESI) calcd C₁₆H₁₇F₅N₂O₆I⁺ [M+H]⁺: 555.0046. Found: 555.0048.

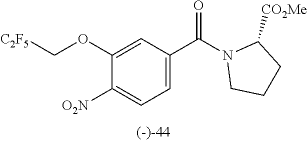

HPLC analysis (Chiralpak IA-3, hexane/PrOH=80/20, 0.8 mL/min, 254 nm; tᵣ(major)=14.90 min, tᵣ (minor)=17.46 min) gave the isomeric composition of the product: 91% e.e., [α]_{D}¹⁹=−41.1 (c=1.00, CHCl₃). ¹H NMR (400 MHz, CDCl₃) δ 7.98-7.84 (m, 1H), 7.42-7.29 and 7.21-7.13 (m, 2H), 4.73-4.48 (m, 3H), 3.80 (s, 3H), 3.68-3.58 (m, 1H), 3.55-3.40 (m, 1H), 2.44-2.24 (m, 1H), 2.15-1.89 (m, 3H). ¹³C NMR (100 MHz, CDCl₃) δ 172.1, 166.6, 150.2, 141.8, 141.1, 126.0, 125.9, 121.2, 120.6, 115.0, 114.7, 66.4 (t, J=28.6 Hz), 61.1, 59.2, 52.5, 52.4, 49.8, 46.7, 31.3, 29.2, 25.2, 22.4. ¹⁹F NMR (376 MHz, CDCl₃) δ −83.4 (s), −123.2 (t, J=11.7 Hz). IR (neat, cm⁻¹) 2958, 1743, 1632, 1531, 1437, 1194. HRMS (ESI) calcd C₁₆H₁₆F₅N₂O₆⁺ [M+H]⁺: 427.0923. Found: 427.0926.

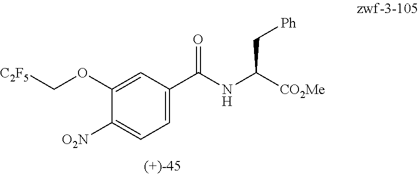

(+)-45 was obtained as white solid from the asymmetric decarboxylation reaction of malonic acid S45 (101.3 mg, 0.2 mmol) for 8 h using the general procedure (83.4 mg, 87% Yield). R_{f}=0.5 (Hexane/EtOAc 2:1). HPLC analysis (Chiralpak IA-3, hexane/PrOH=80/20, 0.8 mL/min, 254 nm; tᵣ (minor)=12.37 min, tᵣ (major)=20.38 min) gave the isomeric composition of the product: 92% e.e., [α]_{D}²¹=+65.5 (c=0.96, CHCl₃). ¹H NMR (400 MHz, CDCl₃) δ 7.87 (d, J=8.4 Hz, 1H), 7.54 (s, 1H), 7.37-7.26 (m, 4H), 7.12 (d, J=7.6 Hz, 2H), 6.69 (d, J=7.6 Hz, 1H), 5.13-5.00 (m, 1H), 4.58 (t, J=12.0 Hz, 2H), 3.82 (s, 3H), 3.32 (dd, J₁=14.0, J₂=5.6 Hz, 1H), 3.23 (dd, J₁=13.8, J₂=5.4 Hz, 1H). ¹³C NMR (100 MHz, CDCl₃) δ 171.8, 164.1, 150.3, 142.2, 139.1, 135.4, 129.2, 128.8, 127.5, 126.1, 120.3, 115.0, 66.3 (t, J=28.2 Hz), 53.7, 52.7, 37.6. ¹⁹F NMR (376 MHz, CDCl₃) δ −83.3 (s), −123.1 (t, J=11.8 Hz). IR (neat, cm⁻¹) 3230, 2973, 1748, 1535, 1195. HRMS (ESI) calcd C₂₀H₁₈F₅N₂O₆⁺ [M+H]⁺: 477.1080. Found: 477.1082.

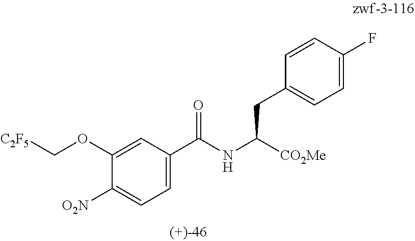

(+)-46 was obtained as white solid from the asymmetric decarboxylation reaction of malonic acid S46 (104.7 mg, 0.2 mmol) for 8 h using the general procedure (88.9 mg, 90% Yield). R_{f}=0.3 (Hexane/EtOAc 4:1). HPLC analysis (Chiralpak IA-3, hexane/ⁱPrOH=80/20, 0.8 mL/min, 254 nm; tᵣ (minor)=19.09 min, tᵣ (major)=23.31 min) gave the isomeric composition of the product: 92% e.e., [α]_{D}²³=+60.1 (c=1.02, CHCl₃). ¹H NMR (400 MHz, CDCl₃) δ 7.85 (d, J=8.4 Hz, 1H), 7.55 (s, 1H), 7.35 (d, J=8.4 Hz, 1H), 7.15-7.05 (m, 2H), 7.04-6.95 (m, 2H), 6.86 (d, J=6.4 Hz, 1H), 5.11-4.95 (m, 1H), 4.59 (t, J=11.8 Hz, 2H), 3.81 (s, 3H), 3.29 (dd, J₁=14.2, J₂=5.8 Hz, 1H), 3.18 (dd, J₁=14.0, J₂=6.0 Hz, 1H). ¹³C NMR (100 MHz, CDCl₃) δ 171.8, 164.2, 162.1 (d, J=244.9 Hz), 150.2, 142.2, 138.8, 131.2 (d, J=3.6 Hz), 130.7 (d, J=8.0 Hz), 126.0, 120.2, 115.6 (d, J=21.7 Hz), 115.0, 66.2 (t, J=28.5 Hz), 53.9, 52.7, 36.8. ¹⁹F NMR (376 MHz, CDCl₃) δ −83.4 (s), −114.9-−114.0 (m), −123.2 (t, J=11.7 Hz). IR (neat, cm⁻¹) 3227, 2963, 1748, 1536, 1194. HRMS (ESI) calcd C₂₀H₁₇F₆N₂O₆⁺ [M+H]⁺: 495.0985. Found: 495.0988.

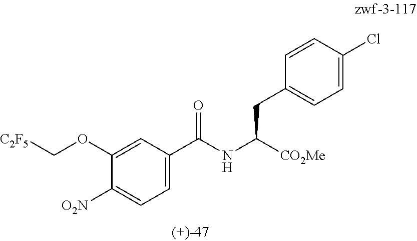

(+)-47 was obtained as white solid from the asymmetric decarboxylation reaction of malonic acid S47 (108.0 mg, 0.2 mmol) for 8 h using the general procedure (90.1 mg, 88% Yield). R_{f}=0.3 (Hexane/EtOAc 4:1). HPLC analysis (Chiralpak IA-3, hexane/ⁱPrOH=70/30, 0.8 mL/min, 254 nm; tᵣ (major)=15.82 min, tᵣ (minor)=20.62 min) gave the isomeric composition of the product: 91% e.e., [α]_{D}²³=+66.8 (c=1.02, CHCl₃). ¹H NMR (400 MHz, CDCl₃) δ 7.85 (d, J=8.4 Hz, 1H), 7.55 (s, 1H), 7.35 (d, J=8.4 Hz, 1H), 7.27 (d, J=8.4 Hz, 2H), 7.06 (d, J=8.4 Hz, 2H), 6.86 (d, J=6.8 Hz, 1H), 5.10-4.97 (m, 1H), 4.59 (t, J=11.8 Hz, 2H), 3.81 (s, 3H), 3.29 (dd, J₁=14.0, J₂=5.6 Hz, 1H), 3.18 (dd, J₁=14.0, J₂=6.0 Hz, 1H). ¹³C NMR (100 MHz, CDCl₃) δ 171.1, 164.1, 150.2, 142.2, 138.8, 134.0, 133.4, 130.5, 128.9, 126.0, 120.2, 115.0, 66.2 (t, J=28.6 Hz), 53.7, 52.8, 37.0. ¹⁹F NMR (376 MHz, CDCl₃) δ −83.3 (s), −123.1 (t, J=12.4 Hz). IR (neat, cm⁻¹) 3226, 2963, 1748, 1536, 1195. HRMS (ESI) calcd C₂₀H₁₇F₅N₂O₆³⁵Cl⁺ [M+H]⁺: 511.0690. Found: 511.0688.

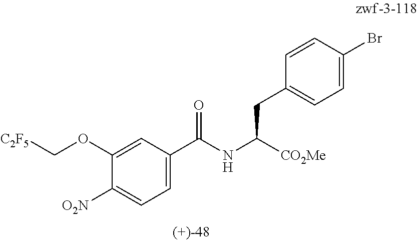

(+)-48 was obtained as white solid from the asymmetric decarboxylation reaction of malonic acid S48 (117.1 mg, 0.2 mmol) for 8 h using the general procedure (104.2 mg, 94% Yield). R_{f}=0.3 (Hexane/EtOAc 4:1). HPLC analysis (Chiralpak IA-3, hexane/PrOH=70/30, 0.8 mL/min, 254 nm; tᵣ (major)=16.95 min, tᵣ (minor)=24.54 min) gave the isomeric composition of the product: 93% e.e., [α]_{D}²³=+65.1 (c=1.00, CHCl₃). ¹H NMR (400 MHz, CDCl₃) δ 7.85 (d, J=8.0 Hz, 1H), 7.55 (s, 1H), 7.42 (d, J=8.0 Hz, 2H), 7.35 (d, J=8.4 Hz, 1H), 7.01 (d, J=8.0 Hz, 2H), 6.87 (d, J=7.6 Hz, 1H), 5.12-4.95 (m, 1H), 4.59 (t, J=11.8 Hz, 2H), 3.81 (s, 3H), 3.28 (dd, J₁=14.0, J₂=5.6 Hz, 1H), 3.16 (dd, J₁=14.0, J₂=6.0 Hz, 1H). ¹³C NMR (100 MHz, CDCl₃) δ 171.7, 164.2, 150.2, 142.2, 138.7, 134.5, 131.8, 130.8, 126.0, 121.4, 120.2, 114.9, 66.2 (t, J=28.5 Hz), 53.7, 52.8, 37.0. ¹⁹F NMR (376 MHz, CDCl₃) δ −83.3 (s), −123.1 (t, J=12.6 Hz). IR (neat, cm⁻¹) 3222, 2964, 1750, 1538, 1195. HRMS (ESI) calcd C₂₀H₁₇F₅N₂O₆⁷⁹Br⁺ [M+H]⁺: 555.0185. Found: 555.0181.

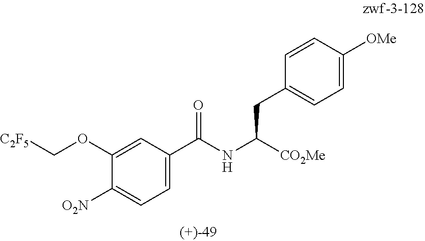

(+)-49 was obtained as white solid from the asymmetric decarboxylation reaction of malonic acid S49 (107.3 mg, 0.2 mmol) for 12 h using the general procedure (92.9 mg, 92% Yield). R_{f}=0.5 (Hexane/EtOAc 2:1). HPLC analysis (Chiralpak IA-3, hexane/ⁱPrOH=80/20, 0.8 mL/min, 254 nm; tᵣ (minor)=19.69 min, tᵣ (major)=26.99 min) gave the isomeric composition of the product: 92% e.e., [α]_{D}²³=+73.3 (c=1.01, CHCl₃). ¹H NMR (400 MHz, CDCl₃) δ 7.88 (d, J=8.4 Hz, 1H), 7.56 (s, 1H), 7.35 (dd, J₁=8.4, J₂=1.6 Hz, 1H), 7.02 (d, J=8.4 Hz, 2H), 6.83 (d, J=8.4 Hz, 2H), 6.88 (d, J=6.8 Hz, 1H), 5.07-4.97 (m, 1H), 4.59 (t, J=11.8 Hz, 2H), 3.81 (s, 3H), 3.78 (s, 3H), 3.25 (dd, J₁=14.0, J₂=5.6 Hz, 1H), 3.17 (dd, J₁=14.0, J₂=5.6 Hz, 1H). ¹³C NMR (100 MHz, CDCl₃) δ 172.0, 164.1, 158.9, 150.2, 142.1, 139.0, 130.2, 127.2, 126.0, 120.3, 114.9, 114.1, 66.2 (t, J=28.2 Hz), 55.2, 53.9, 52.7, 36.7. ¹⁹F NMR (376 MHz, CDCl₃) δ −83.3 (s), −123.1 (t, J=11.8 Hz). IR (neat, cm⁻¹) 3222, 3049, 1751, 1539, 1197. HRMS (ESI) calcd C₂₁H₂₀F₅N₂O₇⁺ [M+H]⁺: 507.1185. Found: 507.1182.

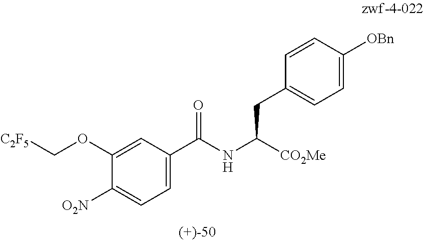

(+)-50 was obtained as colorless oil from the asymmetric decarboxylation reaction of malonic acid S50 (122.7 mg, 0.2 mmol) for 14 h using the general procedure (102.5 mg, 88% Yield). R_{f}=0.5 (Hexane/EtOAc 2:1). HPLC analysis (Chiralpak IA-3, hexane/PrOH=80/20, 0.8 mL/min, 254 nm; tᵣ (minor)=24.25 min, tᵣ (major)=38.49 min) gave the isomeric composition of the product: 94% e.e., [α]_{D}¹⁹=+48.8 (c=1.01, CHCl₃). ¹H NMR (400 MHz, CDCl₃) δ 7.88 (d, J=8.0 Hz, 1H), 7.58 (s, 1H), 7.50-7.27 (m, 6H), 7.02 (d, J=8.0 Hz, 2H), 6.91 (d, J=8.4 Hz, 2H), 6.62 (d, J=6.8 Hz, 1H), 5.16-4.91 (m, 3H), 4.59 (t, J=11.6 Hz, 2H), 3.81 (s, 3H), 3.31-3.12 (m, 2H). ¹³C NMR (100 MHz, CDCl₃) δ 172.0, 164.1, 158.1, 150.2, 142.1, 138.9, 136.7, 130.2, 128.6, 128.0, 127.6, 127.4, 125.9, 120.2, 115.1, 114.9, 69.9, 66.1 (t, J=28.2 Hz), 53.9, 52.7, 36.7. ¹⁹F NMR (376 MHz, CDCl₃) δ −83.3 (s), −123.1 (t, J=11.7 Hz). IR (neat, cm⁻¹) 3230, 3060, 1748, 1539, 1199. HRMS (ESI) calcd C₂₇H₂₄F₅N₂O₇⁺ [M+H]⁺: 583.1498. Found: 583.1497.

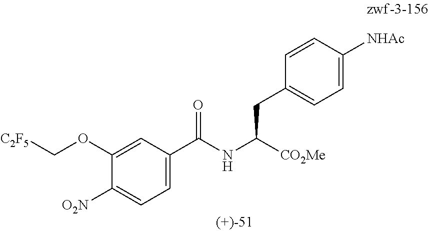

(+)-51 was obtained as white solid from the asymmetric decarboxylation reaction of malonic acid S51 (112.8 mg, 0.2 mmol) for 20 h using the general procedure with 1,4-dioxane as solvent (94.3 mg, 88% Yield). R_{f}=0.4 (Hexane/EtOAc 1:1). HPLC analysis (Chiralpak IA-3, hexane/PrOH=70/30, 0.9 mL/min, 254 nm; tᵣ (minor)=17.07 min, tᵣ(major)=21.99 min) gave the isomeric composition of the product: 90% e.e., [α]_{D}¹⁹=+64.7 (c=1.00, CHCl₃). ¹H NMR (400 MHz, CDCl₃) δ 7.86 (d, J=8.4 Hz, 1H), 7.56 (s, 1H), 7.42 (d, J=8.0 Hz, 2H), 7.36 (d, J=8.0 Hz, 1H), 7.27 (s, 1H), 7.05 (d, J=8.0 Hz, 2H), 6.75 (d, J=7.6 Hz, 1H), 5.11-4.94 (m, 1H), 4.59 (t, J=11.8 Hz, 2H), 3.81 (s, 3H), 3.28 (dd, J₁=13.8, J₂=5.4 Hz, 1H), 3.18 (dd, J₁=14.2, J₂=5.4 Hz, 1H), 2.15 (s, 3H). ¹³C NMR (100 MHz, CDCl₃) δ 171.7, 168.5, 164.3, 150.2, 142.1, 139.0, 137.1, 131.4, 129.7, 126.0, 120.3, 120.2, 114.9, 66.2 (t, J=28.2 Hz), 53.8, 52.7, 37.0, 24.4. ¹⁹F NMR (376 MHz, CDCl₃) δ −83.3 (s), −123.1 (t, J=11.7 Hz). IR (neat, cm⁻¹) 3264, 3065, 1743, 1530, 1194. HRMS (ESI) calcd C₂₂H₂₁F₅N₃O₇⁺ [M+H]⁺: 534.1294. Found: 534.1296.

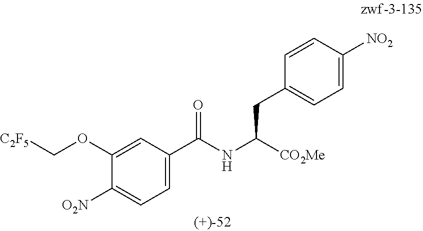

(+)-52 was obtained as white solid from the asymmetric decarboxylation reaction of malonic acid S52 (110.1 mg, 0.2 mmol) for 12 h using the general procedure (94.9 mg, 91% Yield). R_{f}=0.3 (Hexane/EtOAc 2:1). HPLC analysis (Chiralpak IA-3, hexane/PrOH=70/30, 0.9 mL/min, 254 nm; tᵣ (major)=18.83 min, tᵣ (minor)=51.69 min) gave the isomeric composition of the product: 95% e.e., [α]_{D}²⁰=+65.6 (c=1.00, CHCl₃). ¹H NMR (400 MHz, CDCl₃) δ 8.16 (d, J=8.4 Hz, 2H), 7.91 (d, J=8.4 Hz, 1H), 7.61 (s, 1H), 7.39 (d, J=8.0 Hz, 1H), 7.31 (d, J=8.4 Hz, 2H), 6.80 (d, J=6.8 Hz, 1H), 5.16-5.04 (m, 1H), 4.61 (t, J=11.8 Hz, 2H), 3.83 (s, 3H), 3.45 (dd, J₁=14.0, J₂=6.0 Hz, 1H), 3.32 (dd, J₁=13.6, J₂=5.6 Hz, 1H). ¹³C NMR (100 MHz, CDCl₃) δ 171.3, 164.2, 150.4, 147.3, 143.4, 142.4, 138.5, 130.1, 126.1, 123.8, 120.2, 115.2, 66.3 (t, J=28.6 Hz), 53.6, 53.0, 37.6. ¹⁹F NMR (376 MHz, CDCl₃) δ −83.3 (s), −123.1 (t, J=11.7 Hz). IR (neat, cm⁻¹) 3225, 2962, 1751, 1539, 1197. HRMS (ESI) calcd C₂₀H₁₇F₅N₃O₈⁺ [M+H]⁺: 522.0930. Found: 522.0932.

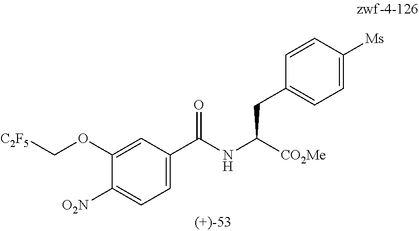

(+)-53 was obtained as white solid from the asymmetric decarboxylation reaction of malonic acid S53 (116.7 mg, 0.2 mmol) for 15 h using the general procedure (102.9 mg, 93% Yield). R_{f}=0.2 (Hexane/EtOAc 1:1). HPLC analysis (Chiralpak IC-3, hexane/ⁱPrOH=70/30, 0.8 mL/min, 254 nm; tᵣ (minor)=57.51 min, tᵣ (major)=63.89 min) gave the isomeric composition of the product: 90% e.e., [α]_{D}¹⁸=+53.8 (c=1.00, CHCl₃). ¹H NMR (500 MHz, CDCl₃) δ 7.92-7.76 (m, 3H), 7.59 (s, 1H), 7.44-7.32 (m, 3H), 7.03 (d, J=7.5 Hz, 1H), 5.14-5.03 (m, 1H), 4.61 (t, J=11.8 Hz, 2H), 3.81 (s, 3H), 3.43 (dd, J₁=14.0, J₂=5.5 Hz, 1H), 3.30 (dd, J₁=14.0, J₂=6.5 Hz, 1H), 3.04 (s, 3H). ¹³C NMR (100 MHz, CDCl₃) δ 171.3, 164.4, 150.2, 142.4, 142.2, 139.4, 138.6, 130.2, 127.6, 126.0, 120.3, 115.0, 66.2 (t, J=28.6 Hz), 53.6, 52.9, 44.3, 37.4. ¹⁹F NMR (376 MHz, CDCl₃) δ −83.3 (s), −123.1 (t, J=11.7 Hz). IR (neat, cm⁻¹) 3351, 2962, 1748, 1530, 1151. HRMS (ESI) calcd C₂₁H₂₀F₅N₂O₈⁺ [M+H]⁺: 555.0855. Found: 555.0857.

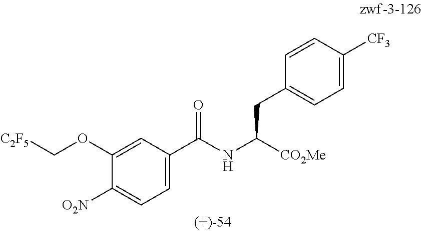

(+)-54 was obtained as white solid from the asymmetric decarboxylation reaction of malonic acid S54 (115.0 mg, 0.2 mmol) for 10 h using the general procedure (96.4 mg, 88% Yield). R_{f}=0.4 (Hexane/EtOAc 2:1). HPLC analysis (Chiralpak IA-3, hexane/PrOH=80/20, 0.8 mL/min, 254 nm; tᵣ (major)=24.44 min, tᵣ (minor)=32.15 min) gave the isomeric composition of the product: 92% e.e., [α]_{D}²³=+57.6 (c=1.00, CHCl₃). ¹H NMR (400 MHz, CDCl₃) δ 7.87 (d, J=8.0 Hz, 1H), 7.66-7.49 (m, 3H), 7.37 (d, J=8.4 Hz, 1H), 7.26 (d, J=8.4 Hz, 2H), 6.85 (d, J=7.2 Hz, 1H), 5.17-5.01 (m, 1H), 4.59 (t, J=11.8 Hz, 2H), 3.82 (s, 3H), 3.39 (dd, J₁=13.6, J₂=5.6 Hz, 1H), 3.27 (dd, J₁=13.8, J₂=5.8 Hz, 1H). ¹³C NMR (100 MHz, CDCl₃) δ 171.5, 164.2, 150.3, 142.3, 139.7, 138.7, 129.7 (q, J=32.3 Hz), 129.6, 126.1, 125.6 (q, J=3.6 Hz), 124.0 (q, J=270.4 Hz), 120.2, 115.0, 66.3 (t, J=28.6 Hz), 53.6, 52.9, 37.4. ¹⁹F NMR (376 MHz, CDCl₃) δ −62.6 (s), −83.4 (s), −123.2 (t, J=11.7 Hz). IR (neat, cm⁻¹) 3223, 3049, 1751, 1539, 1197. HRMS (ESI) calcd C₂₁H₁₇F₈N₂O₆⁺ [M+H]⁺: 545.0953. Found: 545.0956.

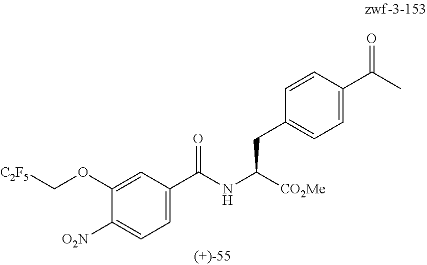

(+)-55 was obtained as white solid from the asymmetric decarboxylation reaction of malonic acid S55 (109.7 mg, 0.2 mmol) for 15 h using the general procedure (92.9 mg, 90% Yield). R_{f}=0.3 (Hexane/EtOAc 2:1). HPLC analysis (Chiralpak IA-3, hexane/PrOH=80/20, 0.8 mL/min, 254 nm; tᵣ (major)=36.17 min, tᵣ (minor)=57.45 min) gave the isomeric composition of the product: 95% e.e., [α]_{D}¹⁹=+74.7 (c=1.01, CHCl₃). ¹H NMR (400 MHz, CDCl₃) δ 7.88 (d, J=8.4 Hz, 2H), 7.82 (d, J=8.0 Hz, 1H), 7.57 (s, 1H), 7.37 (d, J=8.4 Hz, 1H), 7.25 (d, J=8.0 Hz, 2H), 7.06 (d, J=7.6 Hz, 1H), 5.14-5.02 (m, 1H), 4.59 (t, J=11.8 Hz, 2H), 3.82 (s, 3H), 3.39 (dd, J₁=14.0, J₂=5.6 Hz, 1H), 3.26 (dd, J₁=14.0, J₂=6.4 Hz, 1H), 2.57 (s, 3H). ¹³C NMR (100 MHz, CDCl₃) δ 197.7, 171.6, 164.2, 150.2, 142.1, 141.2, 138.7, 136.2, 129.4, 128.7, 125.9, 120.3, 115.0, 66.2 (t, J=28.5 Hz), 53.7, 52.8, 37.6, 26.5. ¹⁹F NMR (376 MHz, CDCl₃) δ −83.3 (s), −123.2 (t, J=11.7 Hz). IR (neat, cm⁻¹) 3240, 3060, 1753, 1534, 1194. HRMS (ESI) calcd C₂₂H₂₀F₅N₂O₇⁺ [M+H]⁺: 519.1185. Found: 519.1186.

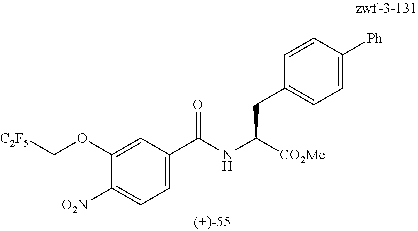

(+)-56 was obtained as white solid from the asymmetric decarboxylation reaction of malonic acid S56 (116.4 mg, 0.2 mmol) for 12 h using the general procedure (99.7 mg, 90% Yield). R_{f}=0.5 (Hexane/EtOAc 2:1). HPLC analysis (Chiralpak IA-3, hexane/ⁱPrOH=70/30, 0.8 mL/min, 254 nm; tᵣ (minor)=11.38 min, tᵣ (major)=16.59 min) gave the isomeric composition of the product: 94% e.e., [α]_{D}²³=+81.9 (c=1.03, CHCl₃). ¹H NMR (400 MHz, CDCl₃) δ 7.89 (d, J=8.4 Hz, 1H), 7.63-7.50 (m, 5H), 7.44 (t, J=7.4 Hz, 2H), 7.40-7.32 (m, 2H), 7.18 (d, J=8.0 Hz, 2H), 6.71 (d, J=7.2 Hz, 1H), 5.16-5.04 (m, 1H), 4.59 (t, J=11.8 Hz, 2H), 3.84 (s, 3H), 3.36 (dd, J₁=14.0, J₂=5.6 Hz, 1H), 3.28 (dd, J₁=14.0, J₂=5.2 Hz, 1H). ¹³C NMR (100 MHz, CDCl₃) δ 171.9, 164.2, 150.2, 142.2, 140.30, 140.26, 138.9, 134.4, 129.6, 128.8, 127.5, 127.4, 126.9, 126.0, 120.3, 115.0, 66.2 (t, J=28.2 Hz), 53.8, 52.7, 37.2. ¹⁹F NMR (376 MHz, CDCl₃) δ −83.3 (s), −123.1 (t, J=11.8 Hz). IR (neat, cm⁻¹) 3222, 2962, 1751, 1539, 1197. HRMS (ESI) calcd C₂₆H₂₂F₅N₂O₆⁺ [M+H]⁺: 553.1393. Found: 553.1390.

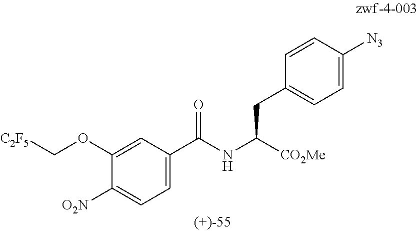

(+)-57 was obtained as white solid from the asymmetric decarboxylation reaction of malonic acid S57 (109.7 mg, 0.2 mmol) for 18 h using the general procedure (95.7 mg, 92% Yield). R_{f}=0.5 (Hexane/EtOAc 2:1). HPLC analysis (Chiralpak ID-3, hexane/ⁱPrOH=70/30, 0.8 mL/min, 254 nm; tᵣ (minor)=9.16 min, tᵣ (major)=10.78 min) gave the isomeric composition of the product: 94% e.e., [α]_{D}¹⁹=+82.2 (c=1.00, CHCl₃). ¹H NMR (500 MHz, CDCl₃) δ 7.85 (d, J=8.0 Hz, 1H), 7.56 (s, 1H), 7.35 (d, J=8.0 Hz, 1H), 7.12 (d, J=7.6 Hz, 2H), 6.96 (d, J=8.0 Hz, 2H), 6.88 (d, J=7.2 Hz, 1H), 5.12-4.96 (m, 1H), 4.59 (t, J=12.0 Hz, 2H), 3.82 (s, 3H), 3.30 (dd, J₁=14.0, J₂=5.6 Hz, 1H), 3.18 (dd, J₁=14.0, J₂=6.0 Hz, 1H). ¹³C NMR (100 MHz, CDCl₃) δ 171.8, 164.1, 150.2, 142.2, 139.3, 138.8, 132.1, 130.5, 126.0, 120.2, 119.3, 115.0, 66.2 (t, J=28.2 Hz), 53.9, 52.7, 37.0. ¹⁹F NMR (376 MHz, CDCl₃) δ −83.3 (s), −123.1 (t, J=11.8 Hz). IR (neat, cm⁻¹) 3235, 3035, 2122, 1748, 1534, 1199. HRMS (ESI) calcd C₂₀H₁₇F₅N₅O₆⁺ [M+H]⁺: 518.1094. Found: 518.1093.

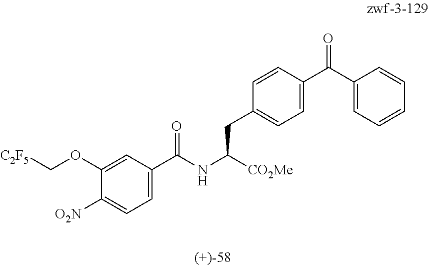

(+)-58 was obtained as white solid from the asymmetric decarboxylation reaction of malonic acid S58 (122.3 mg, 0.2 mmol) for 12 h using the general procedure (107.4 mg, 92% Yield). R_{f}=0.3 (Hexane/EtOAc 2:1). HPLC analysis (Chiralpak IA-3, hexane/PrOH=70/30, 0.8 mL/min, 254 nm; tᵣ (minor)=16.01 min, tᵣ (major)=28.66 min) gave the isomeric composition of the product: 93% e.e., [α]_{D}²³=+61.3 (c=1.02, CHCl₃). ¹H NMR (400 MHz, CDCl₃) δ 7.89-7.81 (m, 1H), 7.81-7.66 (m, 4H), 7.65-7.53 (m, 2H), 7.47 (t, J=7.4 Hz, 2H), 7.38 (d, J=8.0 Hz, 1H), 7.26 (d, J=8.4 Hz, 2H), 7.04-6.87 (m, 1H), 5.19-5.02 (m, 1H), 4.59 (t, J=11.8 Hz, 2H), 3.82 (s, 3H), 3.42 (dd, J₁=13.6, J₂=5.6 Hz, 1H), 3.30 (dd, J₁=13.8, J₂=5.8 Hz, 1H). ¹³C NMR (100 MHz, CDCl₃) δ 196.3, 171.6, 164.2, 150.2, 142.2, 140.5, 138.7, 137.3, 136.6, 132.6, 130.5, 129.9, 129.2, 128.3, 126.0, 120.2, 115.0, 66.2 (t, J=28.5 Hz), 53.7, 52.8, 37.6. ¹⁹F NMR (376 MHz, CDCl₃) δ −83.3 (s), −123.1 (t, J=11.8 Hz). IR (neat, cm⁻¹) 3223, 3049, 1750, 1539, 1197. HRMS (ESI) calcd C₂₇H₂₂F₅N₂O₇⁺ [M+H]⁺: 581.1342. Found: 581.1343.

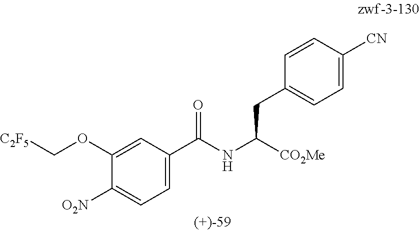

(+)-59 was obtained as white solid from the asymmetric decarboxylation reaction of malonic acid S59 (106.2 mg, 0.2 mmol) for 12 h using the general procedure (82.5 mg, 82% Yield). R_{f}=0.3 (Hexane/EtOAc 2:1). HPLC analysis (Chiralpak IA-3, hexane/PrOH=70/30, 0.8 mL/min, 254 nm; tᵣ (major)=17.91 min, tᵣ (minor)=30.23 min) gave the isomeric composition of the product: 94% e.e., [α]_{D}²⁰=+24.8 (c=1.01, CHCl₃). ¹H NMR (500 MHz, CDCl₃) δ 7.90 (d, J=8.5 Hz, 1H), 7.66-7.53 (m, 3H), 7.39 (dd, J₁=8.5, J₂=1.5 Hz, 1H), 7.26 (d, J=7.5 Hz, 2H), 6.91-6.80 (m, 1H), 5.12-5.03 (m, 1H), 4.61 (t, J=12.0 Hz, 2H), 3.82 (s, 3H), 3.39 (dd, J₁=14.0, J₂=6.0 Hz, 1H), 3.27 (dd, J₁=13.8, J₂=5.8 Hz, 1H). ¹³C NMR (125 MHz, CDCl₃) δ 171.3, 164.1, 150.4, 142.4, 141.3, 138.6, 132.4, 130.0, 126.1, 120.2, 118.4, 115.2, 111.4, 66.3 (t, J=28.0 Hz), 53.6, 52.9, 37.9. ¹⁹F NMR (376 MHz, CDCl₃) δ −83.3 (s), −123.1 (t, J=11.7 Hz). IR (neat, cm⁻¹) 3224, 2962, 1751, 1539, 1197. HRMS (ESI) calcd C₂₁H₁₇F₅N₃O₆⁺ [M+H]⁺: 502.1032. Found: 502.1031.

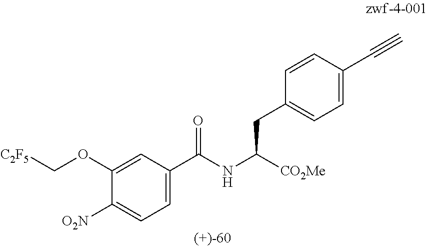

(+)-60 was obtained as white solid from the asymmetric decarboxylation reaction of malonic acid S60 (106.1 mg, 0.2 mmol) for 15 h using the general procedure (90.8 mg, 91% Yield). R_{f}=0.6 (Hexane/EtOAc 2:1). HPLC analysis (Chiralpak IA-3, hexane/PrOH=80/20, 0.8 mL/min, 254 nm; tᵣ (minor)=18.46 min, tᵣ (major)=29.46 min) gave the isomeric composition of the product: 93% e.e., [α]_{D}¹⁹=+88.1 (c=1.03, CHCl₃). ¹H NMR (400 MHz, CDCl₃) δ 7.84 (d, J=8.4 Hz, 1H), 7.53 (s, 1H), 7.42 (d, J=7.6 Hz, 2H), 7.33 (d, J=8.0 Hz, 1H), 7.09 (d, J=7.6 Hz, 2H), 6.83 (d, J=7.6 Hz, 1H), 5.12-4.98 (m, 1H), 4.58 (t, J=12.0 Hz, 2H), 3.81 (s, 3H), 3.32 (dd, J₁=14.0, J₂=5.6 Hz, 1H), 3.20 (dd, J₁=13.6, J₂=6.0 Hz, 1H), 3.09 (s, 1H). ¹³C NMR (100 MHz, CDCl₃) δ 171.7, 164.2, 150.2, 142.2, 138.8, 136.3, 132.4, 129.2, 126.0, 121.3, 120.2, 114.9, 83.0, 77.8, 66.2 (t, J=28.6 Hz), 53.7, 52.8, 37.5. ¹⁹F NMR (376 MHz, CDCl₃) δ −83.3 (s), −123.1 (t, J=11.8 Hz). IR (neat, cm⁻¹) 3303, 3055, 1753, 1355, 1199. HRMS (ESI) calcd C₂₂H₁₈F₅N₂O₆⁺ [M+H]⁺: 501.1080. Found: 501.1078.

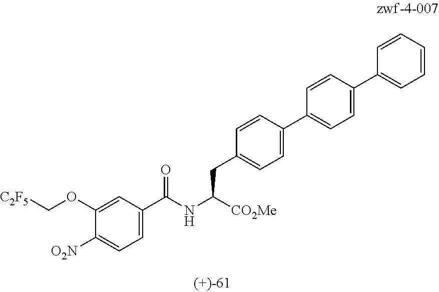

(+)-61 was obtained as white solid from the asymmetric decarboxylation reaction of malonic acid S61 (131.8 mg, 0.2 mmol) for 15 h using the general procedure (120.3 mg, 96% Yield). R_{f}=0.4 (Hexane/EtOAc 2:1). HPLC analysis (Chiralpak IA-3, hexane/PrOH=80/20, 0.8 mL/min, 254 nm; tᵣ (minor)=25.60 min, tᵣ (major)=33.12 min) gave the isomeric composition of the product: 93% e.e., [α]_{D}¹⁸=+81.8 (c=1.00, CHCl₃). ¹H NMR (400 MHz, CDCl₃) δ 7.88 (d, J=8.4 Hz, 1H), 7.72-7.53 (m, 9H), 7.46 (t, J=7.4 Hz, 2H), 7.37 (d, J=7.2 Hz, 2H), 7.20 (d, J=8.0 Hz, 2H), 6.75 (d, J=7.6 Hz, 1H), 5.17-5.04 (m, 1H), 4.59 (t, J=11.8 Hz, 2H), 3.85 (s, 3H), 3.37 (dd, J₁=14.0, J₂=5.6 Hz, 1H), 3.29 (dd, J₁=13.8, J₂=5.4 Hz, 1H). ¹³C NMR (100 MHz, CDCl₃) δ 171.8, 164.2, 150.3, 142.2, 140.5, 140.3, 139.8, 139.1, 139.0, 134.5, 129.7, 128.8, 127.5, 127.4, 127.3, 127.2, 127.0, 126.1, 120.3, 115.1, 66.3 (t, J=28.5 Hz), 53.8, 52.7, 37.3. ¹⁹F NMR (376 MHz, CDCl₃) δ −83.3 (s), −123.1 (t, J=11.7 Hz). IR (neat, cm⁻¹) 3070, 1748, 1530, 1267, 1204. HRMS (ESI) calcd C₃₂H₂₆F₅N₂O₆⁺ [M+H]⁺: 629.1706. Found: 629.1707.

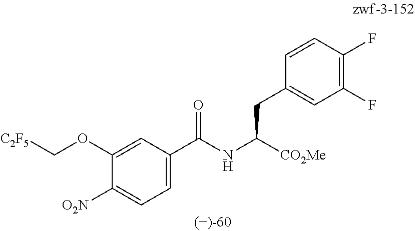

(+)-62 was obtained as white solid from the asymmetric decarboxylation reaction of malonic acid S62 (108.4 mg, 0.2 mmol) for 15 h using the general procedure (96.6 mg, 94% Yield). R_{f}=0.4 (Hexane/EtOAc 2:1). HPLC analysis (Chiralpak IA-3, hexane/PrOH=80/20, 0.8 mL/min, 254 nm; tᵣ (minor)=18.66 min, tᵣ (major)=20.58 min) gave the isomeric composition of the product: 95% e.e., [α]_{D}¹⁸=+62.8 (c=1.00, CHCl₃). ¹H NMR (400 MHz, CDCl₃) δ 7.82 (d, J=8.0 Hz, 1H), 7.55 (s, 1H), 7.36 (d, J=8.4 Hz, 1H), 7.15-6.94 (m, 3H), 6.92-6.83 (m, 1H), 5.11-4.93 (m, 1H), 4.59 (t, J=12.0 Hz, 2H), 3.82 (s, 3H), 3.28 (dd, J₁=14.0, J₂=5.6 Hz, 1H), 3.15 (dd, J₁=14.0, J₂=6.4 Hz, 1H). ¹³C NMR (100 MHz, CDCl₃) δ 171.7, 164.3, 150.2, 149.9 (ddd, J₁=247.2, J₂=54.6, J₃=12.7 Hz), 142.1, 138.6, 132.6 (dd, J₁=5.0, J₂=4.4 Hz), 125.9, 125.2 (dd, J_{1=6.3}, J₂=3.3 Hz), 120.2, 117.7 (dd, J₁=54.9, J₂=17.4 Hz), 114.8, 66.1 (t, J=28.5 Hz), 53.8, 52.8, 36.8. ¹⁹F NMR (376 MHz, CDCl₃) δ −83.4 (s), −123.2 (t, J=11.8 Hz), −137.0-−137.3 (m), −139.4-−137.6 (m). IR (neat, cm⁻¹) 3230, 3055, 1753, 1525, 1204. HRMS (ESI) calcd C₂₀H₁₆F₇N₂O₆⁺ [M+H]⁺: 513.0891. Found: 513.0894.

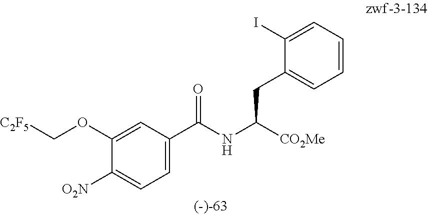

(−)-63 was obtained as white solid from the asymmetric decarboxylation reaction of malonic acid S63 (126.4 mg, 0.2 mmol) for 12 h using the general procedure (112.3 mg, 93% Yield). R_{f}=0.5 (Hexane/EtOAc 2:1). HPLC analysis (Chiralpak IA-3, hexane/PrOH=70/30, 0.8 mL/min, 254 nm; tᵣ (minor)=8.43 min, tᵣ (major)=15.19 min) gave the isomeric composition of the product: 82% e.e. (76% Yield, 98% e.e., single recrystallized from acetone/hexane), [α]_{D}²⁰=−6.9 (c=1.00, CHCl₃ (98% e.e.)). ¹H NMR (400 MHz, CDCl₃) δ 7.96-7.87 (m, 2H), 7.54 (s, 1H), 7.44 (d, J=8.4 Hz, 1H), 7.31 (t, J=7.4 Hz, 1H), 7.24 (d, J=7.6 Hz, 1H), 6.96 (t, J=7.6 Hz, 1H), 6.79 (d, J=8.0 Hz, 1H), 5.20-5.00 (m, 1H), 4.57 (t, J=11.8 Hz, 2H), 3.81 (s, 3H), 3.47 (dd, J_{1=14.0}, J₂=6.0 Hz, 1H), 3.32 (dd, J₁=14.2, J₂=8.6 Hz, 1H). ¹³C NMR (100 MHz, CDCl₃) δ 171.9, 164.4, 150.1, 142.1, 139.8, 139.0, 138.7, 130.3, 129.2, 128.7, 125.9, 120.5, 114.9, 101.1, 66.2 (t, J=28.5 Hz), 53.5, 52.9, 42.2. ¹⁹F NMR (376 MHz, CDCl₃) δ −83.3 (s), −123.2 (t, J=11.8 Hz). IR (neat, cm⁻¹) 3222, 3050, 1751, 1539, 1198. HRMS (ESI) calcd C₂₀H₁₇F₅N₂O₆I⁺ [M+H]⁺: 603.0046. Found: 603.0047.

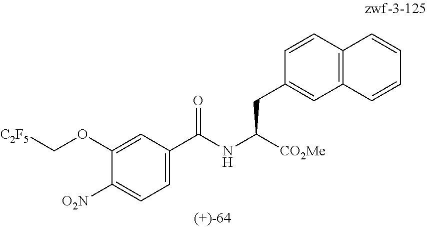

(+)-64 was obtained as white solid from the asymmetric decarboxylation reaction of malonic acid S64 (111.3 mg, 0.2 mmol) for 10 h using the general procedure (95.5 mg, 91% Yield). R_{f}=0.5 (Hexane/EtOAc 2:1). HPLC analysis (Chiralpak IA-3, hexane/PrOH=80/20, 0.8 mL/min, 254 nm; tᵣ (minor)=16.71 min, tᵣ (major)=22.30 min) gave the isomeric composition of the product: 91% e.e., [α]_{D}²³=+69.5 (c=1.02, CHCl₃). ¹H NMR (400 MHz, CDCl₃) δ 7.89-7.67 (m, 4H), 7.58 (s, 1H), 7.53-7.39 (m, 3H), 7.31-7.20 (m, 2H), 6.82 (d, J=7.6 Hz, 1H), 5.21-5.06 (m, 1H), 4.48 (t, J=11.8 Hz, 2H), 3.82 (s, 3H), 3.48 (dd, J₁=13.8, J₂=5.8 Hz, 1H), 3.37 (dd, J₁=14.0, J₂=6.0 Hz, 1H). 13C NMR (100 MHz, CDCl₃) δ 171.9, 164.3, 150.1, 142.1, 139.0, 133.4, 132.9, 132.5, 128.5, 128.1, 127.7, 127.4, 127.0, 126.5, 126.1, 126.0, 120.3, 114.7, 66.1 (t, J=28.6 Hz), 53.8, 52.7, 37.8. ¹⁹F NMR (376 MHz, CDCl₃) δ −83.3 (s), −123.1 (t, J=11.8 Hz). IR (neat, cm⁻¹) 3232, 2975, 1753, 1538, 1198. HRMS (ESI) calcd C₂₄H₂₀F₅N₂O₆⁺ [M+H]⁺: 527.1236. Found: 527.1237.

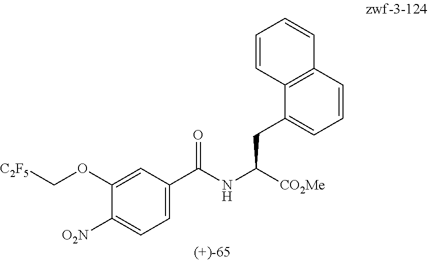

(+)-65 was obtained as white solid from the asymmetric decarboxylation reaction of malonic acid S65 (111.1 mg, 0.2 mmol) for 10 h using the general procedure (99.2 mg, 94% Yield). R_{f}=0.5 (Hexane/EtOAc 2:1). HPLC analysis (Chiralpak IA-3, hexane/ⁱPrOH=80/20, 0.8 mL/min, 254 nm; tᵣ (minor)=12.04 min, tᵣ (major)=21.71 min) gave the isomeric composition of the product: 81% e.e. (81% Yield, 97% e.e., single recrystallized from DCM/hexane), [α]_{D}²³=+6.6 (c=1.02, CHCl₃ (97% e.e.)). ¹H NMR (400 MHz, CDCl₃) δ 8.16-8.02 (m, 1H), 7.93-7.83 (m, 1H), 7.83-7.67 (m, 2H), 7.60-7.45 (m, 2H), 7.40 (t, J=7.4 Hz, 1H), 7.36-7.26 (m, 2H), 7.17 (d, J=8.4 Hz, 1H), 6.79 (d, J=7.2 Hz, 1H), 5.26-5.13 (m, 1H), 4.47 (t, J=11.6 Hz, 2H), 3.85-3.56 (m, 5H). ¹³C NMR (100 MHz, CDCl₃) δ 172.1, 164.4, 150.0, 142.0, 138.9, 133.9, 132.2, 131.9, 129.1, 128.3, 127.5, 126.5, 125.93, 125.87, 125.3, 123.2, 120.4, 114.6, 66.1 (t, J=28.2 Hz), 53.8, 52.6, 34.6. ¹⁹F NMR (376 MHz, CDCl₃) δ −83.3 (s), −123.1 (t, J=11.7 Hz). IR (neat, cm⁻¹) 3230, 2973, 1750, 1538, 1196. HRMS (ESI) calcd C₂₄H₂₀F₅N₂O₆⁺ [M+H]⁺: 527.1236. Found: 527.1234.

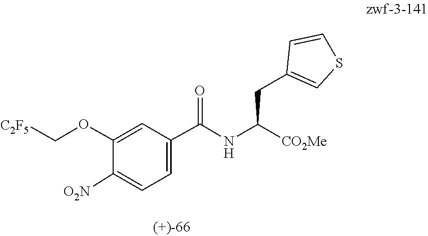

(+)-66 was obtained as white solid from the asymmetric decarboxylation reaction of malonic acid S66 (102.5 mg, 0.2 mmol) for 8 h using the general procedure (90.7 mg, 94% Yield). R_{f}=0.5 (Hexane/EtOAc 2:1). HPLC analysis (Chiralpak IA-3, hexane/PrOH=80/20, 0.8 mL/min, 254 nm; tᵣ (minor)=14.05 min, tᵣ (major)=25.09 min) gave the isomeric composition of the product: 93% e.e., [α]_{D}²⁰=+71.4 (c=1.00, CHCl₃). ¹H NMR (400 MHz, CDCl₃) δ 7.85 (d, J=8.4 Hz, 1H), 7.55 (s, 1H), 7.35 (d, J=8.0 Hz, 1H), 7.30 (dd, J₁=5.0, J₂=3.0 Hz, 1H), 7.02 (d, J=1.6 Hz, 1H), 6.93-6.77 (m, 2H), 5.09-4.95 (m, 1H), 4.59 (t, J=12.0 Hz, 2H), 3.82 (s, 3H), 3.36 (dd, J₁=14.4, J₂=5.2 Hz, 1H), 3.27 (dd, J₁=14.6, J₂=5.8 Hz, 1H). ¹³C NMR (100 MHz, CDCl₃) δ 171.9, 164.2, 150.2, 142.2, 138.9, 135.6, 128.0, 126.5, 126.0, 123.0, 120.3, 114.9, 66.2 (t, J=28.6 Hz), 53.3, 52.8, 32.0. ¹⁹F NMR (376 MHz, CDCl₃) δ −83.3 (s), −123.1 (t, J=11.8 Hz). IR (neat, cm⁻¹) 3228, 2962, 1750, 1538, 1197. HRMS (ESI) calcd C₁₈H₁₆F₅N₂O₆S⁺ [M+H]⁺: 483.0644. Found: 483.0645.

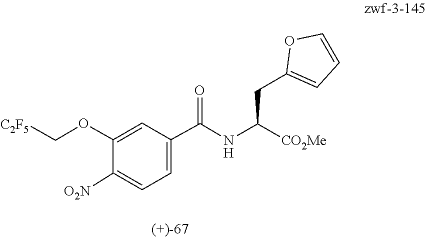

(+)-67 was obtained as white solid from the asymmetric decarboxylation reaction of malonic acid S67 (99.2 mg, 0.2 mmol) for 10 h using the general procedure (84.4 mg, 91% Yield). R_{f}=0.4 (Hexane/EtOAc 2:1). HPLC analysis (Chiralpak IA-3, hexane/PrOH=80/20, 0.8 mL/min, 254 nm; tᵣ (minor)=19.53 min, tᵣ (major)=20.81 min) gave the isomeric composition of the product: 92% e.e., [α]_{D}²⁰=+71.6 (c=0.98, CHCl₃). ¹H NMR (400 MHz, CDCl₃) δ 7.92 (d, J=8.4 Hz, 1H), 7.60 (s, 1H), 7.43 (d, J=8.4 Hz, 1H), 7.34 (d, J=1.2 Hz, 1H), 6.87 (d, J=7.2 Hz, 1H), 6.31 (t, J=2.6 Hz, 1H), 6.11 (d, J=2.8 Hz, 1H), 5.07-4.96 (m, 1H), 4.60 (t, J=11.8 Hz, 2H), 3.83 (s, 3H), 3.42-3.26 (m, 2H). ¹³C NMR (125 MHz, CDCl₃) δ 171.3, 164.3, 150.3, 150.0, 142.5, 142.2, 139.1, 126.0, 120.4, 115.1, 110.5, 108.3, 66.3 (t, J=28.0 Hz), 52.9, 52.3, 30.3. ¹⁹F NMR (471 MHz, CDCl₃) δ −83.3 (s), −123.1 (t, J=12.2 Hz). IR (neat, cm⁻¹) 3225, 2962, 1750, 1539, 1197. HRMS (ESI) calcd C₁₈H₁₆F₅N₂O₇⁺ [M+H]⁺: 467.0872. Found: 467.0871.

### Characterization in Synthetic Applications

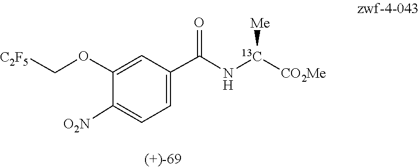

(+)-69 was obtained as white solid from the asymmetric decarboxylation reaction of malonic acid S69 (86.2 mg, 0.2 mmol) for 10 h using the general procedure (73.3 mg, 91% Yield). R_{f}=0.3 (Hexane/EtOAc 2:1). HPLC analysis (Chiralpak IA-3, hexane/PrOH=80/20, 0.8 mL/min, 254 nm; tᵣ (minor)=8.80 min, tᵣ (major)=11.89 min) gave the isomeric composition of the product: 94% e.e., [α]_{D}¹⁷=+24.5 (c=1.00, CHCl₃). ¹H NMR (400 MHz, CDCl₃) δ 7.86 (d, J=8.4 Hz, 1H), 7.60 (s, 1H), 7.48 (d, J=8.0 Hz, 1H), 7.12 (d, J=6.0 Hz, 1H), 4.78 (dquintet, J₁=147.2, J₁=7.1 Hz, 1H), 4.60 (t, J=11.6 Hz, 2H), 3.83 (s, 3H), 1.54 (dd, J₁=6.6, J₂=4.6 Hz, 3H). ¹³C NMR (100 MHz, CDCl₃) δ 173.7 (d, J=61.4 Hz), 164.0, 150.1, 142.1, 138.8, 125.9, 120.4, 114.8, 66.2 (t, J=28.6 Hz), 52.8, 48.8, 18.1 (d, J=34.7 Hz). ¹⁹F NMR (376 MHz, CDCl₃) δ −83.4 (s), −123.3 (t, J=11.7 Hz). IR (neat, cm⁻¹) 3366, 2963, 1714, 1530, 1194. HRMS (ESI) calcd C₁₃¹³CH₁₄F₅N₂O₆⁺ [M+H]⁺: 402.0800. Found: 402.0799.

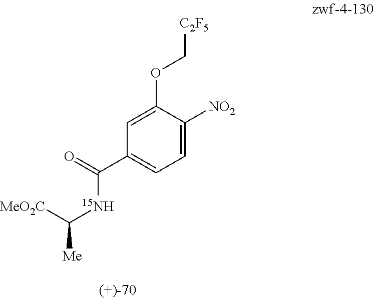

(+)-70 was obtained as white solid from the asymmetric decarboxylation reaction of malonic acid S70 (86.2 mg, 0.2 mmol) for 12 h using the general procedure (72.6 mg, 90% Yield). R_{f}=0.3 (Hexane/EtOAc 2:1). HPLC analysis (Chiralpak IA-3, hexane/PrOH=80/20, 0.8 mL/min, 254 nm; tᵣ (minor)=8.84 min, tᵣ (major)=12.07 min) gave the isomeric composition of the product: 94% e.e., [α]_{D}¹⁷=+20.7 (c=1.00, CHCl₃). ¹H NMR (400 MHz, CDCl₃) δ 7.83 (d, J=8.4 Hz, 1H), 7.58 (d, J=1.2 Hz, 1H), 7.48 (dd, J₁=8.2, J₂=1.4 Hz, 1H), 7.26 (dd, J₁=91.7, J₂=7.2 Hz, 1H), 4.85-4.72 (m, 1H), 4.59 (t, J=11.8 Hz, 2H), 3.83 (s, 3H), 1.54 (dd, J₁=7.2, J₂=2.8 Hz, 3H). ¹³C NMR (100 MHz, CDCl₃) δ 173.9, 164.0 (d, J=16.6 Hz), 150.0, 142.0, 138.6 (d, J=8.7 Hz), 125.8, 120.5, 114.6, 66.1 (t, J=28.6 Hz), 52.8, 48.8 (d, J=12.3 Hz), 18.0. ¹⁹F NMR (376 MHz, CDCl₃) δ −83.4 (s), −123.2 (t, J=11.8 Hz). ¹⁵N NMR (40 MHz, CDCl₃) δ 116.9. IR (neat, cm⁻¹) 3356, 1709, 1543, 1228, 1190. HRMS (ESI) calcd C₁₄H₁₄F₅N¹⁵NO₆ [M+H]⁺: 402.0737. Found: 402.0740.

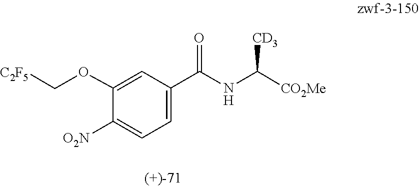

(+)-71 was obtained as white solid from the asymmetric decarboxylation reaction of malonic acid S71 (86.7 mg, 0.2 mmol) for 12 h using the general procedure (72.1 mg, 89% Yield). R_{f}=0.3 (Hexane/EtOAc 2:1). HPLC analysis (Chiralpak IA-3, hexane/PrOH=80/20, 0.8 mL/min, 254 nm; tᵣ (minor)=8.72 min, tᵣ (major)=11.84 min) gave the isomeric composition of the product: 94% e.e., [α]_{D}¹⁸=+21.3 (c=1.02, CHCl₃). ¹H NMR (500 MHz, CDCl₃) δ 7.87 (d, J=8.5 Hz, 1H), 7.61 (s, 1H), 7.48 (d, J=8.0 Hz, 1H), 7.08 (d, J=7.0 Hz, 1H), 4.76 (d, J=7.0 Hz, 1H), 4.60 (t, J=12.0 Hz, 2H), 3.82 (s, 3H). ¹³C NMR (125 MHz, CDCl₃) δ 173.8, 164.2, 149.9, 141.9, 138.7, 125.7, 120.5, 118.3 (qt, J₁=284.4 Hz, J₂=34.1 Hz), 114.5, 111.9 (tq, J₁=254.5 Hz, J₂=38.0 Hz), 66.0 (t, J=27.9 Hz), 52.7, 48.6, 16.9 (hept, J=19.8 Hz). ¹⁹F NMR (376 MHz, CDCl₃) δ −83.3 (s), −123.2 (t, J=11.1 Hz). IR (neat, cm⁻¹) 3361, 2938, 1717, 1530, 1194. HRMS (ESI) calcd C₁₄H₁₁D₃F₅N₂O₆⁺ [M+H]⁺: 404.0955. Found: 404.0958.

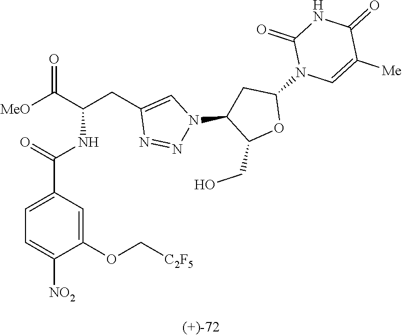

[α]_{D}¹⁸=+15.7 (c=0.95, CHCl₃). ¹H NMR (400 MHz, CD₃OD) δ 8.00 (s, 1H), 7.90 (d, J=8.4 Hz, 1H), 7.86 (s, 1H), 7.76 (s, 1H), 7.57 (d, J=8.0 Hz, 1H), 6.43 (t, J=6.0 Hz, 1H), 5.47-5.32 (m, 1H), 5.05-4.75 (m, 5H), 4.35-4.25 (m, 1H), 3.86 (d, J=12.4 Hz, 1H), 3.80-3.66 (m, 4H), 3.43 (dd, J₁=15.0, J₂=4.6 Hz, 1H), 3.34-3.24 (m, 1H), 2.96-2.80 (m, 1H), 2.77-2.63 (m, 1H), 1.87 (s, 3H). ¹³C NMR (100 MHz, CD₃OD) δ 172.7, 167.5, 166.3, 152.2, 150.9, 144.8, 143.3, 140.2, 138.2, 126.6, 124.2, 122.5, 115.8, 111.7, 86.6, 86.3, 66.9 (t, J=28.3 Hz), 62.1, 61.0, 54.4, 53.1, 38.9, 28.4, 12.5. ¹⁹F NMR (376 MHz, CD₃OD) δ −84.7 (s), −124.6 (t, J=12.4 Hz). IR (neat, cm⁻¹) 3293, 3050, 1670, 1530, 1267, 1199. HRMS (ESI) calcd C₂₆H₂₇F₅N₇O₁₀⁺ [M+H]⁺: 692.1734. Found: 692.1735.

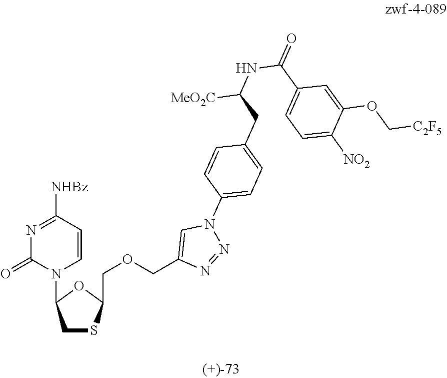

[α]_{D}¹⁸=+28.0 (c=1.02, CHCl₃). ¹H NMR (400 MHz, CDCl₃) δ 8.09 (d, J=7.2 Hz, 2H), 8.02 (s, 1H), 7.82 (d, J=8.0 Hz, 1H), 7.69 (d, J=8.4 Hz, 1H), 7.64-7.55 (m, 3H), 7.50 (t, J=7.4 Hz, 1H), 7.45-7.34 (m, 3H), 7.25 (s, 1H), 7.15 (d, J=7.2 Hz, 1H), 6.39-6.28 (m, 2H), 5.63-5.42 (m, 2H), 5.26 (t, J=3.2 Hz, 1H), 5.12-5.00 (m, 1H), 4.58 (t, J=11.8 Hz, 2H), 4.00 (dd, J₁=12.4, J₂=2.0 Hz, 1H), 3.85 (dd, J_{1=12.6}, J₂=3.8 Hz, 1H), 3.80 (s, 3H), 3.45 (dd, J₁=12.2, J₂=5.4 Hz, 1H), 3.37 (dd, J₁=14.0, J₂=5.6 Hz, 1H), 3.26 (dd, J₁=14.0, J₂=6.0 Hz, 1H), 3.11 (dd, J₁=12.0, J₂=4.8 Hz, 1H), 2.95 (br, 1H), 1.85 (br, 1H). ¹³C NMR (150 MHz, CDCl₃) δ 177.6, 171.6, 164.6, 154.5, 150.0, 149.7, 143.4, 141.9, 138.6, 136.9, 136.6, 135.8, 135.3, 132.7, 130.4, 129.7, 128.2, 125.8, 121.6, 120.5, 114.9, 98.0, 87.1, 86.7, 66.0 (t, J=28.3 Hz), 63.1, 53.9, 52.7, 37.7, 37.5, 36.8. ¹⁹F NMR (376 MHz, CDCl₃) δ −83.3 (s), −123.1 (t, J=11.8 Hz). IR (neat, cm⁻¹) 3318, 3045, 1648, 1541, 1204. HRMS (ESI) calcd C₃₈H₃₄F₅N₈O₁₀S⁺ [M+H]⁺: 889.2033. Found: 889.2030.

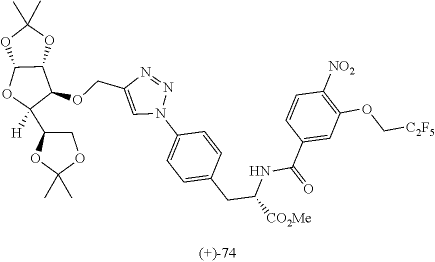

[α]_{D}¹⁸=+17.9 (c=1.02, CHCl₃). ¹H NMR (400 MHz, CDCl₃) δ 8.10 (s, 1H), 7.85 (d, J=8.4 Hz, 1H), 7.67 (d, J=7.6 Hz, 2H), 7.61 (s, 1H), 7.40 (d, J=8.4 Hz, 1H), 7.32 (d, J=8.0 Hz, 2H), 7.10-6.98 (m, 1H), 5.89 (d, J=2.8 Hz, 1H), 5.17-5.04 (m, 1H), 4.88 (q, J=12.8 Hz, 2H), 4.70-4.52 (m, 3H), 4.43-4.30 (m, 1H), 4.18-4.06 (m, 3H), 4.06-3.98 (m, 1H), 3.83 (s, 3H), 3.41 (dd, J₁=14.0, J₂=5.6 Hz, 1H), 3.28 (dd, J₁=13.8, J₂=6.2 Hz, 1H), 1.49 (s, 3H), 1.40 (s, 3H), 1.34 (s, 3H), 1.32 (s, 3H). ¹³C NMR (150 MHz, CDCl₃) δ 171.5, 164.2, 150.2, 145.8, 142.2, 138.7, 136.7, 136.1, 130.5, 126.0, 120.6, 120.2, 115.1, 111.9, 109.1, 105.2, 82.5, 81.6, 81.0, 72.2, 67.4, 66.2 (t, J=28.7 Hz), 63.8, 53.8, 52.8, 37.2, 26.8, 26.7, 26.1, 25.4. ¹⁹F NMR (376 MHz, CDCl₃) δ −83.3 (s), −123.1 (t, J=11.7 Hz). IR (neat, cm⁻¹) 3254, 2982, 1748, 1539, 1204. HRMS (ESI) calcd C₃₅H₃₉F₅N₅O₁₂⁺ [M+H]⁺: 816.2510. Found: 816.2509.

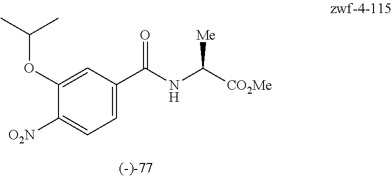 [SMILES: COC(=O)[C@H](C)NC(=O)c1ccc([N+](=O)[O-])c(OC(C)C)c1]

(−)-77 was obtained as white solid from the asymmetric decarboxylation reaction of malonic acid S77 (340.5 mg, 1 mmol) for 12 h using the general procedure with (S)-CPA-8 as catalyst (294.5 mg, 95% Yield). R_{f}=0.5 (Hexane/EtOAc 2:1). HPLC analysis (Chiralpak IA-3, hexane/PrOH=80/20, 0.8 mL/min, 254 nm; tᵣ (major)=8.06 min, tᵣ(minor)=10.33 min) gave the isomeric composition of the product: 88% e.e., (81% Yield, 94% e.e., single recrystallized from DCM/hexane), [α]_{D}¹⁹=−24.5 (c=1.00, CHCl₃ (94% e.e.)). ¹H NMR (400 MHz, CDCl₃) δ 7.78 (d, J=8.0 Hz, 1H), 7.60 (s, 1H), 7.29 (d, J=8.4 Hz, 1H), 6.86 (d, J=6.0 Hz, 1H), 4.87-4.68 (m, 2H), 3.82 (s, 3H), 1.55 (d, J=7.2 Hz, 3H), 1.40 (d, J=5.6 Hz, 6H). ¹³C NMR (100 MHz, CDCl₃) δ 173.4, 164.9, 151.1, 142.6, 138.3, 125.2, 117.6, 115.3, 72.9, 52.6, 48.6, 21.7, 21.6, 18.0. IR (neat, cm⁻¹) 3313, 2987, 1743, 1530, 1262. HRMS (ESI) calcd C₁₄H₁₉N₂O₆⁺ [M+H]⁺: 311.1238. Found: 311.1240.

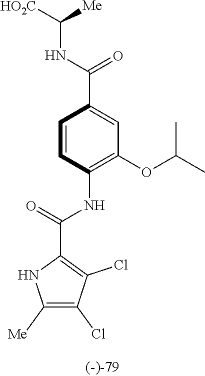

[α]_{D}¹⁹=−10.1 (c=0.90, MeOH). ¹H NMR (400 MHz, CD₃OD) δ 7.64 (s, 1H), 7.57-7.45 (m, 2H), 6.84 (s, 1H), 4.83-4.78 (m, 1H), 4.49-4.39 (m, 1H), 2.33 (s, 3H), 1.48 (d, J=7.2 Hz, 3H), 1.44 (d, J=6.0 Hz, 3H), 1.32 (d, J=6.0 Hz, 3H). ¹³C NMR (150 MHz, CD₃OD) δ 179.8, 168.1, 159.1, 154.8, 137.0, 129.2, 128.6, 125.8, 120.4, 120.1, 115.0, 114.4, 109.4, 72.6, 52.5, 22.4, 22.2, 19.2, 10.1. IR (neat, cm⁻¹) 3351, 2982, 1700, 1500, 1107. HRMS (ESI) calcd C₁₄H₂₂N₃O₅C₁₂⁺ [M+H]⁺: 442.0931. Found: 442.0930.

### Crystal Structures and Crystallographic Data of (+)-17

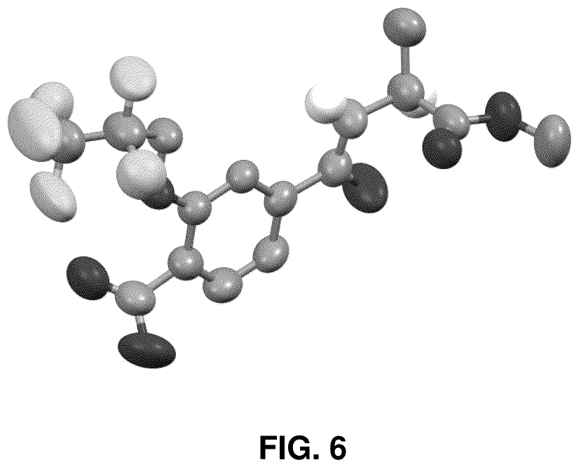

The crystal structure of (+)-17 is shown in FIG. 6. Crystal data for (+)-17 are as follows: C₁₄H₁₃F₅N₂O₆, Mw=400.26, orthorhombic, space group P 2₁, 2₁, 2, final R indices [I>2σ(I)], R₁=0.0588, wR₂=0.2027; R indices (all data), R₁=0.0612, wR₂=0.2083, a=7.0383 (1) Å, b=20.9238 (3) Å, c=11.6281(2) Å, α=90°, β=90°, γ=90°, V=1712.45(5) Å³, T=295 K, Z=4, reflections collected/unique 17002/3594 [Rᵢₙₜ=0.0186], number of observations [>2σ(I)] 3284, parameters: 266. Radiation Cu Kα (λ=1.54184), ρ_{calc}=1.553 g/cm³, flack parameter 0.07(5). CCDC-2206851.

### Decarboxylation Conditions

Compared with enzymatic catalysis, asymmetric decarboxylation using chemical catalysis is underexplored. Chiral organic bases (e.g., 6) have been examined as the catalyst, but amino acids were only obtained in low enantioselectivity. The method accomplishes asymmetric decarboxylation, especially with chiral phosphoric acids (FIG. 1, CPA, 7). These acids not only lie in their high acidity that can rapidly protonate, but also can impose stereocontrol by forming a rich network of hydrogen bonds within their pockets, particularly with heteroatom-rich substrates or intermediates (e.g., FIGS. 1, 8 and 9). As such, the method can address the long-standing challenge not only as a proton donor but also by forming a rich network of hydrogen bonds with the diacid substrates or enol intermediates. It is hypothesized that the CPA-catalyzed pathway can outcompete the background self-decarboxylation by intercepting either the intramolecular CO₂ extrusion (8) or enol protonation (9). For example, the chiral acid may interrupt the internal hydrogen bonding of the malonic acid (8) and initiate the decarboxylation via an intermolecular proton transfer (10). Alternatively, the direct interception of the enol intermediate 9 from the self-decarboxylation is also possible. As shown in FIG. 1, in either scenario, a stereoselective protonation inside the chiral pocket (11) can produce the chiral amino acid (12).

The chiral pocket of phosphoric acids is shown in Scheme 19 below. Chiral phosphoric acids can induce the enantioselectivity of the decarboxylation. However, both the backbone and side chains of the acids are needed for their performance. Optimal enantiocontrol can be achieved with the combination of a spirocyclic skeleton and a pair of mesityl substituents.

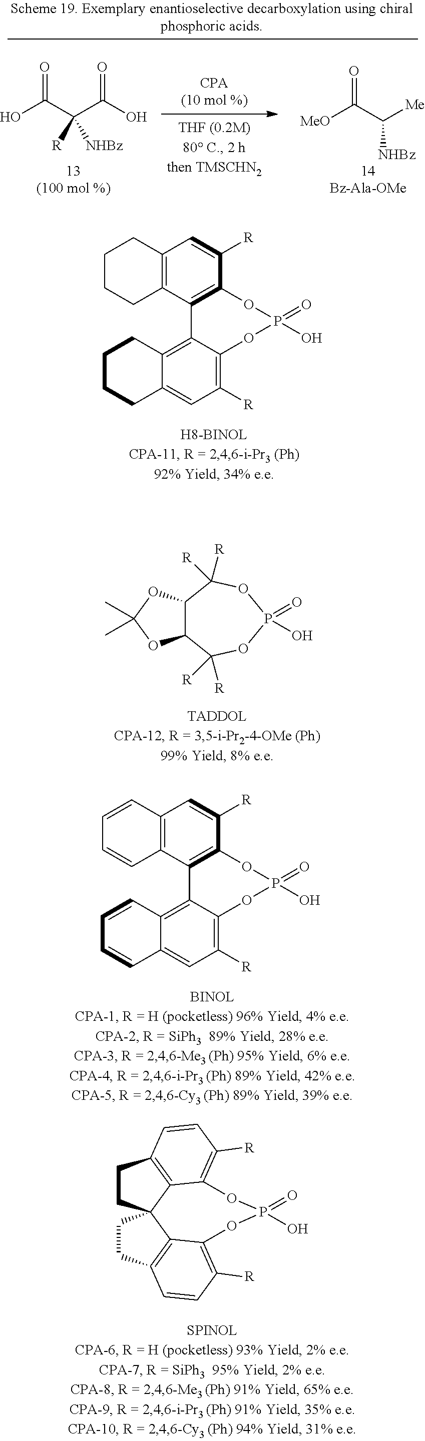

As shown in Scheme 19, while the decarboxylation of diacid 13 using the pocketless BINOL-derived CPA-1 provided benzoylated alanine (14) as a nearly racemic mixture, moderate enantiocontrol can be obtained with a pair of bulky side arms in place (CPA-2 to CPA-5). Meanwhile, the backbone of the acid is equally important in tuning the microenvironment. Particularly, the combination of a spirocyclic scaffold (CPA-6 to CPA-10) and mesityl substituents (CPA-8) induced a much-enhanced enantioselectivity for the decarboxylation. Several other skeletons of phosphoric acids, including H8-BINOL (CPA-11) and TADDOL (CPA-12), were also examined (Table 1).

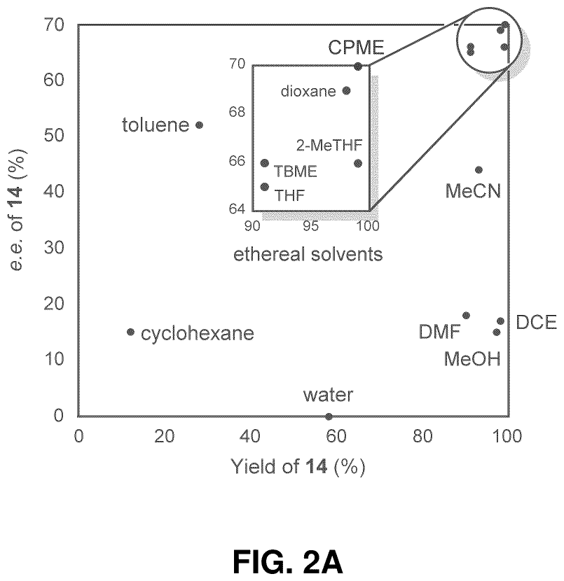

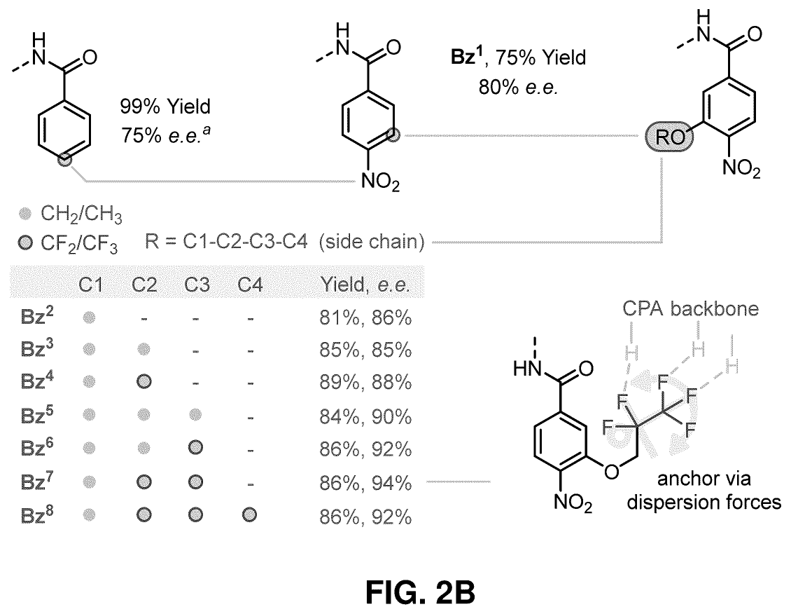

The decarboxylation is influenced by solvents (FIG. 2A and Table 2). In general, polar solvents resulted in a higher rate of decarboxylation compared with nonpolar ones. Solvents of high polarity are prone to give higher decarboxylation rate compared with nonpolar ones due to stabilization of polar transition states. The acceleration may be attributed to the stabilization of polar transition states of the decarboxylative C—C cleavage. However, these polar solvents can also disrupt interactions (e.g., hydrogen bonds) between phosphoric acids and substrates or intermediates, thus giving low enantioselectivity. For example, water and alcohols can diminish the enantioselectivity by disrupting the organized hydrogen bonds and other weaker interactions with phosphoric acids. In contrast, the family of ethereal solvents could strike a balance between the reactivity and enantioselectivity of the decarboxylation, with cyclopentyl methyl ether (CPME), an emerging process solvent with good safety and sustainability, slightly outcompeting other members. The effects of reaction temperature and concentration on decarboxylation is shown in Table 3.

TABLE 1
Screening of chiral phosphoric acids

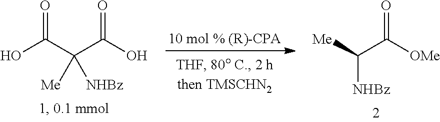

Reaction conditions unless noted otherwise: 1 (0.1 mmol). (R)-CPA (10 mol %), in THF (0.5 mL) at 80° C. for 3 h. The yield was determined by crude ¹H NMR analysis using dibromomethane as the internal standard and e.e. was determined by HPLC analysis of isolated product.

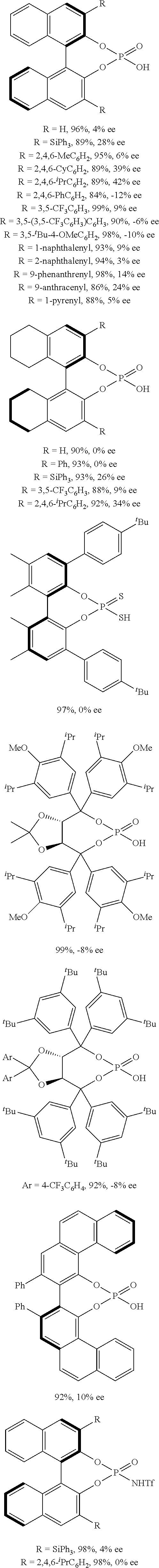

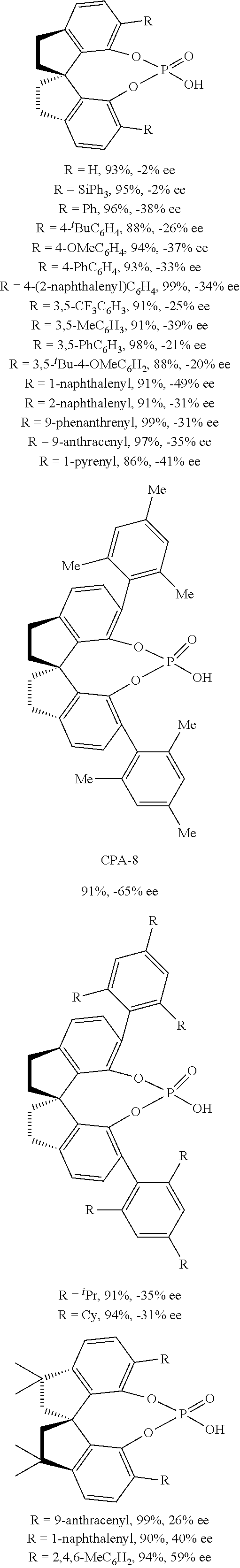

TABLE 2
Solvent effects on decarboxylation

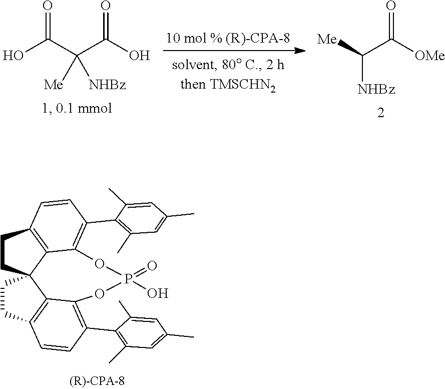

| entry | solvent | Yield and e.e. (%) | recovery of 1 (%) |
|---|---|---|---|
| 1 | Benzene | 31, 50 | 69 |
| 2 | Toluene | 28, 52 | 70 |
| 3 | Mesitylene | 18, 52 | 81 |
| 4 | Benzotrifluoride | 27, 42 | 70 |
| 5 | Chlorobenzene | 69, 41 | 31 |
| 6 | MeOH | 97, 15 | 0 |
| 7 | HFIP | 78, 3 | 0 |
| 8 | Acetic acid | 98, 5 | 0 |
| 9 | Acetone | 95, 59 | 0 |
| 10 | Cyclohexanone | 91, 59 | 0 |
| 11 | Ethyl acetate | 99, 65 | 0 |
| 12 | γ-Butyrolactone | 86, 48 | 0 |
| 13 | CH₃CN | 93, 44 | 0 |
| 14 | CHCl₃ | 95, 31 | 0 |
| 15 | DCE | 98, 17 | 0 |
| 16 | ⁿPrNO₂ | 92, 37 | 0 |
| 17 | NMP | 88, 9 | 0 |
| 18 | DMF | 90, 18 | 0 |
| 19 | DMSO | 88, 8 | 0 |
| 20 | H₂O | 58, 0 | 39 |
| 21 | Cyclohexane | 12, 15 | 87 |
| 22 | Et₂O | 87, 43 | 8 |
| 23 | ^{t}BuOMe | 91, 66 | 0 |
| 24 | THF | 91, 65 | 0 |
| 25 | 2-MeTHF | 99, 66 | 0 |
| 26 | 1,4-Dioxane | 98, 69 | 0 |
| 27 | CPME | 99, 70 | 0 |
| 28 | (MeOCH₂CH₂)₂O | 96, 62 | 0 |

Reaction conditions unless noted otherwise: 1 (0.1 mmol), (R)-CPA-8 (10 mol %), in solvent (0.5 mL) at 80º C. for 2 h.
The yield was determined by crude ¹H NMR analysis using dibromomethane as the internal standard and e.e. was determined by HPLC analysis of isolated product.

TABLE 3
Effects of reaction temperature and concentration on decarboxylation.

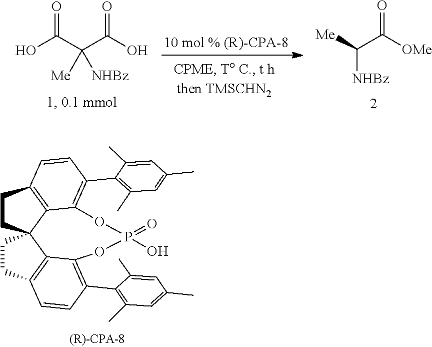

| | | | | Yield and | recovery |
|---|---|---|---|---|---|
| entry | c (M) | T (º C.) | t (h) | e.e. (%) | of 1 (%) |
| 1 | 0.1 | 100 | 2 | 96, 70 | 0 |
| 2 | 0.1 | 90 | 2 | 95, 70 | 0 |
| 3 | 0.1 | 80 | 2 | 99, 70 | 0 |
| 4 | 0.1 | 70 | 2 | 76, 72 | 23 |
| 5 | 0.1 | 50 | 12 | 62, 72 | 36 |
| 6 | 0.2 | 80 | 2 | 99, 70 | 0 |
| 7 | 0.4 | 80 | 2 | 97, 65 | 0 |
| 8 | 0.05 | 80 | 2 | 99, 75 | 0 |

Reaction conditions unless noted otherwise: 1 (0.1 mmol), (R)-CPA-8 (10 mol %), in CPME at T º C. for t h.
The yield was determined by crude ¹H NMR analysis using dibromomethane as the internal standard and e.e. was determined by HPLC analysis of isolated product.
″c (M)″ is the concentration of the malonic acid substrate 1.

The amine protecting group provides another handle for improving the enantiocontrol. A further boost in enantioselectivity was obtained via screening of varied amine protecting groups, indicating its engagement with the chiral phosphoric acid during the enantioinduction (FIG. 2B). As shown in Table 4, compared with other protecting groups, including acetyl, pivalate, and benzyl carbamate, benzamide (Bz) was selected for further derivatization. As shown in FIG. 2B, further derivatization of the benzoyl group showed that a para-nitro (Bz¹) and a meta-methoxy (Bz²) group could join force to improve the enantioselectivity. Both substituents can enhance the acidity of the amide proton, which in return may act as a stronger hydrogen bond donor to strengthen substrate-catalyst interaction. Moreover, it is geometrically possible that an extended side chain of the meta-alkoxyl group could further help anchor the substrate or intermediate inside the acid pocket. Although simple elongation of the alkyl chain is only negligibly beneficial (Bz³ and Bz⁵), the incorporation of multiple fluorides (Bz⁴ and Bz⁶-Bz⁸) raised the enantiocontrol to an excellent level, with pentafluoropropoxy group (Bz⁷) as the top substituent (FIG. 2B). This increment of enantiocontrol by the fluorinated chain may originate from its weak interaction, such as dispersive interaction, with the phosphoric acid and C—H bond-rich skeleton of the phosphoric acid that serves as an additional anchor to fix the conformation of intermediates.

TABLE 4
Effects of amine protecting groups.

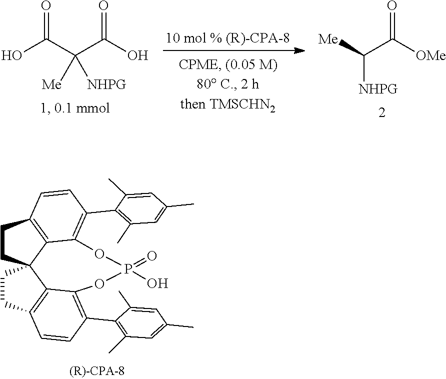

Reaction conditions unless noted otherwise: 1 (0.1 mmol), (R)-CPA-8 (10 mol %), in CPME (2.0 mL) at 80° C. for 2 h. The yield was determined by crude ¹H NMR analysis using dibromomethane as the internal standard and e.e. was determined by HPLC analysis of isolated product. ^{a}The reaction was run for 5 h.

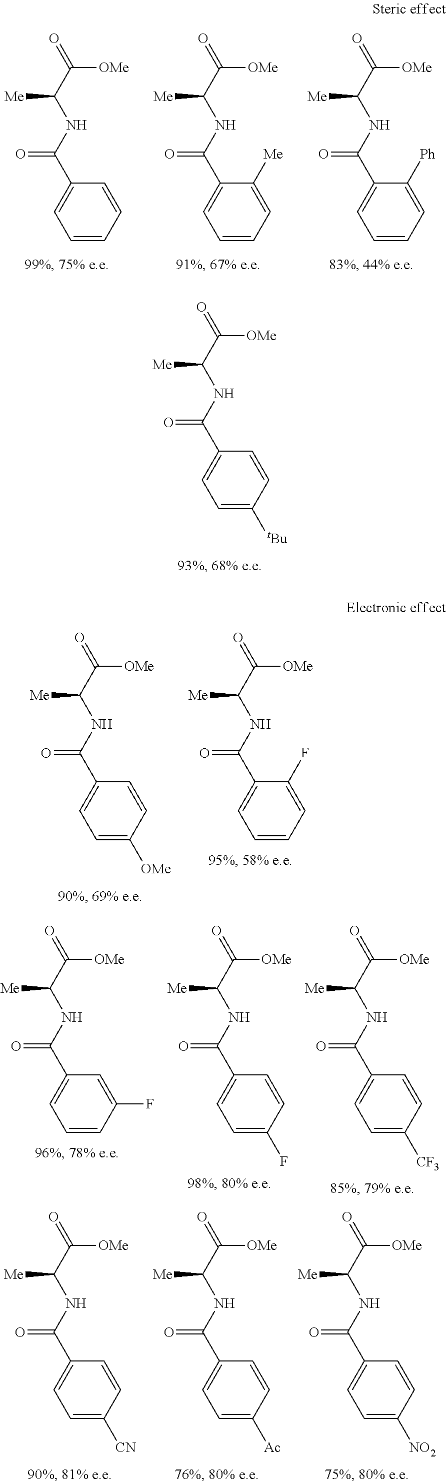

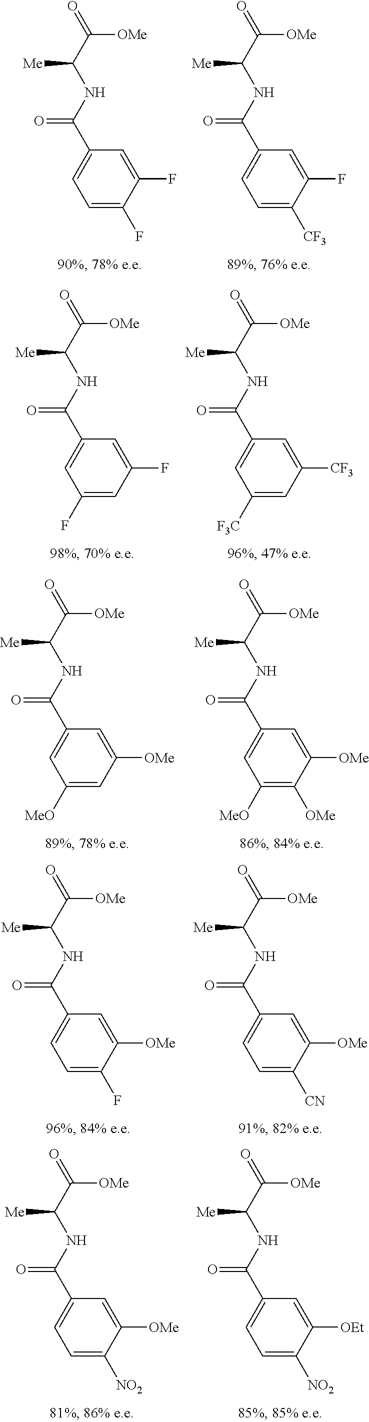

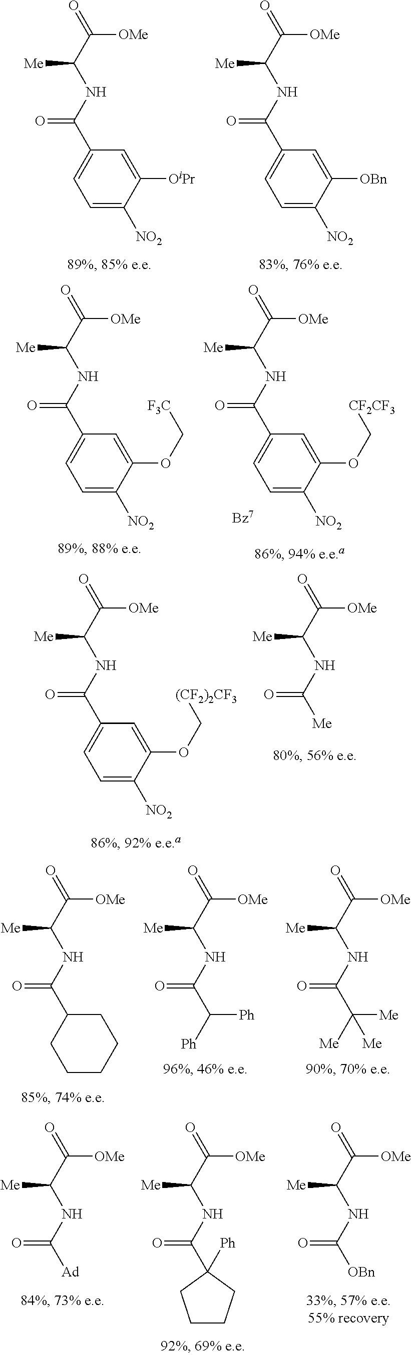

### Asymmetric Malonic Ester Synthesis

Aminomalonic acids bearing the customized protecting group (Bz⁷) can be prepared in a divergent and modular fashion (FIG. 3). Starting from the inexpensive aminomalonate hydrochloride 15, a hectogram-scale benzoyl installation to 16, followed by the unified alkylation and hydrolysis conditions, can generate structurally diverse diacids for decarboxylation. Meanwhile, the commercial availability of phosphoric acids, such as CPA-8 of opposite configurations, allows access to both enantiomers of the product, such as the natural and unnatural alanine derivatives (+)/(−)-17. Besides the facile methylative work-up using trimethylsilyldiazomethane, amino acid hydrochloride salts can be obtained via hydrolysis. When a larger scale decarboxylation under a lower catalyst loading was ensued by treatment with acids, analytically pure alanine salt (18) can be obtained by a simple extraction. The direct preparation of alanine hydrochloride salt (18) on a larger scale using less catalyst showcases the high practicality of the method. This protocol also opens opportunities to recycle the tailored benzoic acid (Bz⁷OH) and phosphoric acid catalyst that remain in the organic phase.

Structurally distinct amino acids have unique functions, and the method can prepare a diversity of them. The chiral phosphoric acid is tolerant of alkyl groups of distinct shapes (19-24). The chiral phosphoric acid can accommodate extended alkyl chains (19-22) and cycloalkyl (23) groups to generate unnatural amino acids, including those isomeric to proteinogenic ones, such as norvaline (20) and norleucine (21). The method also tolerates more crowded secondary alkyl substituents (24), with slightly lower enantioselectivity. The diversity of functional groups that can be introduced to amino acids (25-40) is immense, which can serve as a handle to further modify the amino acids or bring varied functions to peptides or proteins. When unsaturated motifs are present in the side chain, they diversify not only the shape but also further modification of the amino acids. As such, various types of olefins, including monosubstituted (25), 1,1/1,2-disubstituted (26 and 27), and trisubstituted (28) ones, as well as terminal (29) and internal (30) alkynes, can be integrated. Chains with multiple such motifs, including a diene derived from geraniol (31) and an enyne (32), are also compatible. Further transformations of these olefins and alkynes, such as coupling and oxidation reactions, can generate amino acids of higher complexity. These functional groups can also facilitate chemical modification of peptides and proteins via metathesis or click reactions. The functional groups are beyond unsaturated moieties. When a phenyl, indole, or phthalimide was attached to the alkyl chain, unnatural analogs to phenylalanine (33), tryptophan (34), and ornithine (35) were forged, respectively. The reduced enantioselectivity of 35 may result from the imide carbonyls that may interfere with the hydrogen bonding between the phosphoric acid and the enol intermediate. Malonic acids with a nitrile (36), acetal (37), or azide (38) group can also participate to give amino acids that inherit their versatile reactivity. Additionally, the acid catalyst operated smoothly on substrates with polyfunctionalized skeletons derived from cholic acid (39) and estrone (40), and the stereocontrol remained excellent despite their large sizes.

Cyclic amino acids (e.g., 44) are also accessible from the decarboxylation products (e.g., 41) through a sequence involving a pair of versatile intermediates (42 and 43). A route from p-methoxybenzyl ether 41 to proline 44 is demonstrated in FIG. 3D, which can be general to access natural or unnatural cyclic amino acids. Further, given the abundant reactivity of alcohol (42) and alkyl iodide (43), the pair of intermediates in the sequence are of great synthetic value to access functionalized acyclic amino acids.

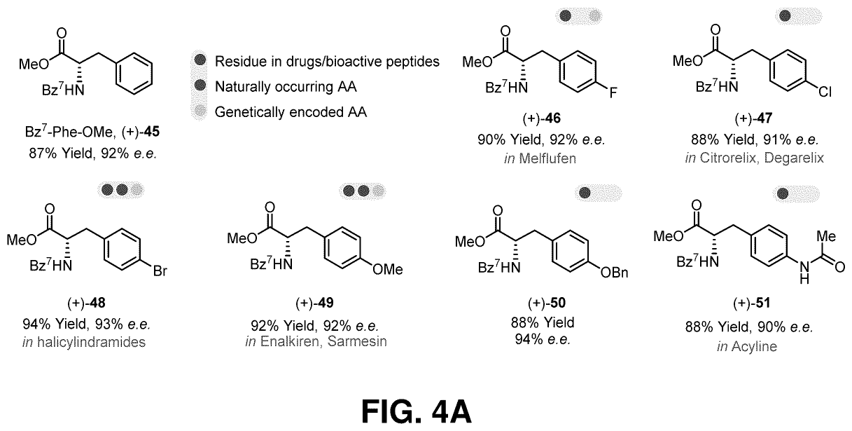

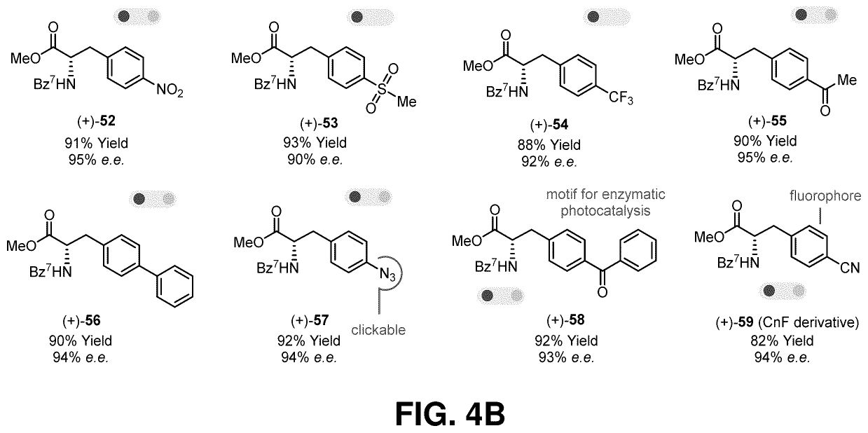

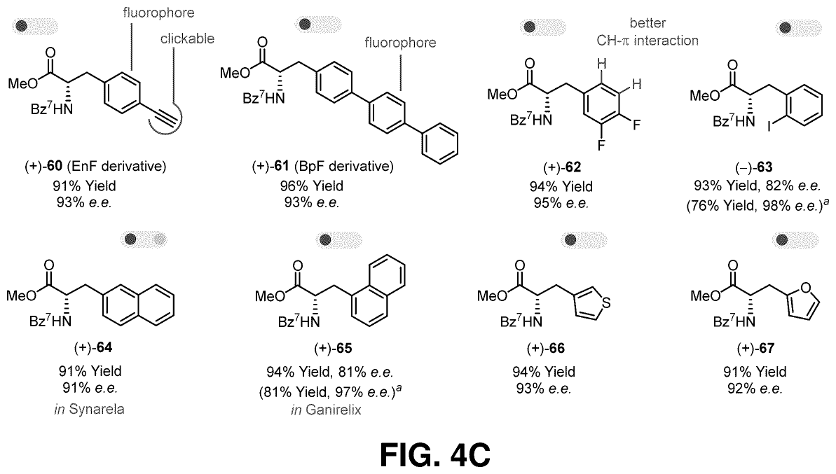

Non-proteinogenic derivatives of phenylalanines are particularly prevalent in bioactive peptide drugs and have been heavily used in multiple areas to introduce diverse functions to proteins with their aryl substituents. The asymmetric synthesis can prepare a library of them, which takes advantage of the vast availability of benzyl bromides and their facile substitution for the preparation of aminomalonic acids, as well as the catalyst's tolerance of substituents with different shapes and electronic properties. As such, phenylalanines with abundant functionalities, including fluorophores (59-61), NMR-active nuclei (46 and 54), and photosensitizers (58), can be accessed. The synthesis of functionalized phenylalanines was further investigated due to their prevalence in drugs and role as a vessel to convey varied functions to peptides and proteins. The availability of assorted benzyl bromides and their facile substitution reactions can provide a reliable supply of functionalized phenylalanines using facilitate asymmetric malonic ester synthesis. Indeed, as shown in FIG. 4, substituents of different shapes and electronic properties (i.e., electron-donating, -withdrawing, and -neutral) of benzyl substrates were all carried along the route to corresponding phenylalanine derivatives (46-56). The diversity of functionality that can be introduced is immense. Besides the NMR-active fluorides (46 and 54) that can assist structural studies of proteins, both partners of the azide-alkyne cycloaddition (57 and 60) are compatible. In addition to an enolizable methyl ketone, amino acid with a benzophenone motif (58) that can allow enzymatic photocatalysis was also obtained. The catalyst's tolerance of aryl nitrile and alkyne, as well as large terphenyl groups allows preparation of non-canonical fluorescent amino acids (59-61). Substitution patterns (62 and 63) and aromatic rings (64-67) other than these para-substituted phenyl groups (46-61) are equally well accommodated. The fluoro substituents in 62 were shown to reinforce CH-71 interaction within peptides by enhancing the acidity of aromatic hydrogens. Meanwhile, the ortho-iodide motif (63) can help derivatize the phenylalanine core via cross coupling. Malonic acids with a fused naphthalene (64-65) or heterocycle (66-67) can also decarboxylate to give non-proteinogenic amino acids. A slight decrease in stereocontrol was observed when an ortho-substituent is present (e.g., 63 and 65), presumably due to the steric repulsion inside the confined catalyst pocket. Nevertheless, a simple recrystallization can enhance the enantioselectivity without a large loss of yield.

### Synthetic Applications

Isotope-labeled amino acids are also accessible (Scheme 20). These structures are of high demand given their diverse application across several fields, including proteomics, pharmacokinetics, and diagnostic radiology, and the asymmetric malonic ester synthesis can provide a modular approach that is highly flexible regarding the identity of isotopes and the targeted positions in amino acids. The bottom-up synthesis can be easily adopted to produce isotope-labeled amino acids. By using a unified synthetic sequence, ¹³C and ¹⁵N isotopes can be introduced via an early-stage oxime formation, while the labeled side chains via a later substitution. For example, the α-¹³C and ¹⁵N isotopes can be introduced via a condensation reaction to oxime using labeled diethyl malonate or sodium nitrite, followed by reduction and benzoylation to α-¹³C- or ¹⁵N-16. On the other hand, the labeled side chains can be readily connected via substitution, as demonstrated by the preparation of d₃-68 (which has the structure of 68 but with CD₃ instead of CH₃, i.e., all the H in the CH₃ of 68 is replaced with deuterium). Subsequently, all three labeled malonic acids were decarboxylated under acid catalysis to give enantio- and isotope-enriched alanines (69-71).

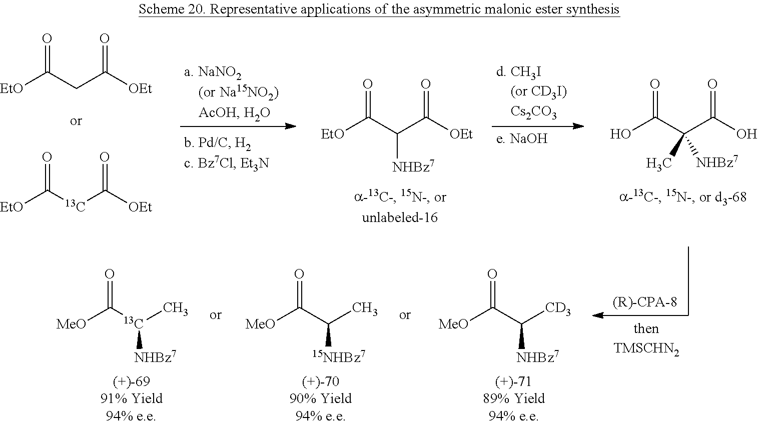

Non-proteinogenic amino acids with an alkyne or azide motif can conjugate with other biomolecules using click chemistry. Both amino acid-antiretroviral drug and -saccharide conjugates were easily prepared. For example, alkyne- or azide-substituted amino acids generated herein (29, 38, 57, and 60) can rapidly conjugate with biologically active cores, demonstrating their application in discovering amino acid- or peptide-based therapeutic agents (Scheme 21). With a generic copper catalyst, the cycloaddition between the propargyl group in 29 and Zidovudine's azide motif proceeded smoothly (72). Another amino acid-antiretroviral drug conjugate was prepared from the azido-containing product 57 and propargyl-tethered Lamivudine (73). Besides, saccharides and amino acids can also connect using the same strategy (74).

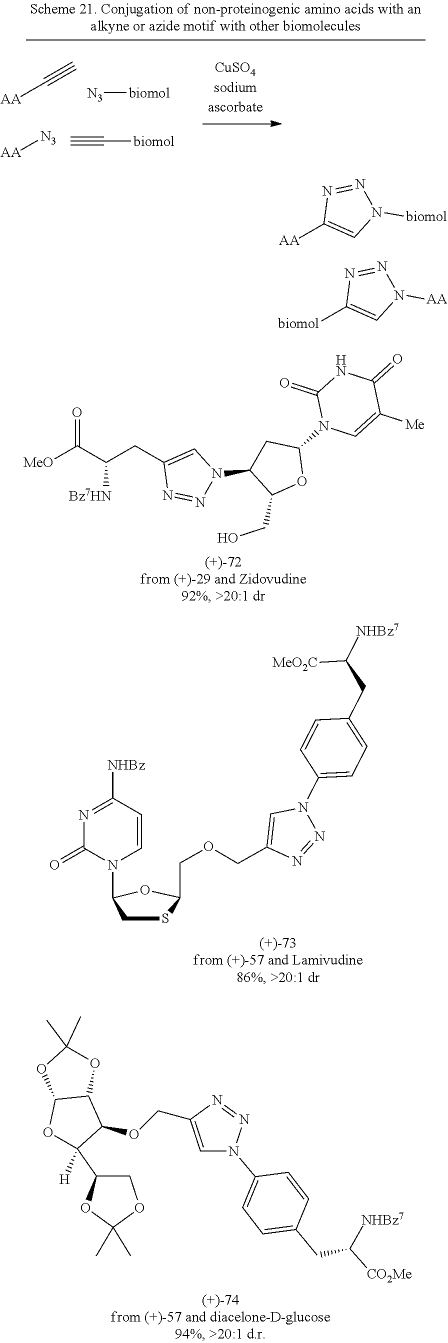

Besides serving as a protecting group, 3-alkoxy-4-nitrobenzamides, once reduced to corresponding anilines, can serve as α-helix mimetics that frequently found in bioactive small molecules and peptides (75, FIG. 5). Thus, a representative decarboxylation/reduction sequence towards these motifs (such as a DNA gyrase B inhibitor (79) containing an unnatural alanine residue) was devised. Starting from the tailored aminomalonic acid 76, asymmetric decarboxylation using (S)-CPA-8 provided an unnatural alanine motif (77) that served as the C-terminus of a helix mimetic. Subsequently, the N-terminus was unmasked by a hydrogenation of the nitro group (78). The following acylation and ester hydrolysis produced DNA gyrase B inhibitor 79.

### CONCLUSIONS

The excellent enantiocontrol of the acid-catalyzed decarboxylation was achieved using chiral phosphoric acids and the tailored protecting group (such as fluorinated benzoyl) of aminomalonic acids. The diverse interactions between the two components, especially the large number of dispersion forces, help anchor the enol intermediate and allow the phosphoric acid to achieve stereoselective protonation. Using the enantioselective decarboxylation reaction, the long-elusive malonate-based synthesis of chiral α-amino acids is now available. The synthetic protocol disclosed herein introduces the side chains of amino acids via the facile alkylation of aminomalonic esters. With subsequent hydrolysis and decarboxylation, the resulting amino acids have diverse shapes and substituents of the side chains and thus can provide distinct properties and functions.

Unless defined otherwise, all technical and scientific terms used herein have the same meanings as commonly understood by one of skill in the art to which the disclosed invention belongs. Publications cited herein and the materials for which they are cited are specifically incorporated by reference. Further, unless otherwise indicated, use of the expression “wt %” refers to “wt/wt %.”

Those skilled in the art will recognize, or be able to ascertain using no more than routine experimentation, many equivalents to the specific embodiments of the invention described herein. Such equivalents are intended to be encompassed by the following claims.

### REFERENCES

J. Wislicenus, Liebigs Ann. Chem. 186, 161-228 (1877).
A. C. Cope, et al., Org. React. 107-331 (1957).
H. O. House, Modern Synthetic Reactions (The Organic Chemistry Monograph Series). 2nd ed (W. A. Benjamin, inc., 1972) pp. 492-623.
S. Gabriel, Ber. 20, 2224-2236 (1887).
M. S. Gibson and R. W. Bradshaw, Angew. Chem., Int. Ed. Engl. 7, 919-930 (1968).
S. K. Mitra, J. Ind. Chem. Soc. 7, 799-802 (1930).
C. Fehr, Angew. Chem. Int. Ed. Engl. 35, 2566-2587 (1996).
K. Miyamoto and Kourist, R. Appl. Microbiol. Biotechnol. 100, 8621-8631 (2016).
M. Wilding, et al., Comprehensive Chirality, H. Yamamoto, & E. M. Carreira, Eds. (Elsevier, 2012) pp. 402-409.
W. H. Richardson and H. E. O'Neal, Comprehensive Chemical Kinetics: Decomposition and Isomerization of Organic Compounds, C. H. Bamford & C. F. H. Tipper Eds. (Elsevier, 1972) pp. 381-565.
K. Okrasa, et al., Angew. Chem. Int. Ed. 48, 7691-7694 (2009).
J. Blanchet, et al., Eur. J. Org. Chem. 5493-5506 (2008).
H. Brunner and M. A. Baur, Eur. J. Org. Chem. 2854-2862 (2003).
M. Rueping, et al., Chem. Soc. Rev. 40, 4539-4549 (2011).
C. Min and D. Seidel, Chem. Soc. Rev. 46, 5889-5902 (2017).
T. Akiyama, Chem. Rev. 107, 5744-5758 (2007).
T. Akiyama and K. Mori, Chem. Rev. 115, 9277-9306 (2015).
M. Terada, Chem. Commun. 4097-4112 (2008).
D. Parmar, et al., Chem. Rev. 114, 9047-9153 (2014).
R. Maji, et al., Chem. Soc. Rev. 47, 1142-1158 (2018).
A. G. Doyle and E. N. Jacobsen, Chem. Rev. 107, 5713-5743 (2007).
J.-H. Xie and Q.-L. Zhou, Acc. Chem. Res. 41, 581-593 (2008).
A. Rahman and X. Lin, Org. Biomol. Chem. 16, 4753-4777 (2018).
G. G. Midyana, et al., Russ. J. Gen. Chem. 80, 944-947 (2010).
I. A. Koppel and V. A. Palm, Advances in Linear Free Energy Relationships (eds. Chapman, N. B. & Shorter, J.) 203-280 (Springer, 1972).
K. Watanabe, et al., Org. Process Res. Dev. 11, 2, 251-258 (2007).
M. S. Messina and H. D. Maynard, Mater. Chem. Front. 4, 1040-1051 (2020).
C. G. Parker and M. R. Pratt, Cell 180, 605-632, (2020).
D. R. Liu and P. G. Schultz, Proc. Natl. Acad. Sci. USA 96, 4780-4785 (1999).
C. C. Liu and P. G. Schultz, Annu. Rev. Biochem. 79, 413-444 (2010).
C. H. Kim, et al., Curr. Opin. Chem. Biol. 17, 412-419 (2013).
M. A. Danielson and J. J. Falke, Annu. Rev. Biophys. Biomol. Struct. 25, 163-195 (1996).
K. E. Arntson and W. C. K. Pomerantz, J. Med. Chem. 59, 5158-5171 (2016).
J. S. Trimble, et al., Nature 611, 709-714 (2022).
N. Sun, et al., Nature 611, 715-720 (2022).
Z. Cheng, et al., Nat. Rev. Chem. 4, 275-290 (2020).
T. Fujita, et al., Tetrahedron Lett. 41, 923-927 (2000).
M. Mann, Nat. Rev. Mol. Cell. Biol. 7, 952-958 (2006).
F. Castellani, et al., Nature 420, 99-102 (2002).
M. T. Lin, et al., J. Am. Chem. Soc. 130, 15768-15769 (2008).
A. Ullah, et al., Eur. J. Med. Chem. 145, 140-153 (2018).
H. Arifian, et al., Molecules 27, 7631-7664 (2022).
A. Shaginian, et al., J. Am. Chem. Soc. 131, 5564-5572 (2009).
M. Durcik, et al., Eur. J. Med. Chem. 154, 117-132 (2018).
W. H. Richardson and H. E. O'Neal, Comprehensive Chemical Kinetics: Decomposition and Isomerization of Organic Compounds; Bamford C. H., Tipper C. F. H., Eds.; Elsevier: Amsterdam, The Netherlands, 1972; Vol. 5, Chapter 4, p 381-565.
W. Huang, et al., Org. Lett. 2020, 22, 3245-3250.
C. J. Easton, et al., J. Aust. J. Chem. 1997, 50, 451-456.
R. H. Verschueren, et al., Org. Biomol. Chem., 2021, 19, 5782-5787.
J. Mao, et al., J. Am. Chem. Soc. 2014, 136, 17622-17668.

We claim:

1. A method for producing chiral amino acids, comprising:
(i) maintaining a first reaction mixture at a first temperature for a first period of time sufficient to form a product,
wherein the first reaction mixture comprises a substrate, a catalyst, and a solvent,
wherein the catalyst is a chiral phosphoric acid, and
wherein the product comprises a chiral amino acid.

2. The method of claim 1, wherein the substrate is a malonic acid.

3. The method of claim 2, wherein the malonic acid has the structure of Formula I or Formula I′: 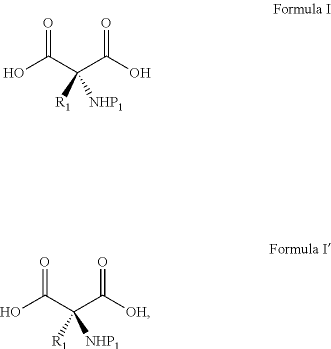
wherein the chiral amino acid in the product has the structure of Formula II or Formula II′: 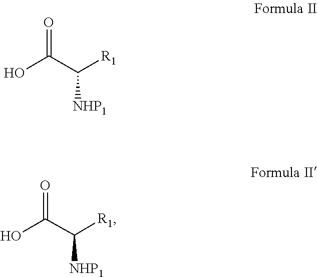
wherein P₁ is an amino protecting group and R₁ is a functional group or biomolecule moiety.

4. The method of claim 1, further comprising:
(ii) adding an alkylation reactant or an acid to the product to form a second reaction mixture, and
(iii) maintaining the second reaction mixture at a second temperature for a second period of time sufficient to form an alkylated product or hydrolysis product,
wherein the alkylation product or hydrolysis product comprises an alkylated chiral amino acid or hydrolyzed chiral amino acid.

5. The method of claim 4, wherein the alkylated chiral amino acid has the structure of Formula VIII or Formula VIII′, and the hydrolyzed chiral amino acid has the structure of Formula V or Formula V′: 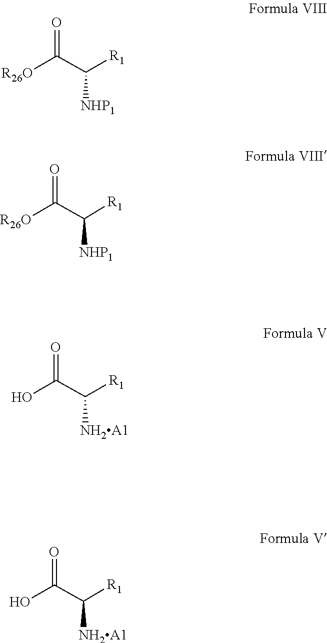
wherein P₁ is an amino protecting group and R₁ is a functional group or biomolecule moiety, R₂₆ is a substituted or unsubstituted alkyl, such as a C₁-C₁₂ unsubstituted alkyl, a C₁-C₁₀ unsubstituted alkyl, a C₁-C₈ unsubstituted alkyl, a C₁-C₆ unsubstituted alkyl, or a C₁-C₄ unsubstituted alkyl, for example, methyl or ethyl, and A1 is an acid molecule (such as HCl),
optionally wherein the alkylated chiral amino acid is a methylated chiral amino acid having the structure of Formula IV or Formula IV′: 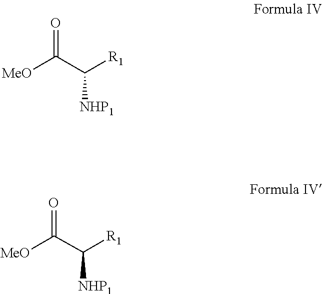
wherein P₁ is an amino protecting group and R₁ is a functional group or biomolecule moiety.

6. The method of claim 4, wherein the alkylation reactant in the second reaction mixture is a methylation reactant (such as trimethylsilyldiazomethane) and the acid in the second reaction mixture is HCl.

7. The method of claim 3, wherein P₁ is 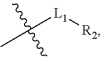
and wherein:
L₁ is 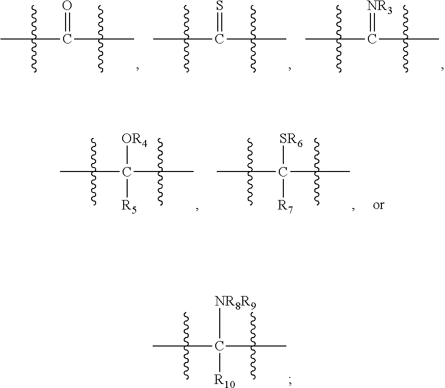
R₂ is hydrogen, hydroxyl, halide, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, carbonyl, cyano, isocyano, nitro, silyl, sulfinyl, sulfonyl, phosphonium, phosphanyl, phosphoryl, or phosphonyl;
R₃-R₁₀ are independently hydrogen or a substituted or unsubstituted alkyl (such as unsubstituted alkyl, for example, C₁-C₁₀ unsubstituted alkyl, C₁-C₈ unsubstituted alkyl, C₁-C₆ unsubstituted alkyl, or C₁-C₄ unsubstituted alkyl); and
the substituents, when present, are independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl,
optionally wherein L₁ is

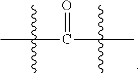 [SMILES: *C(*)=O]

8. The method of claim 7, wherein R₂ is substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclyl, substituted or unsubstituted aralkyl, alkoxy, thiol, amino, amido, or carbonyl.

9. The method of claim 7, wherein R₂ is 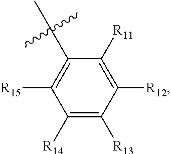
and wherein R₁₁-R₁₅ are independently hydrogen, substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, oxo, silyl, sulfinyl, sulfonyl, phosphonium, phosphanyl, phosphoryl, or phosphonyl.

10. The method of claim 9, wherein R₁₁-R₁₅ are independently hydrogen, halide, unsubstituted alkyl, unsubstituted alkenyl, unsubstituted alkynyl, unsubstituted phenyl, unsubstituted haloalkyl unsubstituted aralkyl, cyano, isocyano, nitro, 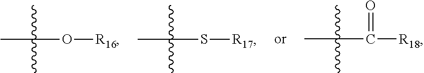
and wherein R₁₆-R₁₈ are independently hydrogen, hydroxyl, —OR′₁₈, unsubstituted alkyl, unsubstituted phenyl, unsubstituted haloalkyl, or unsubstituted aralkyl, and R′₁₈ is unsubstituted alkyl.

11. The method of claim 7, wherein R₂ is: 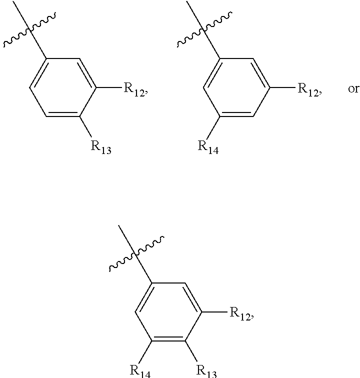
and
wherein R₁₂-R₁₄ are independently halide (such as F, Cl, or I), unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), cyano, isocyano, nitro, 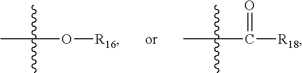
and wherein R₁₆ and R₁₈ are independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), and R′₁₈ is unsubstituted alkyl.

12. The method of claim 3, wherein R₁ is a substituted or unsubstituted alkyl, a substituted or unsubstituted alkenyl, or a substituted or unsubstituted alkynyl, and
wherein the substituents, when present, are independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl.

13. The method of claim 12, wherein the substituents, when present, are independently a biomolecule moiety.

14. The method of claim 1, wherein one or more carbons, one or more nitrogen, and/or one or more hydrogens of the substrate are in the form of ¹³C, ¹⁴C, ¹⁵N, and/or D.

15. The method of claim 1, wherein the chiral phosphoric acid is a Binol phosphoric acid or derivative thereof, an H8 Binol phosphoric acid or derivative thereof, a Spinol phosphoric acid or derivative thereof, a Biphenol phosphoric acid or derivative thereof, a dithiophosphoric acid or derivative thereof, a Taddol phosphoric acid or derivative thereof, a paracyclophane or derivative thereof, a TiPSY phosphoric acid or derivative thereof, or a TRIP phosphoric acid or derivative thereof.

16. The method of claim 1, wherein the chiral phosphoric acid has the structure of Formula III or Formula III′: 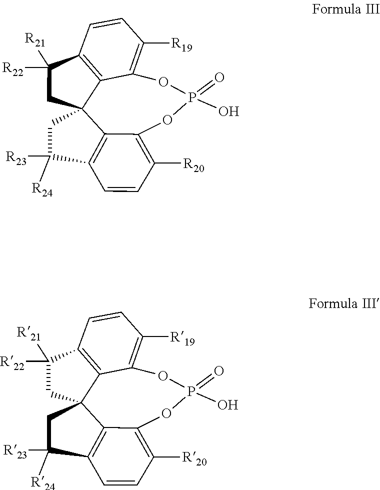
wherein:
R₁₉, R₂₀, R′₁₉, and R′₂₀ are independently substituted or unsubstituted aryl or substituted or unsubstituted polyaryl;
R₂₁-R₂₄ and R′₂₁-R′₂₄ are independently hydrogen or unsubstituted alkyl; and
the substituents, when present, are independently substituted or unsubstituted alkyl, substituted or unsubstituted aryl (such as phenyl), substituted or unsubstituted polyaryl (such as naphthal), substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or 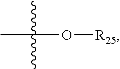
and R₂₅ is substituted or unsubstituted alkyl, substituted or unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), substituted or unsubstituted aryl (such as phenyl), or substituted or unsubstituted aralkyl (such as benzyl).

17. The method of claim 16, wherein at least one of R₁₉ and R₂₀ and at least one of R′₁₉ and R′₂₀ is independently a substituted aryl or substituted polyaryl, and optionally wherein the substituted aryl or polyaryl has three or more substituents (such as 2,4,6-substituted phenyl).

18. The method of claim 1, wherein the chiral phosphoric acid has the structure of

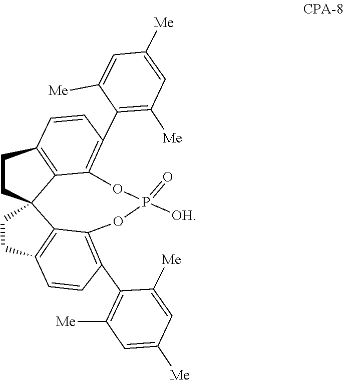

19. The method of claim 1, wherein the catalyst is present in the first reaction mixture in an amount ranging from about 1 mol % to about 20 mol %, from about 1 mol % to about 10 mol %, from about 2 mol % to about 20 mol %, from about 2 mol % to about 10 mol %, or from about 2.5 mol % to about 10 mol %, such as about 10 mol % or about 2.5 mol %.

20. The method of claim 1, wherein the solvent is an ether, optionally wherein the ether is ethyl acetate, ^{t}BuOMe, THF, 2-MeTHF, 1,4-dioxane, cyclopentylmethylether (CPME), or (MeOCH₂CH₂)₂O, or a combination thereof.

21. The method of claim 1, wherein the first reaction mixture is maintained at a temperature ranging from about 50° C. to about 100° C., from about 60° C. to about 100° C., or from about 70° C. to about 100° C., such as about 80° C., for a period of time ranging from about 1 hour to about 12 hours, from about 1 hour to about 10 hours, from about 1 hour to about 8 hours, from about 1 hour to about 5 hours, or from about 1 hour to about 3 hours, such as about 2 hours.

22. The method of claim 4, wherein the second reaction mixture (such as for methylation reaction) is maintained at a temperature ranging from about 50° C. to about 100° C., from about 60° C. to about 100° C., or from about 70° C. to about 100° C., for a period of time ranging from about 30 minutes to about 2 hours or from about 30 minutes to about 1 hour, or
wherein the second reaction mixture (for hydrolysis) is maintained at a temperature ranging from about 90° C. to about 120° C. or from about 90° C. to about 110° C., for a period of time ranging from about 12 hours to about 36 hours or from about 12 hours to about 24 hours.

23. The method of claim 4, wherein after step (i), the product is cooled to room temperature or about 0° C. before adding the alkylation reactant (such as the methylation reactant) or the acid.

24. The method of claim 1, wherein the chiral amino acid has a yield of at least 80% or in a range from about 80% to about 99%, and/or an enantiometric excess (ee) of at least 70%, at least 75%, at least 80%, at least 85%, at least 90%, in a range from about 70% to 100%, from about 75% to 100%, from about 80% to 100%, from about 85% to 100%, from about 90% to 100%, from about 75% to 99%, from about 80% to 99%, from about 85% to 99%, or from about 90% to 99%, as determined by chiral HPLC.

25. The method of claim 1, further comprising purifying the product after step (i) and/or the alkylation or hydrolysis product after step (iii); preparing the substrate prior to step (i); recycling an amino protecting agent and the catalyst after step (i) and/or step (iii); and/or derivatizing the chiral amino acid or alkylated or hydrolyzed chiral amino acid to a derivatized compound after step (i) and/or step (iii), optionally wherein the derivatized compound is a cyclic amino acid, a drug-amino acid conjugate, or a DNA gyrase inhibitor.

26. A chiral amino acid having the structure of Formula VIII or Formula VIII′: 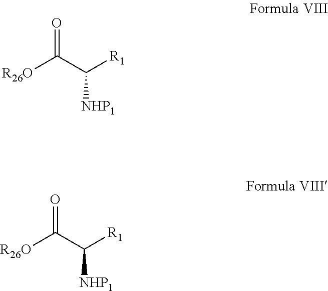
wherein R₂₆ is a substituted or unsubstituted alkyl, such as a C₁-C₁₂ unsubstituted alkyl, a C₁-C₁₀ unsubstituted alkyl, a C₁-C₈ unsubstituted alkyl, a C₁-C₆ unsubstituted alkyl, or a C₁-C₄ unsubstituted alkyl, e.g., methyl or ethyl;
P₁ is 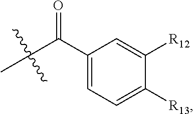
wherein R₁₂ is 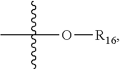
R₁₃ is cyano, isocyano, nitro, or 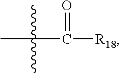
and R₁₆ and R₁₈ are independently hydrogen, hydroxyl, —OR′₁₈, unsubstituted alkyl, unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), or unsubstituted aralkyl (such as benzyl), wherein R′₁₈ is unsubstituted alkyl;
R₁ is a substituted or unsubstituted alkyl, a substituted or unsubstituted alkenyl, or a substituted or unsubstituted alkynyl, wherein the substituents, when present, are independently substituted or unsubstituted alkyl, substituted or unsubstituted alkenyl, substituted or unsubstituted alkynyl, substituted or unsubstituted aryl, substituted or unsubstituted polyaryl, substituted or unsubstituted heteroaryl, substituted or unsubstituted heteropolyaryl, substituted or unsubstituted heterocyclic, substituted or unsubstituted aralkyl, carbonyl, alkoxy, halide, hydroxyl, thiol, cyano, isocyano, nitro, amino, amido, azido, oxo, silyl, sulfinyl, sulfonyl, phosphonium, phosphanyl, phosphoryl, or phosphonyl.

27. The chiral amino acid of claim 26, wherein R₂₆ is methyl or ethyl; R₁₃ is nitro; and R₁₂ is 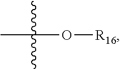
and R₁₆ is an unsubstituted haloalkyl (such as fluoride substituted C₁-C₆ haloalkyl, e.g., —CF₃, —CH₂—CF₃, —CH₂—CH₂—CF₃, —CH₂—CF₂—CF₃, —CH₂—CF₂—CF₂—CF₃), optionally wherein R₁₆ is —CH₂—CH₂—CF₃, —CH₂—CF₂—CF₃, or —CH₂—CF₂—CF₂—CF₃.

28. The chiral amino acid of claim 26, wherein the substituents, when present, are independently substituted or unsubstituted cycloalkyl, substituted or unsubstituted cycloalkenyl, substituted or unsubstituted cycloalkynyl, substituted or unsubstituted aryl, substituted or unsubstituted polyaryl, substituted or unsubstituted heteroaryl, substituted or unsubstituted heteropolyaryl, substituted or unsubstituted heterocyclyl, azido, carbonyl, alkoxy, halide, hydroxyl, thiol, cyano, isocyano, or nitro.

29. The chiral amino acid of claim 26, wherein the substituents, when present, are independently a biomolecule moiety (such as a cholic acid moiety, estrone moiety, etc.).

30. The chiral amino acid of claim 26, wherein R₂₆ is methyl; R₁₃ is nitro; and R₁₂ is 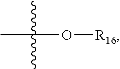
and R₁₆ is —CH₂—CF₂—CF₃.
